(19) United States
(12) Patent Application Publication
(10) Pub. No.: US 2025/0032642 A1
(43) Pub. Date: Jan. 30, 2025

(54) CRISPR-MEDIATED TRANSGENE INSERTION IN NEONATAL CELLS

(71) Applicants: Regeneron Pharmaceuticals, Inc., Tarrytown, NY (US); Intellia Therapeutics, Inc., Cambridge, MA (US)

(72) Inventors: Andrew Baik, Scarsdale, NY (US); Katherine Cygnar, New York, NY (US); Leah Sabin, Goldens Bridge, NY (US); Poulami Samai, New York, NY (US); Philip Calafati, Cambridge, MA (US); Anthony Forget, Cambridge, MA (US)

(21) Appl. No.: 18/835,246

(22) PCT Filed: Feb. 2, 2023
(86) PCT No.: PCT/US23/61854

§ 371 Date: Aug. 1, 2024

Related U.S. Application Data
(60) Provisional application No. 63/369,902, filed on Jul. 29, 2022.
(60) Provisional application No. 63/306,040, filed on Feb. 2, 2022.

(51) Int. Cl. A61K 48/00; A61K 9/127; A61K 9/51; A61K 38/47; A61P 3/00; C12N 9/22; C12N 15/11; C12N 15/86; C12N 15/88; C12N 15/90

(52) U.S. Cl. CPC A61K 48/005; A61K 9/1272; A61K 9/5123; A61K 38/47; A61K 48/0083; A61P 3/00; C12N 9/22; C12N 15/111; C12N 15/86; C12N 15/88; C12N 15/907; C12Y 302/0102; C12N 2310/20; C12N 2750/14143

(57) ABSTRACT

Compositions and methods for inserting a nucleic acid encoding a polypeptide of interest into a target genomic locus in a neonatal cell, a population of neonatal cells, or a neonatal subject or for expressing a nucleic acid encoding a polypeptide of interest in a neonatal cell, a population of neonatal cells, or a neonatal subject are provided. Also provided are neonatal cells or populations of neonatal cells comprising a nucleic acid construct comprising a coding sequence for a polypeptide of interest inserted into a target genomic locus.

### CROSS-REFERENCE TO RELATED APPLICATIONS

This application claims the benefit of U.S. Application No. 63/306,040, filed Feb. 2, 2022, and U.S. Application No. 63/369,902, filed Jul. 29, 2022, each of which is herein incorporated by reference in its entirety for all purposes.

### REFERENCE TO A SEQUENCE LISTING SUBMITTED AS AN XML FILE VIA EMFS WEB

The Sequence Listing written in file 590131SEQLIST.xml is 881 kilobytes, was created on Jan. 26, 2023, and is hereby incorporated by reference.

### BACKGROUND

Gene therapy has long been recognized for its enormous potential in how human diseases are approached and treated. Instead of relying on drugs or surgery, patients with underlying genetic factors can be treated by directly targeting the underlying cause. Furthermore, by targeting the underlying genetic cause, gene therapy can provide the potential to effectively cure patients. However, clinical applications of gene therapy approaches still require improvement in several aspects. In addition, treatment early in life can present additional hurdles due to the unique environment in neonatal patients.

### SUMMARY

Provided are compositions and methods for inserting a nucleic acid encoding a polypeptide of interest into a target genomic locus in a neonatal cell, a population of neonatal cells, or a neonatal subject or for expressing a nucleic acid encoding a polypeptide of interest from a target genomic locus in a neonatal cell, a population of neonatal cells, or a neonatal subject. Also provided are methods of treating an enzyme deficiency, methods of treating a lysosomal storage disease, and methods of preventing or reducing the onset of a sign or symptom of an enzyme deficiency or a lysosomal storage disease in a subject. Also provided are neonatal cells or populations of neonatal cells comprising a nucleic acid construct comprising a coding sequence for a polypeptide of interest inserted into a target genomic locus.

In one aspect, provided are methods of inserting a nucleic acid encoding a polypeptide of interest into a target genomic locus in a neonatal cell or a population of neonatal cells. Some such methods comprise administering to the neonatal cell or the population of neonatal cells: (a) a nucleic acid construct comprising a coding sequence for the polypeptide of interest; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a polypeptide of interest from a target genomic locus in a neonatal cell or a population of neonatal cells. Some such methods comprise administering to the neonatal cell or the population of neonatal cells: (a) a nucleic acid construct comprising a coding sequence for the polypeptide of interest; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the polypeptide of interest is expressed from the modified target genomic locus. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells. In some such methods, the neonatal cell or the population of neonatal cells is from a neonatal subject within 52 weeks after birth. In some such methods, the neonatal cell or the population of neonatal cells is from a human neonatal subject within 24 weeks after birth. In some such methods, the neonatal cell or the population of neonatal cells is from a human neonatal subject within 12 weeks after birth. In some such methods, the neonatal cell or the population of neonatal cells is from a human neonatal subject within 8 weeks after birth. In some such methods, the neonatal cell or the population of neonatal cells is from a human neonatal subject within 4 weeks after birth. In some such methods, the neonatal cell is in vitro or ex vivo or the population of neonatal cells is in vitro or ex vivo. In some such methods, the neonatal cell is in vivo in a neonatal subject or the population of neonatal cells is in vivo in a neonatal subject.

In another aspect, provided are methods of inserting a nucleic acid encoding a polypeptide of interest into a target genomic locus in a neonatal cell or a population of neonatal cells in a neonatal subject. Some such methods comprise administering to the neonatal subject: (a) a nucleic acid construct comprising a coding sequence for the polypeptide of interest; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a polypeptide of interest from a target genomic locus in a neonatal cell or a population of neonatal cells in a neonatal subject. Some such methods comprise administering to the neonatal subject: (a) a nucleic acid construct comprising a coding sequence for the polypeptide of interest; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the polypeptide of interest is expressed from the modified target genomic locus. In another aspect, provided are methods of expressing a polypeptide of interest from a target genomic locus in a neonatal cell in a neonatal subject. Some such methods comprise administering to the neonatal subject: (a) a nucleic acid construct comprising a coding sequence for the polypeptide of interest; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the polypeptide of interest is expressed from the modified target genomic locus, wherein the subject comprises a mutation in a genome in the subject, wherein the mutation results in reduced activity or expression of an endogenous polypeptide having enzymatic activity. In some such methods, the nucleic acid encoding the polypeptide of interest encodes a polypeptide having the enzymatic activity of a wild type polypeptide encoded by the gene in which the subject has a mutation that results in reduced activity or expression of the endogenous polypeptide. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells. In some such methods, the neonatal subject has a lysosomal storage disease characterized by the enzyme deficiency.

In another aspect, provided are methods of treating an enzyme deficiency in a neonatal subject in need thereof. Some such methods comprise administering to the neonatal subject: (a) a nucleic acid construct comprising a coding sequence for a polypeptide of interest, wherein the polypeptide of interest comprises an enzyme to treat the enzyme deficiency; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency. In another aspect, provided are methods of preventing or reducing the onset of a sign or symptom of an enzyme deficiency in a neonatal subject in need thereof. Some such methods comprise administering to the neonatal subject: (a) a nucleic acid construct comprising a coding sequence for a polypeptide of interest, wherein the lysosomal storage disease is characterized by a loss-of-function of the polypeptide of interest; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency. In some such methods, the neonatal subject has a disease of a bleeding disorder characterized by the enzyme deficiency. In some such methods, the bleeding disorder is selected from hemophilia A, hemophilia B, and von Willebrand disease. In some such methods, the neonatal subject has a disease of an inborn error of metabolism characterized by the enzyme deficiency. In some such methods, the neonatal subject has a disease selected from Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, argininosuccinic aciduria, methylmalonic aciduria, type I citrullinemia/argininosuccinate synthetase deficiency, carbamoyl-phosphate synthase 1 deficiency, propionic acidemia, isovaleric acidemia, glutaric academia I, and progressive familial intrahepatic cholestasis, types 2 and 3, Fabry disease, Gaucher disease type I, Gaucher disease type II, Gaucher disease type III, Niemann-Pick disease type A, Niemann-Pick disease type BGM1-gangliosidosis, Sandhoff disease, Tay-Sachs disease, GM2-activator deficiency, GM3-gangliosidosis, metachromatic leukodystrophy, sphingolipid-activator deficiency, Scheie disease, Hurler-Scheie disease, Hurler disease, Hunter disease, Sanfilippo A, Sanfilippo B, Sanfilippo C, Sanfilippo D, Morquio syndrome A, Morquio syndrome B, Maroteaux-Lamy disease, Sly disease, MPS IX, or Pompe disease, In some such methods, the neonatal subject has a lysosomal storage disease characterized by the enzyme deficiency. In another aspect, provided are methods of treating a lysosomal storage disease in a neonatal subject in need thereof. Some such methods comprise administering to the neonatal subject: (a) a nucleic acid construct comprising a coding sequence for a polypeptide of interest, wherein the lysosomal storage disease is characterized by a loss-of-function of the polypeptide of interest; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the polypeptide of interest is expressed from the modified target genomic locus, thereby treating the lysosomal storage disease. In another aspect, provided are methods of preventing or reducing the onset of a sign or symptom of a lysosomal storage disease in a neonatal subject in need thereof. Some such methods comprise administering to the neonatal subject: (a) a nucleic acid construct comprising a coding sequence for a polypeptide of interest, wherein the lysosomal storage disease is characterized by a loss-of-function of the polypeptide of interest; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the lysosomal storage disease.

In some such methods, the neonatal subject is a human subject. In some such methods, the neonatal subject is within 52 weeks after birth. In some such methods, the neonatal subject is a human subject within 24 weeks after birth. In some such methods, the neonatal subject is a human subject within 12 weeks after birth. In some such methods, the neonatal subject is a human subject within 8 weeks after birth. In some such methods, the neonatal subject is a human subject within 4 weeks after birth.

In some such methods, the method results in increased expression of the polypeptide of interest in the subject compared to a method comprising administering an episomal expression vector encoding the polypeptide of interest to a control subject. In some such methods, the method results in increased serum levels of the polypeptide of interest in the subject compared to a method comprising administering an episomal expression vector encoding the polypeptide of interest to a control subject. In some such methods, the expression or activity of the polypeptide of interest is at least 50% of the expression or activity of the polypeptide at a peak level of expression measured for the human subject at six months after the administering. In some such methods, the expression or activity of the polypeptide of interest is at least 50% of the expression or activity of the polypeptide at a peak level of expression measured for the human subject at one year after the administering. In some such methods, the expression or activity of the polypeptide of interest is at least 60% of the expression or activity of the polypeptide at a peak level of expression measured for the human subject at six months after the administering. In some such methods, expression or activity of the polypeptide of interest is at least 50% of the expression or activity of the polypeptide at a peak level of expression measured for the human subject at two years after the administering. In some such methods, the expression or activity of the polypeptide of interest is at least 60% of the expression or activity of the polypeptide at a peak level of expression measured for the human subject at two years after the administering. In some such methods, the expression or activity of the polypeptide of interest is at least 60% of the expression or activity of the polypeptide at a peak level of expression measured for the human subject at six months after the administering.

In some such methods, the method further comprises assessing preexisting AAV immunity in the neonatal subject prior to administering the nucleic acid construct to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

In some such methods, the polypeptide of interest comprises a therapeutic polypeptide. In some such methods, the polypeptide of interest is a secreted polypeptide. In some such methods, the polypeptide of interest comprises a hydrolase, α-galactosidase, β-galactosidase, α-glucosidase, β-glucosidase, saposin-C activator, ceramidase, sphingomyelinase, 0-hexosaminidase, GM2 activator, GM3 synthase, arylsulfatase, sphingolipid activator, α-iduronidase, iduronidase-2-sulfatase, heparin N-sulfatase, N-acetyl-α-glucosaminidase, α-glucosamide N-acetyltransferase, N-acetylglucosamine-6-sulfatase, N-acetylgalactosamine-6-sulfate sulfatase, N-acetylgalactosamine-4-sulfatase, β-glucuronidase, or a hyaluronidase. In some such methods, the polypeptide of interest comprises lysosomal alpha-glucosidase. In some such methods, the polypeptide of interest comprises a sequence at least 90%, at least 91%, at least 92%, at least 93%, at least 94%, at least 95%, at least 96%, at least 97%, at least 98%, or at least 99% identical to the amino acid sequence of SEQ ID NO: 173, optionally wherein the polypeptide of interest is encoded by a codon-optimized and CpG-depleted nucleotide sequence. In some such methods, the coding sequence for the polypeptide of interest comprises a nucleotide sequence at least 90%, at least 91%, at least 92%, at least 93%, at least 94%, at least 95%, at least 96%, at least 97%, at least 98%, or at least 99% identical to a nucleotide sequence selected from SEQ ID NOS: 174-182 and 581-588, optionally selected from SEQ ID NOS: 175-179, wherein the nucleotide sequence is codon-optimized and CpG-depleted. In some such methods, the nucleic acid construct is CpG-depleted. In some such methods, the polypeptide of interest comprises a delivery domain. In some such methods, the polypeptide of interest is delivered to and internalized by skeletal muscle and heart tissue in the subject. In some such methods, the subject has an infantile-onset genetic disorder. In some such methods, the subject wherein the subject has Pompe disease. In some such methods, the subject has a bleeding disorder. In some such methods, the polypeptide of interest is Factor VIII, Factor IX, or von Willebrand factor. In some such methods, the polypeptide of interest is an intracellular polypeptide.

In some such methods, the nucleic acid construct comprises a splice acceptor upstream of the coding sequence for the polypeptide of interest. In some such methods, the nucleic acid construct comprises a polyadenylation signal or sequence downstream of the coding sequence for the polypeptide of interest. In some such methods, the nucleic acid construct comprises a splice acceptor upstream of the coding sequence for the polypeptide of interest, and the nucleic acid construct comprises a polyadenylation signal or sequence downstream of the coding sequence for the polypeptide of interest. In some such methods, the nucleic acid construct does not comprise a homology arm. In some such methods, the nucleic acid construct is inserted into the target genomic locus via non-homologous end joining. In some such methods, the nucleic acid construct comprises homology arms. In some such methods, the nucleic acid construct is inserted into the target genomic locus via homology-directed repair. In some such methods, the nucleic acid construct does not comprise a promoter that drives the expression of the polypeptide of interest. In some such methods, the nucleic acid construct is single-stranded DNA or double-stranded DNA. In some such methods, the nucleic acid construct is single-stranded DNA. In some such methods, the nucleic acid construct is a bidirectional nucleic acid construct. In some such methods, the nucleic acid construct comprises: (I) a first segment comprising the coding sequence for the polypeptide of interest; and (II) a second segment comprising a reverse complement of a second coding sequence for the polypeptide of interest. In some such methods, the nucleic acid construct comprises from 5′ to 3′: a first splice acceptor, the coding sequence for the polypeptide of interest, a first polyadenylation signal or sequence, a reverse complement of a second polyadenylation signal or sequence, the reverse complement of the second coding sequence for the polypeptide of interest, and a reverse complement of a second splice acceptor. In some such methods, the coding sequence for the polypeptide of interest and the second coding sequence for the polypeptide of interest are different.

In some such methods, the nucleic acid construct is in a nucleic acid vector or a lipid nanoparticle. In some such methods, the nucleic acid construct is in the nucleic acid vector. In some such methods, the nucleic acid vector is a viral vector. In some such methods, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such methods, the AAV vector is a single-stranded AAV (ssAAV) vector. In some such methods, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such methods, the AAV vector is a recombinant AAV8 (rAAV8) vector. In some such methods, the AAV vector is a single-stranded rAAV8 vector. In some such methods, the nucleic acid construct is CpG-depleted.

In some such methods, the target genomic locus is an albumin gene, optionally wherein the albumin gene is a human albumin gene. In some such methods, the nuclease target site is in intron 1 of the albumin gene. In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the guide RNA target sequence is in intron 1 of an albumin gene. In some such methods, the albumin gene is a human albumin gene.

In some such methods, the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41. In some such methods, the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such methods, the DNA-targeting segment comprises any one of SEQ ID NOS: 30-61, optionally wherein the DNA-targeting segment comprises any one of SEQ ID NOS: 36, 30, 33, and 41. In some such methods, the DNA-targeting segment consists of any one of SEQ ID NOS: 30-61, optionally wherein the DNA-targeting segment consists of any one of SEQ ID NOS: 36, 30, 33, and 41. In some such methods, the guide RNA comprises any one of SEQ ID NOS: 62-125, optionally wherein the guide RNA comprises any one of SEQ ID NOS: 68, 100, 62, 94, 65, 97, 73, and 105. In some such methods, the DNA-targeting segment comprises at least 17, at least 18, at least 19, or at least 20 contiguous nucleotides of SEQ ID NO: 36. In some such methods, the DNA-targeting segment is at least 90% or at least 95% identical to SEQ ID NO: 36. In some such methods, the DNA-targeting segment comprises SEQ ID NO: 36. In some such methods, the DNA-targeting segment consists of SEQ ID NO: 36. In some such methods, the guide RNA comprises SEQ ID NO: 68 or 100.

In some such methods, the method comprises administering the guide RNA in the form of RNA. In some such methods, the guide RNA comprises at least one modification. In some such methods, the at least one modification comprises a 2′-O-methyl-modified nucleotide. In some such methods, the at least one modification comprises a phosphorothioate bond between nucleotides. In some such methods, the at least one modification comprises a modification at one or more of the first five nucleotides at the 5′ end of the guide RNA. In some such methods, the at least one modification comprises a modification at one or more of the last five nucleotides at the 3′ end of the guide RNA. In some such methods, the at least one modification comprises phosphorothioate bonds between the first four nucleotides at the 5′ end of the guide RNA. In some such methods, the at least one modification comprises phosphorothioate bonds between the last four nucleotides at the 3′ end of the guide RNA. In some such methods, the at least one modification comprises 2′-O-methyl-modified nucleotides at the first three nucleotides at the 5′ end of the guide RNA. In some such methods, the at least one modification comprises 2′-O-methyl-modified nucleotides at the last three nucleotides at the 3′ end of the guide RNA. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such methods, the guide RNA is a single guide RNA (sgRNA). In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such methods, the Cas protein is a Cas9 protein. In some such methods, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the Cas protein is derived from a Streptococcus pyogenes Cas9 protein. In some such methods, the Cas protein comprises the sequence set forth in SEQ ID NO: 11. In some such methods, the nucleic acid encoding the Cas protein is codon-optimized for expression in a mammalian cell or a human cell. In some such methods, the method comprises administering the nucleic acid encoding the Cas protein, wherein the nucleic acid comprises an mRNA encoding the Cas protein. In some such methods, the mRNA encoding the Cas protein comprises at least one modification. In some such methods, the mRNA encoding the Cas protein is modified to comprise a modified uridine at one or more or all uridine positions. In some such methods, the modified uridine is pseudouridine or N1-methyl-pseudouridine, optionally N1-methyl-pseudouridine. In some such methods, the mRNA encoding the Cas protein is fully substituted with pseudouridine or N1-methyl-pseudouridine, optionally N1-methyl-pseudouridine. In some such methods, the modified uridine is pseudouridine. In some such methods, the mRNA encoding the Cas protein is fully substituted with pseudouridine. In some such methods, the mRNA encoding the Cas protein comprises a 5′ cap. In some such methods, the mRNA encoding the Cas protein comprises a polyadenylation sequence. In some such methods, the mRNA encoding the Cas protein comprises the sequence set forth in SEQ ID NO: 2, 1, or 12. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine, comprises a 5′ cap, and comprises a polyadenylation sequence.

In some such methods, the method comprises administering the guide RNA in the form of RNA, and the guide RNA comprises SEQ ID NO: 68 or 100, and wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 2, 1, or 12. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine, comprises a 5′ cap, and comprises a polyadenylation sequence.

In some such methods, the Cas protein or the nucleic acid encoding the Cas protein and the guide RNA or the one or more DNAs encoding the guide RNA are associated with a lipid nanoparticle. In some such methods, the lipid nanoparticle comprises a cationic lipid, a neutral lipid, a helper lipid, and a stealth lipid. In some such methods, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate). In some such methods, the neutral lipid is distearoylphosphatidylcholine or 1,2-distearoyl-sn-glycero-3-phosphocholine (DSPC). In some such methods, the helper lipid is cholesterol. In some such methods, the stealth lipid is 1,2-dimyristoyl-rac-glycero-3-methoxypolyethylene glycol-2000 (PEG2k-DMG). In some such methods, the cationic lipid is Lipid A, the neutral lipid is DSPC, the helper lipid is cholesterol, and the stealth lipid is PEG2k-DMG. In some such methods, the lipid nanoparticle comprises four lipids at the following molar ratios: about 50 mol % Lipid A, about 9 mol % DSPC, about 38 mol % cholesterol, and about 3 mol % PEG2k-DMG.

In some such methods, the albumin gene is a human albumin gene, wherein the method comprises administering the guide RNA in the form of RNA, and the guide RNA comprises SEQ ID NO: 68 or 100, wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 2, 1, or 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine, comprises a 5′ cap, and comprises a polyadenylation sequence, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

In another aspect, provided are a neonatal cell or a population of neonatal cells made by any of the above methods. In another aspect, provided are a neonatal cell or a population of neonatal cells comprising a nucleic acid construct comprising a coding sequence for a polypeptide of interest inserted into a target genomic locus. In another aspect, provided are a cell or a population of cells made by any of the above methods. In another aspect, provided are a cell or a population of cells comprising a nucleic acid construct comprising a coding sequence for a polypeptide of interest inserted into a target genomic locus.

In some such neonatal cells or populations of neonatal cells, the neonatal cell is a liver cell or the population of neonatal cells is a population of liver cells. In some such cells or populations of cells, the cell is a liver cell or the population of cells is a population of liver cells. In some such neonatal cells or populations of neonatal cells, the neonatal cell is a hepatocyte or the population of neonatal cells is a population of hepatocytes. In some such cells or populations of cells, the cell is a hepatocyte or the population of cells is a population of hepatocytes. In some such neonatal cells or populations of neonatal cells, the neonatal cell is a human cell or the population of neonatal cells is a population of human cells. In some such cells or populations of cells, the cell is a human cell or the population of cells is a population of human cells. In some such neonatal cells or populations of neonatal cells, the neonatal cell or the population of neonatal cells is from a neonatal subject within 52 weeks after birth. In some such neonatal cells or populations of neonatal cells, the neonatal cell or the population of neonatal cells is from a human neonatal subject within 24 weeks after birth. In some such neonatal cells or populations of neonatal cells, the neonatal cell or the population of neonatal cells is from a human neonatal subject within 12 weeks after birth. In some such neonatal cells or populations of neonatal cells, the neonatal cell or the population of neonatal cells is from a human neonatal subject within 8 weeks after birth. In some such neonatal cells or populations of neonatal cells, the neonatal cell or the population of neonatal cells is from a human neonatal subject within 4 weeks after birth. In some such neonatal cells or populations of neonatal cells, the neonatal cell is in vitro or ex vivo or the population of neonatal cells is in vitro or ex vivo. In some such neonatal cells or populations of neonatal cells, the neonatal cell is in vivo in a subject or the population of neonatal cells is in vivo. In some such cells or populations of cells, the cell is in vitro or ex vivo or the population of cells is in vitro or ex vivo. In some such cells or populations of cells, the cell is in vivo in a subject or the population of cells is in vivo.

In some such neonatal cells or populations of neonatal cells, the polypeptide of interest is expressed. In some such neonatal cells or populations of neonatal cells, the polypeptide of interest comprises a therapeutic polypeptide, optionally wherein the polypeptide of interest comprises lysosomal alpha-glucosidase. In some such neonatal cells or populations of neonatal cells, the lysosomal alpha-glucosidase comprises the amino acid sequence of SEQ ID NO: 173, optionally wherein the polypeptide of interest is encoded by a nucleic acid is codon-optimized and CpG-depleted nucleotide sequence. In some such neonatal cells or populations of neonatal cells, the lysosomal alpha-glucosidase is encoded by a nucleotide sequence at least 90%, at least 91%, at least 92%, at least 93%, at least 94%, at least 95%, at least 96%, at least 97%, at least 98%, or at least 99% identical to a nucleotide sequence selected from SEQ ID NOS: 174-182 and 581-588, optionally SEQ ID NOS: 175-179, wherein the nucleotide sequence is codon-optimized and CpG-depleted. In some such neonatal cells or populations of neonatal cells, the polypeptide of interest is a secreted polypeptide. In some such neonatal cells or populations of neonatal cells, the polypeptide of interest is an intracellular polypeptide. In some such cells or populations of cells, the polypeptide of interest is expressed. In some such cells or populations of cells, the polypeptide of interest comprises a therapeutic polypeptide, optionally wherein the polypeptide of interest comprises lysosomal alpha-glucosidase. In some such cells or populations of cells, the lysosomal alpha-glucosidase comprises the amino acid sequence of SEQ ID NO: 173, optionally wherein the polypeptide of interest is encoded by a nucleic acid is codon-optimized and CpG-depleted nucleotide sequence. In some such cells or populations of cells, the lysosomal alpha-glucosidase is encoded by a nucleotide sequence at least 90%, at least 91%, at least 92%, at least 93%, at least 94%, at least 95%, at least 96%, at least 97%, at least 98%, or at least 99% identical to a nucleotide sequence selected from SEQ ID NOS: 174-182 and 581-588, optionally SEQ ID NOS: 175-179, wherein the nucleotide sequence is codon-optimized and CpG-depleted. In some such cells or populations of cells, the polypeptide of interest is a secreted polypeptide. In some such cells or populations of cells, the polypeptide of interest is an intracellular polypeptide.

In some such neonatal cells or populations of neonatal cells, the nucleic acid construct comprises a splice acceptor upstream of the coding sequence for the polypeptide of interest. In some such neonatal cells or populations of neonatal cells, the nucleic acid construct comprises a polyadenylation signal or sequence downstream of the coding sequence for the polypeptide of interest. In some such neonatal cells or populations of neonatal cells, the nucleic acid construct comprises a splice acceptor upstream of the coding sequence for the polypeptide of interest, and the nucleic acid construct comprises a polyadenylation signal or sequence downstream of the coding sequence for the polypeptide of interest. In some such neonatal cells or populations of neonatal cells, the nucleic acid construct does not comprise a promoter that drives the expression of the polypeptide of interest, and wherein the coding sequence for the polypeptide of interest is operably linked to an endogenous promoter at the target genomic locus. In some such neonatal cells or populations of neonatal cells, the nucleic acid construct is a bidirectional nucleic acid construct. In some such neonatal cells or populations of neonatal cells, the nucleic acid construct comprises: (I) a first segment comprising the coding sequence for the polypeptide of interest; and (II) a second segment comprising a reverse complement of a second coding sequence for the polypeptide of interest. In some such neonatal cells or populations of neonatal cells, the nucleic acid construct comprises from 5′ to 3′: a first splice acceptor, the coding sequence for the polypeptide of interest, a first polyadenylation signal or sequence, a reverse complement of a second polyadenylation signal or sequence, the reverse complement of the second coding sequence for the polypeptide of interest, and a reverse complement of a second splice acceptor. In some such neonatal cells or populations of neonatal cells, the coding sequence for the polypeptide of interest and the second coding sequence for the polypeptide of interest are different. In some such cells or populations of cells, the nucleic acid construct comprises a splice acceptor upstream of the coding sequence for the polypeptide of interest. In some such cells or populations of cells, the nucleic acid construct comprises a polyadenylation signal or sequence downstream of the coding sequence for the polypeptide of interest. In some such cells or populations of cells, the nucleic acid construct comprises a splice acceptor upstream of the coding sequence for the polypeptide of interest, and the nucleic acid construct comprises a polyadenylation signal or sequence downstream of the coding sequence for the polypeptide of interest. In some such cells or populations of cells, the nucleic acid construct does not comprise a promoter that drives the expression of the polypeptide of interest, and wherein the coding sequence for the polypeptide of interest is operably linked to an endogenous promoter at the target genomic locus. In some such cells or populations of cells, the nucleic acid construct is a bidirectional nucleic acid construct. In some such cells or populations of cells, the nucleic acid construct comprises: (I) a first segment comprising the coding sequence for the polypeptide of interest; and (II) a second segment comprising a reverse complement of a second coding sequence for the polypeptide of interest. In some such cells or populations of cells, the nucleic acid construct comprises from 5′ to 3′: a first splice acceptor, the coding sequence for the polypeptide of interest, a first polyadenylation signal or sequence, a reverse complement of a second polyadenylation signal or sequence, the reverse complement of the second coding sequence for the polypeptide of interest, and a reverse complement of a second splice acceptor. In some such cells or populations of cells, the coding sequence for the polypeptide of interest and the second coding sequence for the polypeptide of interest are different

In some such neonatal cells or populations of neonatal cells, the target genomic locus is an albumin gene, optionally wherein the albumin gene is a human albumin gene. In some such neonatal cells or populations of neonatal cells, the nuclease target site is in intron 1 of the albumin gene. In some such cells or populations of cells, the target genomic locus is an albumin gene, optionally wherein the albumin gene is a human albumin gene. In some such cells or populations of cells, the nuclease target site is in intron 1 of the albumin gene.

### BRIEF DESCRIPTION OF THE FIGURES

FIG. 1 shows a schematic describing different human factor IX (hFIX) insertion templates tested in adult and neonatal mice.

FIG. 2A shows hFIX plasma levels in neonatal mice (n=4-10 per group; male and female) and adult mice (n=5 per group; female) at different time points post-administration of episomal hFIX (Episome), LNP-g666+hFIX-HDR-500 template (HDR500), LNP-g666+hFIX-HDR-800 template (HDR800), and LNP-g666+hFIX-NHEJ template (NHEJ). The administration in neonatal mice occurred at P0 or P1. Saline-injected mice were used as negative controls. Data are shown on a log scale. FIG. 2B shows hFIX plasma levels in neonatal mice (n=4-10 per group; male and female) at different time points post-administration of episomal hFIX (Episome), LNP-g666+hFIX-HDR-500 template (HDR500), LNP-g666+hFIX-HDR-800 template (HDR800), and LNP-g666+hFIX-NHEJ template (NHEJ). The administration in neonatal mice occurred at P0 or P1. Saline-injected mice were used as negative controls. Data are shown on a linear scale.

FIG. 3 shows a schematic of LNP-g9860, which is a lipid nanoparticle containing Cas9 mRNA and sgRNA 9860 targeting human albumin (ALB) intron 1, and a recombinant AAV8 (rAAV8) capsid packaged with an anti-CD63:GAA insertion template.

FIG. 4 shows a schematic of targeting of GAA to the lysosome via fusion to anti-CD63 scFv.

FIG. 5 shows a schematic for CRISPR/Cas9-mediated insertion of an anti-CD63:GAA insertion template at the ALB locus. The human ALB locus is depicted, with the Cas9 cut site denoted with scissors. The splice acceptor site flanking the anti-CD63:GAA transgene in the insertion template is depicted. Following insertion and transcription driven by the endogenous ALB promoter, splicing between ALB exon 1 and the inserted anti-CD63:GAA DNA template occurs, diagrammed in dashed lines, to produce a hybrid ALB-anti-CD63:GAA mRNA. The ALB signal peptide promotes secretion of anti-CD63:GAA and is removed during protein maturation to yield anti-CD63:GAA in plasma.

FIG. 6 shows levels of anti-CD63:GAA in the serum over a 10-month time course following administration of LNP-g666 (1 mg/kg) and a recombinant AAV8 anti-CD63:GAA insertion template (1.2e13 vg/kg) (“Insertion”) or following administration of episomal AAV encoding of anti-CD63:GAA (4e12 vg/kg) (“Episomal”) to adult Pompe disease model male and female mice (n=12; GAA^{-/-}; CD63^{hu/hu}).

FIG. 7 shows glycogen levels in the heart, quadricep, diaphragm, and spinal cord in Pompe disease model mice (GAA^{-/-}; CD63^{hu/hu}) at 10 months after administration of LNP-g666 and a recombinant AAV8 anti-CD63:GAA insertion template or at 10 months after administration of episomal AAV encoding anti-CD63:GAA to adult Pompe disease model male and female mice (n=12; GAA^{-/-}; CD63^{hu/hu}). Wild type GAA mice (GAA^{+/+}; CD63^{hu/hu}; n=4) and untreated Pompe disease model mice (n=4) were used as controls. The horizontal dotted line is the lower limit of detection of the assay.

FIGS. 8A-8B show levels of anti-CD63:GAA in the serum over a 15-month time course following administration of LNP-g666 and a recombinant AAV8 anti-CD63:GAA insertion template (n=10; male and female; “Insertion”) or following administration of episomal AAV encoding of anti-CD63:GAA (n=6; male and female; “Episomal”) to neonatal (P1) Pompe disease model mice (GAA^{-/-}; CD63^{hu/hu}). The horizontal dotted line is the lower limit of detection of the assay. The error bars in FIG. 8A are ±SD, and the error bars in FIG. 8B are ±SEM.

FIG. 9A shows glycogen levels in the heart, quadricep, gastrocnemius, and diaphragm in Pompe disease model mice (GAA^{-/-}; CD63^{hu/hu}) at 3 months after administration of LNP-g666 and a recombinant AAV8 anti-CD63:GAA insertion template (n=5; male and female, “I”) or at 3 months after administration of episomal AAV encoding anti-CD63:GAA (n=3; male and female, “E”) to neonatal (P1) mice. Untreated Pompe disease model mice were used as controls.

FIG. 9B shows glycogen levels in the heart, quadricep, gastrocnemius, diaphragm, cerebrum, and spinal cord in Pompe disease model mice (GAA^{-/-}; CD63^{hu/hu}) at 15 months after administration of LNP-g666 and a recombinant AAV8 anti-CD63:GAA insertion template (n=10; male and female, “I”) or at 15 months after administration of episomal AAV encoding anti-CD63:GAA (n=6; male and female, “E”) to neonatal (P1) mice. Untreated Pompe disease model mice (“U”) and wild type mice (“W”) were used as controls.

FIG. 10 shows grip strength in Pompe disease model mice (GAA^{-/-}; CD63^{hu/hu}) at 15 months after administration of LNP-g666 and a recombinant AAV8 anti-CD63:GAA insertion template (n=10; male and female, “P1 insertion AAV+LNP”) or at 15 months after administration of episomal AAV encoding anti-CD63:GAA (n=6; male and female, “P1 episomal AAV”) to neonatal (P1) mice. Wild type GAA mice (GAA^{+/+}; CD63^{hu/hu}; “Wild type”) and untreated Pompe disease model mice (“Untreated KO”) were used as controls.

FIG. 11 shows a schematic of LNP-g9860, which is a lipid nanoparticle containing Cas9 mRNA and sgRNA 9860 targeting human albumin (ALB) intron 1, and a recombinant AAV8 (rAAV8) capsid packaged with an anti-TfR:GAA insertion template.

FIG. 12 shows a schematic of targeting of GAA through multiple paths via fusion to anti-TfR scFv.

FIG. 13 shows a schematic for CRISPR/Cas9-mediated insertion of an anti-TfR:GAA insertion template at the ALB locus. The human ALB locus is depicted, with the Cas9 cut site denoted with scissors. The splice acceptor site flanking the anti-TfR:GAA transgene in the insertion template is depicted. Following insertion and transcription driven by the endogenous ALB promoter, splicing between ALB exon 1 and the inserted anti-TfR:GAA DNA template occurs, diagrammed in dashed lines, to produce a hybrid ALB-anti-TfR:GAA mRNA. The ALB signal peptide promotes secretion of anti-TfR:GAA and is removed during protein maturation to yield anti-TfR:GAA in plasma.

FIGS. 14A-14C show western blots showing that anti-human TfR antibody clones deliver GAA to the cerebrum of Tfrc^{hum} mice. Each lane=1 mouse. Anti-mouse mTfR:GAA in Wt mice was used as a positive control. Anti-mouse mTfR:GAA in Tfrc^{hum} mice was used as a negative control.

FIG. 15 shows western blots showing that a subset of anti-hTfR antibody clones deliver mature GAA to the brain parenchyma in scfv:GAA format (delivery by HDD). Anti-mouse mTfR:GAA in Wt mice was used as a positive control. Anti-mouse mTfR:GAA in Tfrc^{hum} mice was used as a negative control.

FIG. 16 shows western blots showing that four selected anti-hTfR antibody clones deliver mature GAA to the brain parenchyma in scfv:GAA format (AAV8 episomal liver depot gene therapy). Anti-mouse mTfR:GAA in Wt mice was used as a positive control. Anti-mouse mTfR:GAA in Tfrc^{hum} mice was used as a negative control.

FIG. 17 shows western blots showing that three selected episomal AAV8 liver depot anti-hTfR antibody clones deliver mature GAA to the CNS, heart, and muscle in Gaa^{-/-}/Tfrc^{hum} mice.

FIGS. 18A and 18B show that four selected episomal AAV8 liver depot anti-hTfR antibody clones rescue glycogen storage in CNS, heart, and muscle in Gaa^{-/-}/Tfrc^{hum} mice. Wt untreated mice were a positive control, and Gaa^{-/-} untreated mice were a negative control.

FIG. 18C shows that a selected episomal AAV8 liver depot anti-hTfR antibody clone rescues glycogen storage in dorsal root ganglia (DRGs) in Gaa^{-/-}/Tfrc^{hum} mice. Wt untreated mice were a positive control, and Gaa^{-/-} untreated mice were a negative control.

FIGS. 19A-19D show that three selected episomal AAV8 liver depot anti-hTfR antibody clones rescue glycogen storage in brain thalamus (FIG. 19A), brain cerebral cortex (FIG. 19B), brain hippocampus CA1 (FIG. 19C), and quadricep (FIG. 19D) in Gaa^{-/-}/Tfrc^{hum} mice. Wt untreated mice were a positive control, and Gaa^{-/-} untreated mice were a negative control.

FIG. 20A shows that insertion of anti-hTfR 12847scfv:GAA delivers mature GAA protein to CNS and muscle of Pompe model mice. FIG. 20B shows that insertion of anti-hTfR 12847scfv:GAA rescues glycogen storage in CNS and muscle of Pompe model mice. One Way ANOVA *p<0.01; **p<0.001; ***p<0.0001. Untreated Pompe disease model mice and wild type mice were used as controls. Mice injected with a recombinant AAV8 anti-TfR:GAA episomal template were used as a positive control. Mice injected with a recombinant AAV8 anti-TfR:GAA insertion template without LNP-g666 were used as a negative control.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), proteins, and nucleic acids includes cells, tissues (e.g., liver samples), proteins, and nucleic acids that are relatively purified with respect to other bacterial, viral, cellular, or other components that may normally be present in situ, up to and including a substantially pure preparation of the cells, tissues (e.g., liver samples), proteins, and nucleic acids. The term “isolated” also includes cells, tissues (e.g., liver samples), proteins, and nucleic acids that have no naturally occurring counterpart, have been chemically synthesized and are thus substantially uncontaminated by other cells, tissues (e.g., liver samples), proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous ALB sequence of a human refers to a native ALB sequence that naturally occurs at the ALB locus in the human.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” (i.e., “codon optimized” sequences) takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a polypeptide of interest can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “ALB locus” may refer to the specific location of an ALB gene, ALB DNA sequence, albumin-encoding sequence, or ALB position on a chromosome of the genome of an organism that has been identified as to where such a sequence resides. An “ALB locus” may comprise a regulatory element of an ALB gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “antibody,” as used herein, includes immunoglobulin molecules comprising four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, e.g., BIACORE™ or solution-affinity ELISA. The term “antibody” may encompass any type of antibody, such as e.g. monoclonal or polyclonal. Moreover, the antibody may be or any origin, such as e.g. mammalian or non-mammalian. In one embodiment, the antibody may be mammalian or avian. In a further embodiment, the antibody may be or human origin and may further be a human monoclonal antibody.

The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with this invention include, for example, those that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild type animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, for example, as the result of splicing or connecting the sequences (e.g., V-D-J recombination to form a heavy chain CDR3).

The term “antibody fragment” refers to one or more fragments of an antibody that retain the ability to specifically bind to an antigen. Examples of binding fragments encompassed within the term “antibody fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et al. (1993) Proc. Natl. Acad. Sci. U.S.A. 90:6444-6448; Poljak et al. (1994) Structure 2:1121-1123).

The phrase “Fc-containing protein” includes antibodies, bispecific antibodies, immunoadhesins, and other binding proteins that comprise at least a functional portion of an immunoglobulin CH2 and CH3 region. A “functional portion” refers to a CH2 and CH3 region that can bind a Fc receptor (e.g., an FcyR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 2591 (e.g., V259I), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “antigen-binding protein,” as used herein, refers to a polypeptide or protein (one or more polypeptides complexed in a functional unit) that specifically recognizes an epitope on an antigen, such as a cell-specific antigen and/or a target antigen of the present invention. An antigen-binding protein may be multi-specific. The term “multi-specific” with reference to an antigen-binding protein means that the protein recognizes different epitopes, either on the same antigen or on different antigens. A multi-specific antigen-binding protein of the present invention can be a single multifunctional polypeptide, or it can be a multimeric complex of two or more polypeptides that are covalently or non-covalently associated with one another. The term “antigen-binding protein” includes antibodies or fragments thereof of the present invention that may be linked to or co-expressed with another functional molecule, for example, another peptide or protein. For example, an antibody or fragment thereof can be functionally linked (e.g., by chemical coupling, genetic fusion, non-covalent association or otherwise) to one or more other molecular entities, such as a protein or fragment thereof to produce a bispecific or a multi-specific antigen-binding molecule with a second binding specificity.

As used herein, the term “epitope” refers to the portion of the antigen which is recognized by the multi-specific antigen-binding polypeptide. A single antigen (such as an antigenic polypeptide) may have more than one epitope. Epitopes may be defined as structural or functional. Functional epitopes are generally a subset of structural epitopes and are defined as those residues that directly contribute to the affinity of the interaction between the antigen-binding polypeptide and the antigen. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The term “domain” refers to any part of a protein or polypeptide having a particular function or structure. Preferably, domains of the present invention bind to cell-specific or target antigens. Cell-specific antigen- or target antigen-binding domains, and the like, as used herein, include any naturally occurring, enzymatically obtainable, synthetic, or genetically engineered polypeptide or glycoprotein that specifically binds an antigen.

The term “half-body” or “half-antibody”, which are used interchangeably, refers to half of an antibody, which essentially contains one heavy chain and one light chain. Antibody heavy chains can form dimers, thus the heavy chain of one half-body can associate with heavy chain associated with a different molecule (e.g., another half-body) or another Fc-containing polypeptide. Two slightly different Fc-domains may “heterodimerize” as in the formation of bispecific antibodies or other heterodimers, -trimers, -tetramers, and the like. See Vincent and Murini (2012) Biotechnol. J. 7(12):1444-1450; and Shimamoto et al. (2012) MAbs 4(5):586-91. In one embodiment, the half-body variable domain specifically recognizes the internalization effector and the half body Fc-domain dimerizes with an Fc-fusion protein that comprises a replacement enzyme (e.g., a peptibody).

The term “single-chain variable fragment” or “scFv” includes a single chain fusion polypeptide containing an immunoglobulin heavy chain variable region (VH) and an immunoglobulin light chain variable region (VL). In some embodiments, the VH and VL are connect by a linker sequence of 10 to 25 amino acids. ScFv polypeptides may also include other amino acid sequences, such as CL or CH1 regions. ScFv molecules can be manufactured by phage display or made by directly subcloning the heavy and light chains from a hybridoma or B-cell. See Ahmad et al. (2012) Clin. Dev. Immunol. 2012:980250, herein incorporated by reference in its entirety for all purposes.

As used herein, the term “neonatal” in the context of humans covers human subjects up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

As used herein, a “control” as in a control sample or a control subject is a comparator for a measurement, e.g., a diagnostic measurement of a sign or symptom of a disease. In certain embodiments, a control can be a subject sample from the same subject an earlier time point, e.g., before a treatment intervention. In certain embodiments, a control can be a measurement from a normal subject, i.e., a subject not having the disease of the treated subject, to provide a normal control, e.g., an enzyme concentration or activity in a subject sample. In certain embodiments, a normal control can be a population control, i.e., the average of subjects in the general population. In certain embodiments, a control can be an untreated subject with the same disease. In certain embodiments, a control can be a subject treated with a different therapy, e.g., the standard of care. In certain embodiments, a control can be a subject or a population of subjects from a natural history study of subjects with the disease of the subject being compared. In certain embodiments, the control is matched for certain factors to the subject being tested, e.g., age, gender. In certain embodiments, a control may be a control level for a particular lab, e.g., a clinical lab. Selection of an appropriate control is within the ability of those of skill in the art.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least”, “up to”, or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, “detecting an analyte” and the like is understood as performing an assay in which the analyte can be detected, if present, wherein the analyte is present in an amount above the level of detection of the assay.

As used herein, “loss of function” is understood as an activity not being present, e.g., an enzyme activity not being present, for any reason. In certain embodiments, the absence of activity may be due to the absence of a protein having a function, e.g., protein is not transcribed or translated, protein is translated but not stable or not transported appropriately, either intracellularly or systemically. In certain embodiments, the absence of activity may be due to the presence of a mutation, e.g., point mutation, truncation, abnormal splicing, such that a protein is present, but not functional. A loss of function can be a partial or complete loss of function. In certain embodiments, various degrees of loss of function may be known that result in various conditions, severity of disease, or age of onset. As used herein, a loss of function is preferably not a transient loss of function, e.g., due to a stress response or other response that results in a temporary loss of a functional protein. Therapeutic interventions to correct for a loss of function of a protein may include compensation for the loss of function with the protein that is deficient, or with proteins that compensate for the loss of function, but that have a different sequence or structure than the protein for which the function is lost. It is understood that a loss of function of one protein may be compensated for by providing or altering the activity of another protein in the same biological pathway. In certain embodiments, the protein to compensate for the loss of function includes one or more of a truncation, mutation, or non-native sequence to direct trafficking of the protein, either intracellularly or systemically, to overcome the loss of function of the protein. The therapeutic intervention may or may not correct the loss of function of the protein in all cell types or tissues. The therapeutic intervention may include expression of the protein to compensate for a loss of function at a site remote from where the protein lacking function is typically expressed, e.g., where the deficiency results in dysfunction of a cell or organ. The therapeutic intervention may include expression of the protein in the liver to compensate for a loss of function at a site remote from the liver. A number of genetic mutations have been linked with specific loss of function mutations, in both humans and other species.

As used herein, “enzyme deficiency” is understood as an insufficient level of an enzyme activity due to a loss of function of the protein. An enzyme deficiency can be partial or total, and may result in differences in time of onset or severity of signs or symptoms of the enzyme deficiency depending on the level and site of the loss of function. As used herein, enzyme deficiency is preferably not a transient enzyme deficiency due to stress or other factors. A number of genetic mutations have been linked with enzyme deficiencies, in both humans and other species. In certain embodiments, enzyme deficiencies result in inborn errors of metabolism. In certain embodiments, enzyme deficiencies result in lysosomal storage diseases. In certain embodiments, enzyme deficiencies result in galactosemia. In certain embodiments, enzyme deficiencies result in bleeding disorders.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

Unless otherwise apparent from the context, the term “about” encompasses values ±5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Compositions and methods for inserting a nucleic acid encoding a polypeptide of interest into a target genomic locus in a neonatal cell, a population of neonatal cells, or a neonatal subject or for expressing a nucleic acid encoding a polypeptide of interest in a neonatal cell, a population of neonatal cells, or a neonatal subject are provided. Also provided are methods of treating an enzyme deficiency, methods of treating a lysosomal storage disease, and methods of preventing or reducing the onset of a sign or symptom of an enzyme deficiency or a lysosomal storage disease in a subject. Also provided are neonatal cells or populations of neonatal cells comprising a nucleic acid construct comprising a coding sequence for a polypeptide of interest inserted into a target genomic locus.

The neonatal gene insertion platform described herein has advantages in terms of expression levels, durability of expression, and level of functional rescue of enzyme deficiencies over existing episomal platforms in neonates.

### II. Compositions for Inserting Nucleic Acid Constructs Encoding and for Expressing Polypeptides of Interest in Cells or Neonatal Cells

Provided herein are nucleic acid constructs and compositions that allow insertion of a coding sequence for a polypeptide of interest into a target genomic locus such as an endogenous albumin (ALB) locus and/or expression of the coding sequence for the polypeptide of interest. The nucleic acid constructs and compositions can be used in methods for integration into a target genomic locus and/or expression in a cell or a subject. Also provided are nuclease agents (e.g., targeting an endogenous ALB locus) or nucleic acids encoding nuclease agents to facilitate integration of the nucleic acid constructs into a target genomic locus such as an endogenous ALB locus.

### A. Nucleic Acid Constructs Encoding a Polypeptide of Interest

The compositions and methods described herein include the use of a nucleic acid construct that comprises a coding sequence for a polypeptide of interest (e.g., an exogenous polypeptide coding sequence). The compositions and methods described herein can also include the use of a nucleic acid construct that comprises a polypeptide of interest coding sequence or a reverse complement of the polypeptide of interest coding sequence (e.g., an exogenous polypeptide coding sequence or a reverse complement of the exogenous polypeptide coding sequence). Such nucleic acid constructs can be for insertion into a target genomic locus or into a cleavage site created by a nuclease agent or CRISPR/Cas system as disclosed elsewhere herein. The term cleavage site includes a DNA sequence at which a nick or double-strand break is created by a nuclease agent (e.g., a Cas9 protein complexed with a guide RNA). In some embodiments, a double-stranded break is created by a Cas9 protein complexed with a guide RNA, e.g., a Spy Cas9 protein complexed with a Spy Cas9 guide RNA. In some cases, the polypeptide of interest is an exogenous polypeptide as defined herein.

The length of the nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Some constructs may be inserted so that their expression is driven by the endogenous promoter at the insertion site (e.g., the endogenous ALB promoter when the construct is integrated into the host cell's ALB locus). Such constructs may not comprise a promoter that drives the expression of the polypeptide of interest. For example, the expression of the polypeptide of interest can be driven by a promoter of the host cell (e.g., the endogenous ALB promoter when the transgene is integrated into a host cell's ALB locus). In such cases, the construct may lack control elements (e.g., promoter and/or enhancer) that drive its expression (e.g., a promoterless construct). Nonetheless, in other cases the construct may comprise a promoter and/or enhancer, for example a constitutive promoter or an inducible or tissue-specific (e.g., liver- or platelet-specific) promoter that drives expression of the polypeptide of interest in an episome or upon integration. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. The construct may comprise a sequence encoding a polypeptide of interest downstream of and operably linked to a signal sequence encoding a signal peptide. In some examples, the nucleic acid construct works in homology-independent insertion of a nucleic acid that encodes a polypeptide of interest. Such nucleic acid constructs can work, for example, in non-dividing cells (e.g., cells in which non-homologous end joining (NHEJ), not homologous recombination (HR), is the primary mechanism by which double-stranded DNA breaks are repaired) or dividing cells (e.g., actively dividing cells). Such constructs can be, for example, homology-independent donor constructs. In preferred embodiments, promoters and other regulatory sequences are appropriate for use in humans, e.g., recognized by regulatory factors in human cells, e.g., in human liver cells, and acceptable to regulatory authorities for use in humans.

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a target genomic locus or a cut site in a target DNA sequence for a nuclease agent (e.g., capable of insertion into a safe harbor gene, such as an ALB locus) by non-homologous end joining. For example, such constructs can be inserted into a blunt end double-strand break following cleavage with a nuclease agent (e.g., CRISPR/Cas system, e.g., a SpyCas9 CRISPR/Cas system) as disclosed herein. In a specific example, the construct can be delivered via AAV and can be capable of insertion by non-homologous end joining (e.g., the construct does not comprise a homology arm).

In a particular example, the construct can be inserted via homology-independent targeted integration. For example, the polypeptide of interest coding sequence in the construct can be flanked on each side by a target site for a nuclease agent (e.g., the same target site as in the target DNA sequence for targeted insertion (e.g., in a safe harbor gene), and the same nuclease agent being used to cleave the target DNA sequence for targeted insertion). The nuclease agent can then cleave the target sites flanking the polypeptide of interest coding sequence. In a specific example, the construct is delivered AAV-mediated delivery, and cleavage of the target sites flanking the polypeptide of interest coding sequence can remove the inverted terminal repeats (ITRs) of the AAV. In some instances, the target DNA sequence for targeted insertion (e.g., target DNA sequence in a safe harbor locus such as a gRNA target sequence including the flanking protospacer adjacent motif) is no longer present if the polypeptide of interest coding sequence is inserted into the cut site or target DNA sequence in the correct orientation but it is reformed if the polypeptide of interest coding sequence is inserted into the cut site or target DNA sequence in the opposite orientation. This can help ensure that the polypeptide of interest coding sequence is inserted in the correct orientation for expression.

The constructs disclosed herein can comprise a polyadenylation sequence or polyadenylation tail sequence (e.g., downstream or 3′ of a polypeptide of interest coding sequence). Methods of designing a suitable polyadenylation tail sequence are well-known. The polyadenylation tail sequence can be encoded, for example, as a “poly-A” stretch downstream of the polypeptide of interest coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. For example, the polyadenylation signal can comprise, consist essentially of, or consist of SEQ ID NO: 599, 169, or 161. For example, the polyadenylation signal can comprise, consist essentially of, or consist of SEQ ID NO: 169 or 161. For example, the polyadenylation signal can comprise, consist essentially of, or consist of SEQ ID NO: 169. For example, the polyadenylation signal can comprise, consist essentially of, or consist of SEQ ID NO: 599. In another example, the polyadenylation signal is a bovine growth hormone (BGH) polyadenylation signal or a CpG depleted BGH polyadenylation signal. For example, the polyadenylation signal can comprise, consist essentially of, or consist of SEQ ID NO: 162.

The constructs disclosed herein may also comprise splice acceptor sites (e.g., operably linked to the polypeptide of interest coding sequence, such as upstream or 5′ of the polypeptide of interest coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest (e.g., a GAA splice acceptor). For example, such a splice acceptor can be derived from the human GAA gene. Alternatively, such a splice acceptor can be derived from the mouse GAA gene. Additional suitable splice acceptor sites useful in eukaryotes, including artificial splice acceptors, are well-known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. In a specific example, the splice acceptor is a mouse Alb exon 2 splice acceptor. In a specific example, the splice acceptor can comprise, consist essentially of, or consist of SEQ ID NO: 163.

In some examples, the nucleic acid constructs disclosed herein can be bidirectional constructs, which are described in more detail below. In some examples, the nucleic acid constructs disclosed herein can be unidirectional constructs, which are described in more detail below. Likewise, in some examples, the nucleic acid constructs disclosed herein can be in a vector (e.g., viral vector, such as AAV, or rAAV8) and/or a lipid nanoparticle as described in more detail elsewhere herein.

### (1) Polypeptides of Interest

Any polypeptide of interest may be encoded by the nucleic acid constructs disclosed herein. In one example, the polypeptide of interest is a therapeutic polypeptide (e.g., a polypeptide that is lacking or deficient in a neonatal subject). In one example, the polypeptide of interest is an enzyme.

The polypeptide of interest can be a secreted polypeptide (e.g., a protein that is secreted by the cell and/or is functionally active as a soluble extracellular protein). Alternatively, the polypeptide of interest can be an intracellular polypeptide (e.g., a protein that is not secreted by the cell and is functionally active within the cell, including soluble cytosolic polypeptides).

The polypeptide of interest can be a wild type polypeptide. Alternatively, the polypeptide of interest can be a variant or mutant polypeptide.

In one example, the polypeptide of interest is a liver protein (e.g., a protein that is, endogenously produced in the liver and/or functionally active in the liver). In another example, the polypeptide of interest can be a circulating protein that is produced by the liver. In another example, the polypeptide of interest can be a non-liver protein.

The polypeptide of interest can be an exogenous polypeptide. An “exogenous” polypeptide coding sequence can refer to a coding sequence that has been introduced from an exogenous source to a site within a host cell genome (e.g., at a genomic locus such as a safe harbor locus, including ALB intron 1). That is, the exogenous polypeptide coding sequence is exogenous with respect to its insertion site, and the polypeptide of interest expressed from such an exogenous coding sequence is referred to as an exogenous polypeptide. The exogenous coding sequence can be naturally-occurring or engineered, and can be wild type or a variant. The exogenous coding sequence may include nucleotide sequences other than the sequence that encodes the exogenous polypeptide (e.g., an internal ribosomal entry site). The exogenous coding sequence can be a coding sequence that occurs naturally in the host genome, as a wild type or a variant (e.g., mutant). For example, although the host cell contains the coding sequence of interest (as a wild type or as a variant), the same coding sequence or variant thereof can be introduced as an exogenous source (e.g., for expression at a locus that is highly expressed). The exogenous coding sequence can also be a coding sequence that is not naturally occurring in the host genome, or that expresses an exogenous polypeptide that does not naturally occur in the host genome. An exogenous coding sequence can include an exogenous nucleic acid sequence (e.g., a nucleic acid sequence is not endogenous to the recipient cell), or may be exogenous with respect to its insertion site and/or with respect to its recipient cell.

In one example, the polypeptide of interest is not a Factor IX protein. In one example, the polypeptide of interest is not a multidomain therapeutic protein comprising a CD63-binding delivery domain linked to or fused to a lysosomal alpha-glucosidase (GAA). In one example, the polypeptide of interest is not a multidomain therapeutic protein comprising a CD63-binding delivery domain fused to a lysosomal alpha-glucosidase (GAA). In one example, the polypeptide of interest is not a multidomain therapeutic protein comprising a TfR-binding delivery domain linked to or fused to a GAA. In one example, the polypeptide of interest is not a multidomain therapeutic protein comprising a TfR-binding delivery domain fused to a GAA. In one example, the polypeptide of interest is not a Factor IX protein, is not a multidomain therapeutic protein comprising a CD63-binding delivery domain linked to or fused to a GAA, and is not a multidomain therapeutic protein comprising a TfR-binding delivery domain linked to or fused to a lysosomal alpha-glucosidase. In one example, the polypeptide of interest is not a Factor IX protein, is not a multidomain therapeutic protein comprising a CD63-binding delivery domain fused to a GAA, and is not a multidomain therapeutic protein comprising a TfR-binding delivery domain or fused to a lysosomal alpha-glucosidase.

In one example, the polypeptide of interest is a polypeptide associated with a genetic enzyme deficiency. In certain embodiments, the genetic enzyme deficiency results in infantile onset of disease. In certain embodiments, the genetic enzyme deficiency can be, or routinely is, diagnosed with newborn screening. In certain embodiments, the enzyme deficiency may manifest in various severity of disease such that the age of onset may include an infantile onset form of the disease and a later onset form of the disease (e.g., childhood, adolescent, or adult form of onset).

In one example, the polypeptide of interest is a polypeptide associated with a bleeding disorder, e.g., hemophilia, e.g., hemophilia A or hemophilia B, or von Willebrands disease. In certain embodiments, the polypeptide of interest is Factor VIII, Factor IX, or von Willebrand factor.

In one example, the polypeptide of interest is an enzyme related to inborn errors of metabolism. Such diseases include Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, argininosuccinic aciduria, methylmalonic aciduria, type I citrullinemia/argininosuccinate synthetase deficiency, carbamoyl-phosphate synthase 1 deficiency, propionic acidemia, isovaleric acidemia, glutaric academia I, progressive familial intrahepatic cholestasis, and types 2 and 3. In relation to such diseases, the polypeptide of interest include a hydrolase, α-galactosidase, β-galactosidase, α-glucosidase, (3-glucosidase, saposin-C activator, ceramidase, sphingomyelinase, β-hexosaminidase, GM2 activator, GM3 synthase, arylsulfatase, sphingolipid activator, α-iduronidase, iduronidase-2-sulfatase, heparin N-sulfatase, N-acetyl-α-glucosaminidase, α-glucosamide N-acetyltransferase, N-acetylglucosamine-6-sulfatase, N-acetylgalactosamine-6-sulfate sulfatase, N-acetylgalactosamine-4-sulfatase, β-glucuronidase, or a hyaluronidase. Association of specific diseases with specific enzyme deficiencies are provided herein.

In one example, the polypeptide of interest is a lysosomal alpha-glucosidase (GAA) polypeptide.

### (a) Lysosomal Alpha-Glucosidase (GAA)

Lysosomal alpha-glucosidase (GAA; also known as acid alpha-glucosidase, acid alpha-glucosidase preproprotein, acid maltase, aglucosidase alfa, alpha-1,4-glucosidase, amyloglucosidase, glucoamylase, LYAG) is encoded by GAA. This enzyme is active in lysosomes, where it breaks down glycogen into glucose.

The human GAA gene (NCBI GeneID 2548) encodes a 952 amino acid protein. In the lysosome, human GAA is sequentially processed by proteases to polypeptides of 76-, 19.4-, and 3.9-kDa that remain associated. Further cleavage between R(200) and A(204) inefficiently converts the 76-kDa polypeptide to the mature 70-kDa form with an additional 10.4-kDa polypeptide. GAA maturation increases its affinity for glycogen by 7-10 fold. A signal peptide is encoded by amino acids 1-27, a propeptide encoded by amino acids 28-69, lysosomal alpha-glucosidase after removal of the signal peptide and propeptide is encoded by amino acids 70-952, the 76 kDa lysosomal alpha-glucosidase is encoded by amino acids 123-952, and the 70 kDa lysosomal alpha-glucosidase is encoded by amino acids 204-952.

The GAA expressed from the compositions and methods disclosed herein can be any wild type or variant GAA. In one example, the GAA is a human GAA protein. Human GAA is assigned UniProt reference number P10253. An exemplary amino acid sequence for human GAA is assigned NCBI Accession No. NP_000143.2 and is set forth in SEQ ID NO: 170. An exemplary human GAA mRNA (cDNA) sequence is assigned NCBI Accession No. NM_000152.5 and is set forth in SEQ ID NO: 171. An exemplary human GAA coding sequence is assigned CCDS ID CCDS32760.1 and is set forth in SEQ ID NO: 172. An exemplary mature human GAA amino acid sequence (i.e., the human GAA sequence after removal of the signal peptide and propeptide) starting at amino acid 70 (i.e., GAA 70-952) is set forth in SEQ ID NO: 173. An exemplary coding sequence for GAA 70-952 is set forth in SEQ ID NO: 174.

In some examples, the GAA (e.g., human GAA) is a wild type GAA (e.g., wild type human GAA) sequence or a fragment thereof. For example, the GAA can be a fragment comprising the mature GAA amino acid sequence (i.e., the GAA sequence after removal of the signal peptide and propeptide), a fragment comprising the 77 kDa form of GAA, or a fragment comprising the 70 kDa form of GAA. In a specific example, the GAA can comprise SEQ ID NO: 173 or can be at least 90%, at least 91%, at least 92%, at least 93%, at least 94%, at least 95%, at least 96%, at least 97%, at least 98%, at least 99%, or at least 99.5% identical to SEQ ID NO: 173. In another specific example, the GAA can consist essentially of SEQ ID NO: 173. In another specific example, the GAA can consist of SEQ ID NO: 173.

The GAA coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a GAA coding sequence in a construct disclosed herein has one or more cryptic splice sites mutated or removed. In another example, a GAA coding sequence in a construct disclosed herein has all identified cryptic splice sites mutated or removed. In another example, a GAA coding sequence in a construct disclosed herein has one or more CpG dinucleotides removed (i.e., is CpG depleted). In another example, a GAA coding sequence in a construct disclosed herein has all CpG dinucleotides removed (i.e., is fully CpG depleted). In another example, a GAA coding sequence in a construct disclosed herein is codon optimized (e.g., codon optimized for expression in a human or mammal). In a specific example, a GAA coding sequence in a construct disclosed herein has one or more CpG dinucleotides removed (i.e., is CpG depleted) and has one or more cryptic splice sites mutated or removed. In another specific example, a GAA coding sequence in a construct disclosed herein has all CpG dinucleotides removed and has one or more or all identified cryptic splice sites mutated or removed. In another specific example, a GAA coding sequence in a construct disclosed herein has one or more CpG dinucleotides removed (i.e., is CpG depleted) and is codon optimized (e.g., codon optimized for expression in a human or mammal). In another specific example, a GAA coding sequence in a construct disclosed herein has all CpG dinucleotides removed (i.e., is fully CpG depleted) and is codon optimized (e.g., codon optimized for expression in a human or mammal).

Various GAA coding sequences are provided. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 174-182 and 581-588. In another example, the GAA coding sequence is (or comprises a sequence) at least 95%, at least 96%, at least 97%, at least 98%, at least 99%, at least 99.5%, or 100% identical to any one of SEQ ID NOS: 174-182 and 581-588. In another example, the GAA coding sequence is (or comprises a sequence) at least 99%, at least 99.5%, or 100% identical to any one of SEQ ID NOS: 174-182 and 581-588. In another example, the GAA coding sequence comprises the sequence set forth in any one of SEQ ID NOS: 174-182 and 581-588. In another example, the GAA coding sequence consists essentially of the sequence set forth in any one of SEQ ID NOS: 174-182 and 581-588. In another example, the GAA coding sequence consists of the sequence set forth in any one of SEQ ID NOS: 174-182 and 581-588. Various GAA coding sequences are provided. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 174-182. In another example, the GAA coding sequence is (or comprises a sequence) at least 95%, at least 96%, at least 97%, at least 98%, at least 99%, at least 99.5%, or 100% identical to any one of SEQ ID NOS: 174-182. In another example, the GAA coding sequence is (or comprises a sequence) at least 99%, at least 99.5%, or 100% identical to any one of SEQ ID NOS: 174-182. In another example, the GAA coding sequence comprises the sequence set forth in any one of SEQ ID NOS: 174-182. In another example, the GAA coding sequence consists essentially of the sequence set forth in any one of SEQ ID NOS: 174-182. In another example, the GAA coding sequence consists of the sequence set forth in any one of SEQ ID NOS: 174-182. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176. In another example, the GAA coding sequence is (or comprises a sequence) at least 95%, at least 96%, at least 97%, at least 98%, at least 99%, at least 99.5%, or 100% identical to SEQ ID NO: 176. In another example, the GAA coding sequence is (or comprises a sequence) at least 99%, at least 99.5%, or 100% identical to SEQ ID NO: 176. In another example, the GAA coding sequence comprises the sequence set forth in SEQ ID NO: 176. In another example, the GAA coding sequence consists essentially of the sequence set forth in SEQ ID NO: 176. In another example, the GAA coding sequence consists of the sequence set forth in SEQ ID NO: 176. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

Various codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized). In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 175-182. In another example, the GAA coding sequence is (or comprises a sequence) at least 95%, at least 96%, at least 97%, at least 98%, at least 99%, at least 99.5%, or 100% identical to any one of SEQ ID NOS: 175-182. In another example, the GAA coding sequence is (or comprises a sequence) at least 99%, at least 99.5%, or 100% identical to any one of SEQ ID NOS: 175-182. In another example, the GAA coding sequence comprises the sequence set forth in any one of SEQ ID NOS: 175-182. In another example, the GAA coding sequence consists essentially of the sequence set forth in any one of SEQ ID NOS: 175-182. In another example, the GAA coding sequence consists of the sequence set forth in any one of SEQ ID NOS: 175-182. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 99%, at least 99.5%, or 100% identical to SEQ ID NO: 176. In another example, the GAA coding sequence is (or comprises a sequence) at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 176 and encodes a GAA protein comprising the sequence set forth in SEQ ID NO: 173. In another example, the GAA coding sequence comprises the sequence set forth in SEQ ID NO: 176. In another example, the GAA coding sequence consists essentially of the sequence set forth in SEQ ID NO: 176. In another example, the GAA coding sequence consists of the sequence set forth in SEQ ID NO: 176. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 99%, at least 99.5%, or 100% identical to SEQ ID NO: 174. In another example, the GAA coding sequence is (or comprises a sequence) at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 174 and encodes a GAA protein comprising the sequence set forth in SEQ ID NO: 173. In another example, the GAA coding sequence comprises the sequence set forth in SEQ ID NO: 174. In another example, the GAA coding sequence consists essentially of the sequence set forth in SEQ ID NO: 174. In another example, the GAA coding sequence consists of the sequence set forth in SEQ ID NO: 174. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 181. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 181 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 181 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 181. In another example, the GAA coding sequence is (or comprises a sequence) at least 95%, at least 96%, at least 97%, at least 98%, at least 99%, at least 99.5%, or 100% identical to SEQ ID NO: 181 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 181 and encodes a GAA protein comprising the sequence set forth in SEQ ID NO: 173. In another example, the GAA coding sequence is (or comprises a sequence) at least 99%, at least 99.5%, or 100% identical to SEQ ID NO: 181. In another example, the GAA coding sequence is (or comprises a sequence) at least 99%, at least 99.5%, or 100% identical to SEQ ID NO: 181 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 181 and encodes a GAA protein comprising the sequence set forth in SEQ ID NO: 173. In another example, the GAA coding sequence comprises the sequence set forth in SEQ ID NO: 181. In another example, the GAA coding sequence consists essentially of the sequence set forth in SEQ ID NO: 181. In another example, the GAA coding sequence consists of the sequence set forth in SEQ ID NO: 181. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 180. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 180 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 180 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 180. In another example, the GAA coding sequence is (or comprises a sequence) at least 95%, at least 96%, at least 97%, at least 98%, at least 99%, at least 99.5%, or 100% identical to SEQ ID NO: 180 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 180 and encodes a GAA protein comprising the sequence set forth in SEQ ID NO: 173. In another example, the GAA coding sequence is (or comprises a sequence) at least 99%, at least 99.5%, or 100% identical to SEQ ID NO: 180. In another example, the GAA coding sequence is (or comprises a sequence) at least 99%, at least 99.5%, or 100% identical to SEQ ID NO: 180 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 180 and encodes a GAA protein comprising the sequence set forth in SEQ ID NO: 173. In another example, the GAA coding sequence comprises the sequence set forth in SEQ ID NO: 180. In another example, the GAA coding sequence consists essentially of the sequence set forth in SEQ ID NO: 180. In another example, the GAA coding sequence consists of the sequence set forth in SEQ ID NO: 180. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 178. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 178 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 178 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 178. In another example, the GAA coding sequence is (or comprises a sequence) at least 95%, at least 96%, at least 97%, at least 98%, at least 99%, at least 99.5%, or 100% identical to SEQ ID NO: 178 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 178 and encodes a GAA protein comprising the sequence set forth in SEQ ID NO: 173. In another example, the GAA coding sequence is (or comprises a sequence) at least 99%, at least 99.5%, or 100% identical to SEQ ID NO: 178. In another example, the GAA coding sequence is (or comprises a sequence) at least 99%, at least 99.5%, or 100% identical to SEQ ID NO: 178 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 178 and encodes a GAA protein comprising the sequence set forth in SEQ ID NO: 173. In another example, the GAA coding sequence comprises the sequence set forth in SEQ ID NO: 178. In another example, the GAA coding sequence consists essentially of the sequence set forth in SEQ ID NO: 178. In another example, the GAA coding sequence consists of the sequence set forth in SEQ ID NO: 178. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

Various other GAA coding sequences are provided. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 174 and 581-588. In another example, the GAA coding sequence is (or comprises a sequence) at least 95%, at least 96%, at least 97%, at least 98%, at least 99%, at least 99.5%, or 100% identical to any one of SEQ ID NOS: 174 and 581-588. In another example, the GAA coding sequence is (or comprises a sequence) at least 99%, at least 99.5%, or 100% identical to any one of SEQ ID NOS: 174 and 581-588. In another example, the GAA coding sequence comprises the sequence set forth in any one of SEQ ID NOS: 174 and 581-588. In another example, the GAA coding sequence consists essentially of the sequence set forth in any one of SEQ ID NOS: 174 and 581-588. In another example, the GAA coding sequence consists of the sequence set forth in any one of SEQ ID NOS: 174 and 581-588. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

Various other codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized). In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 581-588. In another example, the GAA coding sequence is (or comprises a sequence) at least 95%, at least 96%, at least 97%, at least 98%, at least 99%, at least 99.5%, or 100% identical to any one of SEQ ID NOS: 581-588. In another example, the GAA coding sequence is (or comprises a sequence) at least 99%, at least 99.5%, or 100% identical to any one of SEQ ID NOS: 581-588. In another example, the GAA coding sequence comprises the sequence set forth in any one of SEQ ID NOS: 581-588. In another example, the GAA coding sequence consists essentially of the sequence set forth in any one of SEQ ID NOS: 581-588. In another example, the GAA coding sequence consists of the sequence set forth in any one of SEQ ID NOS: 581-588. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 176 and encodes a GAA protein comprising the sequence set forth in SEQ ID NO: 173. In another example, the GAA coding sequence is (or comprises a sequence) at least 99%, at least 99.5%, or 100% identical to SEQ ID NO: 176. In another example, the GAA coding sequence is (or comprises a sequence) at least 99%, at least 99.5%, or 100% identical to SEQ ID NO: 176 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 176 and encodes a GAA protein comprising the sequence set forth in SEQ ID NO: 173. In another example, the GAA coding sequence comprises the sequence set forth in SEQ ID NO: 176. In another example, the GAA coding sequence consists essentially of the sequence set forth in SEQ ID NO: 176. In another example, the GAA coding sequence consists of the sequence set forth in SEQ ID NO: 176. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 174 and encodes a GAA protein comprising the sequence set forth in SEQ ID NO: 173. In another example, the GAA coding sequence is (or comprises a sequence) at least 99%, at least 99.5%, or 100% identical to SEQ ID NO: 174. In another example, the GAA coding sequence is (or comprises a sequence) at least 99%, at least 99.5%, or 100% identical to SEQ ID NO: 174 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 174 and encodes a GAA protein comprising the sequence set forth in SEQ ID NO: 173. In another example, the GAA coding sequence comprises the sequence set forth in SEQ ID NO: 174. In another example, the GAA coding sequence consists essentially of the sequence set forth in SEQ ID NO: 174. In another example, the GAA coding sequence consists of the sequence set forth in SEQ ID NO: 174. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 581. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 581 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 581 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 581. In another example, the GAA coding sequence is (or comprises a sequence) at least 95%, at least 96%, at least 97%, at least 98%, at least 99%, at least 99.5%, or 100% identical to SEQ ID NO: 581 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 581 and encodes a GAA protein comprising the sequence set forth in SEQ ID NO: 173. In another example, the GAA coding sequence is (or comprises a sequence) at least 99%, at least 99.5%, or 100% identical to SEQ ID NO: 581. In another example, the GAA coding sequence is (or comprises a sequence) at least 99%, at least 99.5%, or 100% identical to SEQ ID NO: 581 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 581 and encodes a GAA protein comprising the sequence set forth in SEQ ID NO: 173. In another example, the GAA coding sequence comprises the sequence set forth in SEQ ID NO: 581. In another example, the GAA coding sequence consists essentially of the sequence set forth in SEQ ID NO: 581. In another example, the GAA coding sequence consists of the sequence set forth in SEQ ID NO: 581. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 582. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 582 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 582 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 582. In another example, the GAA coding sequence is (or comprises a sequence) at least 95%, at least 96%, at least 97%, at least 98%, at least 99%, at least 99.5%, or 100% identical to SEQ ID NO: 582 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 582 and encodes a GAA protein comprising the sequence set forth in SEQ ID NO: 173. In another example, the GAA coding sequence is (or comprises a sequence) at least 99%, at least 99.5%, or 100% identical to SEQ ID NO: 582. In another example, the GAA coding sequence is (or comprises a sequence) at least 99%, at least 99.5%, or 100% identical to SEQ ID NO: 582 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 582 and encodes a GAA protein comprising the sequence set forth in SEQ ID NO: 173. In another example, the GAA coding sequence comprises the sequence set forth in SEQ ID NO: 582. In another example, the GAA coding sequence consists essentially of the sequence set forth in SEQ ID NO: 582. In another example, the GAA coding sequence consists of the sequence set forth in SEQ ID NO: 582. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 583. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 583 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 583 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 583. In another example, the GAA coding sequence is (or comprises a sequence) at least 95%, at least 96%, at least 97%, at least 98%, at least 99%, at least 99.5%, or 100% identical to SEQ ID NO: 583 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 583 and encodes a GAA protein comprising the sequence set forth in SEQ ID NO: 173. In another example, the GAA coding sequence is (or comprises a sequence) at least 99%, at least 99.5%, or 100% identical to SEQ ID NO: 583. In another example, the GAA coding sequence is (or comprises a sequence) at least 99%, at least 99.5%, or 100% identical to SEQ ID NO: 583 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 583 and encodes a GAA protein comprising the sequence set forth in SEQ ID NO: 173. In another example, the GAA coding sequence comprises the sequence set forth in SEQ ID NO: 583. In another example, the GAA coding sequence consists essentially of the sequence set forth in SEQ ID NO: 583. In another example, the GAA coding sequence consists of the sequence set forth in SEQ ID NO: 583. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 584. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 584 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 584 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 584. In another example, the GAA coding sequence is (or comprises a sequence) at least 95%, at least 96%, at least 97%, at least 98%, at least 99%, at least 99.5%, or 100% identical to SEQ ID NO: 584 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 584 and encodes a GAA protein comprising the sequence set forth in SEQ ID NO: 173. In another example, the GAA coding sequence is (or comprises a sequence) at least 99%, at least 99.5%, or 100% identical to SEQ ID NO: 584. In another example, the GAA coding sequence is (or comprises a sequence) at least 99%, at least 99.5%, or 100% identical to SEQ ID NO: 584 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 584 and encodes a GAA protein comprising the sequence set forth in SEQ ID NO: 173. In another example, the GAA coding sequence comprises the sequence set forth in SEQ ID NO: 584. In another example, the GAA coding sequence consists essentially of the sequence set forth in SEQ ID NO: 584. In another example, the GAA coding sequence consists of the sequence set forth in SEQ ID NO: 584. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 585. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 585 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 585 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 585. In another example, the GAA coding sequence is (or comprises a sequence) at least 95%, at least 96%, at least 97%, at least 98%, at least 99%, at least 99.5%, or 100% identical to SEQ ID NO: 585 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 585 and encodes a GAA protein comprising the sequence set forth in SEQ ID NO: 173. In another example, the GAA coding sequence is (or comprises a sequence) at least 99%, at least 99.5%, or 100% identical to SEQ ID NO: 585. In another example, the GAA coding sequence is (or comprises a sequence) at least 99%, at least 99.5%, or 100% identical to SEQ ID NO: 585 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 585 and encodes a GAA protein comprising the sequence set forth in SEQ ID NO: 173. In another example, the GAA coding sequence comprises the sequence set forth in SEQ ID NO: 585. In another example, the GAA coding sequence consists essentially of the sequence set forth in SEQ ID NO: 585. In another example, the GAA coding sequence consists of the sequence set forth in SEQ ID NO: 585. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 586. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 586 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 586 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 586. In another example, the GAA coding sequence is (or comprises a sequence) at least 95%, at least 96%, at least 97%, at least 98%, at least 99%, at least 99.5%, or 100% identical to SEQ ID NO: 586 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 586 and encodes a GAA protein comprising the sequence set forth in SEQ ID NO: 173. In another example, the GAA coding sequence is (or comprises a sequence) at least 99%, at least 99.5%, or 100% identical to SEQ ID NO: 586. In another example, the GAA coding sequence is (or comprises a sequence) at least 99%, at least 99.5%, or 100% identical to SEQ ID NO: 586 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 586 and encodes a GAA protein comprising the sequence set forth in SEQ ID NO: 173. In another example, the GAA coding sequence comprises the sequence set forth in SEQ ID NO: 586. In another example, the GAA coding sequence consists essentially of the sequence set forth in SEQ ID NO: 586. In another example, the GAA coding sequence consists of the sequence set forth in SEQ ID NO: 586. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 587. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 587 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 587 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 587. In another example, the GAA coding sequence is (or comprises a sequence) at least 95%, at least 96%, at least 97%, at least 98%, at least 99%, at least 99.5%, or 100% identical to SEQ ID NO: 587 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 587 and encodes a GAA protein comprising the sequence set forth in SEQ ID NO: 173. In another example, the GAA coding sequence is (or comprises a sequence) at least 99%, at least 99.5%, or 100% identical to SEQ ID NO: 587. In another example, the GAA coding sequence is (or comprises a sequence) at least 99%, at least 99.5%, or 100% identical to SEQ ID NO: 587 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 587 and encodes a GAA protein comprising the sequence set forth in SEQ ID NO: 173. In another example, the GAA coding sequence comprises the sequence set forth in SEQ ID NO: 587. In another example, the GAA coding sequence consists essentially of the sequence set forth in SEQ ID NO: 587. In another example, the GAA coding sequence consists of the sequence set forth in SEQ ID NO: 587. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 588. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 588 and encodes a GAA protein (or a GAA protein comprising a sequence) at least 99%, at least 99.5%, or 100% identical to SEQ ID NO: 173. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 588 and encodes a GAA protein comprising the sequence set forth in SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 588. In another example, the GAA coding sequence is (or comprises a sequence) at least 95%, at least 96%, at least 97%, at least 98%, at least 99%, at least 99.5%, or 100% identical to SEQ ID NO: 588 and encodes a GAA protein (or a GAA protein comprising a sequence) at least 99%, at least 99.5%, or 100% identical to SEQ ID NO: 173. In another example, the GAA coding sequence is (or comprises a sequence) at least 95%, at least 96%, at least 97%, at least 98%, at least 99%, at least 99.5%, or 100% identical to SEQ ID NO: 588 and encodes a GAA protein comprising the sequence set forth in SEQ ID NO: 173. In another example, the GAA coding sequence is (or comprises a sequence) at least 99%, at least 99.5%, or 100% identical to SEQ ID NO: 588. In another example, the GAA coding sequence is (or comprises a sequence) at least 99%, at least 99.5%, or 100% identical to SEQ ID NO: 588 and encodes a GAA protein (or a GAA protein comprising a sequence) at least 99%, at least 99.5%, or 100% identical to SEQ ID NO: 173. In another example, the GAA coding sequence is (or comprises a sequence) at least 99%, at least 99.5%, or 100% identical to SEQ ID NO: 588 and encodes a GAA protein comprising the sequence set forth in SEQ ID NO: 173. In another example, the GAA coding sequence comprises the sequence set forth in SEQ ID NO: 588. In another example, the GAA coding sequence consists essentially of the sequence set forth in SEQ ID NO: 588. In another example, the GAA coding sequence consists of the sequence set forth in SEQ ID NO: 588. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 173 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 173 (and, e.g., retaining the activity of native GAA). Optionally, the GAA coding sequence in the above examples encodes a GAA protein comprising the sequence set forth in SEQ ID NO: 173. Optionally, the GAA coding sequence in the above examples encodes a GAA protein consisting essentially of the sequence set forth in SEQ ID NO: 173. Optionally, the GAA coding sequence in the above examples encodes a GAA protein consisting of the sequence set forth in SEQ ID NO: 173.

When specific GAA nucleic acid constructs sequences are disclosed herein, they are meant to encompass the sequence disclosed or the reverse complement of the sequence. For example, if a GAA nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the GAA nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (2) Bidirectional Constructs

The nucleic acid constructs disclosed herein can be bidirectional constructs. Such bidirectional constructs can allow for enhanced insertion and expression of encoded polypeptide of interest. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus or a cleavage site or target insertion site, allowing the expression of the polypeptide of interest when inserted in either orientation, thereby enhancing expression efficiency.

A bidirectional construct as disclosed herein can comprise at least two nucleic acid segments, wherein a first segment comprises a first coding sequence for the polypeptide of interest, and a second segment comprises the reverse complement of a second coding sequence for the polypeptide of interest, or vice versa. However, other bidirectional constructs disclosed herein can comprise at least two nucleic acid segments, wherein the first segment comprises a coding sequence for a polypeptide of interest, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the polypeptide of interest or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode a polypeptide of interest, the at least two segments can encode the same polypeptide of interest or different polypeptides of interest. The different polypeptides of interest can be at least about 90%, at least about 95%, at least about 96%, at least about 97%, at least about 98%, at least about 99%, or at least about 99.5% identical. For example, the first segment can encode a wild type polypeptide of interest or fragment thereof, and the second segment can encode a variant of the polypeptide of interest or fragment thereof, or vice versa. Alternatively, the first segment can encode a first variant polypeptide of interest, and the second segment can encode a second variant polypeptide of interest that is different from the first variant polypeptide of interest. Preferably, the two segments encode the same polypeptide of interest (i.e., 100% identical).

Even when the two segments encode the same polypeptide of interest, the coding sequence for the polypeptide of interest in the first segment can differ from the coding sequence for the polypeptide of interest in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second polypeptide of interest coding sequence can be codon optimized or may use one or more alternative codons for one or more amino acids of the same polypeptide of interest (i.e., same amino acid sequence) encoded by the polypeptide of interest coding sequence in the first segment. An alternative codon as used herein refers to variations in codon usage for a given amino acid, and may or may not be a preferred or optimized codon (codon optimized) for a given expression system. Preferred codon usage, or codons that are well-tolerated in a given system of expression are known.

In one example, the second segment comprises a reverse complement of a polypeptide of interest coding sequence that adopts different codon usage from that of the polypeptide of interest coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment can have any percentage of complementarity to the first segment. For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of the polypeptide of interest.

In some cases, the bidirectional nucleic acid construct does not comprise a promoter that drives the expression of the polypeptide of interest. For example, the expression of the polypeptide of interest can be driven by a promoter of the host cell (e.g., the endogenous ALB promoter when the transgene is integrated into a host cell's ALB locus). In other cases, the bidirectional nucleic acid construct can comprise one or more promoters operably linked to the coding sequences for the polypeptide of interest. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first polypeptide of interest coding sequence and/or the reverse complement of a promoter that drives expression of the reverse complement of the second polypeptide of interest coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a polypeptide of interest.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. For example, one polyadenylation signal can be an SV40 polyadenylation signal, and the other polyadenylation signal can be a BGH polyadenylation signal. In a specific example, one polyadenylation signal can comprise, consist essentially of, or consist of SEQ ID NO: 161, and the other polyadenylation signal can comprise, consist essentially of, or consist of SEQ ID NO: 162.

In some bidirectional constructs, both the first segment and the second segment comprise a polyadenylation tail sequence. Methods of designing a suitable polyadenylation tail sequence are known. For example, in some bidirectional constructs, one or both of the first and second segment comprises a polyadenylation tail sequence and/or a polyadenylation signal sequence downstream of an open reading frame (i.e., a polyadenylation tail sequence and/or a polyadenylation signal sequence 3′ of a coding sequence, or a reverse complement of a polyadenylation tail sequence and/or a polyadenylation signal sequence 5′ of a reverse complement of a coding sequence). The polyadenylation tail sequence can be encoded, for example, as a “poly-A” stretch downstream of the polypeptide of interest coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. In some bidirectional constructs, a single bidirectional terminator can be used to terminate RNA polymerase transcription in either the sense or the antisense direction (i.e., to terminate RNA polymerase transcription from both the first segment and the second segment). Examples of bidirectional terminators include the ARO4, TRP1, TRP4, ADH1, CYC1, GAL1, GAL7, and GAL10 terminators.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are mouse Alb exon 2 splice acceptors. In a specific example, both splice acceptors can comprise, consist essentially of, or consist of SEQ ID NO: 163.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are mouse Alb exon 2 splice acceptors.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary bidirectional construct, the second segment is located 3′ of the first segment, the first polypeptide of interest coding sequence and the second polypeptide of interest coding sequence both encode the same human polypeptide of interest, the second polypeptide of interest coding sequence adopts a different codon usage from the codon usage of the first polypeptide of interest coding sequence, the first segment comprises a first polyadenylation signal sequence located 3′ of the first polypeptide of interest coding sequence, the second segment comprises a reverse complement of a second polyadenylation signal sequence located 5′ of the reverse complement of the second polypeptide of interest coding sequence, the first segment comprises a first splice acceptor site located 5′ of the first polypeptide of interest coding sequence, the second segment comprises a reverse complement of a second splice acceptor site located 3′ of the reverse complement of the second polypeptide of interest coding sequence, the nucleic acid construct does not comprise a promoter that drives expression of the first polypeptide of interest or the second polypeptide of interest, and optionally the nucleic acid construct does not comprise a homology arm.

### (3) Unidirectional Constructs

The nucleic acid constructs disclosed herein can be unidirectional constructs. When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the unidirectional constructs, the coding sequence for the polypeptide of interest can be codon-optimized for expression in a host cell. For example, the coding sequence can be codon optimized or may use one or more alternative codons for one or more amino acids of the polypeptide of interest (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The unidirectional constructs disclosed herein can be modified to include any suitable structural feature as needed for any particular use and/or that confers one or more desired functions. For example, the unidirectional nucleic acid constructs disclosed herein need not comprise a homology arm and/or can be, for example, homology-independent donor constructs.

In some cases, the unidirectional nucleic acid construct does not comprise a promoter that drives the expression of polypeptide of interest. For example, the expression of the polypeptide of interest can be driven by a promoter of the host cell (e.g., the endogenous ALB promoter when the transgene is integrated into a host cell's ALB locus). In other cases, the unidirectional nucleic acid construct can comprise one or more promoters operably linked to the coding sequence for the polypeptide of interest. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some unidirectional constructs can comprise a promoter that drives expression of the coding sequence for the polypeptide of interest.

The unidirectional constructs can, in some cases, comprise one or more polyadenylation tail sequences or polyadenylation signal sequences. Some unidirectional constructs can comprise a polyadenylation signal sequence located 3′ of the coding sequence for the polypeptide of interest. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a BGH polyadenylation signal. For example, the polyadenylation signal can be an SV40 polyadenylation signal or a BGH polyadenylation signal. In a specific example, the polyadenylation signal can comprise, consist essentially of, or consist of SEQ ID NO: 161. In another specific example, the polyadenylation signal can comprise, consist essentially of, or consist of SEQ ID NO: 162.

Methods of designing a suitable polyadenylation tail sequence are known. For example, some unidirectional constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the coding sequence for the polypeptide of interest (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The unidirectional constructs can, in some cases, comprise one or more splice acceptor sites. Some unidirectional constructs comprise a splice acceptor site located 5′ of the coding sequence for the polypeptide of interest. In a specific example, the splice acceptor is a mouse Alb exon 2 splice acceptor. In a specific example, the splice acceptor can comprise, consist essentially of, or consist of SEQ ID NO: 163.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from the gene encoding the polypeptide of interest. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The unidirectional constructs can be circular or linear. For example, a unidirectional construct can be linear.

The unidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the unidirectional construct is single-stranded (e.g., single-stranded DNA).

The unidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the unidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary unidirectional construct, the construct comprises a polyadenylation signal sequence located 3′ of the coding sequence for the polypeptide of interest, the construct comprises a splice acceptor site located 5′ of the coding sequence for the polypeptide of interest, and the nucleic acid construct does not comprise a promoter that drives expression of the polypeptide of interest, and optionally the nucleic acid construct does not comprise a homology arm.

### (4) Vectors

The nucleic acid constructs disclosed herein can be provided in a vector for expression or for integration into and expression from a target genomic locus. A vector can comprise additional sequences such as, for example, replication origins, promoters, and genes encoding antibiotic resistance. A vector can also comprise nuclease agent components as disclosed elsewhere herein. For example, a vector can comprise a nucleic acid construct encoding a polypeptide of interest, a CRISPR/Cas system (nucleic acids encoding Cas protein and gRNA), one or more components of a CRISPR/Cas system, or a combination thereof (e.g., a nucleic acid construct and a gRNA). In some cases, a vector comprising a nucleic acid construct encoding a polypeptide of interest does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 158, SEQ ID NO: 159, or SEQ ID NO: 160. Other examples of ITRs comprise one or more mutations compared to SEQ ID NO: 158, SEQ ID NO: 159, or SEQ ID NO: 160 and can be at least 90%, at least 91%, at least 92%, at least 93%, at least 94%, at least 95%, at least 96%, at least 97%, at least 98%, or at least 99% identical to SEQ ID NO: 158, SEQ ID NO: 159, or SEQ ID NO: 160. In some rAAV genomes disclosed herein, the nucleic acid construct is flanked on both sides by the same ITR (i.e., the ITR on the 5′ end, and the reverse complement of the ITR on the 3′ end, such as SEQ ID NO: 158 on the 5′ end and SEQ ID NO: 168 on the 3′ end, or SEQ ID NO: 159 on the 5′ end and SEQ ID NO: 597 on the 3′ end, or SEQ ID NO: 160 on the 5′ end and SEQ ID NO: 598 on the 3′ end). In one example, the ITR on each end can comprise, consist essentially of, or consist of SEQ ID NO: 158 (i.e., SEQ ID NO: 158 on the 5′ end, and the reverse complement on the 3′ end). In another example, the ITR on each end can comprise, consist essentially of, or consist of SEQ ID NO: 159 (i.e., SEQ ID NO: 159 on the 5′ end, and the reverse complement on the 3′ end). In one example, the ITR on at least one end comprises, consists essentially of, or consists of SEQ ID NO: 160. In one example, the ITR on the 5′ end comprises, consists essentially of, or consists of SEQ ID NO: 160. In one example, the ITR on the 3′ end comprises, consists essentially of, or consists of SEQ ID NO: 160. In one example, the ITR on each end can comprise, consist essentially of, or consist of SEQ ID NO: 160 (i.e., SEQ ID NO: 160 on the 5′ end, and the reverse complement on the 3′ end). In other rAAV genomes disclosed herein, the nucleic acid construct is flanked by different ITRs on each end. In one example, the ITR on one end comprises, consists essentially of, or consists of SEQ ID NO: 158, and the ITR on the other end comprises, consists essentially of, or consists of SEQ ID NO: 159. In another example, the ITR on one end comprises, consists essentially of, or consists of SEQ ID NO: 158, and the ITR on the other end comprises, consists essentially of, or consists of SEQ ID NO: 160. In one example, the ITR on one end comprises, consists essentially of, or consists of SEQ ID NO: 159, and the ITR on the other end comprises, consists essentially of, or consists of SEQ ID NO: 160.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y73IF, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### B. Nuclease Agents and CRISPR/Cas Systems

The methods and compositions disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as a safe harbor gene (e.g., ALB) for insertion of a nucleic acid construct as disclosed herein. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the nucleic acid construct is inserted to express the polypeptide of interest. The polypeptide of interest may be exogenous with respect to its insertion site or locus (target gene), such as a safe harbor locus from which polypeptide of interest is not normally expressed. Alternatively, the polypeptide of interest may be non-exogenous with respect to its insertion site, such as insertion into an endogenous locus encoding the polypeptide of interest to correct a defective gene encoding the polypeptide of interest.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ALB gene or locus (e.g., ALB genomic locus) within a cell, or intron 1 of an ALB gene or locus within a cell. In a more specific example, the CRISPR/Cas systems or components of such systems target a human ALB gene or locus or intron 1 of a human ALB gene or locus within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ALB gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ALB gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### (1) Target Genomic Loci and Albumin (ALB)

Any target genomic locus capable of expressing a gene can be used, such as a safe harbor locus (safe harbor gene, such as ALB) or an endogenous GAA locus. The nucleic acid construct can be integrated into any part of the target genomic locus. For example, the nucleic acid construct can be inserted into an intron or an exon of a target genomic locus or can replace one or more introns and/or exons of a target genomic locus. In a specific example, the nucleic acid construct can be integrated into an intron of the target genomic locus, such as the first intron of the target genomic locus (e.g., ALB intron 1). See, e.g., WO 2020/082042, US 2020/0270617, WO 2020/082041, US 2020/0268906, WO 2020/082046, and US 2020/0289628, each of which is herein incorporated by reference in its entirety for all purposes. Constructs integrated into a target genomic locus can be operably linked to an endogenous promoter at the target genomic locus (e.g., the endogenous ALB promoter).

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67, 328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In a specific example, a safe harbor locus is a locus within the genome wherein a gene may be inserted without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). The safe harbor locus can allow overexpression of an exogenous gene without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). A desirable safe harbor locus may be one in which expression of the inserted gene sequence is not perturbed by read-through expression from neighboring genes. The safe harbor may be a human safe harbor (e.g., for a liver tissue or hepatocyte host cell).

In a specific example, the target genomic locus is an ALB locus, such as intron 1 of an ALB locus. In a more specific example, the target genomic locus is a human ALB locus, such as intron 1 of a human ALB locus (e.g., SEQ ID NO: 4).

### (2) Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 8 (encoded by the DNA sequence set forth in SEQ ID NO: 9). An exemplary SpCas9 mRNA (cDNA) sequence is set forth in SEQ ID NO: 10. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPRI locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence comprises, consists essentially of, or consists of SEQ ID NO: 11. An exemplary SpCas9 mRNA sequence encoding that SpCas9 protein sequence comprises, consists essentially of, or consists of SEQ ID NO: 12. Another exemplary SpCas9 mRNA sequence encoding that SpCas9 protein sequence comprises, consists essentially of, or consists of SEQ ID NO: 1. Another exemplary SpCas9 mRNA sequence encoding that SpCas9 protein sequence comprises SEQ ID NO: 2. An exemplary SpCas9 coding sequence comprises, consists essentially of, or consists of SEQ ID NO: 3.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCasl2e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ(CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposes Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 13) or PKKKRRV (SEQ ID NO: 14). The NLS may be a bipartite sequence, such as the NLS of nucleoplasmin, KRPAATKKAGQAKKKK (SEQ ID NO: 15). In a specific example, a single PKKKRKV (SEQ ID NO: 13) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate 2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, the nuclease agent can be CRISPR/Cas9, and a Cas9 mRNA and a gRNA targeting an intron 1 of an endogenous human ALB locus can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

### (3) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 16) or GUUUUAGAGCUAUGCUGUUUUG (SEQ ID NO: 17). Any of the DNA-targeting segments disclosed herein can be joined to the 5′ end of SEQ ID NO: 16 or 17 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of AGCAUAGCAAGUUAAAAUAAGGCUAGUCCGUUAUCAACUUGAAAAAGUGGCACC GAGUCGGUGCUUU (SEQ ID NO: 18), AAACAGCAUAGCAAGUUAAAAUAAGGCUAGUCCGUUAUCAACUUGAAAAAGUGG CACCGAGUCGGUGCUUUU (SEQ ID NO: 19), or GUUGGAACCAUUCAAAACAGCAUAGCAAGUUAAAAUAAGGCUAGUCCGUUAUCA ACUUGAAAAAGUGGCACCGAGUCGGUGC (SEQ ID NO: 20).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 30-61. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 30-61.

As another example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 36, 30, 33, and 41.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 36. Alternatively, a guide RNA targeting intron 1 of a human ALB gene can comprise a DNA-targeting segment that is at least 90% or at least 95% identical to the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 36.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 30. Alternatively, a guide RNA targeting intron 1 of a human ALB gene can comprise a DNA-targeting segment that is at least 90% or at least 95% identical to the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 30.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 33. Alternatively, a guide RNA targeting intron 1 of a human ALB gene can comprise a DNA-targeting segment that is at least 90% or at least 95% identical to the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 33.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 41. Alternatively, a guide RNA targeting intron 1 of a human ALB gene can comprise a DNA-targeting segment that is at least 90% or at least 95% identical to the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 41.

TABLE 2
Human ALB Intron 1 Guide Sequences.

| Guide Sequence | SEQ ID NO: |
|---|---|
| GAGCAACCUCACUCUUGUCU | 30 |
| AUGCAUUUGUUUCAAAAUAU | 31 |
| UGCAUUUGUUUCAAAAUAUU | 32 |
| AUUUAUGAGAUCAACAGCAC | 33 |
| GAUCAACAGCACAGGUUUUG | 34 |
| UUAAAUAAAGCAUAGUGCAA | 35 |
| UAAAGCAUAGUGCAAUGGAU | 36 |
| UAGUGCAAUGGAUAGGUCUU | 37 |
| UACUAAAACUUUAUUUUACU | 38 |
| AAAGUUGAACAAUAGAAAAA | 39 |
| AAUGCAUAAUCUAAGUCAAA | 40 |
| UAAUAAAAUUCAAACAUCCU | 41 |
| GCAUCUUUAAAGAAUUAUUU | 42 |
| UUUGGCAUUUAUUUCUAAAA | 43 |
| UGUAUUUGUGAAGUCUUACA | 44 |
| UCCUAGGUAAAAAAAAAAAA | 45 |
| UAAUUUUCUUUUGCGCACUA | 46 |
| UGACUGAAACUUCACAGAAU | 47 |
| GACUGAAACUUCACAGAAUA | 48 |
| UUCAUUUUAGUCUGUCUUCU | 49 |
| AUUAUCUAAGUUUGAAUAUA | 50 |
| AAUUUUUAAAAUAGUAUUCU | 51 |
| UGAAUUAUUCUUCUGUUUAA | 52 |
| AUCAUCCUGAGUUUUUCUGU | 53 |
| UUACUAAAACUUUAUUUUAC | 54 |
| ACCUUUUUUUUUUUUUACCU | 55 |
| AGUGCAAUGGAUAGGUCUUU | 56 |
| UGAUUCCUACAGAAAAACUC | 57 |
| UGGGCAAGGGAAGAAAAAAA | 58 |
| CCUCACUCUUGUCUGGGCAA | 59 |
| ACCUCACUCUUGUCUGGGCA | 60 |
| UGAGCAACCUCACUCUUGUC | 61 |

TABLE 3
Human ALB Intron 1 sgRNA Sequences.

| Full Sequence | Full Sequence Modified |
|---|---|
| GAGCAACCUCACUCUUGUCUGUUU | mG*mA*mG*CAACCUCACUCUUGUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 62) | U*mU*mU (SEQ ID NO: 94) |
| AUGCAUUUGUUUCAAAAUAUGUUU | mA*mU*mG*CAUUUGUUUCAAAAUAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 63) | U*mU*mU (SEQ ID NO: 95) |
| UGCAUUUGUUUCAAAAUAUUGUUU | mU*mG*mC*AUUUGUUUCAAAAUAUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 64) | U*mU*mU (SEQ ID NO: 96) |
| AUUUAUGAGAUCAACAGCACGUUU | mA*mU*mU*UAUGAGAUCAACAGCACGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 65) | U*mU*mU (SEQ ID NO: 97) |
| GAUCAACAGCACAGGUUUUGGUUU | mG*mA*mU*CAACAGCACAGGUUUUGGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 66) | U*mU*mU (SEQ ID NO: 98) |
| UUAAAUAAAGCAUAGUGCAAGUUU | mU*mU*mA*AAUAAAGCAUAGUGCAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 67) | U*mU*mU (SEQ ID NO: 99) |
| UAAAGCAUAGUGCAAUGGAUGUUU | mU*mA*mA*AGCAUAGUGCAAUGGAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 68) | U*mU*mU (SEQ ID NO: 100) |
| UAGUGCAAUGGAUAGGUCUUGUUU | mU*mA*mG*UGCAAUGGAUAGGUCUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 69) | U*mU*mU (SEQ ID NO: 101) |
| UACUAAAACUUUAUUUUACUGUUU | mU*mA*mC*UAAAACUUUAUUUUACUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 70) | U*mU*mU (SEQ ID NO: 102) |
| AAAGUUGAACAAUAGAAAAAGUUU | mA*mA*mA*GUUGAACAAUAGAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 71) | U*mU*mU (SEQ ID NO: 103) |
| AAUGCAUAAUCUAAGUCAAAGUUU | mA*mA*mU*GCAUAAUCUAAGUCAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 72) | U*mU*mU (SEQ ID NO: 104) |
| UAAUAAAAUUCAAACAUCCUGUUU | mU*mA*mA*UAAAAUUCAAACAUCCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 73) | U*mU*mU (SEQ ID NO: 105) |
| GCAUCUUUAAAGAAUUAUUUGUUU | mG*mC*mA*UCUUUAAAGAAUUAUUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 74) | U*mU*mU (SEQ ID NO: 106) |
| UUUGGCAUUUAUUUCUAAAAGUUU | mU*mU*mU*GGCAUUUAUUUCUAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 75) | U*mU*mU (SEQ ID NO: 107) |
| UGUAUUUGUGAAGUCUUACAGUUU | mU*mG*mU*AUUUGUGAAGUCUUACAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 76) | U*mU*mU (SEQ ID NO: 108) |
| UCCUAGGUAAAAAAAAAAAAGUUU | mU*mC*mC*UAGGUAAAAAAAAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 77) | U*mU*mU (SEQ ID NO: 109) |
| UAAUUUUCUUUUGCGCACUAGUUU | mU*mA*mA*UUUUCUUUUGCGCACUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 78) | U*mU*mU (SEQ ID NO: 110) |
| UGACUGAAACUUCACAGAAUGUUU | mU*mG*mA*CUGAAACUUCACAGAAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 79) | U*mU*mU (SEQ ID NO: 111) |
| GACUGAAACUUCACAGAAUAGUUU | mG*mA*mC*UGAAACUUCACAGAAUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 80) | U*mU*mU (SEQ ID NO: 112) |
| UUCAUUUUAGUCUGUCUUCUGUUU | mU*mU*mC*AUUUUAGUCUGUCUUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 81) | U*mU*mU (SEQ ID NO: 113) |
| AUUAUCUAAGUUUGAAUAUAGUUU | mA*mU*mU*AUCUAAGUUUGAAUAUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 82) | U*mU*mU (SEQ ID NO: 114) |
| AAUUUUUAAAAUAGUAUUCUGUUU | mA*mA*mU*UUUUAAAAUAGUAUUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 83) | U*mU*mU (SEQ ID NO: 115) |
| UGAAUUAUUCUUCUGUUUAAGUUU | mU*mG*mA*AUUAUUCUUCUGUUUAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 84) | U*mU*mU (SEQ ID NO: 116) |
| AUCAUCCUGAGUUUUUCUGUGUUU | mA*mU*mC*AUCCUGAGUUUUUCUGUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 85) | U*mU*mU (SEQ ID NO: 117) |
| UUACUAAAACUUUAUUUUACGUUU | mU*mU*mA*CUAAAACUUUAUUUUACGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 86) | U*mU*mU (SEQ ID NO: 118) |
| ACCUUUUUUUUUUUUUACCUGUUU | mA*mC*mC*UUUUUUUUUUUUUACCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 87) | U*mU*mU (SEQ ID NO: 119) |
| AGUGCAAUGGAUAGGUCUUUGUUU | mA*mG*mU*GCAAUGGAUAGGUCUUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 88) | U*mU*mU (SEQ ID NO: 120) |
| UGAUUCCUACAGAAAAACUCGUUU | mU*mG*mA*UUCCUACAGAAAAACUCGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 89) | U*mU*mU (SEQ ID NO: 121) |
| UGGGCAAGGGAAGAAAAAAAGUUU | mU*mG*mG*GCAAGGGAAGAAAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 90) | U*mU*mU (SEQ ID NO: 122) |
| CCUCACUCUUGUCUGGGCAAGUUU | mC*mC*mU*CACUCUUGUCUGGGCAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 91) | U*mU*mU (SEQ ID NO: 123) |
| ACCUCACUCUUGUCUGGGCAGUUU | mA*mC*mC*UCACUCUUGUCUGGGCAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 92) | U*mU*mU (SEQ ID NO: 124) |
| UGAGCAACCUCACUCUUGUCGUUU | mU*mG*mA*GCAACCUCACUCUUGUCGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 93) | U*mU*mU (SEQ ID NO: 125) |

TABLE 4
Mouse Alb Intron 1 Guide Sequences.

| Guide Sequence | SEQ ID NO: |
|---|---|
| CACUCUUGUCUGUGGAAACA | 164 |

TABLE 5
Mouse Alb Intron 1 sgRNA Sequences.

| Full Sequence | Full Sequence Modified |
|---|---|
| CACUCUUGUCUGUGGAAACAGUUU | mC*mA*mC*UCUUGUCUGUGGAAACAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 166) | U*mU*mU (SEQ ID NO: 167) |

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

(version 1; SEQ ID NO: 21)
GUUUUAGAGCUAGAAAUAGCAAGUUAAAAUAAGGCUAGUCCGUUAUCAAC
UUGAAAAAGUGGCACCGAGUCGGUGCU;
(version 2; SEQ ID NO: 22)
GUUGGAACCAUUCAAAACAGCAUAGCAAGUUAAAAUAAGGCUAGUCCGUU
AUCAACUUGAAAAAGUGGCACCGAGUCGGUGC;
(version 3; SEQ ID NO: 23)
GUUUUAGAGCUAGAAAUAGCAAGUUAAAAUAAGGCUAGUCCGUUAUCAAC
UUGAAAAAGUGGCACCGAGUCGGUGC;
and
(version 4; SEQ ID NO: 24)
GUUUAAGAGCUAUGCUGGAAACAGCAUAGCAAGUUUAAAUAAGGCUAGUC
CGUUAUCAACUUGAAAAAGUGGCACCGAGUCGGUGC;
(version 5; SEQ ID NO: 25)
GUUUUAGAGCUAGAAAUAGCAAGUUAAAAUAAGGCUAGUCCGUUAUCAAC
UUGAAAAAGUGGCACCGAGUCGGUGCUUUUUUU;
(version 6; SEQ ID NO: 26)
GUUUUAGAGCUAGAAAUAGCAAGUUAAAAUAAGGCUAGUCCGUUAUCAAC
UUGAAAAAGUGGCACCGAGUCGGUGCUUUU;
(version 7; SEQ ID NO: 27)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUU;
or
(version 8; SEQ ID NO: 28)
GUUUUAGAGCUAGAAAUAGCAAGUUAAAAUAAGGCUAGUCCGUUAUCAAC
UUGGCACCGAGUCGGUGC.

In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide. For example, the totality of N residues comprise a human ALB intron 1 DNA-targeting segment as described herein (e.g., the sequence set forth in SEQ ID NO: 29, wherein the N residues are replaced with the DNA-targeting segment of any one of SEQ ID NOS: 30-61, the DNA-targeting segment of any one of SEQ ID NOS: 36, 30, 33, and 41, or the DNA-targeting segment of SEQ ID NO: 36. For example, a modified gRNA can comprise the sequence set forth in any one of SEQ ID NOS: 94-125, the sequence set forth in any one of SEQ ID NOS: 100, 94, 97, and 105, or the sequence set forth in SEQ ID NO: 100 in Table 3. The terms “mA,” “mC,” “mU,” and “mG” denote a nucleotide (A, C, U, and G, respectively) that has been modified with 2′-O-Me. The symbol “*” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting intron 1 of a human ALB gene can comprise, consist essentially of, or consist of the sequence set forth in any one of SEQ ID NOS: 62-125. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 62-125. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 62-125. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 62-125.

As another example, a guide RNA targeting intron 1 of a human ALB gene can comprise, consist essentially of, or consist of the sequence set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 68, 100, 62, 94, 65, 97, 73, and 105.

As another example, a guide RNA targeting intron 1 of a human ALB gene can comprise, consist essentially of, or consist of the sequence set forth in SEQ ID NO: 68 or 100. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 68 or 100. Alternatively, a guide RNA targeting intron 1 of a human ALB gene can comprise, consist essentially of, or consist of a sequence that is at least 90% or at least 95% identical to the DNA-targeting segment set forth in SEQ ID NO: 68 or 100. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 68 or 100.

As another example, a guide RNA targeting intron 1 of a human ALB gene can comprise, consist essentially of, or consist of the sequence set forth in SEQ ID NO: 62 or 94. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 62 or 94. Alternatively, a guide RNA targeting intron 1 of a human ALB gene can comprise, consist essentially of, or consist of a sequence that is at least 90% or at least 95% identical to the DNA-targeting segment set forth in SEQ ID NO: 62 or 94. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 62 or 94.

As another example, a guide RNA targeting intron 1 of a human ALB gene can comprise, consist essentially of, or consist of the sequence set forth in SEQ ID NO: 65 or 97. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 65 or 97. Alternatively, a guide RNA targeting intron 1 of a human ALB gene can comprise, consist essentially of, or consist of a sequence that is at least 90% or at least 95% identical to the DNA-targeting segment set forth in SEQ ID NO: 65 or 97. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 65 or 97.

As another example, a guide RNA targeting intron 1 of a human ALB gene can comprise, consist essentially of, or consist of the sequence set forth in SEQ ID NO: 73 or 105. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 73 or 105. Alternatively, a guide RNA targeting intron 1 of a human ALB gene can comprise, consist essentially of, or consist of a sequence that is at least 90% or at least 95% identical to the DNA-targeting segment set forth in SEQ ID NO: 73 or 105. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 73 or 105.

### (4) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N2 is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 5) or N₂₀NGG (SEQ ID NO: 6). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 7) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 5-7, including the 5′ G or GG and the 3′ GG or NGG. Yet other guide RNA target sequences plus PAMs can have between 14 and 20 nucleotides in length of SEQ ID NOS: 5-7.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting intron 1 of a human ALB gene can target the guide RNA target sequence set forth in any one of SEQ ID NOS: 126-157. As another example, a guide RNA targeting intron 1 of a human ALB gene can target at least 17, at least 18, at least 19, or at least 20 contiguous nucleotides of the guide RNA target sequence set forth in any one of SEQ ID NOS: 126-157.

As another example, a guide RNA targeting intron 1 of a human ALB gene can target the guide RNA target sequence set forth in any one of SEQ ID NOS: 132, 126, 129, and 137. As another example, a guide RNA targeting intron 1 of a human ALB gene can target at least 17, at least 18, at least 19, or at least 20 contiguous nucleotides of the guide RNA target sequence set forth in any one of SEQ ID NOS: 132, 126, 129, and 137.

As another example, a guide RNA targeting intron 1 of a human ALB gene can target the guide RNA target sequence set forth in SEQ ID NO: 132. As another example, a guide RNA targeting intron 1 of a human ALB gene can target at least 17, at least 18, at least 19, or at least 20 contiguous nucleotides of the guide RNA target sequence set forth in SEQ ID NO: 132.

As another example, a guide RNA targeting intron 1 of a human ALB gene can target the guide RNA target sequence set forth in SEQ ID NO: 126. As another example, a guide RNA targeting intron 1 of a human ALB gene can target at least 17, at least 18, at least 19, or at least 20 contiguous nucleotides of the guide RNA target sequence set forth in SEQ ID NO: 126.

As another example, a guide RNA targeting intron 1 of a human ALB gene can target the guide RNA target sequence set forth in SEQ ID NO: 129. As another example, a guide RNA targeting intron 1 of a human ALB gene can target at least 17, at least 18, at least 19, or at least 20 contiguous nucleotides of the guide RNA target sequence set forth in SEQ ID NO: 129.

As another example, a guide RNA targeting intron 1 of a human ALB gene can target the guide RNA target sequence set forth in SEQ ID NO: 137. As another example, a guide RNA targeting intron 1 of a human ALB gene can target at least 17, at least 18, at least 19, or at least 20 contiguous nucleotides of the guide RNA target sequence set forth in SEQ ID NO: 137.

TABLE 6
Human ALB Intron 1 Guide RNA Target Sequences.

| Guide RNA Target Sequence | SEQ ID NO: |
|---|---|
| GAGCAACCTCACTCTTGTCT | 126 |
| ATGCATTTGTTTCAAAATAT | 127 |
| TGCATTTGTTTCAAAATATT | 128 |
| ATTTATGAGATCAACAGCAC | 129 |
| GATCAACAGCACAGGTTTTG | 130 |
| TTAAATAAAGCATAGTGCAA | 131 |
| TAAAGCATAGTGCAATGGAT | 132 |
| TAGTGCAATGGATAGGTCTT | 133 |
| TACTAAAACTTTATTTTACT | 134 |
| AAAGTTGAACAATAGAAAAA | 135 |
| AATGCATAATCTAAGTCAAA | 136 |
| TAATAAAATTCAAACATCCT | 137 |
| GCATCTTTAAAGAATTATTT | 138 |
| TTTGGCATTTATTTCTAAAA | 139 |
| TGTATTTGTGAAGTCTTACA | 140 |
| TCCTAGGTAAAAAAAAAAAA | 141 |
| TAATTTTCTTTTGCGCACTA | 142 |
| TGACTGAAACTTCACAGAAT | 143 |
| GACTGAAACTTCACAGAATA | 144 |
| TTCATTTTAGTCTGTCTTCT | 145 |
| ATTATCTAAGTTTGAATATA | 146 |
| AATTTTTAAAATAGTATTCT | 147 |
| TGAATTATTCTTCTGTTTAA | 148 |
| ATCATCCTGAGTTTTTCTGT | 149 |
| TTACTAAAACTTTATTTTAC | 150 |
| ACCTTTTTTTTTTTTTACCT | 151 |
| AGTGCAATGGATAGGTCTTT | 152 |
| TGATTCCTACAGAAAAACTC | 153 |
| TGGGCAAGGGAAGAAAAAAA | 154 |
| CCTCACTCTTGTCTGGGCAA | 155 |
| ACCTCACTCTTGTCTGGGCA | 156 |
| TGAGCAACCTCACTCTTGTC | 157 |

TABLE 7
Mouse Alb Intron 1 Guide RNA Target Sequences.

| Guide RNA Target Sequence | SEQ ID NO: |
|---|---|
| CACTCTTGTCTGTGGAAACA | 165 |

### (5) Lipid Nanoparticles Comprising Nuclease Agents

Lipid nanoparticles comprising the nuclease agents (e.g., CRISPR/Cas systems) are also provided. The lipid nanoparticles can alternatively or additionally comprise a nucleic acid construct encoding a polypeptide of interest as disclosed herein. For example, the lipid nanoparticles can comprise a nuclease agent (e.g., CRISPR/Cas system), can comprise a nucleic acid construct encoding a polypeptide of interest, or can comprise both a nuclease agent (e.g., a CRISPR/Cas system) and a nucleic acid construct encoding a polypeptide of interest. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine or 1,2-distearoyl-sn-glycero-3-phosphocholine (DSPC). In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct encoding a polypeptide of interest as described elsewhere herein. In certain LNPs, the cargo can include an mRNA encoding a Cas nuclease, such as Cas9, a guide RNA or a nucleic acid encoding a guide RNA, and a nucleic acid construct encoding a polypeptide of. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMPE), or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%. The mol-% of the helper lipid may be, for example, from about 30 mol-% to about 60 mol-%. The mol-% of the neutral lipid may be, for example, from about 1 mol-% to about 20 mol-%. The mol-% of the stealth lipid may be, for example, from about 1 mol-% to about 10 mol-%

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100. The N/P ratio can also be from about 4 to about 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25. Alternatively, the LNP formulation can include a ratio of Cas mRNA to gRNA nucleic acid of from about 2:1 to about 1:2. In specific examples, the ratio of Cas mRNA to gRNA can be about 2:1.

In some LNPs, the cargo can comprise a nucleic acid construct encoding a polypeptide of interest and gRNA. The nucleic acid construct encoding a polypeptide of interest and gRNAs can be in different ratios. For example, the LNP formulation can include a ratio of nucleic acid construct to gRNA nucleic acid ranging from about 25:1 to about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020 (DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### (6) Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 158, SEQ ID NO: 159, or SEQ ID NO: 160. Other examples of ITRs comprise one or more mutations compared to SEQ ID NO: 158, SEQ ID NO: 159, or SEQ ID NO: 160 and can be at least 90%, at least 91%, at least 92%, at least 93%, at least 94%, at least 95%, at least 96%, at least 97%, at least 98%, or at least 99% identical to SEQ ID NO: 158, SEQ ID NO: 159, or SEQ ID NO: 160. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end, such as SEQ ID NO: 158 on the 5′ end and SEQ ID NO: 168 on the 3′ end, or SEQ ID NO: 159 on the 5′ end and SEQ ID NO: 597 on the 3′ end, or SEQ ID NO: 160 on the 5′ end and SEQ ID NO: 598 on the 3′ end). In one example, the ITR on each end can comprise, consist essentially of, or consist of SEQ ID NO: 158 (i.e., SEQ ID NO: 158 on the 5′ end, and the reverse complement on the 3′ end). In another example, the ITR on each end can comprise, consist essentially of, or consist of SEQ ID NO: 159 (i.e., SEQ ID NO: 159 on the 5′ end, and the reverse complement on the 3′ end). In one example, the ITR on at least one end comprises, consists essentially of, or consists of SEQ ID NO: 160. In one example, the ITR on the 5′ end comprises, consists essentially of, or consists of SEQ ID NO: 160. In one example, the ITR on the 3′ end comprises, consists essentially of, or consists of SEQ ID NO: 160. In one example, the ITR on each end can comprise, consist essentially of, or consist of SEQ ID NO: 160 (i.e., SEQ ID NO: 160 on the 5′ end, and the reverse complement on the 3′ end). In one example, the ITR on each end can comprise, consist essentially of, or consist of SEQ ID NO: 160. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 158, and the ITR on the other end comprises, consists essentially of, or consists of SEQ ID NO: 159. In another example, the ITR on one end comprises, consists essentially of, or consists of SEQ ID NO: 158, and the ITR on the other end comprises, consists essentially of, or consists of SEQ ID NO: 160. In one example, the ITR on one end comprises, consists essentially of, or consists of SEQ ID NO: 159, and the ITR on the other end comprises, consists essentially of, or consists of SEQ ID NO: 160.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include a nucleic acid construct encoding a polypeptide of interest. In certain AAVs, the cargo can include a nucleic acid (e.g., DNA) encoding a Cas nuclease, such as Cas9, a DNA encoding a guide RNA (or multiple guide RNAs), and a nucleic acid construct encoding a polypeptide of interest.

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### C. Cells or Animals or Genomes

Cells or animals (i.e., subjects) comprising any of the above compositions (e.g., nucleic acid construct encoding a polypeptide of interest, nuclease agents, vectors, lipid nanoparticles, or any combination thereof) are also provided herein. Such cells or animals (or genomes) can be produced by the methods disclosed herein. For example, the cells or animals can comprise any of the nucleic acid constructs encoding a polypeptide of interest described herein, any of the nuclease agents disclosed herein, or both. Such cells or animals (or genomes) can be neonatal cells or animals (or genomes). Alternatively, such cells or animals (or genomes) can be non-neonatal cells or animals (or genomes).

A neonatal subject (e.g., animal) can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals.

Neonatal cells can be cells of any neonatal subject. For example, they can be of a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals.

In some such cells or animals or genomes, the nucleic acid construct encoding a polypeptide of interest can be genomically integrated at a target genomic locus, such as a safe harbor locus (e.g., an ALB locus or a human ALB locus, such as intron 1 of an ALB locus or a human ALB locus). In some such cells, animals, or genomes, the polypeptide of interest encoded by the nucleic acid construct is expressed in the cell, animal, or genome. For example, if the nucleic acid construct encoding a polypeptide of interest is integrated into an ALB locus (e.g., intron 1 of a human ALB locus), the polypeptide of interest can be expressed from the ALB locus. The coding sequence for the polypeptide of interest can be operably linked to an endogenous promoter at the target genomic locus upon integration into the target genomic locus, or it can be operably linked to an exogenous promoter present in the nucleic acid construct. If the nucleic acid construct is a bidirectional nucleic acid construct disclosed herein, the neonatal genome, neonatal cell, or neonatal animal can express the first polypeptide of interest or can express the second polypeptide of interest. In some neonatal genomes, neonatal cells, or neonatal animals, the target genomic locus is an ALB locus. For example, the nucleic acid construct can be genomically integrated in intron 1 of the endogenous ALB locus. Endogenous ALB exon 1 can then splice into the coding sequence for the polypeptide of interest in the nucleic acid construct.

The target genomic locus at which the nucleic acid construct is stably integrated can be heterozygous for the nucleic acid construct encoding a polypeptide of interest or homozygous for the nucleic acid construct encoding a polypeptide of interest. A diploid organism has two alleles at each genetic locus. Each pair of alleles represents the genotype of a specific genetic locus. Genotypes are described as homozygous if there are two identical alleles at a particular locus and as heterozygous if the two alleles differ.

The cells, neonatal, or genomes can be from any suitable species, such as eukaryotic cells or eukaryotes, or mammalian cells or mammals (e.g., non-human mammalian cells or non-human mammals, or human cells or humans). A mammal can be, for example, a non-human mammal, a human, a rodent, a rat, a mouse, or a hamster. Other non-human mammals include, for example, non-human primates, e.g., monkeys and apes. The term “non-human” excludes humans. Examples include, but are not limited to, human cells/humans, rodent cells/rodents, mouse cells/mice, rat cells/rats, and non-human primate cells/non-human primates. In a specific example, the cell is a human cell or the animal is a human. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the neonatal cells can be liver cells, such as hepatocytes (e.g., mouse, non-human primate, or human hepatocytes).

The cells provided herein can be normal, healthy cells, or can be diseased or mutant-bearing cells. For example, the cells can have a deficiency of the polypeptide of interest or can be from a subject with deficiency of the polypeptide of interest. For example, the cells can have a GAA deficiency, can carry a mutation that results in a GAA deficiency, or can be from a subject with a GAA deficiency carrying a mutation that results in a GAA deficiency, or Pompe disease. In some embodiments, the cells are of a neonatal subject.

The cells provided herein can be dividing cells (e.g., actively dividing cells). Alternatively, the cells provided herein can be non-dividing cells.

### III. Therapeutic Methods and Methods for Introducing, Integrating, or Expressing a Nucleic Acid Encoding a Polypeptide of Interest in Cells or Subjects

The nucleic acid constructs and compositions disclosed herein can be used in methods of inserting or integrating a nucleic acid encoding a polypeptide of interest into a target genomic locus or methods of expressing a polypeptide of interest in a cell, in a population of cells, or in a subject (e.g., in a neonatal cell, in a population of neonatal cells, or in a neonatal subject).

The cells or populations of cells in the methods disclosed herein can be neonatal cells or populations of neonatal cells, and the subjects in the methods disclosed herein can be neonatal subjects in some methods. A neonatal subject can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject. In other methods, the cells or populations of cells are not neonatal cells and are not populations of neonatal cells, and the subjects are not neonatal subjects.

In one example, provided herein are methods of introducing a nucleic acid construct encoding a polypeptide of interest into a cell or a population of cells, such as a cell or a population of cells in a subject (e.g., neonatal cell or a population of neonatal cells, such as a neonatal cell or a population of neonatal cells in a neonatal subject). Such methods can comprise administering any of the nucleic acid constructs described herein (or any of the compositions comprising a nucleic acid construct described herein, including, for example, vectors or lipid nanoparticles) to the cell, the population of cells, or the subject (e.g., the neonatal cell, the population of neonatal cells, or the neonatal subject). In some methods, the nucleic acid construct or composition comprising the nucleic acid construct can be administered together with a nuclease agent (simultaneously or sequentially in any order) described herein. The nuclease agent can cleave a nuclease target sequence within a target genomic locus (e.g., target gene) (e.g., to create a cleavage site), and the nucleic acid construct can be inserted into the target genomic locus (e.g., into the cleavage site) to create a modified target genomic locus. The polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence (e.g., to create a cleavage site), the nucleic acid construct can be inserted into ALB intron 1 (e.g., into the cleavage site) to create a modified ALB gene, and polypeptide of interest can be expressed from the modified ALB gene.

In one example, provided herein are methods of inserting a nucleic acid construct encoding a polypeptide of interest into a target genomic locus in a cell or a population of cells, such as a cell or a population of cells in a subject (e.g., in a neonatal cell or a population of neonatal cells, such as a neonatal cell or a population of neonatal cells in a neonatal subject). Such methods can comprise administering any of the nucleic acid constructs described herein (or any of the compositions comprising a nucleic acid construct described herein, including, for example, vectors or lipid nanoparticles) to the cell, the population of cells, or the subject (e.g., the neonatal cell, the population of neonatal cells, or the neonatal subject). In some methods, the nucleic acid construct or composition comprising the nucleic acid construct can be administered together with a nuclease agent (simultaneously or sequentially in any order) described herein. The nuclease agent can cleave a nuclease target sequence within a target genomic locus (e.g., target gene) (e.g., to create a cleavage site), and the nucleic acid construct can be inserted into the target genomic locus (e.g., into the cleavage site) to create a modified target genomic locus. The polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence (e.g., to create a cleavage site), the nucleic acid construct can be inserted into ALB intron 1 (e.g., into the cleavage site) to create a modified ALB gene, and polypeptide of interest can be expressed from the modified ALB gene.

In another example, provided herein are methods of expressing a polypeptide of interest from a target genomic locus in a cell, a population of cells, or a subject (e.g., in a neonatal cell, a population of neonatal cells, or a neonatal subject). Such methods can comprise administering any of the nucleic acid constructs described herein (or any of the compositions comprising a nucleic acid construct described herein, including, for example, vectors or lipid nanoparticles) to the cell, the population of cells, or the subject (e.g., to the neonatal cell, the population of neonatal cells, or the neonatal subject). In some methods, the nucleic acid construct can be administered together (simultaneously or sequentially in any order) with a nuclease agent described herein. The nuclease agent can cleave a nuclease target sequence within a target genomic locus (e.g., target gene) (e.g., to create a cleavage site), the nucleic acid construct can be inserted into the target genomic locus (e.g., into the cleavage site) to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence (e.g., to create a cleavage site), the nucleic acid construct can be inserted into the target genomic locus (e.g., into the cleavage site) to create a modified ALB gene, and the polypeptide of interest can be expressed from the modified ALB gene.

In some methods, the subject comprises a mutation in a genome in the subject, wherein the mutation results in reduced activity or expression of an endogenous polypeptide having enzymatic activity. In some methods, the nucleic acid encoding the polypeptide of interest encodes a polypeptide having the enzymatic activity of a wild type polypeptide encoded by the gene in which the subject has a mutation that results in reduced activity or expression of the endogenous polypeptide.

In any of the above methods, the cells (e.g., neonatal cells) can be from any suitable species, such as eukaryotic cells or mammalian cells (e.g., non-human mammalian cells or human cells). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples of cells (e.g., neonatal cells) include, but are not limited to, human cells, rodent cells, mouse cells, rat cells, and non-human primate cells. In a specific example, the cell (e.g., neonatal cell) is a human cell. Likewise, cells (e.g., neonatal cells) can be any suitable type of cell. In a specific example, the cell (e.g., neonatal cell) is a liver cell such as a hepatocyte (e.g., a human liver cell or human hepatocyte).

The cells (e.g., neonatal cells) can be isolated cells (e.g., in vitro), ex vivo cells, or can be in vivo within an animal (i.e., in a subject or a neonatal subject). In a specific example, the cell or neonatal cell is in vivo (in a subject or neonatal subject). Similarly, the cells or neonatal cells can also be primary somatic cells or cells that are not a primary somatic cell. Somatic cells include any cell that is not a gamete, germ cell, gametocyte, or undifferentiated stem cell. For example, the neonatal cells can be liver cells, such as hepatocytes (e.g., mouse, non-human primate, or human hepatocytes).

The cells (e.g., neonatal cells) provided herein can be normal, healthy cells, or can be diseased or mutant-bearing cells. In certain embodiments, the cells may demonstrate a loss of function, e.g., a loss of enzyme function.

The nucleic acid constructs and compositions disclosed herein can also be used in methods of treating an enzyme deficiency and methods of treating a lysosomal storage disease in a subject (e.g., a neonatal subject). The nucleic acid constructs and compositions disclosed herein can also be used in methods of preventing or reducing the onset of a sign or symptom of an enzyme deficiency and or a lysosomal storage disease in a subject (e.g., a neonatal subject).

The nucleic acid constructs and compositions disclosed herein can also be used in methods of treating a genetic disease that can be detected, including those that are routinely screened for, in newborn screening in a subject (e.g., a neonatal subject). The nucleic acid constructs and compositions disclosed herein can also be used in methods of preventing or reducing the onset of a sign or symptom of such diseases in a subject (e.g., a neonatal subject).

The nucleic acid constructs and compositions disclosed herein can also be used in methods of treating inborn errors of metabolism in a subject (e.g., a neonatal subject). The nucleic acid constructs and compositions disclosed herein can also be used in methods of preventing or reducing the onset of a sign or symptom of diseases associated with inborn errors of metabolism in a subject (e.g., a neonatal subject).

The nucleic acid constructs and compositions disclosed herein can also be used in methods of treating bleeding disorders in a subject (e.g., a neonatal subject). The nucleic acid constructs and compositions disclosed herein can also be used in methods of preventing or reducing the onset of a sign or symptom of bleeding disorders in a subject (e.g., a neonatal subject).

The compositions disclosed herein (e.g., nucleic acid constructs encoding a polypeptide of interest, or nucleic acid constructs in combination with the nuclease agents (e.g., CRISPR/Cas systems) are useful for the treatment of enzyme deficiencies or lysosomal storage diseases and/or ameliorating at least one symptom associated with enzyme deficiencies or lysosomal storage diseases. Likewise, the compositions disclosed herein can be used for the preparation of a pharmaceutical composition or medicament for treating a subject (e.g., a neonatal subject) having an enzyme deficiency or lysosomal storage disease. The terms “treat,” “treated,” “treating,” and “treatment,” include the administration of the nucleic acid constructs disclosed herein (e.g., together with a nuclease agent disclosed herein) to subjects to prevent or delay the onset of the symptoms, complications, or biochemical indicia of a disease, alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease. It is understood that a number of lysosomal storage diseases or inborn diseases of metabolism are possible to diagnose before the presence of symptoms, or diagnosed through routine newborn screening programs, including pilot programs. Some include diagnosis based on the presence of a biomarker, e.g., a metabolite or enzyme in a subject sample, e.g., a blood or urine sample. In some embodiments, diagnosis is confirmed by genetic analysis for the presence of genetic mutations associated with the disease. As used herein, treatment includes treatments with the compositions and methods provided herein to a subject who meets diagnostic criteria of the presence, or absence, of a biomarker, either alone or in combination with a genetic diagnosis, prior to the development of signs or symptoms of the disease.

Enzyme-deficiency diseases that can be treated include non-lysosomal storage disease such as Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, argininosuccinic aciduria, methylmalonic aciduria, type I citrullinemia/argininosuccinate synthetase deficiency, carbamoyl-phosphate synthase 1 deficiency, propionic acidemia, isovaleric acidemia, glutaric academia I, progressive familial intrahepatic cholestasis, types 2 and 3, and lysosomal storage diseases. An enzyme deficiency refers expression and/or activity levels of the enzyme being lower in the subject (e.g., neonatal subject) than normal enzyme expression and/or activity levels, such that the normal functions of the enzyme are not fully carried out in the subject. Routine and pilot newborn screening programs are in place for many enzyme deficiency diseases as treatment is often most effective when started as soon after birth as possible. Screening can be performed on different subject samples depending on the screening test, e.g., urine, dried blood spot. Some preliminary screening tests require follow up analysis to confirm a diagnosis, e.g., genetic sequencing. Such screening and diagnostic methods are well known in the art. Sequencing may indicate a later onset form of the disease that may be managed by screening and delayed intervention at the discretion of a health care professional. As used herein, a subject is considered to have an enzyme deficiency disease if the subject has required signs indicative of the deficiency, e.g., reduced activity level, the presence or absence of a metabolite indicating the presence of disease, or mutations demonstrated by genetic sequencing, prior to the presence of symptoms of the disease, e.g., muscle weakness, failure to thrive. Therefore, administration of the compositions provided herein is understood as treatment of the disease.

Lysosomal storage diseases include any disorder resulting from a defect in lysosome function. Currently, approximately fifty lysosomal storage disorders have been identified, the most well-known of which include Tay-Sachs, Gaucher, and Niemann-Pick disease. The pathogeneses of the diseases are ascribed to the buildup of incomplete degradation products in the lysosome, usually due to loss of protein function. Lysosomal storage diseases are caused by loss-of-function or attenuating variants in the proteins whose normal function is to degrade or coordinate degradation of lysosomal contents. The proteins affiliated with lysosomal storage diseases include enzymes, receptors and other transmembrane proteins (e.g., NPC1), post-translational modifying proteins (e.g., sulfatase), membrane transport proteins, and non-enzymatic cofactors and other soluble proteins (e.g., GM2 ganglioside activator). Thus, lysosomal storage diseases encompass more than those disorders caused by defective enzymes per se, and include any disorder caused by any molecular defect. Thus, as used herein, the term “enzyme” is meant to encompass those other proteins associated with lysosomal storage diseases.

Lysosomal storage diseases are a class of rare diseases that affect the degradation of myriad substrates in the lysosome. Those substrates include sphingolipids, mucopolysaccharides, glycoproteins, glycogen, and oligosaccharides, which can accumulate in the cells of those with disease leading to cell death. Organs affected by lysosomal storage diseases include the central nervous system (CNS), the peripheral nervous system (PNS), lungs, liver, bone, skeletal and cardiac muscle, and the reticuloendothelial system.

Lysosomal storage diseases include sphingolipidoses, a mucopolysaccharidoses, and glycogen storage diseases. In some embodiments, the lysosomal storage disease is any one or more of Fabry disease, Gaucher disease type I, Gaucher disease type II, Gaucher disease type III, Niemann-Pick disease type A, Niemann-Pick disease type BGM1-gangliosidosis, Sandhoff disease, Tay-Sachs disease, GM2-activator deficiency, GM3-gangliosidosis, metachromatic leukodystrophy, sphingolipid-activator deficiency, Scheie disease, Hurler-Scheie disease, Hurler disease, Hunter disease, Sanfilippo A, Sanfilippo B, Sanfilippo C, Sanfilippo D, Morquio syndrome A, Morquio syndrome B, Maroteaux-Lamy disease, Sly disease, MPS IX, and Pompe disease. Enzymes (which include proteins that are not per se catalytic) associated with lysosomal storage diseases include for example any and all hydrolases, α-galactosidase, β-galactosidase, α-glucosidase, β-glucosidase, saposin-C activator, ceramidase, sphingomyelinase, β-hexosaminidase, GM2 activator, GM3 synthase, arylsulfatase, sphingolipid activator, α-iduronidase, iduronidase-2-sulfatase, heparin N-sulfatase, N-acetyl-α-glucosaminidase, α-glucosamide N-acetyltransferase, N-acetylglucosamine-6-sulfatase, N-acetylgalactosamine-6-sulfate sulfatase, N-acetylgalactosamine-4-sulfatase, β-glucuronidase, hyaluronidase, and the like.

The nature of the molecular lesion affects the severity of the disease in many cases. Complete loss-of-function tends to be associated with prenatal or neonatal onset, and involves severe symptoms; partial loss-of-function is associated with milder (relatively) and later-onset disease. Generally, only a small percentage of activity needs to be restored to have to correct metabolic defects in deficient cells. Table 8 lists some of the more common lysosomal storage diseases and their associated loss-of-function proteins.

TABLE 8
Lysosomal Storage Diseases.

| | | Involved |
|---|---|---|
| Class | Disease | Enzyme/Protein |
| Sphingo- | Fabry disease | α-Galactosidase A |
| lipidoses | Farber lipogranulomatosis | Ceramidase |
| | Gaucher disease type I | β-Glucosidase |
| | Gaucher disease | Saposin-C activator |
| | types II and III | |
| | Niemann-Pick diseases | Sphingomyelinase |
| | types A and B | |
| | GM1-gangliosidosis | β-Galactosidase |
| | GM2-gangliosidosis | β-Hexosaminidase |
| | (Sandhoff) | A and B |
| | GM2-gangliosidosis | β-Hexosaminidase A |
| | (Tay-Sachs) | |
| | GM2-gangliosidosis | GM2-activator protein |
| | (GM2-activator deficiency) | |
| | GM3-gangliosidosis | GM3 synthase |
| | Metachromatic leukodystrophy | Arylsulfatase A |
| | Sphingolipid-activator | Sphingolipid activator |
| | deficiency | |
| Mucopoly- | MPS I (Scheie, Hurler-Scheie, | α-Iduronidase |
| saccharidoses | and Hurler disease) | |
| | MPS II (Hunter) | Iduronidase-2-sulfatase |
| | MPS IIIA (Sanfilippo A) | Heparan N-sulfatase |
| | MPS IIIB (Sanfilippo B) | N-acetyl-α- |
| | | glucosaminidase |
| | MPS IIIC (Sanfilippo C) | Acetyl-CoA; |
| | | α-glucosamide |
| | | N-acetyltransferase |
| | MPS IIID (Sanfilippo D) | N-acetylglucosamine- |
| | | 6-sulfatase |
| | MPS IVA | N-acetylgalactosamine- |
| | (Morquio syndrome A) | 6-sulfate sulfatase |
| | MPS IVB | β-Galactosidase |
| | (Morquio syndrome B) | |
| | MPS VI (Maroteaux-Lamy) | N-acetylgalactosamine- |
| | | 4-sulfatase |
| | | (arylsulfatase B) |
| | MPS VII (Sly disease) | β-Glucuronidase |
| | MPS IX | Hyaluronidase |
| Glycogen | Pompe (glycogen | α-Glucosidase 2 |
| storage | storage disease type II) | |
| disease | | |
| Lipid | Lysosomal acid | Lysosomal acid lipase |
| metabolism | lipase deficiency | |
| | (LAL-D; Wolman disease) | |

Lysosomal storage diseases can be categorized according to the type of product that accumulates within the defective lysosome. Sphingolipidoses are a class of diseases that affect the metabolism of sphingolipids, which are lipids containing fatty acids linked to aliphatic amino alcohols. The accumulated products of sphingolipidoses include gangliosides (e.g., Tay-Sachs disease), glycolipids (e.g., Fabry's disease), and glucocerebrosides (e.g., Gaucher's disease).

Mucopolysaccharidoses are a group of diseases that affect the metabolism of glycosaminoglycans (GAGS or mucopolysaccharides), which are long unbranched chains of repeating disaccharides that help build bone, cartilage, tendons, corneas, skin and connective tissue. The accumulated products of mucopolysaccharidoses include heparan sulfate, dermatan sulfate, keratin sulfate, various forms of chondroitin sulfate, and hyaluronic acid. For example, Morquio syndrome A is due to a defect in the lysosomal enzyme galactose-6-sulfate sulfatase, which results in the lysosomal accumulation of keratin sulfate and chondroitin 6-sulfate.

Glycogen storage diseases result from a cell's inability to metabolize (make or break-down) glycogen. Glycogen metabolism is moderated by various enzymes or other proteins including glucose-6-phosphatase, acid alpha-glucosidase, glycogen de-branching enzyme, glycogen branching enzyme, muscle glycogen phosphorylase, liver glycogen phosphorylase, muscle phosphofructokinase, phosphorylase kinase, glucose transporter, aldolase A, beta-enolase, and glycogen synthase. An exemplar lysosomal storage/glycogen storage disease is Pompe disease, in which defective acid alpha-glucosidase causes glycogen to accumulate in lysosomes. Symptoms include hepatomegaly, muscle weakness, heart failure, and in the case of the infantile variant, death by age two.

Provided herein are methods of treating an enzyme deficiency (e.g., bleeding disorder, inborn error of metabolism, e.g., lysosomal storage disease) in a subject in need thereof (e.g., a neonatal subject). The lysosomal storage disease can be any type of lysosomal storage disease. Examples of lysosomal storage disease is described in more detail elsewhere herein. Such methods can comprise administering any of the nucleic acid constructs described herein (or any of the compositions comprising a nucleic acid construct described herein, including, for example, vectors or lipid nanoparticles) to the subject such that a therapeutically effective level of polypeptide of interest expression or a therapeutically effective level of circulating polypeptide of interest is achieved in the subject, for example, wherein the polypeptide of interest is the enzyme in the enzyme deficiency or an enzyme having the same activity as the enzyme in the enzyme deficiency. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The polypeptide of interest coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and polypeptide of interest can be expressed from the modified ALB gene (e.g., such that a therapeutically effective level of polypeptide of interest expression or a therapeutically effective level of circulating polypeptide of interest is achieved in the subject).

Also provided are methods of treating a lysosomal storage disease, for example, in a subject in need thereof (e.g., a neonatal subject). The lysosomal storage disease can be any type of lysosomal storage disease. Examples of lysosomal storage disease are described in more detail elsewhere herein. Such methods can comprise administering any of the nucleic acid constructs described herein (or any of the compositions comprising a nucleic acid construct described herein, including, for example, vectors or lipid nanoparticles) to the subject such that a therapeutically effective level of, for example, polypeptide of interest expression or a therapeutically effective level of circulating polypeptide of interest is achieved in the subject, or a polypeptide having the same activity as the polypeptide of interest, wherein the lysosomal storage disease is characterized by loss-of-function of the polypeptide of interest. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The polypeptide of interest coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and polypeptide of interest can be expressed from the modified ALB gene (e.g., such that a therapeutically effective level of polypeptide of interest expression or a therapeutically effective level of circulating polypeptide of interest is achieved in the subject).

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of a lysosomal storage disease may comprise alleviating symptoms of the lysosomal storage. Lysosomal storage diseases are described in detail above and can refer to a disorder caused by a missing or defective gene or polypeptide.

Also provided are methods of preventing or reducing the onset of a sign or symptom of an enzyme deficiency in a subject (e.g., a neonatal subject) in need thereof (e.g., a subject with a lysosomal storage disease characterized by the enzyme deficiency). By preventing is meant the sign or symptom of the enzyme deficiency never becomes present. For example, the methods can prevent or reduce the onset of a sign or symptom of an enzyme deficiency compared to an untreated control subject. The lysosomal storage disease can be any type of lysosomal storage disease. Examples of lysosomal storage disease is described in more detail elsewhere herein. Such methods can comprise administering any of the nucleic acid constructs described herein (or any of the compositions comprising a nucleic acid construct described herein, including, for example, vectors or lipid nanoparticles) to the subject such that a therapeutically effective level of polypeptide of interest expression or a therapeutically effective level of circulating polypeptide of interest is achieved in the subject, wherein the polypeptide of interest is the enzyme in the enzyme deficiency. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The polypeptide of interest coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and polypeptide of interest can be expressed from the modified ALB gene (e.g., such that a therapeutically effective level of polypeptide of interest expression or a therapeutically effective level of circulating polypeptide of interest is achieved in the subject

Also provided are methods of preventing or reducing the onset of a sign or symptom of a lysosomal storage disease in a subject (e.g., a neonatal subject) in need thereof. By preventing is meant the sign or symptom of the lysosomal storage disease never becomes present. For example, the methods can prevent or reduce the onset of a sign or symptom of a lysosomal storage disease compared to an untreated control subject. The lysosomal storage disease can be any type of lysosomal storage disease. Examples of lysosomal storage disease is described in more detail elsewhere herein. Such methods can comprise administering any of the nucleic acid constructs described herein (or any of the compositions comprising a nucleic acid construct described herein, including, for example, vectors or lipid nanoparticles) to the subject such that a therapeutically effective level of polypeptide of interest expression or a therapeutically effective level of circulating polypeptide of interest is achieved in the subject, wherein the polypeptide of interest is the enzyme in the enzyme deficiency. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The polypeptide of interest coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and polypeptide of interest can be expressed from the modified ALB gene (e.g., such that a therapeutically effective level of polypeptide of interest expression or a therapeutically effective level of circulating polypeptide of interest is achieved in the subject

In some methods, a therapeutically effective amount of the nucleic acid construct or the composition comprising the nucleic acid construct or the combination of the nucleic acid construct and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

The subject (e.g., neonatal subject) in any of the above methods can be from any suitable species, such as a eukaryote or a mammal. A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples of suitable species include, but are not limited to, humans, rodents, mice, rats, and non-human primates. In a specific example, the subject or neonatal subject is a human.

Any target genomic locus capable of expressing a gene can be used in the methods described herein, such as a safe harbor locus (safe harbor gene). Such loci are described in more detail elsewhere herein. In a specific example, the target genomic locus can be an endogenous ALB locus, such as an endogenous human ALB locus. For example, the nucleic acid construct can be genomically integrated in intron 1 of the endogenous ALB locus. Endogenous ALB exon 1 can then splice into the coding sequence for the polypeptide of interest in the nucleic acid construct.

Targeted insertion of the nucleic acid construct comprising the polypeptide of interest coding sequence into a target genomic locus, and particularly an endogenous ALB locus, offers multiple advantages. Such methods result in stable modification to allow for stable, long-term expression of the polypeptide of interest. With respect to the ALB locus, such methods are able to utilize the endogenous ALB promoter and regulatory regions to achieve therapeutically effective levels of expression. For example, the coding sequence for the polypeptide of interest in the nucleic acid construct can comprise a promoterless gene, and the inserted nucleic acid construct can be operably linked to an endogenous promoter in the target genomic locus (e.g., ALB locus). Use of an endogenous promoter is advantageous because it obviates the need for inclusion of a promoter in the nucleic acid construct, allowing packaging of larger transgenes that may not normally package efficiently (e.g., in AAV). Alternatively, the coding sequence in the nucleic acid construct can be operably linked to an exogenous promoter in the nucleic acid construct. Examples of types of promoters that can be used are disclosed elsewhere herein.

Optionally, some or all of the endogenous gene (e.g., endogenous ALB gene) at the target genomic locus can be expressed upon insertion of the coding sequence for the polypeptide of interest from the nucleic acid construct. Alternatively, in some methods, none of the endogenous gene at the target genomic locus is expressed. As one example, the modified target genomic locus (e.g., modified ALB locus) after integration of the nucleic acid construct can encode a chimeric protein comprising an endogenous secretion signal (e.g., albumin secretion signal) and the polypeptide of interest encoded by the nucleic acid construct. In another example, the first intron of an ALB locus can be targeted. The secretion signal peptide of ALB is encoded by exon 1 of the ALB gene. In such a scenario, a promoterless cassette bearing a splice acceptor and the polypeptide of interest coding sequence will support expression and secretion of the polypeptide of interest. Splicing between endogenous ALB exon 1 and the integrated coding sequence for the polypeptide of interest creates a chimeric mRNA and protein including the endogenous ALB sequence encoded by exon 1 operably linked to the polypeptide of interest encoded by the integrated nucleic acid construct.

The nucleic acid construct can be inserted into the target genomic locus by any means, including homologous recombination (HR) and non-homologous end joining (NHEJ) as described elsewhere herein. In a specific example, the nucleic acid construct is inserted by NHEJ (e.g., does not comprise a homology arm and is inserted by NHEJ).

In another specific example, the nucleic acid construct can be inserted via homology-independent targeted integration (e.g., directional homology-independent targeted integration). For example, the coding sequence for the polypeptide of interest in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the polypeptide of interest coding sequence. In a specific example, the nucleic acid construct is delivered AAV-mediated delivery, and cleavage of the target sites flanking the polypeptide of interest coding sequence can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the polypeptide of interest coding sequence is inserted into the target genomic locus in the correct orientation but it is reformed if the polypeptide of interest coding sequence is inserted into the target genomic locus in the opposite orientation. This can help ensure that the polypeptide of interest coding sequence is inserted in the correct orientation for expression.

In any of the above methods, the nucleic acid construct encoding the polypeptide of interest can be administered simultaneously with the nuclease agent (e.g., CRISPR/Cas system) or not simultaneously (e.g., sequentially in any combination). For example, in a method comprising administering a composition comprising the nucleic acid construct and a nuclease agent, they can be administered separately. For example, the nucleic acid construct can be administered prior to the nuclease agent, subsequent to the nuclease agent, or at the same time as the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

Any suitable methods of administering nucleic acid constructs and nuclease agents to cells can be used, particularly methods of administering to the liver, and examples of such methods are described in more detail elsewhere herein. In methods of treatment or in methods of targeting a cell (e.g., neonatal cell) in vivo in a subject (e.g., neonatal subject), the nucleic acid construct can be inserted in particular types of cells in the subject. The method and vehicle for introducing the nucleic acid construct and/or the nuclease agent into the subject can affect which types of cells in the subject are targeted. In some methods, for example, the nucleic acid construct is inserted into a target genomic locus (e.g., an endogenous ALB locus) in liver cells, such as hepatocytes. Methods and vehicles for introducing such constructs and nuclease agents into the subject or neonatal subject (including methods and vehicles that target the liver or hepatocytes, such as lipid nanoparticle-mediated delivery and AAV-mediated delivery (e.g., rAAV8-mediated delivery) and intravenous injection), are disclosed in more detail elsewhere herein.

In any of the above methods, the nucleic acid construct and the nuclease agent (e.g., CRISPR/Cas system) can be administered using any suitable delivery system and known method. The nuclease agent components and nucleic acid construct (e.g., the guide RNA, Cas protein, and nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which a nucleic acid construct is inserted following cleavage by a gene-editing system (e.g., a Cas protein), the gene-editing system (e.g., Cas protein) can cleave the target genomic locus to create a single-strand break (nick) or double-strand break, and the cleaved or nicked locus can be repaired by insertion of the nucleic acid construct via non-homologous end joining (NHEJ)-mediated insertion or homology-directed repair. Optionally, repair with the nucleic acid construct removes or disrupts the guide RNA target sequence(s) so that alleles that have been targeted cannot be re-targeted by the CRISPR/Cas reagents.

As explained in more detail elsewhere herein, the nucleic acid constructs can comprise deoxyribonucleic acid (DNA) or ribonucleic acid (RNA), they can be single-stranded or double-stranded, and they can be in linear or circular form. The nucleic acid constructs can be naked nucleic acids or can be delivered by viruses, such as AAV. In a specific example, the nucleic acid construct can be delivered via AAV and can be capable of insertion into the target genomic locus (e.g., a safe harbor gene, an ALB gene, or intron 1 of an ALB gene) by non-homologous end joining (e.g., the nucleic acid construct can be one that does not comprise a homology arm).

Some nucleic acid constructs are capable of insertion by non-homologous end joining. In some cases, such nucleic acid constructs do not comprise a homology arm. For example, such nucleic acid constructs can be inserted into a blunt end double-strand break following cleavage with a Cas protein. In a specific example, the nucleic acid construct can be delivered via AAV and can be capable of insertion by non-homologous end joining (e.g., the nucleic acid construct can be one that does not comprise a homology arm).

In another example, the nucleic acid construct can be inserted via homology-independent targeted integration. For example, the nucleic acid construct can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid insert. In a specific example, the nucleic acid construct is delivered AAV-mediated delivery, and cleavage of the target sites flanking the nucleic acid insert can remove the inverted terminal repeats (ITRs) of the AAV. In some methods, the target site in the target genomic locus (e.g., a guide RNA target sequence including the flanking protospacer adjacent motif) is no longer present if the nucleic acid insert is inserted into the target genomic locus in the correct orientation but it is reformed if the nucleic acid insert is inserted into the target genomic locus in the opposite orientation.

The methods disclosed herein can comprise introducing or administering into a subject or neonatal subject (e.g., an animal or mammal, such as a human) or cell or neonatal cell a nucleic acid construct encoding a polypeptide of interest and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, a nucleic acid construct can be introduced prior to the introduction of a Cas protein and a guide RNA, or it can be introduced following introduction of the Cas protein and the guide RNA (e.g., the nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery. In preferred embodiments, promotors are accepted by regulatory authorities for use in humans. In certain embodiments, promotors drive expression in a liver cell.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct. In certain LNPs, the cargo can include an mRNA encoding a Cas nuclease, such as Cas9, a guide RNA or a nucleic acid encoding a guide RNA, and a nucleic acid construct. LNPs for use in the methods are described in more detail elsewhere herein.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or nucleic acid construct encoding a polypeptide of interest). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, systemic routes of administration such as parenteral administration, e.g., intravenous, subcutaneous, intra-arterial, or intramuscular. In a specific example, administration in vivo is intravenous.

Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The methods disclosed herein can increase polypeptide of interest levels and/or polypeptide of interest activity levels in a cell or neonatal cell or subject or neonatal subject (e.g., circulating, serum, or plasma levels in a subject or neonatal subject) and can comprise measuring polypeptide of interest levels and/or activity levels in a cell or neonatal cell or subject or neonatal subject (e.g., circulating, serum, or plasma levels in a subject or neonatal subject). In one example, the effectiveness of the treatment in a subject can be assessed by measuring serum or plasma polypeptide of interest activity, wherein an increase in the subject's or neonatal subject's plasma level and/or activity of polypeptide of interest indicates effectiveness of the treatment.

In some methods, the subject (e.g., neonatal subject) is a subject (e.g., neonatal subject) with a polypeptide of interest deficiency such that expression and/or activity levels of the polypeptide of interest are lower in the subject (e.g., neonatal subject) than normal polypeptide of interest expression and/or activity levels.

In some methods, polypeptide of interest activity and/or expression levels (e.g., plasma or serum levels) in a subject (e.g., neonatal subject) are increased to about or at least about 2%, about or at least about 10%, about or at least about 25%, about or at least about 50%, about or at least about 75%, or at least about 100%, or more, of normal level. In certain embodiments, the level of expression or activity is measured in a cell or tissue in which a sign or symptom of the loss of function is present. For example, when the loss of function results in muscle dysfunction, the level or activity of the polypeptide of interest is measured in a muscle cell. It is understood that depending on the exogenous protein, the level of activity of the exogenous protein may not compare 1:1 with a native protein based on weight. In such embodiment, the relative activity of the exogenous protein and the native protein can be compared. In certain embodiments, the loss of function is nearly complete such that a relative activity cannot be determined. In certain embodiments, the comparison is made to an appropriate control subject. Selection of an appropriate control subject is within the ability of those of skill in the art. In certain embodiments, the level of expression is sufficient to treat at least one sign or symptom resulting from the loss of function of the protein.

In some methods, the method increases expression and/or activity of the polypeptide of interest over the subject's baseline expression and/or activity (i.e., expression and/or activity prior to administration). In some methods, polypeptide of interest activity and/or expression levels (e.g., plasma or serum levels) in a subject (e.g., neonatal subject) are increased by about or at least about 10%, about or at least about 25%, about or at least about 50%, about or at least about 75%, or about or at least about 100%, or more, as compared to the subject's polypeptide of interest activity and/or expression levels (e.g., plasma or serum levels) before administration (i.e., the subject's baseline levels). It is understood that depending on the exogenous protein, the level of activity of the exogenous protein may not compare 1:1 with a native protein based on weight. In such embodiment, the relative activity of the exogenous protein and the native protein can be compared. In certain embodiments, the loss of function is nearly complete such that a relative activity cannot be determined. In certain embodiments, the level of expression is sufficient to treat at least one sign or symptom resulting from the loss of function of the protein.

In some methods, the method increases expression and/or activity of the polypeptide of interest over the cell's or population's baseline expression and/or activity (i.e., expression and/or activity prior to administration). In some methods, polypeptide of interest activity and/or expression levels (e.g., protein levels) in a cell or neonatal cell or population of cells or neonatal cells (e.g., liver cells, or hepatocytes) are increased by about or at least about 10%, about or at least about 25%, about or at least about 50%, about or at least about 75%, about or at least about 100%, or more, as compared to the polypeptide of interest activity and/or protein levels before administration. It is understood that depending on the exogenous protein, the level of activity of the exogenous protein may not compare 1:1 with a native protein based on weight. In such embodiment, the relative activity of the exogenous protein and the native protein can be compared. In certain embodiments, the loss of function is nearly complete such that a relative activity cannot be determined. In certain embodiments, the level of expression is sufficient to treat at least one sign or symptom resulting from the loss of function of the protein.

Some methods comprise expressing a therapeutically effective amount of the polypeptide of interest (e.g., achieving a therapeutically effective level of circulating polypeptide of interest activity in an individual). The specific level of expression required depends, for example, on the degree of the loss of function, e.g., partial or complete, and the particular disease or condition to be treated, e.g., what percent of normal activity is required for the deficiency to not manifest signs or symptoms of the disease. Methods to diagnose and monitor diseases and conditions related to loss of function, diseases related to enzyme deficiencies, are known in the art. Some methods comprise achieving polypeptide of interest activity or expression levels of at least about 5% to about 50% of normal or at least about 50% to about 150% of normal.

In a specific example, the activity level of the plasma or serum polypeptide of interest levels in a subject (e.g., neonatal subject) are increased to about 5% to about 200% of normal plasma or serum polypeptide of interest activity levels (e.g., to or about 100% of normal plasma polypeptide of interest levels).

In a specific example, the polypeptide of interest activity levels in a subject (e.g., neonatal subject) are increased to no more than about 300%, no more than about 250%, no more than about 200%, or no more than about 150% of normal polypeptide of interest activity levels. In a specific example, the plasma polypeptide of interest levels in a subject are increased to no more than about 300%, no more than about 250%, no more than about 200%, or no more than about 150% of normal plasma polypeptide of interest levels.

In some methods, the method results in increased expression of the polypeptide of interest in the subject (e.g., neonatal subject) compared to a method comprising administering an episomal expression vector encoding the polypeptide of interest in a control subject. In some methods, the method results in increased serum levels of the polypeptide of interest in the subject (e.g., neonatal subject) compared to a method comprising administering an episomal expression vector encoding the polypeptide of interest to a control subject.

In some methods in which the subject did not express the polypeptide of interest prior to treatment, the method results in expression of the polypeptide of interest at a detectable level above zero, e.g., at a statistically significant level, a clinically relevant level.

Some methods comprise achieving a durable or sustained effect in a human, such as an at least at least 8 weeks, at least 24 weeks, for example, at least 1 year (52 weeks), or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased polypeptide of interest activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of polypeptide of interest in a human is achieved by at least 7 days, at least 14 days, or at least 28 days, optionally at least 56 days, at least 80 days, or at least 96 days. In additional methods, the method comprises maintaining polypeptide of interest activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the polypeptide of interest can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the polypeptide of interest can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the polypeptide of interest is maintained at a level higher than the expression or activity of the polypeptide of interest prior to treatment (i.e., the subject's baseline). In some methods, expression or activity of the polypeptide of interest is considered sustained if it is maintained at a therapeutically effective level of expression or activity. Relative durations, in other organisms, are understood based, e.g., on life span and developmental stages, are covered within the disclosure above. In some methods, expression or activity of the polypeptide of interest is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the polypeptide, e.g., weekly, monthly, particularly early after administration, e.g., within the first six months. Periodic measurements may establish that the effect on expression or activity is sustained at, e.g. 6 months after administration, one year after administration, or two years after administration. In some methods in neonatal subjects, the expression of the polypeptide of interest is sustained when the neonatal subject becomes an adult. In some methods, the expression of the polypeptide of interest is sustained for the lifetime of the subject or neonatal subject.

In some methods, the expression or activity of the polypeptide of interest is at least 50% of the expression or activity of the polypeptide at a peak level of expression measured for the human subject at 24 weeks after the administering. In some methods, the expression or activity of the polypeptide of interest is at least 50% of the expression or activity of the polypeptide at a peak level of expression measured for the human subject at one year after the administering. In some methods, the expression or activity of the polypeptide of interest is at least 60% of the expression or activity of the polypeptide at a peak level of expression measured for the human subject at 24 weeks after the administering. In some methods, expression or activity of the polypeptide of interest is at least 50% of the expression or activity of the polypeptide at a peak level of expression measured for the human subject at two years after the administering. In some methods, the expression or activity of the polypeptide of interest is at least 60% of the expression or activity of the polypeptide at a peak level of expression measured for the human subject at 2 years after the administering. In some methods, the expression or activity of the polypeptide of interest is at least 60% of the expression or activity of the polypeptide at a peak level of expression measured for the human subject at 24 weeks after the administering.

In some methods involving insertion into an ALB locus, the subject's (e.g., neonatal subject's) circulating albumin levels or cell's (e.g., neonatal cell's) albumin levels are normal. Such methods may comprise maintaining the subject's (e.g., neonatal subject's) circulating albumin levels or the cell's (e.g., neonatal cell's) albumin levels within ±5%, ±10%, ±15%, ±20%, or ±50% of normal circulating albumin levels or normal albumin levels. In some methods, the subject's (e.g., neonatal subject's) or cell's (e.g., neonatal cell's) albumin levels are unchanged as compared to the albumin levels of untreated individuals by at least week 4, at least week 8, at least week 12, or at least week 20. In some methods, the subject's (e.g., neonatal subject's) or cell's (e.g., neonatal cell's) albumin levels transiently drop and then return to normal levels. In particular, the methods may comprise detecting no significant alterations in levels of plasma albumin.

In some methods, the method further comprises assessing preexisting anti-AAV (e.g., anti-AAV8) immunity in a subject prior to administering any of the nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 9
Description of Sequences.

| SEQ | | |
|---|---|---|
| ID | | |
| NO | Type | Description |
| 1 | RNA | Cas9 mRNA |
| 2 | RNA | Cas9 mRNA CDS |
| 3 | DNA | Cas9 CDS |
| 4 | DNA | Human ALB Intron 1 |
| 5 | DNA | Guide RNA Target Sequence Plus PAM v1 |
| 6 | DNA | Guide RNA Target Sequence Plus PAM v2 |
| 7 | DNA | Guide RNA Target Sequence Plus PAM v3 |
| 8 | Protein | SpCas9 Protein V1 |
| 9 | DNA | SpCas9 DNA V1 |
| 10 | DNA | SpCas9 mRNA (cDNA) |
| 11 | Protein | SpCas9 Protein V2 |
| 12 | RNA | SpCas9 mRNA V2 |
| 13 | Protein | SV40 NLS v1 |
| 14 | Protein | SV40 NLS v2 |
| 15 | Protein | Nucleoplasmin NLS |
| 16 | RNA | crRNA Tail v1 |
| 17 | RNA | crRNA Tail v2 |
| 18 | RNA | TracrRNA v1 |
| 19 | RNA | TracrRNA v2 |
| 20 | RNA | TracrRNA v3 |
| 21 | RNA | gRNA Scaffold v1 |
| 22 | RNA | gRNA Scaffold v2 |
| 23 | RNA | gRNA Scaffold v3 |
| 24 | RNA | gRNA Scaffold v4 |
| 25 | RNA | gRNA Scaffold v5 |
| 26 | RNA | gRNA Scaffold v6 |
| 27 | RNA | gRNA Scaffold v7 |
| 28 | RNA | gRNA Scaffold v8 |
| 29 | RNA | Modified gRNA Scaffold |
| 30-61 | RNA | Human ALB Intron 1 Guide Sequences |
| 62-125 | RNA | Human ALB Intron 1 sgRNA Sequences |
| 126-157 | DNA | Human ALB Intron 1 Guide RNA Target |
| | | Sequences |
| 158 | DNA | ITR 145 |
| 159 | DNA | ITR 141 |
| 160 | DNA | ITR 130 |
| 161 | DNA | SV40 polyA |
| 162 | DNA | bGH polyA |
| 163 | DNA | Mouse Alb exon 2 Splice Acceptor |
| 164 | RNA | Mouse Alb Intron 1 Guide Sequence g666 |
| 165 | DNA | Mouse Alb Intron 1 Guide RNA Target |
| | | Sequence g666 |
| 166-167 | RNA | Mouse Alb Intron 1 sgRNA Sequences g666 |
| 168 | DNA | ITR 145 Reverse Complement |
| 169 | DNA | SV40 polyA v2 |
| 170 | Protein | Human GAA Protein (NP_000143.2) |
| 171 | DNA | Human GAA cDNA/mRNA (NM_000152.5) |
| 172 | DNA | Human GAA CDS (CCDS32760.1) |
| 173 | Protein | Human GAA (70-952) Protein |
| 174 | DNA | Human GAA (70-952) CDS |
| 175 | DNA | Human GAA (70-952) CDS-DC-0 |
| 176 | DNA | Human GAA (70-952) CDS-GA-0 |
| 177 | DNA | Human GAA (70-952) CDS-GS-0 |
| 178 | DNA | Human GAA (70-952) CDS-GS-0v2 |
| 179 | DNA | Human GAA (70-952) CDS-GS-1 |
| 180 | DNA | Human GAA (70-952) CDS-GS-44 |
| 181 | DNA | Human GAA (70-952) CDS-GS-50 |
| 182 | DNA | Human GAA (70-952) CDS- RE-8 |
| 183 | Protein | 12450 anti-CD63 scFv |
| 184-215 | Protein | Additional anti-TfR scFv:GAA Sequences |
| 216-537 | DNA & | Domains in Anti-hTfR Antibodies, Antigen- |
| | Protein | Binding Fragments or scFv Molecules |
| 538-569 | Protein | Anti-hTfR scFvs |
| 570 | Protein | 12799B-2xG4S-GAA |
| 571 | Protein | 12839B-2xG4S-GAA |
| 572 | Protein | 12843B-2xG4S-GAA |
| 573 | Protein | 12847B-2xG4S-GAA |
| 574 | DNA | 12799B-2xG4S-GAA |
| 575 | DNA | 12839B-2xG4S-GAA |
| 576 | DNA | 12843B-2xG4S-GAA |
| 577 | DNA | 12847B-2xG4S-GAA |
| 578 | DNA | 12450 anti-CD63 scFv CDS |
| 579 | Protein | 12450 anti-CD63 scFv:GAA (70-952) |
| | | Fusion Protein |
| 580 | DNA | 12450 anti-CD63 scFv:GAA (70-952) |
| | | Fusion Protein CDS |
| 581 | DNA | Human GAA (70-952) CDS-1-DC |
| 582 | DNA | Human GAA (70-952) CDS-1-GS |
| 583 | DNA | Human GAA (70-952) CDS-2-DC |
| 584 | DNA | Human GAA (70-952) CDS-2-GS |
| 585 | DNA | Human GAA (70-952) CDS-3-DC |
| 586 | DNA | Human GAA (70-952) CDS-3-GS |
| 587 | DNA | Human GAA (70-952) CDS-4-DC |
| 588 | DNA | Human GAA (70-952) CDS-4-GS |
| 589 | Protein | GAA_1 77 |
| 590 | Protein | GAA_2 22 |
| 591 | Protein | GAA_3 61 |
| 592 | DNA | 12799 anti-TfR scFv |
| 593 | DNA | 12839 anti-TfR scFv |
| 594 | DNA | 12843 anti-TfR scFv |
| 595 | DNA | 12847 anti-TfR scFv |
| 596 | Protein | Optional N-Terminal Sequence |
| 597 | DNA | ITR 141 Reverse Complement |
| 598 | DNA | ITR 130 Reverse Complement |
| 599 | DNA | SV40 polyA V3 |

### EXAMPLES

### Example 1. Development of System for Neonatal Insertion into Albumin Locus in Liver

A system for nuclease-mediated insertion (e.g., CRISPR/Cas) of a transgene into a specific locus (e.g., albumin intron 1) was developed to produce durable expression of the transgene, including when administered to neonates. Exemplary components of the system, including those used in subsequent examples, are described in more detail below.

### Single Guide RNA Design and Selection

The ALB locus was selected as the insertion site for the DNA templates. A list of single guide RNAs (sgRNAs) was generated that target human ALB intron 1. See Table 10. Candidate sgRNAs were synthesized and formulated into lipid nanoparticles (LNPs) with Cas9 mRNA for evaluation in vitro and in vivo.

TABLE 10
Human ALB Intron 1 Guide RNAs.

| | SEQ ID | | | SEQ ID |
|---|---|---|---|---|
| | NO (DNA- | SEQ ID NO | SEQ ID NO | NO (Guide |
| Guide | Targeting | (Unmodified | (Modified | RNA Target |
| RNA | Segment) | sgRNA) | sgRNA) | Sequence) |
| G009844 | 30 | 62 | 94 | 126 |
| G009851 | 31 | 63 | 95 | 127 |
| G009852 | 32 | 64 | 96 | 128 |
| G009857 | 33 | 65 | 97 | 129 |
| G009858 | 34 | 66 | 98 | 130 |
| G009859 | 35 | 67 | 99 | 131 |
| G009860 | 36 | 68 | 100 | 132 |
| G009861 | 37 | 69 | 101 | 133 |
| G009866 | 38 | 70 | 102 | 134 |
| G009867 | 39 | 71 | 103 | 135 |
| G009868 | 40 | 72 | 104 | 136 |
| G009874 | 41 | 73 | 105 | 137 |
| G012747 | 42 | 74 | 106 | 138 |
| G012748 | 43 | 75 | 107 | 139 |
| G012749 | 44 | 76 | 108 | 140 |
| G012750 | 45 | 77 | 109 | 141 |
| G012751 | 46 | 78 | 110 | 142 |
| G012752 | 47 | 79 | 111 | 143 |
| G012753 | 48 | 80 | 112 | 144 |
| G012754 | 49 | 81 | 113 | 145 |
| G012755 | 50 | 82 | 114 | 146 |
| G012756 | 51 | 83 | 115 | 147 |
| G012757 | 52 | 84 | 116 | 148 |
| G012758 | 53 | 85 | 117 | 149 |
| G012759 | 54 | 86 | 118 | 150 |
| G012760 | 55 | 87 | 119 | 151 |
| G012761 | 56 | 88 | 120 | 152 |
| G012762 | 57 | 89 | 121 | 153 |
| G012763 | 58 | 90 | 122 | 154 |
| G012764 | 59 | 91 | 123 | 155 |
| G012765 | 60 | 92 | 124 | 156 |
| G012766 | 61 | 93 | 125 | 157 |

LNPs were first screened in primary human hepatocytes (PHH) using a bidirectional nanoluc-encoding AAV insertion template as a reporter. LNPs that supported targeted insertion of nanoluc were identified by measuring nanoluc protein secreted into the supernatant of PHH cultures. Candidates that passed initial PHH screening were then tested for their ability to support in vivo gene insertion. Top candidates from in vivo studies were functionally evaluated for off-target cutting.

LNP-g9860, which is formulated with ALB-targeting sgRNA 9860, described in more detail below, was selected based on supporting robust transgene expression levels across multiple platforms (primary human and non-human primate hepatocytes, ALB humanized mice, and non-human primates), lack of confirmed off-target sites, translation across species, lack of common human SNPs in the target site, low variability of transgene expression within groups, and performance across a dose range. The target site of sgRNA 9860 is conserved in cynomolgus monkeys. LNP-g9860 had no detectable off-target sites in the human genome (targeted amplicon sequencing performed in two lots of primary human hepatocytes at saturating levels of editing failed to validate any locus other than on-target at ALB) and supported transgene expression via insertion in primary human and non-human primate hepatocytes, ALB humanized mice, and non-human primates.

### LNP-g9860

LNP-g9860 was developed for use in targeting human ALB intron 1. LNP-g9860 is a lipid nanoparticle that includes a sgRNA of 100 nucleotides in length (g9860) and Cas9-encoding mRNA, each of which is described further below, encapsulated in an LNP comprised of four different lipids. The Cas9 protein, expressed from the Cas9 mRNA, is directed to cleave the DNA when sgRNA 9860 binds to the targeted complementary DNA sequence associated with a PAM. The composition of the LNP is summarized in Table 11. LNP-g9860 comprises four lipids at the following molar ratios: 50 mol % Lipid A, 9 mol % DSPC, 38 mol % cholesterol, and 3 mol % PEG2k-DMG and is formulated in aqueous buffer composed of 50 mM Tris-HCl, 45 mM NaCl, 5% (w/v) sucrose, at pH 7.4. The N:P ratio is about 6, and the gRNA:Cas9 mRNA ratio is about 1:2 by weight.

TABLE 11
Lipid Nanoparticle (LNP-g9860) Composition.

| Component | Description |
|---|---|
| Active Pharmaceutical | Cas9 mRNA |
| Components | sgRNA (gRNA9860) |
| Lipid Excipients | Lipid A: (9Z,12Z)-3-((4,4- |
| | bis(octyloxy)butanoyl)oxy)-2- |
| | ((((3-(diethylamino)propoxy) |
| | carbonyl)oxy)methyl)propyl |
| | octadeca-9,12-dienoate |
| | Cholesterol |
| | DSPC |
| | PEG2K-DMG |
| Other Excipients | Tris, NaCl, Sucrose |
| | WFI |

Single guide RNA. The single guide RNA (sgRNA 9860) used in LNP-g9860 is a 100-mer oligonucleotide containing a 20-nucleotide sequence that is complementary to the target region in intron 1 of the human ALB gene. The target sequence recognized by g9860 is conserved in the cynomolgus monkey mfAlb gene intron 1. The sequence for g9860 is set forth in SEQ ID NOs: 68 and 100. Chemical modifications are incorporated into the 100-mer during synthesis, which include phosphorothioate (PS) linkages at the 5′- and 3′-end of the sgRNA and 2′-O-methyl modifications to some of the sugars of the RNA.

Cas9 mRNA. The Cas9 messenger RNA (mRNA) used in LNP-g9860 is based on the Cas9 protein sequence from Streptococcus pyogenes. The Cas9-encoding mRNA (SEQ ID NO: 1, with a coding sequence (CDS) set forth in SEQ ID NO: 2), is approximately 4400 nucleotides in length. The sequence contains a 5′ cap, a 5′ untranslated region (UTR), an open reading frame (ORF) encoding the Cas9 protein, a 3′ UTR, and a polyA tail. The 5′ cap is generated co-transcriptionally by use of a synthetic cap analogue structure, known as anti-reverse cap analogue (ARCA). The uracils in the mRNA sequence have been completely replaced by a modified N¹ methylpseudouridine during the in vitro transcription. The 5′ end of the mRNA has a synthetic cap analog structure. The poly-A tail is approximately 100 nucleotides.

### LNP-g666

LNP-g666 was developed for use in targeting mouse Alb intron 1. LNP-g666 is the same as LNP-g9860, except human-albumin-targeting g9860 is replaced with g666, a guide RNA targeting mouse albumin intron 1. The sequence for g666 is set forth in SEQ ID NOS: 166 and 167.

rAAV8 Vector

A recombinant AAV8 (rAAV8) vector was developed to carry the DNA insertion templates. The rAAV8 vector carrying the DNA insertion templates is a non-replicating vector that is an AAV-based vector derived from AAV serotype 8. The genome is a single-stranded deoxyribonucleic acid (DNA), comprising inverted terminal repeats (ITR) at each end. The ITRs flank the promoterless insertion template. The AAV ITRs flanking the cassette were derived from AAV2. The DNA insertion templates delivered by rAAV8 vector can be designed as promoterless templates, thus relying on the targeted ALB locus promoter for expression.

### Example 2. Durable Human FIX Protein Expression After Insertion in Neonatal Mice

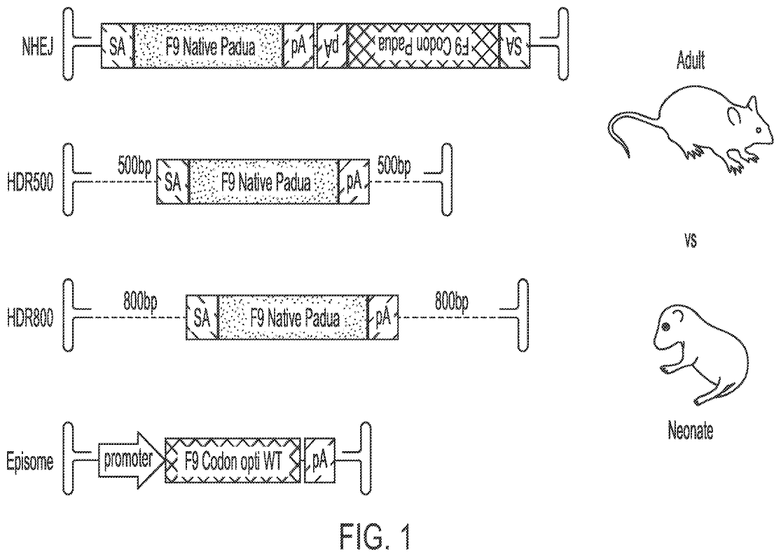

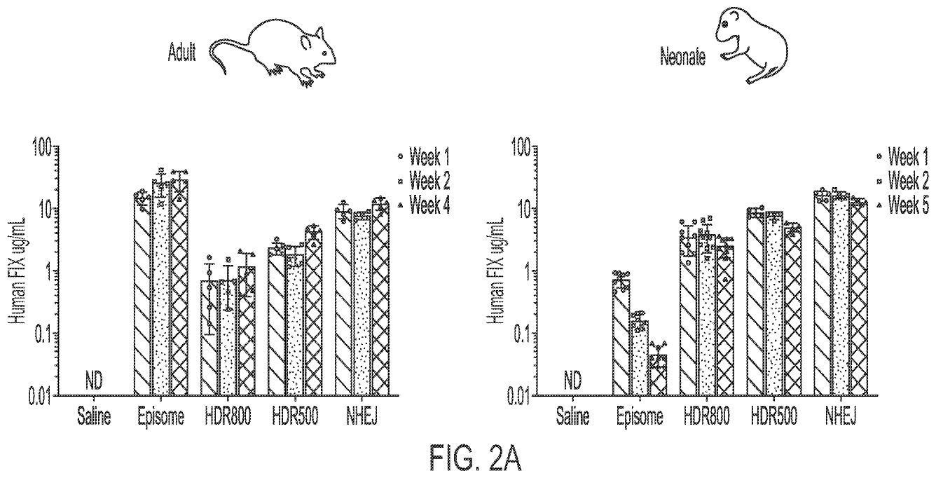

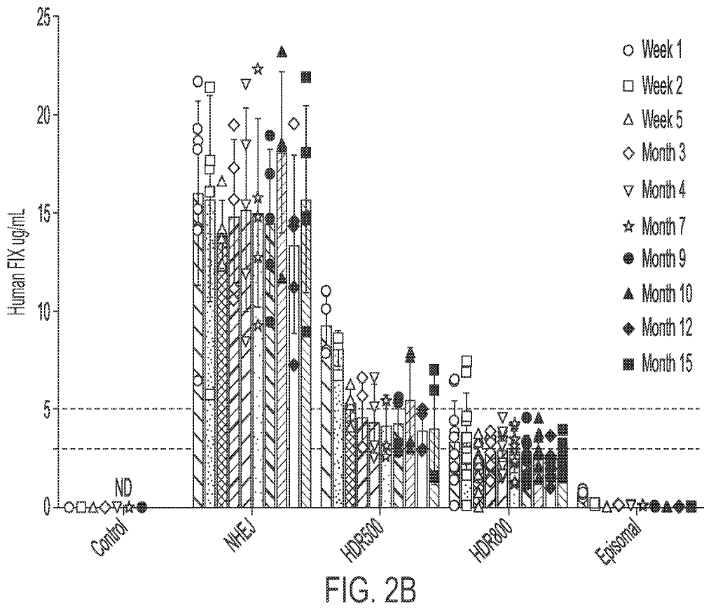

To compare episome-mediated expression versus insertion-mediated expression in adult and neonatal mice, and to compare different DNA repair pathways in adult and neonatal mice, we compared hFIX serum levels following administration of a hFIX episome (expression driven by hAAT promoter), a bidirectional hFIX NHEJ insertion template, a hFIX HDR insertion template with homology arms of 500 bp, and a hFIX HDR insertion template with homology arms of 800 bp. See FIG. 1. Neonatal C57BL/6 mice were dosed at P0 or P1 with the following: (1) 4 mg/kg of LNP-g666 and 3e9 vg/mouse of rAAV8 with the hFIX-HDR-500 template; (2) 4 mg/kg of LNP-g666 and 3e9 vg/mouse of rAAV8 with the hFIX-HDR-800 template; (3) 4 mg/kg of LNP-g666 and 3e9 vg/mouse of rAAV8 with the hFIX-NHEJ template; or (4) 3e9 vg/mouse of rAAV8 episomal template. Saline-injected mice were used as a negative control. The hFIX coding sequence in the episomal AAV was a codon-optimized sequence encoding wild type human F9. The hFIX coding sequence in the two HDR constructs was the native human F9 coding sequence with the Padua mutation (R338L). Blood was collected and plasma prepared at 1 week, 2 weeks, and 5 weeks post-dosing. hFIX levels were measured by human FIX ELISA. The experiment was then repeated in adult C57BL/6 mice, with the adult mice being dosed with the following: (1) 0.8 mg/kg of LNP-g666 and 2e10 vg/mouse of rAAV8 with the hFIX-HDR-500 template; (2) 0.8 mg/kg of LNP-g666 and 2e10 vg/mouse of rAAV8 with the hFIX-HDR-800 template; (3) 0.8 mg/kg of LNP-g666 and 2e10 vg/mouse of rAAV8 with the hFIX-NHEJ template; or (4) 2e10 vg/mouse of rAAV8 episomal template. Saline-injected mice were used as a negative control. Blood was collected and plasma prepared at 1 week, 2 weeks, and 4 weeks post-dosing. The results are shown in FIGS. 2A-2B and Tables 12-14. Episome-mediated expression was low even at the first time point compared to insertion-mediated expression in neonates and was lost over time in neonates. The opposite was observed in adult mice: episome-mediated expression was higher at the first time point and subsequent time points compared to insertion-mediated expression in adult mice. These results confirmed what was observed in a previous similar experiment (data not shown). In contrast to the results in the neonatal mice, hFIX levels stayed steady in adult mice with both episomal and insertion constructs, with the episomal construct giving the highest expression. See FIGS. 2A-2B and Tables 12-14.

TABLE 12
Human FIX Serum Levels (μg/mL) in Neonatal Mice.

| | Episome | | | | | | | | |
|---|---|---|---|---|---|---|---|---|---|
| W 1 | 0.9 | 0.56 | 0.48 | 0.52 | 0.96 | 0.67 | 0.93 | 0.98 | 0.77 |
| W 2 | 0.2 | 0.11 | 0.12 | 0.14 | 0.15 | 0.17 | 0.22 | 0.21 | 0.15 |
| W 5 | 0.06 | 0.03 | 0.03 | 0.07 | 0.07 | 0.03 | 0.04 | 0.04 | 0.04 |

| | NHEJ | | | | |
|---|---|---|---|---|---|
| W 1 | 14.22 | 19.28 | 14.12 | 18.67 | 21.69 |
| W 2 | 16.08 | 17.25 | 16.07 | 17.66 | 21.39 |
| W 5 | 12.29 | 12.41 | 14.18 | 16.61 | 13.71 |

| | HDR500 | | | |
|---|---|---|---|---|
| W 1 | 11.03 | 8.02 | 7.86 | 10.11 |
| W 2 | 8.29 | 6.74 | 8.63 | 8.61 |
| W 5 | 6.27 | 4.09 | 5.24 | 5.49 |

| | HDR800 | | | | | | | | | |
|---|---|---|---|---|---|---|---|---|---|---|
| W 1 | 3.86 | 6.44 | 6.52 | 4.43 | | 1.41 | 2 | 3.58 | 2.71 | 2.07 |
| W 2 | 3.54 | 6.9 | 7.46 | 4.44 | 4.64 | 1.65 | 2.45 | 3.55 | 2.83 | 2.5 |
| W 5 | 2.4 | 3.49 | 3.48 | 3.33 | 3.77 | 0.77 | 2.19 | 2.7 | 2.5 | 1.68 |

TABLE 13
Human FIX Serum Levels (μg/mL) in Adult Mice.

| | Episome | | | | |
|---|---|---|---|---|---|
| W1 | 16.69 | 15.57 | 18.97 | 14.17 | 9.82 |
| W2 | 25.39 | 23.07 | 41.53 | 27.53 | 12.07 |
| W4 | 39.97 | 26.61 | 39.63 | 28.5 | 14.28 |
| | NHEJ | | | | |
| W1 | 8.1 | 9.52 | 13.09 | 6.55 | 9.2 |
| W2 | 9.15 | 8.12 | 8.18 | 6.46 | 7.27 |
| W4 | 14.86 | 10.86 | 13.85 | 8.42 | 13 |
| | HDR500 | | | | |
| W1 | 2.23 | 2.5 | 1.89 | 3.14 | 1.85 |
| W2 | 1.2 | 2.62 | 1.55 | 2.41 | 1.34 |
| W4 | 3.83 | 5.39 | 4.89 | 4.62 | 2.75 |
| | HDR800 | | | | |
| W1 | 1.65 | 0.94 | 0.26 | 0.54 | 0.14 |
| W2 | 0.69 | 1.52 | 0.24 | 0.67 | 0.46 |
| W4 | 2.13 | 1.87 | 0.49 | 0.94 | 0.43 |

TABLE 14
Human FIX Serum Levels (μg/mL) in Neonatal Mice.

| | | Wk 1 | Wk 2 | Wk 5 | Month 3 | Month 4 | Month 7 | Month 9 | Month 10 | Month 12 | Month 15 |
|---|---|---|---|---|---|---|---|---|---|---|---|
| | | hF9 | hF9 | hF9 | hF9 | hF9 | hF9 | hF9 | hF9 | hF9 | hF9 |
| Mice # | Group # | ug/mL | ug/mL | ug/mL | ug/mL | ug/mL | ug/mL | ug/mL | ug/mL | ug/mL | ug/mL |
| 1 | Ctrl 1 | ND | ND | ND | ND | ND | ND | ND | ND | ND | ND |
| 2 | Ctrl 2 | ND | ND | ND | ND | ND | ND | ND | ND | ND | ND |
| 3 | Ctrl 3 | ND | ND | ND | ND | ND | ND | ND | ND | ND | ND |
| 4 | Ctrl 4 | ND | ND | ND | ND | ND | ND | ND | ND | ND | ND |
| 5 | Ctrl 5 | ND | ND | ND | ND | ND | ND | ND | ND | ND | ND |
| 6 | Ctrl 6 | ND | ND | ND | ND | ND | ND | ND | ND | ND | ND |
| 7 | Ctrl 7 | ND | ND | ND | ND | ND | ND | ND | ND | ND | ND |
| 8 | Ctrl 8 | ND | ND | ND | ND | ND | ND | ND | ND | ND | ND |
| 9 | HDR500-1 | 11.03 | 8.29 | 6.27 | 3.04 | 3.12 | 3.13 | 3.32 | 3.35 | 2.91 | 1.60 |
| 10 | HDR500-3 | 8.02 | 6.74 | 4.09 | 3.05 | 2.51 | 2.59 | 2.80 | 2.98 | 2.95 | 1.54 |
| 11 | HDR500-4 | 7.86 | 8.63 | 5.24 | 6.61 | 5.11 | 5.44 | 5.62 | 7.64 | 4.76 | 5.97 |
| 12 | HDR500-5 | 10.11 | 8.61 | 5.49 | 5.68 | 6.60 | 5.43 | 5.33 | 7.89 | 5.00 | 7.00 |
| 13 | Episomal 1 | 0.90 | 0.20 | 0.06 | 0.16 | 0.14 | 0.12 | 0.04 | 0.05 | 0.04 | 0.04 |
| 14 | Episomal 2 | 0.56 | 0.11 | 0.03 | 0.09 | 0.09 | 0.08 | 0.03 | 0.03 | 0.04 | 0.06 |
| 15 | Episomal 3 | 0.48 | 0.12 | 0.03 | 0.11 | 0.09 | 0.09 | 0.02 | 0.04 | 0.02 | 0.03 |
| 16 | Episomal 4 | 0.52 | 0.14 | 0.07 | 0.14 | 0.11 | 0.09 | 0.03 | 0.02 | 0.02 | 0.05 |
| 17 | Episomal 5 | 0.96 | 0.15 | 0.07 | NA (mouse died) | | | NA (mouse died) | | | |
| 18 | Episomal 6 | 0.67 | 0.17 | 0.03 | 0.13 | 0.11 | 0.11 | 0.03 | 0.04 | 0.00 | 0.04 |
| 19 | Episomal 7 | 0.93 | 0.22 | 0.04 | 0.13 | 0.09 | 0.09 | 0.09 | 0.05 | 0.04 | 0.03 |
| 20 | Episomal 8 | 0.98 | 0.21 | 0.04 | 0.14 | 0.10 | 0.11 | 0.06 | 0.03 | 0.04 | 0.05 |
| 21 | Episomal 9 | 0.77 | 0.15 | 0.04 | 0.14 | 0.15 | 0.12 | 0.05 | 0.03 | 0.05 | 0.07 |
| 22 | HDR800-1 | 3.86 | 3.54 | 2.40 | 2.08 | 2.26 | 2.91 | 1.66 | 2.09 | 1.98 | 1.77 |
| 23 | HDR800-2 | 6.44 | 6.90 | 3.49 | 3.47 | 3.36 | 3.49 | ND | 2.92 | 2.24 | 2.31 |
| 24 | HDR800-3 | 6.52 | 7.46 | 3.48 | 2.80 | 3.62 | 3.45 | 2.71 | 2.70 | 1.95 | 2.54 |
| 25 | HDR800-4 | 4.43 | 4.44 | 3.33 | 3.59 | 4.55 | 4.14 | 3.22 | 3.75 | 2.51 | 3.28 |
| 26 | HDR800-5 | NA | 4.64 | 3.77 | 3.47 | 3.82 | 4.26 | 4.57 | 4.56 | 3.65 | 3.95 |
| 27 | HDR800-6 | 1.41 | 1.65 | 0.77 | 1.54 | 1.48 | 1.19 | 3.35 | 1.50 | 1.47 | 1.60 |
| 28 | HDR800-7 | 2.00 | 2.45 | 2.19 | 3.32 | 3.11 | 2.26 | 2.37 | 2.75 | 2.63 | 2.29 |
| 29 | HDR800-8 | 3.58 | 3.55 | 2.70 | 2.16 | 2.09 | 2.36 | 1.58 | 2.04 | 1.59 | 2.08 |
| 30 | HDR800-9 | 0.09 | 0.10 | 0.03 | NA (mouse died) | | | NA (mouse died) | | | |
| 31 | HDR800-10 | 2.71 | 2.83 | 2.50 | 3.87 | 3.36 | 3.12 | 2.52 | 3.58 | 2.45 | 2.80 |
| 32 | HDR800-11 | 2.07 | 2.50 | 1.68 | 1.35 | 1.66 | 1.28 | 1.15 | 1.45 | 1.01 | 1.50 |
| 33 | NHEJ 1 | 6.45 | 5.75 | NA (mouse died) | | | | NA (mouse died) | | | |
| 34 | NHEJ 2 | 14.22 | 16.08 | 12.29 | 10.58 | 8.44 | 9.27 | 9.45 | 11.67 | 7.24 | 8.95 |
| 35 | NHEJ 3 | 19.28 | 17.25 | 12.41 | 15.67 | 18.44 | 15.77 | 14.70 | 18.38 | 14.58 | 14.80 |
| 36 | NHEJ 4 | 15.19 | NA (mouse died) | | | | | NA (Mouse died) | | | |
| 37 | NHEJ 5 | 14.12 | 16.07 | 14.18 | 17.28 | 15.39 | 14.79 | 16.99 | 18.47 | 14.32 | 14.68 |
| 38 | NHEJ 6 | 18.67 | 17.66 | 16.61 | 19.46 | 21.53 | 22.32 | 18.93 | 23.21 | 19.53 | 21.90 |
| 39 | NHEJ 7 | 18.23 | NA (mouse died) | | | | | NA (Mouse died) | | | |
| 40 | NHEJ 8 | 21.69 | 21.39 | 13.71 | 11.15 | 11.88 | 12.72 | 12.36 | 18.53 | 11.19 | 18.07 |

These experiments showed that expression of inserted F9 is durable in neonatal livers, indicating that insertion of F9 templates into the albumin locus can result in durable expression in neonatal subjects. These genome integration provided durable expression that was maintained throughout the experiment in neonatal mice.

### Example 3. Development of Neonatal Insertion System and Reagents for Treatment of Pompe Disease

A system for nuclease-mediated insertion (e.g., CRISPR/Cas) of an anti-CD63:GAA transgene or an anti-TfR:GAA transgene into a specific locus (e.g., albumin intron 1) was developed to produce durable expression of anti-CD63:GAA or anti-TfR:GAA, including when administered to neonates.

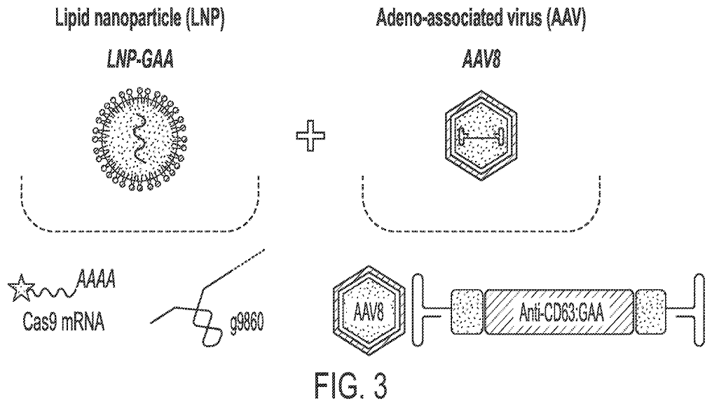

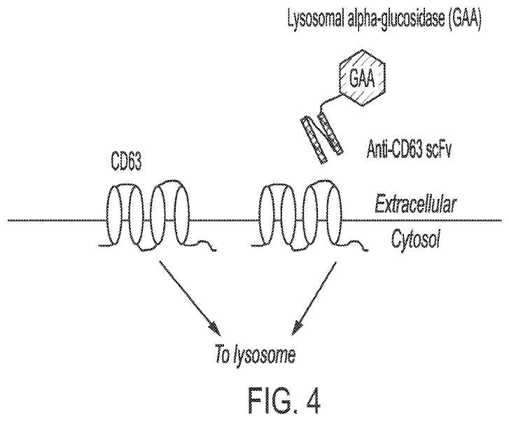

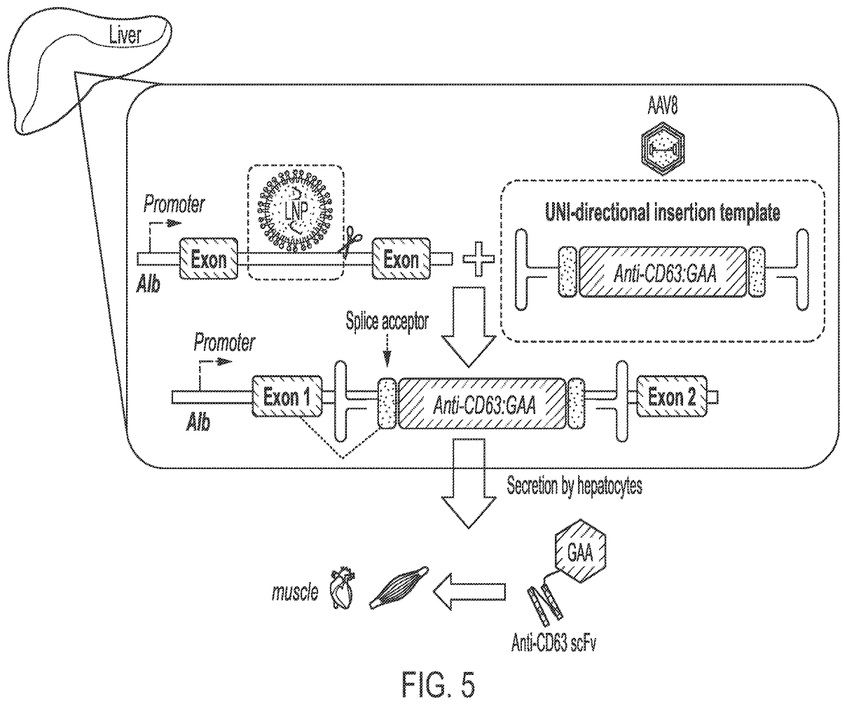

Exemplary components of the system for insertion for anti-CD63:GAA, including those used in subsequent examples, are described in more detail below. See FIGS. 3-5. The anti-CD63:GAA DNA template in the working examples described below is brought into the liver by a recombinant AAV8 vector, and the CRISPR/Cas9 RNA components (Cas9 mRNA and sgRNA) are delivered to the liver by LNP-mediated delivery (FIGS. 3 and 5). The anti-CD63:GAA protein produced by the liver is targeted to lysosomes in the muscle by targeting CD63, which is a rapidly internalizing protein highly expressed in the muscle. See FIG. 4. Single guide RNA, LNP-g9860, Cas9 mRNA, and LNP-g666 design and selection were as described in Example 1.

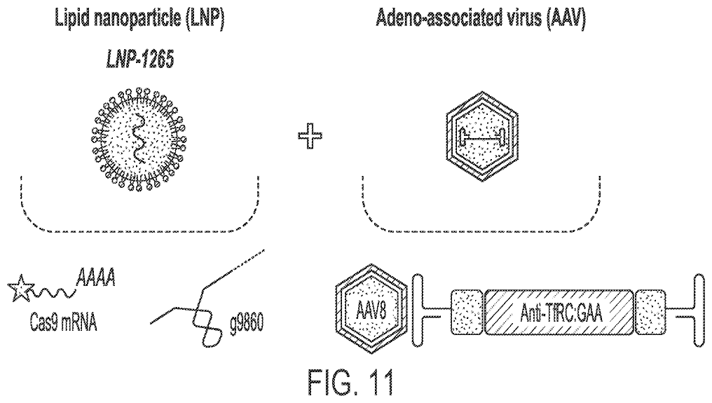

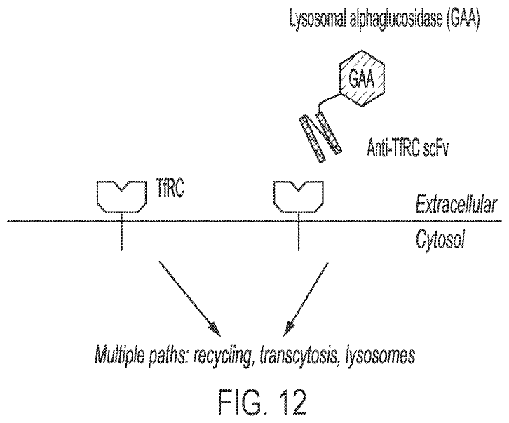

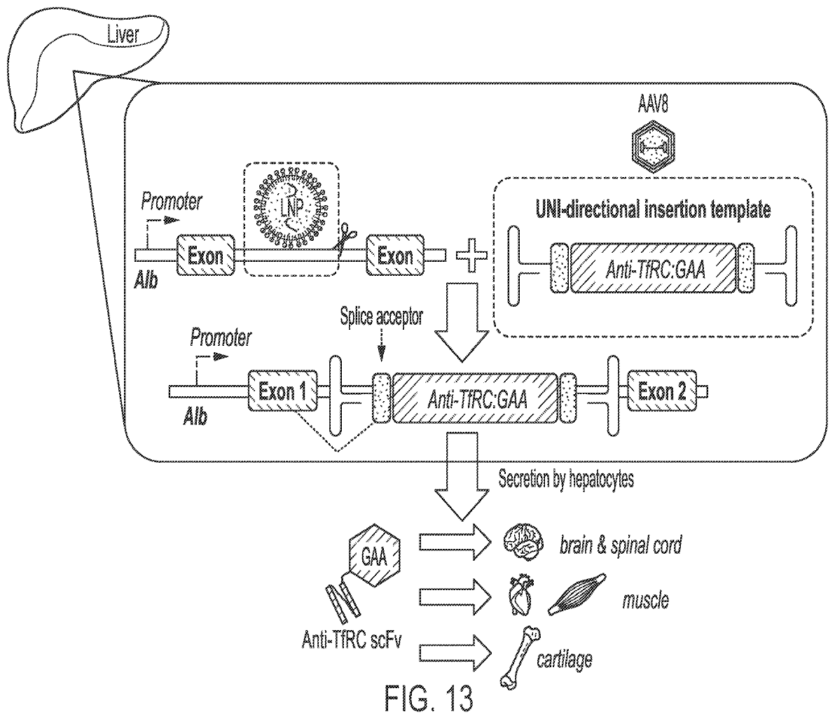

Exemplary components of the system for anti-TfR:GAA, including those used in subsequent examples, are described in more detail below. See FIGS. 11-13. The anti-TfR:GAA DNA templates in the working examples described below are brought into the liver by a recombinant AAV8 vector, and the CRISPR/Cas9 RNA components (Cas9 mRNA and sgRNA) are delivered to the liver by LNP-mediated delivery (FIGS. 11 and 13). The anti-TfR:GAA protein produced by the liver is targeted the muscle and CNS by targeting TfR, which is expressed in muscle and on brain endothelial cells. Transcytosis of TfR in these cells enables blood-brain-barrier crossing. See FIG. 12. Single guide RNA, LNP-g9860, Cas9 mRNA, and LNP-g666 design and selection were as described in Example 1.

### DNA Template Design and Selection

We engineered a DNA template for insertion of a nucleic encoding anti-CD63:GAA fusions in which the C-terminus of a single-chain fragment variable (scFv) is fused to the N-terminus of amino acids 70-952 of GAA with a glycine-serine linker. The GAA (70-952) sequence is set forth in SEQ ID NO: 173 and is encoded by the sequence set forth in SEQ ID NO: 174. The DNA template is set forth in SEQ ID NO: 580 and encodes the fusion protein set forth in SEQ ID NO: 579. A splice acceptor site is encoded upstream of the anti-CD63:GAA transgene, and a polyadenylation sequence is encoded downstream of the anti-CD63:GAA transgene. The splice acceptor sequence at the 5′ end of the transgene was derived from mouse Alb exon 2 splice acceptor. The polyadenylation sequence at the 3′ end of the transgene was derived from simian virus 40 (SV40).

We engineered DNA templates for insertion of a nucleic encoding anti-TfR:GAA fusions in which the C-terminus of a single-chain fragment variable (scFv) is fused to the N-terminus of amino acids 70-952 of GAA with a glycine-serine linker. The GAA (70-952) sequence is set forth in SEQ ID NO: 173 and is encoded by the sequence set forth in SEQ ID NO: 174. A splice acceptor site is encoded upstream of the anti-TfR:GAA transgene, and a polyadenylation sequence is encoded downstream of the anti-TfR:GAA transgene. The splice acceptor sequence at the 5′ end of the transgene was derived from mouse Alb exon 2 splice acceptor. The polyadenylation sequence at the 3′ end of the transgene was derived from simian virus 40 (SV40).

rAAV8 Vector

A recombinant AAV8 (rAAV8) vector was developed to carry the DNA insertion templates. The rAAV8 vector carrying the anti-CD63:GAA DNA template (REGV044) is a non-replicating vector that is an AAV-based vector derived from AAV serotype 8. The genome is a single-stranded deoxyribonucleic acid (DNA), comprising inverted terminal repeats (ITR) at each end. The ITRs flank the anti-CD63:GAA promoterless insertion template. The AAV ITRs flanking the cassette were derived from AAV2. The anti-CD63:GAA DNA template delivered by rAAV8 vector was designed as a promoterless template, thus relying on the targeted ALB locus promoter for expression.

The rAAV8 vector carrying the anti-TfR:GAA DNA template is a non-replicating vector that is an AAV-based vector derived from AAV serotype 8. The genome is a single-stranded deoxyribonucleic acid (DNA), comprising inverted terminal repeats (ITR) at each end. The ITRs flank the anti-TfR:GAA promoterless insertion template. The AAV ITRs flanking the cassette were derived from AAV2. The anti-TfR:GAA DNA template delivered by rAAV8 vector was designed as a promoterless template, thus relying on the targeted ALB locus promoter for expression.

### Example 4. Durable Alpha-Glucosidase (GAA) Expression after Insertion of Anti-CD63:GAA DNA Template in Neonatal Mice

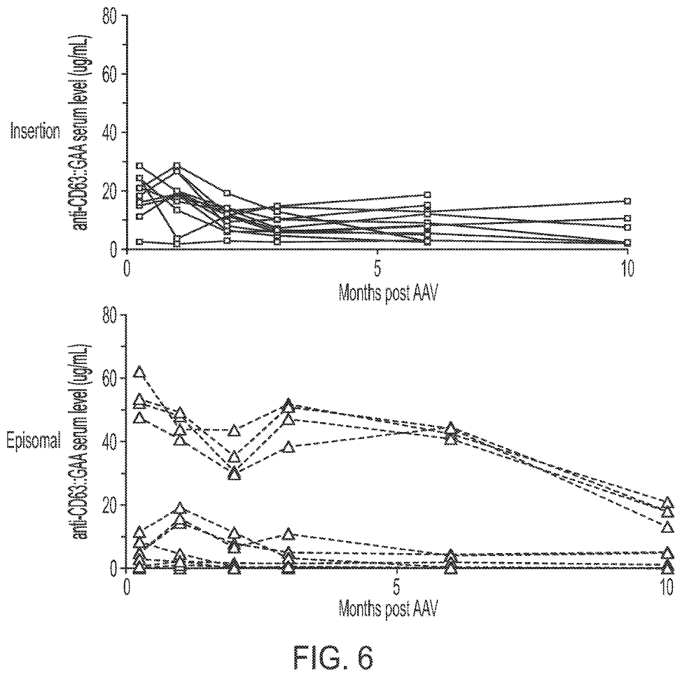

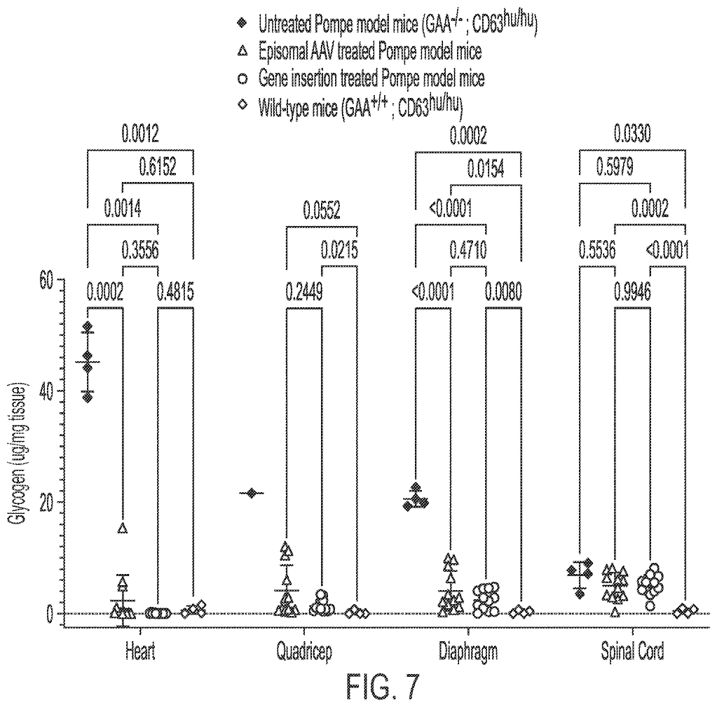

We next engineered a DNA template for insertion of a nucleic encoding anti-CD63:GAA fusions in which the C-terminus of an anti-CD63 single-chain fragment variable (scFv) is fused to the N-terminus of GAA with a glycine-serine linker (described above). We tested the anti-CD63:GAA insertion template in a Pompe disease (PD) mouse model, Gaa−/−; Cd63^{hu/hu}, where Gaa was replaced by LacZ and the protein-coding region of the Cd63 locus was replaced with its human counterpart. Adult (2-month old) male and female Gaa^{-/-}; Cd63^{hu/hu} mice (62.5% C57BL/6, 37.5% 129Sv) were dosed intravenously with the following: (1) 4e12 vg/kg recombinant AAV8 encoding anti-CD63:GAA (REGV042); or (2) 1 mg/kg LNP-g666 and 1.2e13 vg/kg recombinant AAV8 anti-CD63:GAA insertion template (REGV044). REGV042 is an episomal AAV that uses a hSerpina1 enhancer and a mTTR promoter to give hepatocyte-specific expression of anti-CD63:GAA, which further includes a human albumin signal peptide. The anti-CD63:GAA coding sequences were identical in REGV042 and REGV044 and are set forth in SEQ ID NO: 580. Untreated Gaa^{-/-}; Cd63^{hu/hu} mice and wild type mice were used as controls. Blood was collected and serum prepared at 7 days, 30 days, 2 months, 3 months, 6 months, and 10 months post-administration, and tissues were collected at 10 months post-administration. Anti-CD63:GAA serum levels were quantified using a plate-based sandwich ELISA that detects the scFv portion of the molecule. Anti-CD63:GAA purified protein was used as a protein standard for quantification. Data are shown in FIG. 6 and Tables 15-16. At 10 months post-administration, animals were sacrificed, and glycogen levels were quantified in muscle tissue lysates of the sacrificed animals. Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA). As shown in FIG. 7 and Tables 17-19, glycogen was significantly reduced to near wild type levels in both the episomal group and the insertion group in heart, quadricep, and diaphragm in adult mice.

TABLE 15
Serum Levels of Anti-CD63:GAA in μg/mL in Insertion Adult Group.

| Months | M1 | M2 | M3 | M4 | M5 | F1 | F2 | F3 | F4 | F5 | F6 | F7 |
|---|---|---|---|---|---|---|---|---|---|---|---|---|
| 0.25 | 21.06 | 2.6 | 14.93 | 24.37 | 24.39 | 16.08 | 11.21 | 18.35 | 28.54 | 20.94 | | |
| 1 | 16.43 | 1.85 | 17.92 | 13.45 | 3.7 | 18.88 | 19.37 | 26.6 | 19.8 | 28.72 | 26.63 | 19.97 |
| 2 | 13.15 | 2.95 | 13.82 | 6.06 | 11.51 | 6.45 | 9.5 | 8.05 | 11.61 | 19.2 | 12.07 | 14.09 |
| 3 | 14.49 | 2.53 | 7.22 | 6.04 | 5.75 | 4.62 | 10.19 | 5.75 | 14.8 | 12.93 | 6.61 | 10.26 |
| 6 | 12.92 | 3.13 | 12.08 | 8.18 | 5.54 | 2.48 | 9.08 | 8 | 18.66 | 2.78 | 5.03 | 15.15 |
| 10 | 16.5 | 2 | 7.5 | 10.6 | 2.3 | | 2.4 | | | | | |

*Cells without data were due to lost samples post-collection.

TABLE 16
Serum Levels of Anti-CD63:GAA in μg/mL in Episomal Adult Group.

| Months | M1 | M2 | M3 | M4 | M5 | M6 | M7 | F1 | F2 | F3 | F4 | F5 |
|---|---|---|---|---|---|---|---|---|---|---|---|---|
| 0.25 | 62.13 | 52.18 | 47.7 | 53.55 | 4.9 | 4.85 | 2.92 | 11.55 | 0 | 0 | 8.37 | 0.6 |
| 1 | 43.85 | 48.12 | 40.68 | 49.22 | 14.41 | 15.74 | 2.21 | 19.18 | 0 | 1.09 | 4.57 | 2.25 |
| 2 | 43.68 | 30.59 | 29.79 | 35.37 | 7.88 | 6.71 | 1.67 | 11.27 | 0 | 0.51 | 0.54 | 0 |
| 3 | 51.86 | 47.18 | 38.41 | 50.98 | 5.05 | 10.89 | 1.54 | 3.26 | 0 | 0 | 0.19 | 0.6 |
| 6 | 42.49 | 40.95 | 44.62 | 44.2 | 4.42 | 4.03 | 1.93 | 0.35 | 0 | 0 | 0.17 | 0.17 |
| 10 | 20.9 | 17.9 | 18 | 13.1 | 5.2 | 4.9 | 1.2 | 0.1 | 0 | 0 | 0 | 0 |

TABLE 17
Glycogen Levels in Insertion Adult Group.

| | M1 | M2 | M3 | M4 | M5 | F1 | F2 | F3 | F4 | F5 | F6 | F7 |
|---|---|---|---|---|---|---|---|---|---|---|---|---|
| Heart | 0.02 | 0.02 | 0.02 | 0.01 | 0.22 | 0.02 | 0.02 | 0.19 | 0.04 | 0.02 | 0.04 | 0.02 |
| Quadricep | 0.43 | 3.24 | 0.47 | 0.79 | 2.12 | 0.5 | 2.32 | 0.82 | 1.06 | 3.45 | 0.83 | 0.89 |
| Diaphragm | 2.85 | 1.51 | 0.34 | 2.24 | 4.75 | 0.37 | 4.06 | 0.9 | 4.47 | 4.56 | 2.86 | 0.04 |
| Spinal Cord | 4.28 | 8.11 | 5.82 | 1.38 | 3.41 | 5.46 | 6.68 | 5.63 | 4 | 4.62 | 5.57 | 7.02 |

TABLE 18
Glycogen Levels in Episomal Adult Group.

| | M1 | M2 | M3 | M4 | M5 | M6 | M7 | F1 | F2 | F3 | F4 | F5 |
|---|---|---|---|---|---|---|---|---|---|---|---|---|
| Heart | 0.18 | 0.13 | 0.22 | 0.02 | 0.18 | 0.02 | 0 | 1.04 | 15.39 | 4.99 | 5.74 | 0.13 |
| Quadricep | 0.87 | 0.54 | 0.72 | 0.27 | 0.71 | 0.85 | 2.78 | 6.09 | 12.05 | 10.46 | 11.31 | 2.89 |
| Diaphragm | 2.39 | 1.62 | 3.41 | 1.22 | 2.13 | 0.32 | 0.75 | 6.41 | 9.94 | 9.7 | 8.58 | 2.89 |
| Spinal Cord | 0.3 | 2.63 | 3.37 | 3.19 | 2.96 | 5.99 | 6.1 | 4.66 | 6.46 | 7.9 | 8.15 | 7.67 |

TABLE 19
Glycogen Levels in Control Adult Groups.

| | Untreated Pompe mice (GAA−/−; CD63 hu/hu) | | | | Wild-type (GAA+/+ CD63hu/hu) | | | |
|---|---|---|---|---|---|---|---|---|
| | M1 | M2 | F1 | F2 | M1 | M2 | F1 | F2 |
| Heart | 51.59 | 44.11 | 46.31 | 38.79 | 0.12 | 0.85 | 1.57 | 0.02 |
| Quadricep | 21.63 | | | | 0 | 0.74 | 0 | 0 |
| Diaphragm | 20.55 | 19.89 | 19.29 | 22.63 | 0 | 0.7 | 0.42 | 0.02 |
| Spinal Cord | 9.05 | 3.5 | 7.17 | 7.78 | 0 | 0.77 | 0.96 | 0.05 |

*Cells without data were due to experimental error.

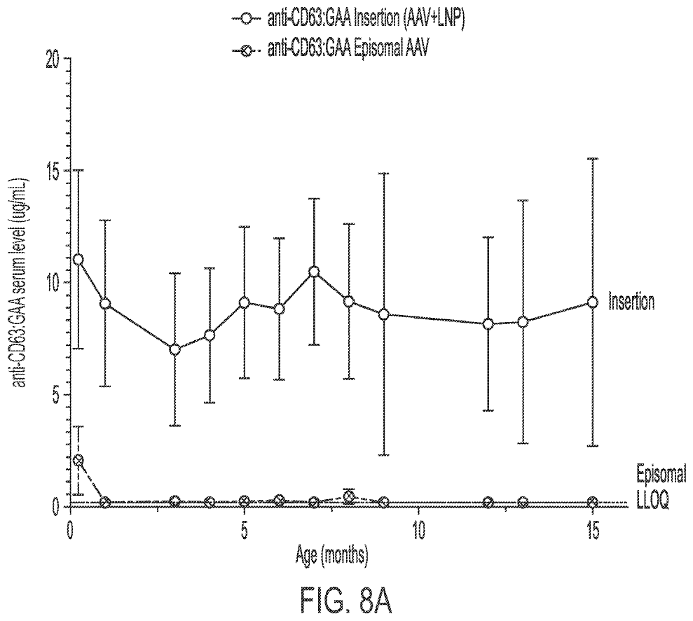

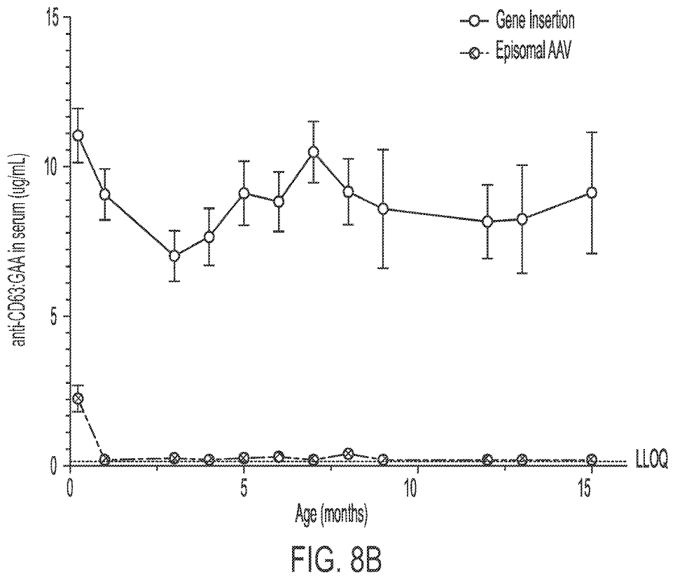

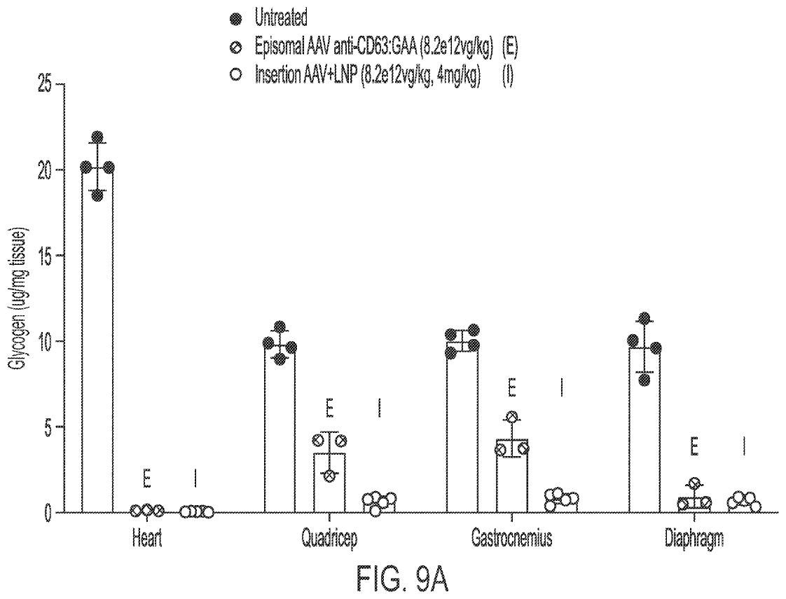

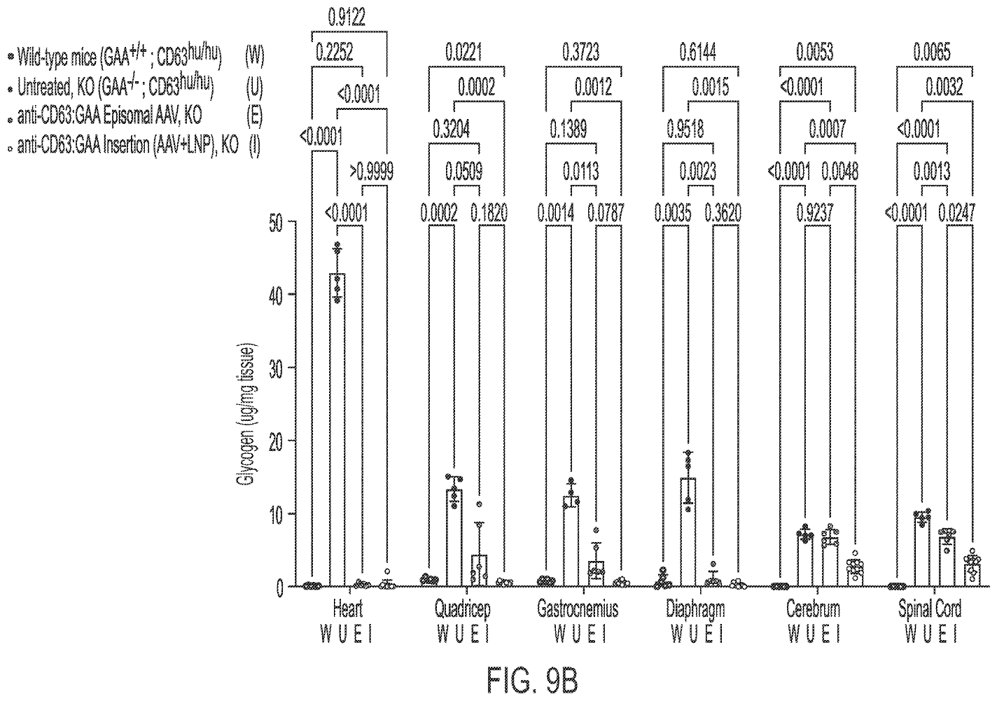

Similar experiments were then performed in which neonatal Gaa^{-/-}; Cd63^{hu/hu} mice (62.5% C57BL/6, 37.5% 129Sv) were dosed intravenously at P1 with the following: (1) 8.2e12 vg/kg recombinant AAV8 encoding anti-CD63:GAA (REGV042); or (2) 4 mg/kg LNP-g666, and 8.2e12 vg/kg recombinant AAV8 anti-CD63:GAA insertion template (REGV044). Untreated Gaa^{-/-}; Cd63^{hu/hu} mice and wild type mice were used as controls. Blood was collected and serum prepared at 7 days, 30 days, 2 months, 3 months, 4 months, 5 months, 6 months, 7 months, 8 months, 9 months, 12 months, 13 months, and 15 months, and tissues were collected at 3 months and 15 months post-administration. As shown in FIGS. 8A-8B and Tables 20-21, in contrast to what was observed when adult mice were dosed, the serum anti-CD63:GAA levels were stable over the 15-month time course in the insertion group, but the episomal group started out lower and dropped off to below the lower limit of quantification using the serum ELISA assay within 1 month when neonatal mice were dosed. Similarly, as shown in FIG. 9A and Table 22, glycogen storage at 3 months was normalized to wild type levels in heart, quadricep, gastrocnemius, and diaphragm in the insertion group, but not in the episomal group. Likewise, as shown in FIG. 9B and Table 23, glycogen storage at 15 months was normalized to wild type levels in heart, quadricep, gastrocnemius, and diaphragm in the insertion group, and glycogen storage was partially corrected in CNS tissues in the insertion group but not the episomal group.

TABLE 20
Serum Anti-CD63:GAA Levels (μg/mL) in Neonatal Mice with Insertion Group.

| | Insertion (AAV + LNP) | | | | | | | | | | | | | | | | | | |
|---|---|---|---|---|---|---|---|---|---|---|---|---|---|---|---|---|---|---|---|
| Months | * | * | ** | ** | ** | | | | | | | | ** | | | | * | * | * |
| 0.23 | 18.3 | 14.4 | 15.9 | 12.2 | 12.8 | 8.7 | 5.9 | 15.1 | 9.5 | 14.6 | 8.2 | 11.6 | 5.7 | 8.4 | 14 | 13.7 | 8.8 | 8.6 | 3.4 |
| 1 | 13.2 | 14.8 | 13.9 | 9 | 10.3 | 5.4 | 3.9 | 11.1 | 6.1 | 11.2 | 8 | 9.9 | 4.7 | 9.4 | 11.5 | 8.7 | 10.2 | 11.1 | 0 |
| 3 | 10.2 | 10.9 | | | | 3.5 | 2.3 | 10.3 | 5.8 | 7.2 | 6.9 | 8.2 | 2.7 | 7.9 | 14.7 | 5.5 | 7.2 | 6.2 | 2.8 |
| 4 | | | | | | 5.2 | 2.8 | 8.7 | 7.2 | 8.7 | 6.8 | 8.4 | | 7.4 | 14.5 | 6.8 | | | |
| 5 | | | | | | 7.1 | 4.1 | 12.1 | 6.6 | 10.2 | 8.1 | 9 | | 8.5 | 16.6 | 8.8 | | | |
| 6 | | | | | | 6.9 | 4.2 | 14.8 | 7 | 10.2 | 7 | 7.9 | | 9.2 | 13.1 | 8 | | | |
| 7 | | | | | | 8.9 | 4.9 | 17.5 | 11.2 | 10.4 | 9.9 | 10 | | 9.1 | 13.4 | 9.6 | | | |
| 8 | | | | | | 8.5 | 4.4 | 17 | 6 | 8.9 | 8.4 | 8.5 | | 8.1 | 12.5 | 9.3 | | | |
| 9 | | | | | | 5.9 | 3.1 | 10.7 | 5.2 | 25.4 | 7.5 | 5.9 | | 6.5 | 9.4 | 6.2 | | | |
| 12 | | | | | | 6.7 | 3.6 | 10.3 | 6.8 | 17.8 | 6.8 | 7.3 | | 6.7 | 9.7 | 5.9 | | | |
| 13 | | | | | | 4.6 | 3.7 | 17.4 | 4.2 | 17.0 | N/A | 6.1 | | 5.3 | 10.3 | 5.6 | | | |
| 15 | | | | | | 5.1 | 3.8 | 18.4 | 4.0 | 18.3 | 7.6 | 3.9 | | 5.5 | 18.0 | 6.6 | | | |

* Mouse sacrificed at 3 months for 3-month glycogen assay
** Mouse died

TABLE 21
Serum Anti-CD63:GAA Levels (μg/mL)
in Neonatal Mice with Episomal Group.

| | Episomal AAV | | | | | | | | | | |
|---|---|---|---|---|---|---|---|---|---|---|---|
| Months | * | * | * | ** | | | ** | | | | |
| 0.23 | 0 | 0 | 1.9 | 0 | 2.2 | 4.1 | 2.5 | 2.7 | 3.7 | 3.4 | 3.6 |
| 1 | 0 | 0 | 0 | 0 | 0 | 0 | 0 | 0 | 0 | 0 | 0 |
| 3 | 0 | 0 | 0 | 0 | 0 | 0 | 0 | 0.3 | 0.4 | 0.5 | 0 |
| 4 | | | | 0 | 0 | 0 | 0 | 0 | 0 | 0 | 0 |
| 5 | | | | | 0 | 0 | 0 | 0 | 0 | 0.6 | 0 |
| 6 | | | | | 0 | 0 | 0 | 0 | 0.5 | 0.6 | 0 |
| 7 | | | | | 0 | 0 | 0 | 0 | 0 | 0 | 0 |
| 8 | | | | | 0 | 0.5 | 0 | 0 | 0.5 | 1 | 0 |
| 9 | | | | | 0 | 0 | 0 | 0 | 0 | 0 | 0 |
| 12 | | | | | 0 | 0 | 0 | 0 | 0 | 0 | 0 |
| 13 | | | | | 0 | 0 | 0 | 0 | 0 | 0 | 0 |
| 15 | | | | | 0 | 0 | | 0 | 0 | 0 | 0 |

* Mouse sacrificed

TABLE 22
Glycogen Levels (μg/mg Tissue) in Neonatal Mice.

| | Untreated KO | | | | Episomal AAV | | | Gene Insertion | | | | |
|---|---|---|---|---|---|---|---|---|---|---|---|---|
| Heart | 20.16 | 18.52 | 20.14 | 21.92 | 0.16 | 0.1 | 0.1 | 0.08 | 0.06 | 0.08 | 0.04 | 0.02 |
| Quadricep | 8.96 | 10.83 | 9.89 | 9.63 | 4.18 | 2.13 | 4.21 | 0.89 | 0.77 | 0.82 | 0.1 | 0.58 |
| Gastrocnemius | 10.39 | 10.65 | 9.31 | 9.77 | 5.58 | 3.66 | 3.75 | 1.04 | 0.83 | 1.12 | 0.38 | 0.73 |
| Diaphragm | 10.05 | 7.73 | 11.33 | 9.6 | 0.5 | 0.59 | 1.7 | 0.57 | 0.7 | 0.85 | 0.34 | 0.9 |

TABLE 23
Glycogen Levels (μg/mg Tissue) in Neonatal Mice.

| | WT | | | | | | | | | |
|---|---|---|---|---|---|---|---|---|---|---|
| Heart | 0.18 | 0.03 | 0.09 | 0.12 | 0.04 | 0.04 | | | | |
| Quadricep | 0.95 | 0.77 | 0.86 | 1.01 | 1.35 | 0.85 | | | | |
| Gastrocnemius | 0.8 | 0.68 | 0.61 | 1.02 | 1.06 | 0.53 | | | | |
| Diaphragm | 0.6 | 0.12 | 2.24 | 1.25 | 0.22 | 0.03 | | | | |
| Cerebrum | 0.02664 | 0 | 0 | 0.04436 | 0 | 0.00255 | | | | |
| Spinal Cord | 0.01999 | 0 | 0 | 0 | 0.026 | 0 | | | | |
| | Untreated | | | | | | | | | |
| Heart | 40.73 | 42.15 | 45.87 | 46.84 | 39.15 | | | | | |
| Quadricep | 12.42 | 11.02 | 14.71 | 15.05 | 13.43 | | | | | |
| Gastrocnemius | * | 10.99 | 11.6 | 14.56 | 12.86 | | | | | |
| Diaphragm | 16.49 | 10.57 | 18.33 | 17.3 | 11.87 | | | | | |
| Cerebrum | 7.28 | 6.26 | 7.02 | 6.98 | 8.23 | | | | | |
| Spinal Cord | 8.46 | 10.34 | 9.37 | 9.94 | 9.49 | | | | | |
| | P1 episomal | | | | | | | | | |
| Heart | 0.42 | 0.17 | 0.69 | 0.21 | 0.09 | 0.09 | | | | |
| Quadricep | 11.27 | 8.47 | 1.85 | 2.72 | 1.43 | 0.96 | | | | |
| Gastrocnemius | 7.73 | 5.36 | 2.02 | 2.18 | 2.03 | 1.84 | | | | |
| Diaphragm | 3.09 | 0.8 | 0.74 | 0.76 | 0.33 | 0.42 | | | | |
| Cerebrum | 8.26 | 5.63 | 7.25 | 7.48 | 5.87 | 6.33 | | | | |
| Spinal Cord | 6.42 | 4.93 | 7.46 | 7.13 | 7.61 | 7.55 | | | | |
| | P1 insertion | | | | | | | | | |
| Heart | 0.04 | 0.12 | 0.13 | 0.06 | 0.25 | 2.1 | 0 | 0.05 | 0.04 | 0.07 |
| Quadricep | 0.6 | 0.64 | 0.68 | 0.54 | 0.58 | 0.61 | 0.27 | 0.85 | 0.35 | 0.5 |
| Gastrocnemius | 0.43 | 0.65 | 0.51 | 0.51 | 0.46 | 0.9 | 0.27 | 1.06 | 0.24 | 0.43 |
| Diaphragm | 0.16 | 0.29 | 0.08 | 0.12 | 0.39 | 0.77 | 0 | 0 | 0.59 | 0.12 |
| Cerebrum | 3.09 | 4.59 | 1.18 | 3.06 | 1.87 | 2.42 | 3.33 | 2.2 | 2.39 | 3.29 |
| Spinal Cord | 3.61 | 4.89 | 1.88 | 3.55 | 1.03 | 3.79 | 3.39 | 3.72 | 2.23 | 3.42 |

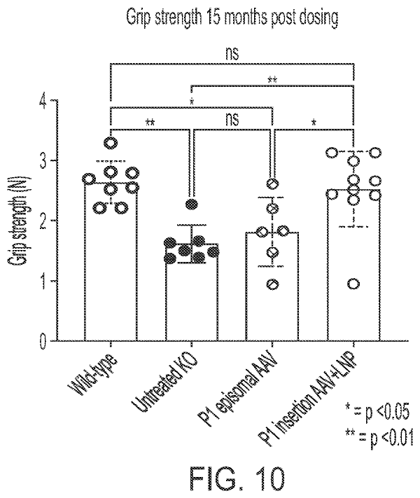

To assess whether the improved glycogen reduction observed with the insertion template in neonatal mice translated into improved muscle function, the mice were tested on grip strength apparatuses at 15 months post-administration. Limb grip strength was measured with a force meter (Columbus Instruments, Columbus, OH, USA). All tests were performed in triplicate. Mice treated with the insertion template showed significantly improved performance compared to mice treated with the episomal construct on the grip strength test. In fact, the grip strength in the insertion group tracked closely with that of wild type mice at 15 months post-treatment, whereas there was no difference in the grip strength in the episomal group tracked compared to the untreated group. See FIG. 10 and Table 24. These results show that, in neonatal mice, the insertion approach shows vastly improved durability of expression compared to the episomal approach, and better substrate reduction, indicating that insertion is the superior approach for pediatric indications.

TABLE 24
Grip Strength (Newtons) in Neonatal Mice.

| Wild- | Untreated | P1 insertion | P1 |
|---|---|---|---|
| type | KO | AAV + LNP | episomal AAV |
| 2.21 | 1.39 | 2.44 | 1.83 |
| 2.21 | 1.63 | 2.34 | 1.47 |
| 2.52 | 1.48 | 2.51 | 0.94 |
| 2.55 | 1.66 | 2.68 | 1.81 |
| 2.81 | 1.37 | 3.13 | 2.2 |
| 2.69 | 2.27 | 0.95 | 2.61 |
| 3.29 | 1.5 | 2.99 | |
| 2.79 | | 2.42 | |
| | | 2.66 | |
| | | 3.13 | |

An experiment was performed in which 4-month old Gaa^{-/-}; Alb^{hu/hu} mice (n=3) were dosed intravenously with 7.5e10 vg/mouse recombinant AAV8 anti-CD63:GAA insertion template (REGV044) and 1 mg/kg LNP-g9860 in order to validate that anti-CD63:GAA can be inserted into mice humanized for albumin using human albumin gRNA. Blood was collected and serum prepared at 7 days, 14 days, 35 days, and 60 days post-administration. GAA serum levels up to ˜3 μg/mL were observed and were maintained over the time course (data not shown), confirming that anti-CD63:GAA can be inserted into mice humanized for albumin using human albumin gRNA.

In summary, the combination of the highly precise and targeted CRISPR/Cas9 technology delivered by LNP and the anti-CD63:GAA DNA template delivered by the selected rAAV8 vector allows for long-term expression of anti-CD63:GAA protein from hepatocytes and delivery to muscle cells affected in PD, potentially providing a life-long effective treatment to PD patients, including neonatal patients.

These results show that, in neonatal mice, the insertion approach shows vastly improved durability of expression compared to the episomal approach, indicating that insertion is the superior approach in neonatal subjects.

### Example 5. Durable Alpha-Glucosidase (GAA) Expression after Insertion of Anti-TfR:GAA DNA Template in Neonatal Mice

Anti-human transferrin receptor (hTfR) antibodies were generated and screened for the ability to bind hTfR and for lack of strong blocking of human transferrin-hTfR binding. Based on this initial analysis, 32 variable sequences were chosen. See Table 25.

TABLE 25
Domains in Anti-hTfR Antibodies, Antigen-binding Fragments
(e.g., Fabs) or scFv Molecules in Fusion Proteins.

| | | HC- | HC- | | | | LC- | LC- | | | |
|---|---|---|---|---|---|---|---|---|---|---|---|
| | anti-hTfR | VR | VR | | | | VR | VR | | | |
| # | Molecule | NT | AA | HCDR1 | HCDR2 | HCDR3 | NT | AA | LCDR1 | LCDR2 | LCDR3 |
| 1 | 31874B | 216 | 217 | 218 | 219 | 220 | 221 | 222 | 223 | 224 | 225 |
| 2 | 31863B | 226 | 227 | 228 | 229 | 230 | 231 | 232 | 233 | 234 | 235 |
| 3 | 69348 | 236 | 237 | 238 | 239 | 240 | 241 | 242 | 243 | 244 | 245 |
| 4 | 69340 | 246 | 247 | 248 | 249 | 250 | 251 | 252 | 253 | 254 | 255 |
| 5 | 69331 | 256 | 257 | 258 | 259 | 260 | 261 | 262 | 263 | 264 | 265 |
| 6 | 69332 | 266 | 267 | 268 | 269 | 270 | 271 | 272 | 273 | 274 | 275 |
| 7 | 69326 | 276 | 277 | 278 | 279 | 280 | 281 | 282 | 283 | 284 | 285 |
| 8 | 69329 | 286 | 287 | 288 | 289 | 290 | 291 | 292 | 293 | 294 | 295 |
| 9 | 69323 | 296 | 297 | 298 | 299 | 300 | 301 | 302 | 303 | 304 | 305 |
| 10 | 69305 | 306 | 307 | 308 | 309 | 310 | 311 | 312 | 313 | 314 | 315 |
| 11 | 69307 | 316 | 317 | 318 | 319 | 320 | 321 | 322 | 323 | 324 | 325 |
| 12 | 12795B | 326 | 327 | 328 | 329 | 330 | 331 | 332 | 333 | 334 | 335 |
| 13 | 12798B | 336 | 337 | 338 | 339 | 340 | 341 | 342 | 343 | 344 | 345 |
| 14 | 12799B | 346 | 347 | 348 | 349 | 350 | 351 | 352 | 353 | 354 | 355 |
| 15 | 12801B | 356 | 357 | 358 | 359 | 360 | 361 | 362 | 363 | 364 | 365 |
| 16 | 12802B | 366 | 367 | 368 | 369 | 370 | 371 | 372 | 373 | 374 | 375 |
| 17 | 12808B | 376 | 377 | 378 | 379 | 380 | 381 | 382 | 383 | 384 | 385 |
| 18 | 12812B | 386 | 387 | 388 | 389 | 390 | 391 | 392 | 393 | 394 | 395 |
| 19 | 12816B | 396 | 397 | 398 | 399 | 400 | 401 | 402 | 403 | 404 | 405 |
| 20 | 12833B | 406 | 407 | 408 | 409 | 410 | 411 | 412 | 413 | 414 | 415 |
| 21 | 12834B | 416 | 417 | 418 | 419 | 420 | 421 | 422 | 423 | 424 | 425 |
| 22 | 12835B | 426 | 427 | 428 | 429 | 430 | 431 | 432 | 433 | 434 | 435 |
| 23 | 12847B | 436 | 437 | 438 | 439 | 440 | 441 | 442 | 443 | 444 | 445 |
| 24 | 12848B | 446 | 447 | 448 | 449 | 450 | 451 | 452 | 453 | 454 | 455 |
| 25 | 12843B | 456 | 457 | 458 | 459 | 460 | 461 | 462 | 463 | 464 | 465 |
| 26 | 12844B | 466 | 467 | 468 | 469 | 470 | 471 | 472 | 473 | 474 | 475 |
| 27 | 12845B | 476 | 477 | 478 | 479 | 480 | 481 | 482 | 483 | 484 | 485 |
| 28 | 12839B | 486 | 487 | 488 | 489 | 490 | 491 | 492 | 493 | 494 | 495 |
| 29 | 12841B | 496 | 497 | 498 | 499 | 500 | 501 | 502 | 503 | 504 | 505 |
| 30 | 12850B | 506 | 507 | 508 | 509 | 510 | 511 | 512 | 513 | 514 | 515 |
| 31 | 69261 | 516 | 517 | 518 | 519 | 520 | 521 | 522 | 523 | 524 | 525 |
| 32 | 69263 | 526 | 527 | 528 | 529 | 530 | 531 | 532 | 533 | 534 | 535 |

31874B
HCVR (V_{H}) Nucleotide Sequence
(SEQ ID NO: 216)
GAGGTGCAGCTGGTGGAGTCTGGGGGAGGCTTGGTACAGCCTGGGGGGTCCCTGAGACTCTCCTGTGC
AGCCTCTGGATTCGCCTTTAGCAGCTATGCCATGACCTGGGTCCGACAGGCTCCAGGGAAGGGGCTGG
AGTGGGTCTCAGTTATCAGTGGTACTGGTGGTAGTACATACTACGCAGACTCCGTGAAGGGCCGGTTC
ACCATCTCCAGAGACAATTCCAAGAACACGCTGTATCTACAAATGAACAGCCTGAGAGCCGAGGACA
CGGCCGTATATTACTGTGCGAAAGGGGGAGCAGCTCGTAGAATGGAATACTTCCAGTACTGGGGCCAG
GGCACCCTGGTCACCGTCTCCTCA
HCVR (V_{H}) Amino Acid Sequence
(SEQ ID NO: 217)
EVQLVESGGGLVQPGGSLRLSCAASGFAFSSYAMTWVRQAPGKGLEWVSVISGTGGSTYYADSVKGRFTI
SRDNSKNTLYLQMNSLRAEDTAVYYCAKGGAARRMEYFQYWGQGTLVTVSS
(SEQ ID NO: 218)
HCDR1: GFAFSSYA
(SEQ ID NO: 219)
HCDR2: ISGTGGST
(SEQ ID NO: 220)
HCDR3: AKGGAARRMEYFQY
LCVR (V_{L}) Nucleotide Sequence
(SEQ ID NO: 221)
GACATCCAGATGACCCAGTCTCCATCCTCCCTGTCTGCATCTGTAGGAGACAGAGTCACCATCACTTGC
CGGGCGAGTCAGGGCATTAGCAATTATTTAGCCTGGTATCAGCAGAAACCAGGGAAAGTTCCTAACCT
CCTTATCTATGCTGCATCCACTTTGCAATCAGGGGTCCCATCTCGATTCAGTGGCAGTGGATCTGGGAC
AGATTTCACTCTCACCATCAGCAGCCTGCAGCCTGAAGATGTTGCAACTTATTACTGTCAAAAGTATAA
CAGTGCCCCTCTCACTTTCGGCGGAGGGACCAAGGTGGAGATCAAA
LCVR (V_{L}) Amino Acid Sequence
(SEQ ID NO: 222)
DIQMTQSPSSLSASVGDRVTITCRASQGISNYLAWYQQKPGKVPNLLIYAASTLQSGVPSRFSGSGSGTDFT
LTISSLQPEDVATYYCQKYNSAPLTFGGGTKVEIK
(SEQ ID NO: 223)
LCDR1: QGISNY
(SEQ ID NO: 224)
LCDR2: AAS
(SEQ ID NO: 225)
LCDR3: QKYNSAPLT
31863B
HCVR (V_{H}) Nucleotide Sequence
(SEQ ID NO: 226)
GAGGTGCAGCTGGTGGAGTCTGGGGGAGGCTTGGTACAGCCTGGGGGGTCCCTGAGACTCTCCTGTGC
AGCCTCTGGATTCACCTTTAACAGCTATGCCATGACCTGGGTCCGCCAGGCTCCAGGGAAGGGGCTGG
AGTGGGTCTCATTTATTGGTGGTAGTACTGGTAACACATACTACGCAGGCTCCGTGAAGGGCCGGTTC
ACCATCTCCAGCGACAATTCCAAGAAGACGCTGTATCTGCAAATGAACAGCCTGAGAGCCGAGGACA
CGGCCGTATATTACTGTGCGAAAGGGGGAGCAGCTCGTAGAATGGAATACTTCCAGCACTGGGGCCA
GGGCACCCTGGTCACCGTCTCCTCA
HCVR (V_{H}) Amino Acid Sequence
(SEQ ID NO: 227)
EVQLVESGGGLVQPGGSLRLSCAASGFTFNSYAMTWVRQAPGKGLEWVSFIGGSTGNTYYAGSVKGRFTI
SSDNSKKTLYLQMNSLRAEDTAVYYCAKGGAARRMEYFQHWGQGTLVTVSS
(SEQ ID NO: 228)
HCDR1: GFTFNSYA
(SEQ ID NO: 229)
HCDR2: IGGSTGNT
(SEQ ID NO: 230)
HCDR3: AKGGAARRMEYFQH
LCVR (V_{L}) Nucleotide Sequence
(SEQ ID NO: 231)
GACATCCAGATGACCCAGTCTCCATCCTCCCTGTCTGCATCTATAGGAGACAGAGTCACCATCACTTGC
CGGGCGAGTCAGGGCATTAGCAATTATTTAGCCTGGTATCAACAGAAACCAGGGAAAGTTCCTAAGCT
CCTGATCTATGCTGCATCCACTTTGCAATCAGGGGTCCCATCTCGGTTCAGTGGCAGTGGATCTGGGAC
AGATTTCACTCTCACCATCAGCAGCCTGCAGCCTGAAGATGTTGCAACTTATTACTGTCAAAACCATAA
CAGTGTCCCTCTCACTTTCGGCGGAGGGACCAAGGTGGAGATCAAA
LCVR (V_{L}) Amino Acid Sequence
(SEQ ID NO: 232)
DIQMTQSPSSLSASIGDRVTITCRASQGISNYLAWYQQKPGKVPKLLIYAASTLQSGVPSRFSGSGSGTDFTL
TISSLQPEDVATYYCONHNSVPLTFGGGTKVEIK
(SEQ ID NO: 233)
LCDR1: QGISNY
(SEQ ID NO: 234)
LCDR2: AAS
(SEQ ID NO: 235)
LCDR3: QNHNSVPLT
69348
HCVR (V_{H}) Nucleotide Sequence
(SEQ ID NO: 236)
CAGGTGCAGCTGGTGGAGTCTGGGGGAGGCGTGGTCCAGCCTGGGAGGTCCCTGAGACTCTCCTGTGC
AGCGTCTGGATTCACCTTCACTACCTATGGCATGCACTGGGTCCGCCAGGCTCCAGGCAAGGGGCTGG
AGTGGGTGGCTGTTATATGGTATGATGGAAGTAATAAATATTATGGAGACTCCGTGAAGGGCCGATTC
ACCATCTCCAGAGACAATTCCAAGAACACACTGTATCTGCAAATGAACAGCCTGAGAGTCGACGACAC
GGCTGTTTATTACTGTACGAGAACCCATGGCTATACCAGGTCGTCGGACGGTTTTGACTACTGGGGCC
AGGGAACCCTGGTCACCGTCTCCTCA
HCVR (V_{H}) Amino Acid Sequence
(SEQ ID NO: 237)
QVQLVESGGGVVQPGRSLRLSCAASGFTFTTYGMHWVRQAPGKGLEWVAVIWYDGSNKYYGDSVKGRF
TISRDNSKNTLYLQMNSLRVDDTAVYYCTRTHGYTRSSDGFDYWGQGTLVTVSS
(SEQ ID NO: 238)
HCDR1: GFTFTTYG
(SEQ ID NO: 239)
HCDR2: IWYDGSNK
(SEQ ID NO: 240)
HCDR3: TRTHGYTRSSDGFDY
LCVR (V_{L}) Nucleotide Sequence
(SEQ ID NO: 241)
GACATCCAGATGACCCAGTCTCCATCCTCCCTGTCTGCATCTGTAGGAGACAGAGTCACCATCACTTGC
CGGGCAAGTCAGAGCATTAGAAATGTTTTAGGCTGGTTTCAGCAGAAACCAGGGAAAGCCCCTCAGC
GCCTGATCTATGCTGCATCCAGTTTGCAAAGTGGGGTCCCATCAAGGTTCAGCGGCAGTGGATCTGGG
ACAGAATTCACTCTCACAATCAGCAGCCTACAGCCTGAAGATTTTGCAACTTATTACTGTCTACAGCAT
AATTTTTACCCGCTCACTTTCGGCGGAGGGACCAAGGTGGAGATCAAA
LCVR (V_{L}) Amino Acid Sequence
(SEQ ID NO: 242)
DIQMTQSPSSLSASVGDRVTITCRASQSIRNVLGWFQQKPGKAPQRLIYAASSLQSGVPSRFSGSGSGTEFTL
TISSLQPEDFATYYCLQHNFYPLTFGGGTKVEIK
(SEQ ID NO: 243)
LCDR1: QSIRNV
(SEQ ID NO: 244)
LCDR2: AAS
(SEQ ID NO: 245)
LCDR3: LQHNFYPLT
69340
HCVR (V_{H}) Nucleotide Sequence
(SEQ ID NO: 246)
GAAGTGCAGCTGGTGGAGTCTGGGGGAGGCTTGGTACAGCCTGGCAGGTCCCTGAGACTCTCCTGTGC
AGCCTCTGGATTCACCTTTGATGATAAAGCCATGCACTGGGTCCGGCAAGTTCCAGGGAAGGGCCTGG
AATGGATCTCAGGTATTAGTTGGAATAGTGGTACTATAGGCTATGCGGACTCTGTGAAGGGCCGATTC
ATCATCTCCAGAGACAACGCCAAGAACTCCCTGTATCTACAAATGAACAGTCTGAGAGCTGAGGACAC
GGCCTTGTATTACTGCGCAAAAGATGGAGATACCAGTGGCTGGTACTGGTACGGTTTGGACGTCTGGG
GCCAAGGGACCACGGTCACCGTCTCCTCA
HCVR (V_{H}) Amino Acid Sequence
(SEQ ID NO: 247)
EVQLVESGGGLVQPGRSLRLSCAASGFTFDDKAMHWVRQVPGKGLEWISGISWNSGTIGYADSVKGRFIIS
RDNAKNSLYLQMNSLRAEDTALYYCAKDGDTSGWYWYGLDVWGQGTTVTVSS
(SEQ ID NO: 248)
HCDR1: GFTFDDKA
(SEQ ID NO: 249)
HCDR2: ISWNSGTI
(SEQ ID NO: 250)
HCDR3: AKDGDTSGWYWYGLDV
LCVR (V_{L}) Nucleotide Sequence
(SEQ ID NO: 251)
GAAATTGTGTTGACACAGTCTCCTGCCACCCTGTCTTTGTCTCCAGGGGAAAGAGCCACCCTCTCCTGC
AGGGCCAGTCAGAGTGTTAGCAGCTACTTAGCCTGGTACCAACAGAAACCTGGCCAGGCTCCCAGGCT
CCTCATCCATGATGTATCCAACAGGGCCACTGGCATCCCAGCCAGGTTCAGTGGCAGTGGGTCTGGGA
CAGACTTCACTCTCACCATCAGCAGTCTAGAGCCTGAAGATTTTGTAGTTTATTACTGTCAGCAGCGTA
GCGACTGGCCCATCACCTTCGGCCAAGGGACACGACTGGAGATTAAA
LCVR (V_{L}) Amino Acid Sequence
(SEQ ID NO: 252)
EIVLTQSPATLSLSPGERATLSCRASQSVSSYLAWYQQKPGQAPRLLIHDVSNRATGIPARFSGSGSGTDFTL
TISSLEPEDFVVYYCQQRSDWPITFGQGTRLEIK
(SEQ ID NO: 253)
LCDR1: QSVSSY
(SEQ ID NO: 254)
LCDR2: DVS
(SEQ ID NO: 255)
LCDR3: QQRSDWPIT
69331
HCVR (V_{H}) Nucleotide Sequence
(SEQ ID NO: 256)
CAGGTGCAGCTGGTGGAGTCTGGGGGAGGCGTGGTCCAGCCTGGGAGGTCCCTGAGACTCTCCTGTAT
AGCCTCTGGATTCACCTTCAGTGTCTATGGCATTCACTGGGTCCGCCAGGCTCCAGGCAAGGGGCTGG
AGTGGATGGCAGTAATATCACATGATGGAAATATTAAACACTATGCAGACTCCGTGAAGGGCCGATTC
ACCATCTCCAGAGACAATTCCAAGAACACGCTGTATCTTCAAATTAACAGCCTGAGAACTGAGGACAC
GGCTGTGTATTACTGTGCGAAAGATACCTGGAACTCCCTTGATACTTTTGATATCTGGGGCCAAGGGA
CAATGGTCACCGTCTCTTCA
HCVR (V_{H}) Amino Acid Sequence
(SEQ ID NO: 257)
QVQLVESGGGVVQPGRSLRLSCIASGFTFSVYGIHWVRQAPGKGLEWMAVISHDGNIKHYADSVKGRFTIS
RDNSKNTLYLQINSLRTEDTAVYYCAKDTWNSLDTFDIWGQGTMVTVSS
(SEQ ID NO: 258)
HCDR1: GFTFSVYG
(SEQ ID NO: 259)
HCDR2: ISHDGNIK
(SEQ ID NO: 260)
HCDR3: AKDTWNSLDTFDI
LCVR (V_{L}) Nucleotide Sequence
(SEQ ID NO: 261)
GACATCCAGTTGACCCAGTCTCCATCCTCCCTGTCTGCATCTGTAGGAGACAGAGTCACCATCACTTGC
TGGGCCAGTCAGGGCATTAGCAGTTATTTAGCCTGGTATCAGCAAAAACCAGGGAAAGCCCCTAAGCT
CCTGATCTATGCTGCATCCACTTTGCAAAGTGGGGTCCCATCAAGGTTCAGCGGCAGTGGATCTGGGA
CAGAATTCACTCTCACAATCAGCAGCCTGCAGCCTGAAGATTTTGCAACTTATTACTGTCAACAGCTTA
ATAGTTACCCTCTCACTTTCGGCGGAGGGACCAAGGTGGAGATCAAA
LCVR (V_{L}) Amino Acid Sequence
(SEQ ID NO: 262)
DIQLTQSPSSLSASVGDRVTITCWASQGISSYLAWYQQKPGKAPKLLIYAASTLQSGVPSRFSGSGSGTEFTL
TISSLQPEDFATYYCQQLNSYPLTFGGGTKVEIK
(SEQ ID NO: 263)
LCDR1: QGISSY
(SEQ ID NO: 264)
LCDR2: AAS
(SEQ ID NO: 265)
LCDR3: QQLNSYPLT
69332
HCVR (V_{H}) Nucleotide Sequence
(SEQ ID NO: 266)
CAGGTCACCTTGAGGGAGTCTGGTCCCGCGCTGGTGAAACCCTCACAGACCCTCACACTGACCTGCAC
CTTCTCTGGATTCTCACTCAACACTTATGGGATGTTTGTGAGCTGGATCCGTCAGCCTCCAGGGAAGGC
CCTAGAGTGGCTTGCACACATTCATTGGGATGATGATAAATACTACAGCACATCTCTGAAGACCAGGC
TCACCATCTCCAAGGACACCTCCAAAAACCAGGTGGTCCTTACAATGACCAACATGGACCCTGTGGAC
ACAGCCACGTATTATTGTGCACGGGGGCACAATAATTTGAACTACATCATCCACTGGGGCCAGGGAAC
CCTGGTCACCGTCTCCTCA
HCVR (V_{H}) Amino Acid Sequence
(SEQ ID NO: 267)
QVTLRESGPALVKPSQTLTLTCTFSGFSLNTYGMFVSWIRQPPGKALEWLAHIHWDDDKYYSTSLKTRLTIS
KDTSKNQVVLTMTNMDPVDTATYYCARGHNNLNYIIHWGQGTLVTVSS
(SEQ ID NO: 268)
HCDR1: GFSLNTYGMF
(SEQ ID NO: 269)
HCDR2: IHWDDDK
(SEQ ID NO: 270)
HCDR3: ARGHNNLNYIIH
LCVR (V_{L}) Nucleotide Sequence
(SEQ ID NO: 271)
GCCATCCAGATGACCCAGTCTCCATCCTCCCTGTCTGCATCTGTAGGAGACAGAGTCACCATCACTTGC
CGGGCAAGTCAGGGCATTAGAAATGATTTAGGCTGGTATCAGCAGAAACCAGGGAAAGCCCCTAAGC
TCCTGATCTATGCTGCATCCACTTTACAAAGTGGGGTCCCATCAAGGTTCAGCGGCAGTGGATCTGGC
ACAGATTTCACTCTCACCATCAGCAGCCTGCAGCCTGAAGATTTTGCAACTTATTACTGTCTACAAGAT
TACAATTACCCATTCACTTTCGGCCCTGGGACCAAAGTGGATATCAAA
LCVR (V_{L}) Amino Acid Sequence
(SEQ ID NO: 272)
AIQMTQSPSSLSASVGDRVTITCRASQGIRNDLGWYQQKPGKAPKLLIYAASTLQSGVPSRFSGSGSGTDFT
LTISSLQPEDFATYYCLQDYNYPFTFGPGTKVDIK
(SEQ ID NO: 273)
LCDR1: QGIRND
(SEQ ID NO: 274)
LCDR2: AAS
(SEQ ID NO: 275)
LCDR3: LQDYNYPFT
69326
HCVR (V_{H}) Nucleotide Sequence
(SEQ ID NO: 276)
GAGGTGCAGCTGGTGGAGTCTGGGGGAGGCTTGGTACAGCCTGGAGGGTCCCTGAGACTCTCCTGTGC
AGTCTCTGGATTCATCTTCAGTAGTTATGAAATGAACTGGGTCCGCCAGGCTCCAGGGAAGGGGCTGG
AGTGGGTTTCATACATTAGTAGTAGTGGTAGTACCATATTCTACGCAGACTCTGTGAAGGGCCGATTC
ACCATCTCCAGAGACAACGCCAAGAACTCACTGTATCTGCAAATGAACAGCCTGAGAGCCGAGGACA
CGGCTGTTTATTACTGTGTGTCTGGAGTGGTCCTTTTTGATGTCTGGGGCCAAGGGACAATGGTCACCG
TCTCTTCA
HCVR (V_{H}) Amino Acid Sequence
(SEQ ID NO: 277)
EVQLVESGGGLVQPGGSLRLSCAVSGFIFSSYEMNWVRQAPGKGLEWVSYISSSGSTIFYADSVKGRFTISR
DNAKNSLYLQMNSLRAEDTAVYYCVSGVVLFDVWGQGTMVTVSS
(SEQ ID NO: 278)
HCDR1: GFIFSSYE
(SEQ ID NO: 279)
HCDR2: ISSSGSTI
(SEQ ID NO: 280)
HCDR3: VSGVVLFDV
LCVR (V_{L}) Nucleotide Sequence
(SEQ ID NO: 281)
GAAATAGTGATGACGCAGTCTCCAGCCACCCTGTCTGTGTCTCCGGGGGAAAGAGCCACCCTCTCCTG
CAGGGCCAGTCAGAGTGTTAGCAGCAACTTTGCCTGGTACCAACAGAAACCTGGCCAGGCTCCCAGGC
TCCTCATCTATAGTGCATCCTCCAGGGCCACTGGTATCCCAGTCAGGTTCAGTGGCAGTGGGTCTGGGA
CAGAGTTCACTCTCACCATCAGCAGCCTGCAGTCTGAAGATTTTGCAGTTTATTACTGTCAGCAGTATA
ATATCTGGCCTCGGACGTTCGGCCAAGGGACCAAGGTGGAAATCAAA
LCVR (V_{L}) Amino Acid Sequence
(SEQ ID NO: 282)
EIVMTQSPATLSVSPGERATLSCRASQSVSSNFAWYQQKPGQAPRLLIYSASSRATGIPVRFSGSGSGTEFTL
TISSLQSEDFAVYYCQQYNIWPRTFGQGTKVEIK
(SEQ ID NO: 283)
LCDR1: QSVSSN
(SEQ ID NO: 284)
LCDR2: SAS
(SEQ ID NO: 285)
LCDR3: QQYNIWPRT
69329
HCVR (V_{H}) Nucleotide Sequence
(SEQ ID NO: 286)
GAGGTGCAGCTGGTGGAGTCTGGGGGAGGCTTGGTCCAGCCTGGGGGGTCCCTGAGACTCTCCTGTGC
AGCCTCTGGATTCACCTTTAGTAACTATTGGATGACCTGGGTCCGCCAGGCTCCAGGGAAGGGGCTGG
AGTGGGTGGCCAACATAAAGGAAGATGGAAGTGAGAAAGACTATGTGGACTCTGTGAAGGGCCGATT
CACCATCTCCAGAGACAACGCCAAGAACTCACTGTATCTGCAAATGAACAGCCTGAGAGGCGAGGAC
ACGGCTGTGTATTACTGTGCGAGAGATGGGGAGCAGCTCGTCGATTACTACTACTACTACGTTATGGA
CGTCTGGGGCCAAGGGACCACGGTCACCGTCTCCTCA
HCVR (V_{H}) Amino Acid Sequence
(SEQ ID NO: 287)
EVQLVESGGGLVQPGGSLRLSCAASGFTFSNYWMTWVRQAPGKGLEWVANIKEDGSEKDYVDSVKGRFT
ISRDNAKNSLYLQMNSLRGEDTAVYYCARDGEQLVDYYYYYVMDVWGQGTTVTVSS
(SEQ ID NO: 288)
HCDR1: GFTFSNYW
(SEQ ID NO: 289)
HCDR2: IKEDGSEK
(SEQ ID NO: 290)
HCDR3: ARDGEQLVDYYYYYVMDV
LCVR (V_{L}) Nucleotide Sequence
(SEQ ID NO: 291)
GACATCCAGATGACCCAGTCTCCATCTTCCGTGTCTGCATCTGTAGGAGACAGAGTCACCATCACTTGT
CGGGCGAGTCAGGGTATTAGCAGCTGGTTAGCCTGGTATCAGCAGAAACCAGGGAAAGCCCCTAAGC
TCCTGATCTATGCTGCATCCAGTTTGCAAAGTGGGGTCCCATCAAGGTTCAGCGGCAGTGGATCTGGG
ACAGATTTCACTCTCACCATCAGCAGCCTGCAGCCTGAAGATTTTGCAACTTACTATTGTCAAAAGGCT
AACAGTTTCCCGTACACTTTTGGCCAGGGGACCAAGCTGGAGATCAAA
LCVR (V_{L}) Amino Acid Sequence
(SEQ ID NO: 292)
DIQMTQSPSSVSASVGDRVTITCRASQGISSWLAWYQQKPGKAPKLLIYAASSLQSGVPSRFSGSGSGTDFT
LTISSLQPEDFATYYCQKANSFPYTFGQGTKLEIK
(SEQ ID NO: 293)
LCDR1: QGISSW
(SEQ ID NO: 294)
LCDR2: AAS
(SEQ ID NO: 295)
LCDR3: QKANSFPYT
69323 (REGN16816 anti-hTfR scFv: hGAA)
HCVR (V_{H}) Nucleotide Sequence
(SEQ ID NO: 296)
GAAGTGCAGCTGGTGGAGTCTGGGGGAGGCTTGGTACAGCCTGGCAGGTCCCTGAGACTCTCCTGTGC
AGCCTCTGGATTCACCTTTGATGACTATGCCATGCACTGGGTCCGGCAAGCTCCAGGGAAGGGCCTGG
AGTGGGTCTCAGGTATTAGTTGGAATAGTGGTTACATAGGCTATGCGGACTCTGTGAAGGGCCGATTC
ACCATCTCCAGAGACAACGCCGAGAACTCCCTACATCTGCAAATGAACAGTCTGAGAGCTGAGGACA
CGGCCTTGTATTACTGTGCAAGAGGGGGATCTACTCTGGTTCGGGGAGTTAAGGGAGGCTACTACGGT
ATGGACGTCTGGGGCCAAGGGACCACGGTCACCGTCTCCTCA
HCVR (V_{H}) Amino Acid Sequence
(SEQ ID NO: 297)
EVQLVESGGGLVQPGRSLRLSCAASGFTFDDYAMHWVRQAPGKGLEWVSGISWNSGYIGYADSVKGRFTI
SRDNAENSLHLQMNSLRAEDTALYYCARGGSTLVRGVKGGYYGMDVWGQGTTVTVSS
(SEQ ID NO: 298)
HCDR1: GFTFDDYA
(SEQ ID NO: 299)
HCDR2: ISWNSGYI
(SEQ ID NO: 300)
HCDR3: ARGGSTLVRGVKGGYYGMDV
LCVR (V_{L}) Nucleotide Sequence
(SEQ ID NO: 301)
GACATCCAGATGACCCAGTCTCCATCCTCCCTGTCTGCATCTGTAGGAGACAGAGTCACCATCACTTGC
CGGGCAAGTCAGAGCATAAGTAGCTATTTAAATTGGTATCAGCAGAAACCAGGTAAAGCCCCTAAGG
TCCTGATCTATGCTGCATCCAGTTTGCAAAGTGGGGTCCCATCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTATTCCGCTCACTTTCGGCGGAGGGACCAAGGTGGAGATCAAA
LCVR (V_{L}) Amino Acid Sequence
(SEQ ID NO: 302)
DIQMTQSPSSLSASVGDRVTITCRASQSISSYLNWYQQKPGKAPKVLIYAASSLQSGVPSRFSGSGSGTDFTL
TISSLQPEDFATYYCQQSYSIPLTFGGGTKVEIK
(SEQ ID NO: 303)
LCDR1: QSISSY
(SEQ ID NO: 304)
LCDR2: AAS
(SEQ ID NO: 305)
LCDR3: QQSYSIPLT
69305
HCVR (V_{H}) Nucleotide Sequence
(SEQ ID NO: 306)
CAGGTGCAGCTGGTGGAGTCTGGGGGAGGCGTGGTCCAGCCTGGGAGGTCCCTGAGACTCTCCTGTGC
AGCGTCTGGATTCACCTTCAGTAGCTATGGCATGCACTGGGTCCGCCAGGCTCCAGGCAAGGGGCTGG
AGTGGGTGGCAGTTATATGGTATGATGGAAGTAATAAATACTATGCAGACTCCGTGAAGGGCCGATTC
ACCATCTCCAGAGACATTTCCAAGAACACGCTGTATCTGCAAATGAACAGCCTGAGAGCCGAGGACAC
GGCCGTATATTACTGTGCGGGTCAACTGGATCTCTTCTTTGACTACTGGGGCCAGGGAACCCTGGTCAC
CGTCTCCTCA
HCVR (V_{H}) Amino Acid Sequence
(SEQ ID NO: 307)
QVQLVESGGGVVQPGRSLRLSCAASGFTFSSYGMHWVRQAPGKGLEWVAVIWYDGSNKYYADSVKGRF
TISRDISKNTLYLQMNSLRAEDTAVYYCAGQLDLFFDYWGQGTLVTVSS
(SEQ ID NO: 308)
HCDR1: GFTFSSYG
(SEQ ID NO: 309)
HCDR2: IWYDGSNK
(SEQ ID NO: 310)
HCDR3: AGQLDLFFDY
LCVR (V_{L}) Nucleotide Sequence
(SEQ ID NO: 311)
GACATCCAGATGACCCAGTCTCCATCCTCCCTGTCTGCATCTGTAGGAGACAGAGTCACCATCACTTGC
CGGGCAAGTCAGAGCATTGACAGGTATTTAAATTGGTATCGGCAGAAACCAGGGAAAGCCCCTAAGC
TCCTGATCTATACTACATCCAGTTTGCAAAGTGGGGTCCCATCAAGGTTCAGTGGCAGTGGATCTGGG
ACAGATTTCACTCTCACCCTCAGCAGTCTGCAGCCTGAAGATTTTGCAACTTACTACTGTCAGCAGAGT
TACAGTCCCCCGCTCACTTTCGGCGGAGGGACCAAGGTGGAGATCAAA
LCVR (V_{L}) Amino Acid Sequence
(SEQ ID NO: 312)
DIQMTQSPSSLSASVGDRVTITCRASQSIDRYLNWYRQKPGKAPKLLIYTTSSLQSGVPSRFSGSGSGTDFTL
TLSSLQPEDFATYYCQQSYSPPLTFGGGTKVEIK
(SEQ ID NO: 313)
LCDR1: QSIDRY
(SEQ ID NO: 314)
LCDR2: TTS
(SEQ ID NO: 315)
LCDR3: QQSYSPPLT
69307 (REGN16817 anti-hTfR scFv: hGAA)
HCVR (V_{H}) Nucleotide Sequence
(SEQ ID NO: 316)
GAGGTGCAGCTGGTGGAGTCTGGGGGAGGCTTGGTCCAGCCTGGGGGGTCCCTGAGACTCTCCTGTAC
AGCCTCTGGATTCACCTTTAGTAACTATTGGATGACCTGGGTCCGCCAGGCTCCAGGGAAGGGGCTGG
AGTGGGTGGCCAACATAAAGGAAGATGGAAGTGAGAAAGAGTATGTGGACTCTGTGAAGGGCCGGTT
CACCATCTCCAGAGACAACGCCAAGAATTCACTGTATCTGCAAATGAACAGCCTGAGAGGCGAGGAC
ACGGCTGTATATTACTGTGCGAGAGATGGGGAGCAGCTCGTCGATTACTATTACTACTACGTTATGGA
CGTCTGGGGCCAAGGGACCACGGTCACCGTCTCCTCA
HCVR (V_{H}) Amino Acid Sequence
(SEQ ID NO: 317)
EVQLVESGGGLVQPGGSLRLSCTASGFTFSNYWMTWVRQAPGKGLEWVANIKEDGSEKEYVDSVKGRFTI
SRDNAKNSLYLQMNSLRGEDTAVYYCARDGEQLVDYYYYYVMDVWGQGTTVTVSS
(SEQ ID NO: 318)
HCDR1: GFTFSNYW
(SEQ ID NO: 319)
HCDR2: IKEDGSEK
(SEQ ID NO: 320)
HCDR3: ARDGEQLVDYYYYYVMDV
LCVR (V_{L}) Nucleotide Sequence
(SEQ ID NO: 321)
GACATCCAGATGACCCAGTCTCCATCTTCCGTGTCTGCATCTGTTGGAGACAGAGTCACCATCACTTGT
CGGGCGAGTCAGGGTATTAGCAGCTGGTTAGCCTGGTATCAGCAGAAACCAGGGAAAGCCCCTAAGC
TCCTGATCTATGCTGCATCCAGTTTGCAAAGTGGGGTCCCATCAAGGTTCAGCGGCAGTGGATCTGGG
ACAGATTTCACTCTCACCATCAGCAGCCTGCAGCCTGAAGATTTTGCAACTTACTATTGTCAAAAGGCT
GACAGTCTCCCGTACGCTTTTGGCCAGGGGACCAAGCTGGAGATCAAA
LCVR (V_{L}) Amino Acid Sequence
(SEQ ID NO: 322)
DIQMTQSPSSVSASVGDRVTITCRASQGISSWLAWYQQKPGKAPKLLIYAASSLQSGVPSRFSGSGSGTDFT
LTISSLQPEDFATYYCQKADSLPYAFGQGTKLEIK
(SEQ ID NO: 323)
LCDR1: QGISSW
(SEQ ID NO: 324)
LCDR2: AAS
(SEQ ID NO: 325)
LCDR3: QKADSLPYA
12795B
HCVR (V_{H}) Nucleotide Sequence
(SEQ ID NO: 326)
GAGGTGCAGCTGGTGGAGTCTGGGGGAGGCTTGGTTCAGCCTGGGGGGTCCCTGAGACTCTCCTGTGC
AACCTCTGGATTCACCTTTACCAGCTATGACATGAAGTGGGTCCGCCAGGCTCCAGGGCTGGGCCTGG
AGTGGGTCTCAGCTATTAGTGGTAGTGGTGGTAACACATACTACGCAGACTCCGTGAAGGGCCGGTTC
ACCATCTCCAGAGACAATTCCAGGAACACGCTGTATCTGCAAATGAACAGCCTGAGAGCCGAGGACA
CGGCCGTATATTACTGTACGAGGTCCCATGACTTCGGTGCCTTCGACTACTTTGACTACTGGGGCCAGG
GAACCCTGGTCACCGTCTCCTCA
HCVR (V_{H}) Amino Acid Sequence
(SEQ ID NO: 327)
EVQLVESGGGLVQPGGSLRLSCATSGFTFTSYDMKWVRQAPGLGLEWVSAISGSGGNTYYADSVKGRFTI
SRDNSRNTLYLQMNSLRAEDTAVYYCTRSHDFGAFDYFDYWGQGTLVTVSS
(SEQ ID NO: 328)
HCDR1: GFTFTSYD
(SEQ ID NO: 329)
HCDR2: ISGSGGNT
(SEQ ID NO: 330)
HCDR3: TRSHDFGAFDYFDY
LCVR (V_{L}) Nucleotide Sequence
(SEQ ID NO: 331)
GACATCCAGATGACCCAGTCTCCATCCTCCCTGTCTGCATCTGTGGGAGACAGAGTCACCATCACTTGC
CGGGCAAGTCAGGGCATTAGAGATCATTTTGGCTGGTATCAGCAGAAACCAGGGAAAGCCCCTAAGC
GCCTGATCTATGCTGCATCCAGTTTGCACAGTGGGGTCCCATCAAGGTTCAGCGGCAGTGGATCTGGG
ACAGAATTCACTCTCACAATCAGCAGCTTGCAGCCTGAAGATTTTGCAACCTATTACTGTCTACAGTAT
GATACTTACCCGCTCACTTTCGGCGGAGGGACCAAGGTGGAGATCAAA
LCVR (V_{L}) Amino Acid Sequence
(SEQ ID NO: 332)
DIQMTQSPSSLSASVGDRVTITCRASQGIRDHFGWYQQKPGKAPKRLIYAASSLHSGVPSRFSGSGSGTEFTL
TISSLQPEDFATYYCLQYDTYPLTFGGGTKVEIK
(SEQ ID NO: 333)
LCDR1: QGIRDH
(SEQ ID NO: 334)
LCDR2: AAS
(SEQ ID NO: 335)
LCDR3: LQYDTYPLT
12798B (REGN17078 Fab; REGN17072 scFv; REGN16818 anti-hTfR scFv: hGAA)
HCVR (V_{H}) Nucleotide Sequence
(SEQ ID NO: 336)
GAAGTGCAGCTGGTGGAGTCTGGGGGAGACTTGGTACAGCCTGGCAGGTCCCTGAGACTCTCCTGTGC
AGCCTCTGGATTCACCTTTGATGATTATGCCATGCACTGGGTCCGGCAAGCTCCAGGGAAGGGCCTGG
AGTGGGTCTCAGGTATTAGTTGGAATAGTGCTACCAGAGTCTATGCGGACTCTGTGAAGGGCCGATTC
ACCATCTCCAGAGACAACGCCAAGAATTTCCTGTATCTGCAAATGAACAGTCTGAGATCTGAGGACAC
GGCCTTGTATCACTGTGCAAAAGATATGGATATCTCGCTAGGGTACTACGGTTTGGACGTCTGGGGCC
AAGGGACCACGGTCACCGTCTCCTCA
HCVR (V_{H}) Amino Acid Sequence
(SEQ ID NO: 337)
EVQLVESGGDLVQPGRSLRLSCAASGFTFDDYAMHWVRQAPGKGLEWVSGISWNSATRVYADSVKGRFT
ISRDNAKNFLYLQMNSLRSEDTALYHCAKDMDISLGYYGLDVWGQGTTVTVSS
(SEQ ID NO: 338)
HCDR1: GFTFDDYA
(SEQ ID NO: 339)
HCDR2: ISWNSATR
(SEQ ID NO: 340)
HCDR3: AKDMDISLGYYGLDV
LCVR (V_{L}) Nucleotide Sequence
(SEQ ID NO: 341)
GAAATAGTGATGACGCAGTCTCCAGCCACCCTGTCTGTGTCTCCAGGGGAAAGAGCCACCCTCTCCTG
CAGGGCCAGTCAGACTGTTAGCAGCAACTTAGCCTGGTATCAGCAGAAACCTGGCCAGGCTCCCAGGC
TCCTCATCTATGGTTCATCCTCCAGGGCCACTGGTATCCCAGCCAGGTTCAGTGGCAGTGGGTCTGGGA
CAGAGTTCACTCTCACCATCAGCAGCCTGCAGTCTGAAGATTTTGCAGTTTATTACTGTCAGCAGTATA
ATAACTGGCCTCCCTACACTTTTGGCCAGGGGACCAAGCTGGAGATCAAA
LCVR (V_{L}) Amino Acid Sequence
(SEQ ID NO: 342)
EIVMTQSPATLSVSPGERATLSCRASQTVSSNLAWYQQKPGQAPRLLIYGSSSRATGIPARFSGSGSGTEFTL
TISSLQSEDFAVYYCQQYNNWPPYTFGQGTKLEIK
(SEQ ID NO: 343)
LCDR1: QTVSSN
(SEQ ID NO: 344)
LCDR2: GSS
(SEQ ID NO: 345)
LCDR3: QQYNNWPPYT
12799B (REGN17079 Fab; REGN17073 scFv; REGN16819 anti-hTfR scFv: hGAA)
HCVR (V_{H}) Nucleotide Sequence
(SEQ ID NO: 346)
CAGATCACCTTGAAGGAGTCTGGTCCTACGCTGGTGAAACCCACACAGACCCTCACGCTGACCTGCAC
CTTCTCTGGGTTCTCACTCAGCACTAGTGGAGTGGGTGTGGTCTGGATCCGTCAGCCCCCCGGAAAGG
CCCTGGAGTGGCTTGCACTCATTTATTGGAATGATCATAAGCGGTACAGCCCATCTCTGGGGAGCAGG
CTCACCATCACCAAGGACACCTCCAAAAACCAGGTGGTCCTTACAATGACCAACATGGACCCTGTGGA
CACAGCCACATATTACTGTGCACACTACAGTGGGAGCTATTCCTACTACTACTATGGTTTGGACGTCTG
GGGCCAAGGGACCACGGTCACCGTCTCCTCA
HCVR (V_{H}) Amino Acid Sequence
(SEQ ID NO: 347)
QITLKESGPTLVKPTQTLTLTCTFSGFSLSTSGVGVVWIRQPPGKALEWLALIYWNDHKRYSPSLGSRLTITK
DTSKNQVVLTMTNMDPVDTATYYCAHYSGSYSYYYYGLDVWGQGTTVTVSS
(SEQ ID NO: 348)
HCDR1: GFSLSTSGVG
(SEQ ID NO: 349)
HCDR2: IYWNDHK
(SEQ ID NO: 350)
HCDR3: AHYSGSYSYYYYGLDV
LCVR (V_{L}) Nucleotide Sequence
(SEQ ID NO: 351)
GACATCCAGATGACCCAGTCTCCATCTTCCGTGTCTGCATCTGTAGGAGACAGAGTCACCATCACTTGT
CGGGCGAGTCAGGGTATTGCCAGCTGGTTAGCCTGGTATCAGCAGAAACCAGGGAAAGCCCCTGAGC
TCCTGATCTATGCTGCATCCAGTTTGCAAGGTGGGGTCCCATCAAGGTTCAGCGGCAGTGGATCTGGG
ACAGATTTCACTCTCACCATCAGCAGCCTGCAGCCTGAAGATTTTGCAATTTACTATTGTCAACAGGCT
AACTATTTCCCGTGGACGTTCGGCCAAGGGACCAAGGTGGAAATCAAA
LCVR (V_{L}) Amino Acid Sequence
(SEQ ID NO: 352)
DIQMTQSPSSVSASVGDRVTITCRASQGIASWLAWYQQKPGKAPELLIYAASSLQGGVPSRFSGSGSGTDFT
LTISSLQPEDFAIYYCQQANYFPWTFGQGTKVEIK
(SEQ ID NO: 353)
LCDR1: QGIASW
(SEQ ID NO: 354)
LCDR2: AAS
(SEQ ID NO: 355)
LCDR3: QQANYFPWT
12801B
HCVR (V_{H}) Nucleotide Sequence
(SEQ ID NO: 356)
GAGGTGCAGCTGTTGGAGTCTGGGGGAGCCTTGGTACAGCCTGGGGGGTCCCTGAGACTCTCCTGTGC
AGCCTCTGGATTCACCTTTACCTCCTATGCCATGCACTGGGTCCGCCAGGCTCCAGGGAAGGGTCTGG
AGTGGGTCTCATCTATTAGAGGTAGTGGTGGTGGCACATACTCCGCAGACTCCGTGAAGGGCCGGTTC
ACCATCTCCAGAGACAATTCCAGGGACACTCTATATCTGCAAATGAACAGTGTGAGAGCCGAGGACAC
GGCCGTTTATTACTGTGCGAGGTCCCATGACTACGGTGCCTTCGACTTCTTTGACTACTGGGGCCAGGG
AACCCTGGTCACCGTCTCCTCA
HCVR (V_{H}) Amino Acid Sequence
(SEQ ID NO: 357)
EVQLLESGGALVQPGGSLRLSCAASGFTFTSYAMHWVRQAPGKGLEWVSSIRGSGGGTYSADSVKGRFTIS
RDNSRDTLYLQMNSVRAEDTAVYYCARSHDYGAFDFFDYWGQGTLVTVSS
(SEQ ID NO: 358)
HCDR1: GFTFTSYA
(SEQ ID NO: 359)
HCDR2: IRGSGGGT
(SEQ ID NO: 360)
HCDR3: ARSHDYGAFDFFDY
LCVR (V_{L}) Nucleotide Sequence
(SEQ ID NO: 361)
GACATCCAGATGACCCAGTCTCCATCCTCCCTGTCTGCATCTGTAGGAGACAGAGTCACCATCACTTGC
CGGGCAAGTCAGGGCATTAGAACTGATTTAGGCTGGTATCAGCAGAAACCAGGGAAAGCCCCTAAGC
GCCTGATCTATGCTGCATCCAGTTTGCAAAGTGGGGTCCCATCAAGGTTCAGCGGCAGTGGATCTGGG
ACAGAATTCACTCTCACAATCAGCAGCCTGCGGCCTGAAGATTTTGCAACTTTTTACTGTCTACAGTAT
AATAGTTACCCGCTCACTTTCGGCGGAGGGACCAAGGTGGAGATCAAA
LCVR (V_{L}) Amino Acid Sequence
(SEQ ID NO: 362)
DIQMTQSPSSLSASVGDRVTITCRASQGIRTDLGWYQQKPGKAPKRLIYAASSLQSGVPSRFSGSGSGTEFTL
TISSLRPEDFATFYCLQYNSYPLTFGGGTKVEIK
(SEQ ID NO: 363)
LCDR1: QGIRTD
(SEQ ID NO: 364)
LCDR2: AAS
(SEQ ID NO: 365)
LCDR3: LQYNSYPLT
12802B (REGN16820 anti-hTfR scFv: hGAA)
HCVR (V_{H}) Nucleotide Sequence
(SEQ ID NO: 366)
CAGGTGCAGCTGGTGGAGTCTGGGGGAGGCTTGGTCAAGCCTGGAGGGTCCCTGAGACTCTCCTGTGC
AGCCTCTGGATTCACCTTCAGTGACTACTTCATGAGCTGGATCCGCCAGGCTCCAGGGAAGGGGCTGG
AGTGGGTTTCATACATTAGTAGTACTGGTAGTACCATAAATTATGCAGACTCTGTGAAGGGCCGATTC
ACCATCTCCAGGGACAATGTCAAGAATTCACTGTATCTGCAAATGACCAGCCTGAGAGTCGAGGACAC
GGCCGTGTATTACTGTACGAGAGATAACTGGAACTATGAATACTGGGGCCAGGGAACCCTGGTCACCG
TCTCCTCA
HCVR (V_{H}) Amino Acid Sequence
(SEQ ID NO: 367)
QVQLVESGGGLVKPGGSLRLSCAASGFTFSDYFMSWIRQAPGKGLEWVSYISSTGSTINYADSVKGRFTISR
DNVKNSLYLQMTSLRVEDTAVYYCTRDNWNYEYWGQGTLVTVSS
(SEQ ID NO: 368)
HCDR1: GFTFSDYF
(SEQ ID NO: 369)
HCDR2: ISSTGSTI
(SEQ ID NO: 370)
HCDR3: TRDNWNYEY
LCVR (V_{L}) Nucleotide Sequence
(SEQ ID NO: 371)
GAAATAGTGATGACGCAGTCTCCAGCCACCCTGTCTGTGTCTCCAGGGGAAAGAGCCACCCTCTCCTG
CAGGGCCAGTCAGAGTGTTAGCATCAACTTAGCCTGGTACCAGCAGAAACCTGGCCAGGCTCCCAGGC
TCCTCATCTTTGTTGCATCCACCAGGGCCACTGGTATCCCAGCCAGGTTCAGTGGCAGTGGGTCTGGGA
CAGAGTTCACTCTCACCATCAGCAGCCTGCAGTCTGAAGATTTTGCAACTTATTACTGTCAGCAGTATG
ATATCTGGCCGTACACTTTTGGCCAGGGGACCAAGCTGGAGATCAAA
LCVR (V_{L}) Amino Acid Sequence
(SEQ ID NO: 372)
EIVMTQSPATLSVSPGERATLSCRASQSVSINLAWYQQKPGQAPRLLIFVASTRATGIPARFSGSGSGTEFTL
TISSLQSEDFATYYCQQYDIWPYTFGQGTKLEIK
(SEQ ID NO: 373)
LCDR1: QSVSIN
(SEQ ID NO: 374)
LCDR2: VAS
(SEQ ID NO: 375)
LCDR3: QQYDIWPYT
12808B
HCVR (V_{H}) Nucleotide Sequence
(SEQ ID NO: 376)
CAGCTGCAGCTGCAGGAGTCGGGCCCAGGACTGGTGAAGCCTTCGGAGACCCTGTCCCTCACCTGCAC
TGTGTCTGGTGAATCCATCAGCAGTAATACTTACTACTGGGGCTGGATCCGCCAGCCCCCAGGGAAGG
GGCTGGAATGGATTGGGAGTATCGATTATAGTGGGACCACCAATTATAACCCGTCCCTCAAGAGTCGA
GTCACCATATCCGTAGACACGTCCAGGAATCACTTCTCCCTGAGGCTGAGGTCTGTGACCGCCGCAGA
CACGGCTGTGTATTACTGTGCGAGAGAGTGGGGAAACTACGGCTACTATTACGGTATGGACGTTTGGG
GCCAAGGGACCACGGTCACCGTCTCCTCA
HCVR (V_{H}) Amino Acid Sequence
(SEQ ID NO: 377)
QLQLQESGPGLVKPSETLSLTCTVSGESISSNTYYWGWIRQPPGKGLEWIGSIDYSGTTNYNPSLKSRVTISV
DTSRNHFSLRLRSVTAADTAVYYCAREWGNYGYYYGMDVWGQGTTVTVSS
(SEQ ID NO: 378)
HCDR1: GESISSNTYY
(SEQ ID NO: 379)
HCDR2: IDYSGTT
(SEQ ID NO: 380)
HCDR3: AREWGNYGYYYGMDV
LCVR (V_{L}) Nucleotide Sequence
(SEQ ID NO: 381)
GACATCCAGATGACCCAGTCTCCATCCTCCCTGTCTGCATCTGTAGGAGACAGAGTCACCATCAATTGC
CGGGCAAGTCAGGGCATTAGAAATGATTTAGGCTGGTATCAGCAGAAACCAGGGAAAGCCCCTAAGC
GCCTGATCTATGCTGCATCCAGTTTGCAAAGTGGGGTCCCATTAAGGTTCAGTGGCAGTGGATCTGGG
ACAGAATTCACTCTCACAATCAACAACCTGCAGCCTGAAGATTTTGCAACTTATTACTGTCTATCGCAT
AATAGTTACCCGTGGACGTTCGGCCAAGGGACCAAGGTGGAAATCAAA
LCVR (V_{L}) Amino Acid Sequence
(SEQ ID NO: 382)
DIQMTQSPSSLSASVGDRVTINCRASQGIRNDLGWYQQKPGKAPKRLIYAASSLQSGVPLRFSGSGSGTEFT
LTINNLQPEDFATYYCLSHNSYPWTFGQGTKVEIK
(SEQ ID NO: 383)
LCDR1: QGIRND
(SEQ ID NO: 384)
LCDR2: AAS
(SEQ ID NO: 385)
LCDR3: LSHNSYPWT
12812B (REGN16821 anti-hTfR scFv: hGAA)
HCVR (V_{H}) Nucleotide Sequence
(SEQ ID NO: 386)
CAGGTGCAGCTGGTGCAGTCTGGGGCTGAGGTGAAGAAGCCTGGGTCCTCGGTGAGGGTCTCCTGCAA
GGCTTCTAGAGGCACCTTCAGCAGCTATGCTATCAGCTGGGTGCGACAGGCCCCTGGACAAGGCCTTG
AGTGGATGGGAGGGATCATCCCCATCTTTGGTACAGCAAACTACGCACAGAAGTTCCTGGCCAGAGTC
ACGATTACCGCGGACGAATCCACGAGCACAGCCTACATGGAGCTGAGCAGCCTGAGATCTGAGGACA
CGGCCGTGTATTACTGTGCGAGAGAGAAGGGGTGGAACTACTTTGACTACTGGGGCCAGGGAACCCTG
GTCACCGTCTCCTCA
HCVR (V_{H}) Amino Acid Sequence
(SEQ ID NO: 387)
QVQLVQSGAEVKKPGSSVRVSCKASRGTFSSYAISWVRQAPGQGLEWMGGIIPIFGTANYAQKFLARVTIT
ADESTSTAYMELSSLRSEDTAVYYCAREKGWNYFDYWGQGTLVTVSS
(SEQ ID NO: 388)
HCDR1: RGTFSSYA
(SEQ ID NO: 389)
HCDR2: IIPIFGTA
(SEQ ID NO: 390)
HCDR3: AREKGWNYFDY
LCVR (V_{L}) Nucleotide Sequence
(SEQ ID NO: 391)
GACATCCAGATGACCCAGTCTCCACCTTCCGTGTCTGCATCTGTAGGAGACAGAGTCACCATCACTTGT
CGGGCGAGTCAGGGTATTAGCAGCTGGTTAGCCTGGTATCAGCAGAAACCAGGGAAAGCCCCTAAAC
TCCTGATCTATGCTGCATCCAGTTTGCAAAGTGGGGTCCCATCAAGGTTCAGCGGCAGTGGATCTGGG
ACAGATTTCACTCTCACCATCAGCAGCCTGCAGCCTGAAGATTTTGCAACTTACTATTGTCAACAGGCT
AACAGTTTCCCTCGGACGTTCGGCCAAGGGACCAAGGTGGAAATCAAA
LCVR (V_{L}) Amino Acid Sequence
(SEQ ID NO: 392)
DIQMTQSPPSVSASVGDRVTITCRASQGISSWLAWYQQKPGKAPKLLIYAASSLQSGVPSRFSGSGSGTDFT
LTISSLQPEDFATYYCQQANSFPRTFGQGTKVEIK
(SEQ ID NO: 393)
LCDR1: QGISSW
(SEQ ID NO: 394)
LCDR2: AAS
(SEQ ID NO: 395)
LCDR3: QQANSFPRT
12816B
HCVR (V_{H}) Nucleotide Sequence
(SEQ ID NO: 396)
CAGGTGCAGCTGGTGGAGTCTGGGGGAGGCTTGGTCAAGCCTGGAGGGTCCCTGAGACTCTCCTGTGC
AGCCTCTGGATTCACCTTCAGTGACTACTACATGAACTGGATCCGCCAGGCTCCAGGGAAGGGGCTGG
AGTGGGTTTCATACATTAGTAGTAGTGGGACTACCATATACTACGCAGACTCTGTGAAGGGCCGATTC
ACCATCTCCAGGGACAACGCCAAGAAATCACTGTATCTGGAGATGAACAGCCTCAGAGCCGAGGACA
CGGCCGTGTACTACTGTGCGAGAGAGGGGTACGGTAATGACTACTATTACTACGGTATAGACGTCTGG
GGCCAAGGGACCACGGTCACCGTCTCCTCA
HCVR (V_{H}) Amino Acid Sequence
(SEQ ID NO: 397)
QVQLVESGGGLVKPGGSLRLSCAASGFTFSDYYMNWIRQAPGKGLEWVSYISSSGTTIYYADSVKGRFTIS
RDNAKKSLYLEMNSLRAEDTAVYYCAREGYGNDYYYYGIDVWGQGTTVTVSS
(SEQ ID NO: 398)
HCDR1: GFTFSDYY
(SEQ ID NO: 399)
HCDR2: ISSSGTTI
(SEQ ID NO: 400)
HCDR3: AREGYGNDYYYYGIDV
LCVR (V_{L}) Nucleotide Sequence
(SEQ ID NO: 401)
GATATTGTGATGACTCAGTCTCCACTCTCCCTGCCCGTCACCCCTGGAGAGCCGGCCTCCATCTCCTGC
AGGTCTAGTCAGAGCCTCCTGCATGGTAATGGATACAACTATTTGACTTGGTACCTGCAGAAGCCAGG
GCAGTCTCCACAGCTCCTGATCTATTTGGGTTCTAATCGGGCCTCCGGGGTCCCTGACAGGTTCAGTGG
CAGTGGATCAGGCACAGATTTTACACTGAAAATAAGCAGAGTGGAGGCTGAGGATGTTGGGGTTTATT
ACTGCATGCAAGCTCTACAAACTCCGTACACTTTTGGCCAGGGGACCAAGCTGGAGATCAAA
LCVR (V_{L}) Amino Acid Sequence
(SEQ ID NO: 402)
DIVMTQSPLSLPVTPGEPASISCRSSQSLLHGNGYNYLTWYLQKPGQSPQLLIYLGSNRASGVPDRFSGSGSG
TDFTLKISRVEAEDVGVYYCMQALQTPYTFGQGTKLEIK
(SEQ ID NO: 403)
LCDR1: QSLLHGNGYNY
(SEQ ID NO: 404)
LCDR2: LGS
(SEQ ID NO: 405)
LCDR3: MQALQTPYT
12833B
HCVR (V_{H}) Nucleotide Sequence
(SEQ ID NO: 406)
CAGGTGCAGCTGGTGGAGTCTGGGGGAGGCGTGGTCCAGCCTGGGAGGTCCCTGAGACTCTCCTGTGC
AGCCTCTGGATTCACCTTCAGTAGCTTTGGCATGCACTGGGTCCGCCAGGCTCCAGGCAAGGGGCTGG
AGTGGGTGATATTTATATCATATGATGGAAGTGATAAATACTATGCAGACTCCGTGAAGGGCCGATTC
GCCATCTCCAGAGACAGTTCCAAGAACACGCTATATCTGCAAATGAACAGCCTGAGAGCTGAGGACA
CGGCTGTGTATTACTGTGCGAAAGAAAACGGTATTTTGACTGATTCCTACGGTATGGACGTCTGGGGC
CAAGGGACCACGGTCACCGTCTCCTCA
HCVR (V_{H}) Amino Acid Sequence
(SEQ ID NO: 407)
QVQLVESGGGVVQPGRSLRLSCAASGFTFSSFGMHWVRQAPGKGLEWVIFISYDGSDKYYADSVKGRFAI
SRDSSKNTLYLQMNSLRAEDTAVYYCAKENGILTDSYGMDVWGQGTTVTVSS
(SEQ ID NO: 408)
HCDR1: GFTFSSFG
(SEQ ID NO: 409)
HCDR2: ISYDGSDK
(SEQ ID NO: 410)
HCDR3: AKENGILTDSYGMDV
LCVR (V_{L}) Nucleotide Sequence
(SEQ ID NO: 411)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 412)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 413)
LCDR1: QSISSY
(SEQ ID NO: 414)
LCDR2: AAS
(SEQ ID NO: 415)
LCDR3: QQSYSTPPIT
12834B
HCVR (V_{H}) Nucleotide Sequence
(SEQ ID NO: 416)
CAGGTTCAGCTGGTGCAGTCTGGAGCTGAGGTGAAGAAGCCTGGGGCCTCTGTGAAGGTCTCCTGCAA
GGCTTCTGGTTACACCTTTACCAGCTATGGTATCAGCTGGGTGCGACAGGCCCCTGGACAAGGGCTTG
AGTGGATGGGATGGATCAGTGTTTACCATGGTAACACAAACTATGCACAGAAGTTCCAGGGCAGAGTC
ACCATGACCACAGACACATCCACGAGCACAGCCTACATGGAGCTGAGGAGCCTGAGATCTGACGACA
CGGCCGTGTATTACTGTGCGAGAGAGGGGTATTACGATTTTTGGAGTGGTTATTACCCTTTTGACTACT
GGGGCCAGGGAACCCTGGTCACCGTCTCCTCA
HCVR (V_{H}) Amino Acid Sequence
(SEQ ID NO: 417)
QVQLVQSGAEVKKPGASVKVSCKASGYTFTSYGISWVRQAPGQGLEWMGWISVYHGNTNYAQKFQGRV
TMTTDTSTSTAYMELRSLRSDDTAVYYCAREGYYDFWSGYYPFDYWGQGTLVTVSS
(SEQ ID NO: 418)
HCDR1: GYTFTSYG
(SEQ ID NO: 419)
HCDR2: ISVYHGNT
(SEQ ID NO: 420)
HCDR3: AREGYYDFWSGYYPFDY
LCVR (V_{L}) Nucleotide Sequence
(SEQ ID NO: 421)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 422)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 423)
LCDR1: QSISSY
(SEQ ID NO: 424)
LCDR2: AAS
(SEQ ID NO: 425)
LCDR3: QQSYSTPPIT
12835B
HCVR (V_{H}) Nucleotide Sequence
(SEQ ID NO: 426)
GAGGTGCAGCTGGTGGAGTCTGGGGGAGGCTTGATACAACCTGGAGGGTCCCTGAGACTCTCCTGTGA
AGCCTCTGGATTCACCTTCAGAAATTATGAAATGAATTGGGTCCGCCAGGCTCCAGGGAAGGGGCTGG
AGTGGGTTTCATATATTAGTAGTAGTGGTAATATGAAAGACTACGCAGAGTCTGTGAAGGGCCGATTC
ACCATCTCCAGAGACAATGTCAAGAATTCACTGCAGCTGCAAATGAACAGCCTGAGAGTCGAGGACA
CGGCTGTTTATTACTGTGCGAGAGACGAGTTTCCTTACGGAATGGACGTCTGGGGCCAAGGGACCACG
GTCACCGTCTCCTCA
HCVR (V_{H}) Amino Acid Sequence
(SEQ ID NO: 427)
EVQLVESGGGLIQPGGSLRLSCEASGFTFRNYEMNWVRQAPGKGLEWVSYISSSGNMKDYAESVKGRFTIS
RDNVKNSLQLQMNSLRVEDTAVYYCARDEFPYGMDVWGQGTTVTVSS
(SEQ ID NO: 428)
HCDR1: GFTFRNYE
(SEQ ID NO: 429)
HCDR2: ISSSGNMK
(SEQ ID NO: 430)
HCDR3: ARDEFPYGMDV
LCVR (V_{L}) Nucleotide Sequence
(SEQ ID NO: 431)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 432)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 433)
LCDR1: QSISSY
(SEQ ID NO: 434)
LCDR2: AAS
(SEQ ID NO: 435)
LCDR3: QQSYSTPPIT
12847B (REGN17083 anti-hTfR Fab; REGN17077 anti-hTfR scFv; REGN16826 anti-hTfR scFv: hGAA)
HCVR (V_{H}) Nucleotide Sequence
(SEQ ID NO: 436)
GAAGTGCAGCTGGTGGAGTCTGGGGGAGGCTTGGTTCAGCCTGGCAGGTCCCTGAGACTCTCCTGTGC
AGCCTCTGGATTCACCTTTGATGATTATGCCATGAACTGGGTCCGGCAAGCTCCAGGGAAGGGCCTGG
AGTGGGTCTCAGGTATTAGTTGGAGTAGTGGTAGCATGGACTATGCGGACTCTGTGAAGGGCCGATTC
ACCATCTCCAGAGACAACGCCAAAAACTCCCTGTATCTGCAAATGAACAGTCTGAGAACTGAGGACAC
GGCCTTATATTACTGTGCAAAAGCTAGGGAAGTTGGAGACTACTACGGTATGGACGTCTGGGGCCAAG
GGACCACGGTCACCGTCTCCTCA
HCVR (V_{H}) Amino Acid Sequence
(SEQ ID NO: 437)
EVQLVESGGGLVQPGRSLRLSCAASGFTFDDYAMNWVRQAPGKGLEWVSGISWSSGSMDYADSVKGRFT
ISRDNAKNSLYLQMNSLRTEDTALYYCAKAREVGDYYGMDVWGQGTTVTVSS
(SEQ ID NO: 438)
HCDR1: GFTFDDYA
(SEQ ID NO: 439)
HCDR2: ISWSSGSM
(SEQ ID NO: 440)
HCDR3: AKAREVGDYYGMDV
LCVR (V_{L}) Nucleotide Sequence
(SEQ ID NO: 441)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 442)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 443)
LCDR1: QSISSY
(SEQ ID NO: 444)
LCDR2: AAS
(SEQ ID NO: 445)
LCDR3: QQSYSTPPIT
12848B (REGN16827 anti-hTfR scFv: hGAA)
HCVR (V_{H}) Nucleotide Sequence
(SEQ ID NO: 446)
GAAGTGCAGCTGGTGGAGTCTGGGGGAGGCTTGGTACAGCCTGGCAGGTCCCTGACACTCTCCTGTGC
AGCCTCTGGATTCACCTTTGATAATTTTGGCATGCACTGGGTCCGGCAAGGTCCAGGGAAGGGCCTGG
AATGGGTCTCAGGTCTTACTTGGAATAGTGGTGTCATAGGCTATGCGGACTCTGTGAAGGGCCGATTC
ACCATCTCCAGAGACAACGCCAAGAACTCCCTGTATCTGCAAATGAACAGTCTGAGACCTGAGGACAC
GGCCTTATATTACTGTGCAAAAGATATACGGAATTACGGCCCCTTTGACTACTGGGGCCAGGGAACCC
TGGTCACCGTCTCCTCA
HCVR (V_{H}) Amino Acid Sequence
(SEQ ID NO: 447)
EVQLVESGGGLVQPGRSLTLSCAASGFTFDNFGMHWVRQGPGKGLEWVSGLTWNSGVIGYADSVKGRFTI
SRDNAKNSLYLQMNSLRPEDTALYYCAKDIRNYGPFDYWGQGTLVTVSS
(SEQ ID NO: 448)
HCDR1: GFTFDNFG
(SEQ ID NO: 449)
HCDR2: LTWNSGVI
(SEQ ID NO: 450)
HCDR3: AKDIRNYGPFDY
LCVR (V_{L}) Nucleotide Sequence
(SEQ ID NO: 451)
GAAATTGTGTTGACGCAGTCTCCAGGCACCCTGTCTTTGTCTCCAGGGGAAAGAGCCACCCTCTCCTGC
AGGGCCAGTCAGAGTGTTAGCAGCAGCTACTTAGCCTGGTACCAGCAGAAACCTGGCCAGGCTCCCAG
GCTCCTCATCTATGGTGCATCCAGCAGGGCCACTGGCATCCCAGACAGGTTCAGTGGCAGTGGGTCTG
GGACAGACTTCACTCTCACCATCAGCAGACTGGAGCCTGAAGATTTTGCAGTGTATTACTGTCAGCAG
TATGGTAGCTCACCTTGGACGTTCGGCCAAGGGACCAAGGTGGAAATCAAA
LCVR (V_{L}) Amino Acid Sequence
(SEQ ID NO: 452)
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSPWTFGQGTKVEIK
(SEQ ID NO: 453)
LCDR1: QSVSSSY
(SEQ ID NO: 454)
LCDR2: GAS
(SEQ ID NO: 455)
LCDR3: QQYGSSPWT
12843B (REGN17075 anti-hTfR scFv; REGN16824 anti-hTfR scFv:hGAA; REGN17081 anti-hTfR Fab)
HCVR (V_{H}) Nucleotide Sequence
(SEQ ID NO: 456)
GAGGTGCAGCTGGTGGAGTCTGGGGGAGGCTTAGTACAGCCTGGAGGGTCCCTAAGACTCTCCTGTGC
AGCCTCTGGATTCACCTTCAATATTTTTGAAATGAACTGGGTCCGCCAGGCTCCAGGGAAGGGGCTGG
AGTGGATTTCCTACATTAGTAGTCGTGGAACTACCACATACTACGCAGACTCTGTGAGGGGCCGATTC
ACCATCTCCAGAGACAACGCCAAGAACTCACTGTATCTGCAAATGAACAGCCTGAGAGCCGAGGACA
CGGCTGTTTATTACTGTGCGAGAGATTATGAAGCAACAATCCCTTTTGACTTCTGGGGCCAGGGAACC
CTGGTCACCGTCTCCTCA
HCVR (V_{H}) Amino Acid Sequence
(SEQ ID NO: 457)
EVQLVESGGGLVQPGGSLRLSCAASGFTFNIFEMNWVRQAPGKGLEWISYISSRGTTTYYADSVRGRFTISR
DNAKNSLYLQMNSLRAEDTAVYYCARDYEATIPFDFWGQGTLVTVSS
(SEQ ID NO: 458)
HCDR1: GFTFNIFE
(SEQ ID NO: 459)
HCDR2: ISSRGTTT
(SEQ ID NO: 460)
HCDR3: ARDYEATIPFDF
LCVR (V_{L}) Nucleotide Sequence
(SEQ ID NO: 461)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 462)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 463)
LCDR1: QSISSY
(SEQ ID NO: 464)
LCDR2: AAS
(SEQ ID NO: 465)
LCDR3: QQSYSTPPIT
12844B
HCVR (V_{H}) Nucleotide Sequence
(SEQ ID NO: 466)
GAGGTGCAGCTGGTGGAGTCTGGGGGAAGTGTGGTACGGCCTGGGGGGTCCCTGAGACTCTCCTGTGA
AGCCTCTGGATTCACCTTTGATGATTATGGCATGAGCTGGGTCCGCCAAGATCCAGGGAAGGGGCTGG
AGTGGGTCTCTGGTATTAATTGGAATGGTGATAGAACAAATTATGCAGACTCTGTGAAGGGCCGATTC
ATCATTTCCAGAGACAACGCCAAGAACTCTGTGTATCTACAAATGAACAGTCTGAGAGCGGAGGACTC
GGCCTTGTATCACTGTGCGAGAGATCAGGGACTCGGAGTGGCAGCTACCCTTGACTACTGGGGCCAGG
GAACCCTGGTCACCGTCTCCTCA
HCVR (V_{H}) Amino Acid Sequence
(SEQ ID NO: 467)
EVQLVESGGSVVRPGGSLRLSCEASGFTFDDYGMSWVRQDPGKGLEWVSGINWNGDRTNYADSVKGRFII
SRDNAKNSVYLQMNSLRAEDSALYHCARDQGLGVAATLDYWGQGTLVTVSS
(SEQ ID NO: 468)
HCDR1: GFTFDDYG
(SEQ ID NO: 469)
HCDR2: INWNGDRT
(SEQ ID NO: 470)
HCDR3: ARDQGLGVAATLDY
LCVR (V_{L}) Nucleotide Sequence
(SEQ ID NO: 471)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 472)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 473)
LCDR1: QSISSY
(SEQ ID NO: 474)
LCDR2: AAS
(SEQ ID NO: 475)
LCDR3: QQSYSTPPIT
12845B (REGN17082 Fab; REGN17076 scFv; REGN16825 anti-hTfR scFv: hGAA)
HCVR (V_{H}) Nucleotide Sequence
(SEQ ID NO: 476)
GAGGTGCAGCTGGTGGAGTCTGGGGGAGGCTTGGTACAGCCTGGAGGGTCCCTGAGACTCTCCTGTGC
AGCCTCTGGATTCACCGTCAGTAATTATGAAATGAACTGGGTCCGCCAGGCTCCAGGGAAGGGGCTGG
AGTGGGTTTCATACATTAGTAGTAGTACCAGTAACATATACTACGCAGACTCTGTGAAGGGCCGATTC
ACCATCTCCAGAGACAACGCCGAGAACTCACTGTATCTGCAGATGAACAGCCTGAGAGTCGAGGACA
CGGCTGTTTATTACTGTGTGAGAGATGGGATTGTAGTAGTTCCAGTTGGTCGTGGATACTACTATTACG
GTTTGGACGTCTGGGGCCAAGGGACCACGGTCACCGTCTCCTCA
HCVR (V_{H}) Amino Acid Sequence
(SEQ ID NO: 477)
EVQLVESGGGLVQPGGSLRLSCAASGFTVSNYEMNWVRQAPGKGLEWVSYISSSTSNIYYADSVKGRFTIS
RDNAENSLYLQMNSLRVEDTAVYYCVRDGIVVVPVGRGYYYYGLDVWGQGTTVTVSS
(SEQ ID NO: 478)
HCDR1: GFTVSNYE
(SEQ ID NO: 479)
HCDR2: ISSSTSNI
(SEQ ID NO: 480)
HCDR3: VRDGIVVVPVGRGYYYYGLDV
LCVR (V_{L}) Nucleotide Sequence
(SEQ ID NO: 481)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 482)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 483)
LCDR1: QSISSY
(SEQ ID NO: 484)
LCDR2: AAS
(SEQ ID NO: 485)
LCDR3: QQSYSTPPIT
12839B (REGN17080 Fab; REGN17074 scFv; REGN16822 anti-hTfR scFv: hGAA)
HCVR (V_{H}) Nucleotide Sequence
(SEQ ID NO: 486)
CAGGTGCAGCTGGTGGAGTCTGGGGGAGGCGTGGTCCAGCCTGGAAGGTCCCTGAGACTCTCCTGCGC
AGCCTCTGGATTCCCCTTTAGTAATTATGTCATGTATTGGGTCCGCCAGGCTCCAGGCAAGGGGCTGGA
GTGGGTGGCTCTTATTTTTTTTGACGGAAAGAAAAACTATCATGCAGACTCCGTGAAGGGCCGATTCA
CCATAACCAGAGACAATTCCAAAAATATGTTATATCTGCAAATGAACAGCCTGAGACCTGAGGACGCG
GCTGTGTATTACTGTGCGAAAATCCATTGTCCTAATGGTGTATGTTACAAGGGGTATTACGGAATGGA
CGTCTGGGGCCAAGGGACCACGGTCACCGTCTCCTCA
HCVR (V_{H}) Amino Acid Sequence
(SEQ ID NO: 487)
QVQLVESGGGVVQPGRSLRLSCAASGFPFSNYVMYWVRQAPGKGLEWVALIFFDGKKNYHADSVKGRFT
ITRDNSKNMLYLQMNSLRPEDAAVYYCAKIHCPNGVCYKGYYGMDVWGQGTTVTVSS
(SEQ ID NO: 488)
HCDR1: GFPFSNYV
(SEQ ID NO: 489)
HCDR2: IFFDGKKN
(SEQ ID NO: 490)
HCDR3: AKIHCPNGVCYKGYYGMDV
LCVR (V_{L}) Nucleotide Sequence
(SEQ ID NO: 491)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 492)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 493)
LCDR1: QSISSY
(SEQ ID NO: 494)
LCDR2: AAS
(SEQ ID NO: 495)
LCDR3: QQSYSTPPIT
12841B (REGN16823 anti-hTfR scFv: hGAA)
HCVR (V_{H}) Nucleotide Sequence
(SEQ ID NO: 496)
GAGGTGCAGCTGGTGGAGTCTGGGGGAGGCTTGGTCCAGCCTGGGGGGTCCCTAAGACTCTCCTGTGC
AGCCTCTGGATTCACCTTTAGTAACTATTGGATGAACTGGGTCCGCCAGGCTCCAGGGAAGGGACTGG
AGTGGGTGGCCAATATAAAAGAAGATGGAGGTAAGAAATTGTATGTGGACTCTGTGAAGGGCCGATT
CACCATCTCCAGAGACAACGCCAAGAACTCACTGTTTCTGCAAATGAACAGCCTGAGAGCCGAGGAC
ACGGCTGTGTATTATTGTGCGAGAGAAGATACAACTTTGGTTGTGGACTACTACTACTACGGTATGGA
CGTCTGGGGCCAAGGGACCACGGTCACCGTCTCCTCA
HCVR (V_{H}) Amino Acid Sequence
(SEQ ID NO: 497)
EVQLVESGGGLVQPGGSLRLSCAASGFTFSNYWMNWVRQAPGKGLEWVANIKEDGGKKLYVDSVKGRF
TISRDNAKNSLFLQMNSLRAEDTAVYYCAREDTTLVVDYYYYGMDVWGQGTTVTVSS
(SEQ ID NO: 498)
HCDR1: GFTFSNYW
(SEQ ID NO: 499)
HCDR2: IKEDGGKK
(SEQ ID NO: 500)
HCDR3: AREDTTLVVDYYYYGMDV
LCVR (V_{L}) Nucleotide Sequence
(SEQ ID NO: 501)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
LCVR (V_{L}) Amino Acid Sequence
(SEQ ID NO: 502)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 503)
LCDR1: QSISSY
(SEQ ID NO: 504)
LCDR2: AAS
(SEQ ID NO: 505)
LCDR3: QQSYSTPPIT
12850B (REGN16828 anti-hTfR scFv: hGAA)
HCVR (V_{H}) Nucleotide Sequence
(SEQ ID NO: 506)
CAGGTCCAGCTGGTGCAGTCTGGGGCTGAGGTGAAGAAGCCTGGGTCCTCGGTGAAGGTCTCCTGCAA
GGCTTCTGGAGGCACCTTCAACACCTATGCTATCACCTGGGTGCGACAGGCCCCTGGACAAGGGCTTG
AATGGATGGGGGGAATCATCCCTATCTCTGGCATAGCAGAGTACGCACAGAAGTTCCAGGGCAGAGT
CACGATCACCACGGATGACTCCTCGACCACAGCCTACATGGAACTGAACAGTCTGAGATCTGAGGACA
CGGCCGTGTATTACTGTGCGAGCTGGAACTACGCACTCTACTACTTCTACGGTATGGACGTCTGGGGCC
GAGGGACCACGGTCACCGTCTCCTCA
HCVR (V_{H}) Amino Acid Sequence
(SEQ ID NO: 507)
QVQLVQSGAEVKKPGSSVKVSCKASGGTFNTYAITWVRQAPGQGLEWMGGIIPISGIAEYAQKFQGRVTIT
TDDSSTTAYMELNSLRSEDTAVYYCASWNYALYYFYGMDVWGRGTTVTVSS
(SEQ ID NO: 508)
HCDR1: GGTFNTYA
(SEQ ID NO: 509)
HCDR2: IIPISGIA
(SEQ ID NO: 510)
HCDR3: ASWNYALYYFYGMDV
LCVR (V_{L}) Nucleotide Sequence
(SEQ ID NO: 511)
GAAATTGTGTTGACGCAGTCTCCAGGCACCCTGTCTTTGTCTCCAGGGGAAAGAGCCACCCTCTCCTGC
AGGGCCAGTCAGAGTGTTAGCAGCAGCTACTTAGCCTGGTACCAGCAGAAACCTGGCCAGGCTCCCAG
GCTCCTCATCTATGGTGCATCCAGCAGGGCCACTGGCATCCCAGACAGGTTCAGTGGCAGTGGGTCTG
GGACAGACTTCACTCTCACCATCAGCAGACTGGAGCCTGAAGATTTTGCAGTGTATTACTGTCAGCAG
TATGGTAGCTCACCTTGGACGTTCGGCCAAGGGACCAAGGTGGAAATCAAA
LCVR (V_{L}) Amino Acid Sequence
(SEQ ID NO: 512)
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSPWTFGQGTKVEIK
(SEQ ID NO: 513)
LCDR1: QSVSSSY
(SEQ ID NO: 514)
LCDR2: GAS
(SEQ ID NO: 515)
LCDR3: QQYGSSPWT
69261
HCVR (V_{H}) Nucleotide Sequence
(SEQ ID NO: 516)
CAGGTGCAGCTGGTGGAGTCTGGGGGAGGCTTGGTCAAGCCTGGAGGGTCCCTGAGACTCTCCTGTGC
AGCCTCTGGATTCACCTTCAGTGTCTATTACATGAACTGGATCCGCCAGGCTCCAGGGAAGGGCCTGG
AGTGGGTTTCATACATTAGTAGTAGTGGTAGTACCATATACTACGCAGACTCTGTGAAGGGCCGATTC
ACCATCTCCAGGGACAACGCCAAGAACTCACTGTATCTCCAAATGAACAGTCTGAGAGCCGAGGACA
CGGCCGTATATTACTGTGGGAGAGAAGGGTATAGTGGGACTTATTCTTATTACGGTATGGACGTCTGG
GGCCAAGGGACCACGGTCACCGTCTCCTCA
HCVR (V_{H}) Amino Acid Sequence
(SEQ ID NO: 517)
QVQLVESGGGLVKPGGSLRLSCAASGFTFSVYYMNWIRQAPGKGLEWVSYISSSGSTIYYADSVKGRFTIS
RDNAKNSLYLQMNSLRAEDTAVYYCGREGYSGTYSYYGMDVWGQGTTVTVSS
(SEQ ID NO: 518)
HCDR1: GFTFSVYY
(SEQ ID NO: 519)
HCDR2: ISSSGSTI
(SEQ ID NO: 520)
HCDR3: GREGYSGTYSYYGMDV
LCVR (V_{L}) Nucleotide Sequence
(SEQ ID NO: 521)
GATATTGTGATGACTCAGTCTCCACTCTCCCTGCCCGTCACCCCTGGAGAGCCGGCCTCCATCTCCTGC
AGGTCTAGTCAGAGCCTCCTGCATAGTAATGGATACAACTATTTGGATTGGTACCTGCAGAAGCCAGG
GCAGTCTCCACAGTTCCTGATCTATTTGGGTTCTAATCGGGCCTCCGGGGTCCCTGACAGGTTCAGTGG
CAGTGGATCAGGCACAGATTTTACACTGAAAATCAACAGAGTGGAGGCTGAGGATGTTGGGGTTTATT
ACTGCATGCAAGCTCTACAAACTCCGTACACTTTTGGCCAGGGGACCAAGCTGGAGATCAAA
LCVR (V_{L}) Amino Acid Sequence
(SEQ ID NO: 522)
DIVMTQSPLSLPVTPGEPASISCRSSQSLLHSNGYNYLDWYLQKPGQSPQFLIYLGSNRASGVPDRFSGSGSG
TDFTLKINRVEAEDVGVYYCMQALQTPYTFGQGTKLEIK
(SEQ ID NO: 523)
LCDR1: QSLLHSNGYNY
(SEQ ID NO: 524)
LCDR2: LGS
(SEQ ID NO: 525)
LCDR3: MQALQTPYT
69263
HCVR (V_{H}) Nucleotide Sequence
(SEQ ID NO: 526)
GAAGTGCAGCTGGTGGAGTCTGGGGGAGGGTTGGTACAGCCTGGCAGGTCCCTGAGACTCTCCTGTGC
AGTCTCTGGATTCACCTTTGATGATTATGCCATGCACTGGGTCCGGCAAGCTCCAGGGAAGGGCCTGG
AGTGGGTCTCAGGTATTAGTTGGAATAGTGGTACCAGAGGATATGCGGACTCTGTGAAGGGCCGATTC
ACCATCTCCAGAGACAACGCCAAGAACTCCCTGTATCTGCAAATGAACAGTCTGAGAGGTGAGGACA
CGGCCTTGTATTACTGTGTAAAAGATATTACGATATCCCCCAACTACTACGGTATGGACGTCTGGGGCC
AAGGGACCACGGTCACCGTCTCCTCA
HCVR (V_{H}) Amino Acid Sequence
(SEQ ID NO: 527)
EVQLVESGGGLVQPGRSLRLSCAVSGFTFDDYAMHWVRQAPGKGLEWVSGISWNSGTRGYADSVKGRFT
ISRDNAKNSLYLQMNSLRGEDTALYYCVKDITISPNYYGMDVWGQGTTVTVSS
(SEQ ID NO: 528)
HCDR1: GFTFDDYA
(SEQ ID NO: 529)
HCDR2: ISWNSGTR
(SEQ ID NO: 530)
HCDR3: VKDITISPNYYGMDV
LCVR (V_{L}) Nucleotide Sequence
(SEQ ID NO: 531)
GACATCCAGATGACCCAGTCTCCATCCTCCCTGTCTGCATCTGTAGGAGACAGAGTCACCATCACTTGC
CGGGCGAGTCAGGACATTAGCCATTATTCAGCCTGGTATCAGCAGAAACCAGGGAAACTTCCTAACCT
CCTGATCTATGCTGCATCCACTTTGCAATCAGGGGTCCCATCTCGGTTCAGTGGCAGTGGATCTGGGAC
AGATTTCTCTCTCACCACCAGCAGCCTGCAGCCTGAAGATGTTGCAACTTATTACTGTCAAAAGTATAA
CAGTGTCCCTCTCACTTTCGGCGGAGGGACCAAGGTGGAGATCAAA
LCVR (V_{L}) Amino Acid Sequence
(SEQ ID NO: 532)
DIQMTQSPSSLSASVGDRVTITCRASQDISHYSAWYQQKPGKLPNLLIYAASTLQSGVPSRFSGSGSGTDFSL
TTSSLQPEDVATYYCQKYNSVPLTFGGGTKVEIK
(SEQ ID NO: 533)
LCDR1: QDISHY
(SEQ ID NO: 534)
LCDR2: AAS
(SEQ ID NO: 535)
LCDR3: QKYNSVPLT

TABLE 26
Anti-hTfR scFv Molecules in Fusion Proteins.

| | SEQ | |
|---|---|---|
| Antibody | ID | |
| clone | NO | Amino acid sequence (Vk-3xG4S-Vh) |
| 12795B | 538 | DIQMTQSPSSLSASVGDRVTITCRASQGIRDHFGWYQQKPGKAPKRLIYAASSLHSGVPSRFS |
| | | GSGSGTEFTLTISSLQPEDFATYYCLQYDTYPLTFGGGTKVEIKGGGGSGGGGSGGGGSEVQ |
| | | LVESGGGLVQPGGSLRLSCATSGFTFTSYDMKWVRQAPGLGLEWVSAISGSGGNTYYADSV |
| | | KGRFTISRDNSRNTLYLQMNSLRAEDTAVYYCTRSHDFGAFDYFDYWGQGTLVTVSS |
| 12798B | 539 | EIVMTQSPATLSVSPGERATLSCRASQTVSSNLAWYQQKPGQAPRLLIYGSSSRATGIPARFS |
| | | GSGSGTEFTLTISSLQSEDFAVYYCQQYNNWPPYTFGQGTKLEIKGGGGSGGGGSGGGGSEV |
| | | QLVESGGDLVQPGRSLRLSCAASGFTFDDYAMHWVRQAPGKGLEWVSGISWNSATRVYAD |
| | | SVKGRFTISRDNAKNFLYLQMNSLRSEDTALYHCAKDMDISLGYYGLDVWGQGTTVTVSS |
| 12799B | 540 | DIQMTQSPSSVSASVGDRVTITCRASQGIASWLAWYQQKPGKAPELLIYAASSLQGGVPSRF |
| | | SGSGSGTDFTLTISSLQPEDFAIYYCQQANYFPWTFGQGTKVEIKGGGGSGGGGSGGGGSQIT |
| | | LKESGPTLVKPTQTLTLTCTFSGFSLSTSGVGVVWIRQPPGKALEWLALIYWNDHKRYSPSL |
| | | GSRLTITKDTSKNQVVLTMTNMDPVDTATYYCAHYSGSYSYYYYGLDVWGQGTTVTVSS |
| 12801B | 541 | DIQMTQSPSSLSASVGDRVTITCRASQGIRTDLGWYQQKPGKAPKRLIYAASSLQSGVPSRFS |
| | | GSGSGTEFTLTISSLRPEDFATFYCLQYNSYPLTFGGGTKVEIKGGGGSGGGGSGGGGSEVQL |
| | | LESGGALVQPGGSLRLSCAASGFTFTSYAMHWVRQAPGKGLEWVSSIRGSGGGTYSADSVK |
| | | GRFTISRDNSRDTLYLQMNSVRAEDTAVYYCARSHDYGAFDFFDYWGQGTLVTVSS |
| 12802B | 542 | EIVMTQSPATLSVSPGERATLSCRASQSVSINLAWYQQKPGQAPRLLIFVASTRATGIPARFSG |
| | | SGSGTEFTLTISSLQSEDFATYYCQQYDIWPYTFGQGTKLEIKGGGGSGGGGSGGGGSQVQL |
| | | VESGGGLVKPGGSLRLSCAASGFTFSDYFMSWIRQAPGKGLEWVSYISSTGSTINYADSVKG |
| | | RFTISRDNVKNSLYLQMTSLRVEDTAVYYCTRDNWNYEYWGQGTLVTVSS |
| 12808B | 543 | DIQMTQSPSSLSASVGDRVTINCRASQGIRNDLGWYQQKPGKAPKRLIYAASSLQSGVPLRFS |
| | | GSGSGTEFTLTINNLQPEDFATYYCLSHNSYPWTFGQGTKVEIKGGGGSGGGGSGGGGSQLQ |
| | | LQESGPGLVKPSETLSLTCTVSGESISSNTYYWGWIRQPPGKGLEWIGSIDYSGTTNYNPSLK |
| | | SRVTISVDTSRNHFSLRLRSVTAADTAVYYCAREWGNYGYYYGMDVWGQGTTVTVSS |
| 12812B | 544 | DIQMTQSPPSVSASVGDRVTITCRASQGISSWLAWYQQKPGKAPKLLIYAASSLQSGVPSRFS |
| | | GSGSGTDFTLTISSLQPEDFATYYCQQANSFPRTFGQGTKVEIKGGGGSGGGGSGGGGSQVQ |
| | | LVQSGAEVKKPGSSVRVSCKASRGTFSSYAISWVRQAPGQGLEWMGGIIPIFGTANYAQKFL |
| | | ARVTITADESTSTAYMELSSLRSEDTAVYYCAREKGWNYFDYWGQGTLVTVSS |
| 12816B | 545 | DIVMTQSPLSLPVTPGEPASISCRSSQSLLHGNGYNYLTWYLQKPGQSPQLLIYLGSNRASGV |
| | | PDRFSGSGSGTDFTLKISRVEAEDVGVYYCMQALQTPYTFGQGTKLEIKGGGGSGGGGSGG |
| | | GGSQVQLVESGGGLVKPGGSLRLSCAASGFTFSDYYMNWIRQAPGKGLEWVSYISSSGTTIY |
| | | YADSVKGRFTISRDNAKKSLYLEMNSLRAEDTAVYYCAREGYGNDYYYYGIDVWGQGTTV |
| | | TVSS |
| 12833B | 546 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSQVQ |
| | | LVESGGGVVQPGRSLRLSCAASGFTFSSFGMHWVRQAPGKGLEWVIFISYDGSDKYYADSV |
| | | KGRFAISRDSSKNTLYLQMNSLRAEDTAVYYCAKENGILTDSYGMDVWGQGTTVTVSS |
| 12834B | 547 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSQVQ |
| | | LVQSGAEVKKPGASVKVSCKASGYTFTSYGISWVRQAPGQGLEWMGWISVYHGNTNYAQ |
| | | KFQGRVTMTTDTSTSTAYMELRSLRSDDTAVYYCAREGYYDFWSGYYPFDYWGQGTLVT |
| | | VSS |
| 12835B | 548 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSEVQ |
| | | LVESGGGLIQPGGSLRLSCEASGFTFRNYEMNWVRQAPGKGLEWVSYISSSGNMKDYAESV |
| | | KGRFTISRDNVKNSLQLQMNSLRVEDTAVYYCARDEFPYGMDVWGQGTTVTVSS |
| 12839B | 549 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSQVQ |
| | | LVESGGGVVQPGRSLRLSCAASGFPFSNYVMYWVRQAPGKGLEWVALIFFDGKKNYHADS |
| | | VKGRFTITRDNSKNMLYLQMNSLRPEDAAVYYCAKIHCPNGVCYKGYYGMDVWGQGTTV |
| | | TVSS |
| 12841B | 550 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSEVQ |
| | | LVESGGGLVQPGGSLRLSCAASGFTFSNYWMNWVRQAPGKGLEWVANIKEDGGKKLYVD |
| | | SVKGRFTISRDNAKNSLFLQMNSLRAEDTAVYYCAREDTTLVVDYYYYGMDVWGQGTTV |
| | | TVSS |
| 12843B | 551 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSEVQ |
| | | LVESGGGLVQPGGSLRLSCAASGFTFNIFEMNWVRQAPGKGLEWISYISSRGTTTYYADSVR |
| | | GRFTISRDNAKNSLYLQMNSLRAEDTAVYYCARDYEATIPFDFWGQGTLVTVSS |
| 12844B | 552 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSEVQ |
| | | LVESGGSVVRPGGSLRLSCEASGFTFDDYGMSWVRQDPGKGLEWVSGINWNGDRTNYADS |
| | | VKGRFIISRDNAKNSVYLQMNSLRAEDSALYHCARDQGLGVAATLDYWGQGTLVTVSS |
| 12845B | 553 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSEVQ |
| | | LVESGGGLVQPGGSLRLSCAASGFTVSNYEMNWVRQAPGKGLEWVSYISSSTSNIYYADSV |
| | | KGRFTISRDNAENSLYLQMNSLRVEDTAVYYCVRDGIVVVPVGRGYYYYGLDVWGQGTTV |
| | | TVSS |
| 12847B | 554 | DIQMTQSPSSLSASVGDRVTITCRASQSISSYLNWYQQKPGKAPKLLIYAASSLQSGVPSRFS |
| | | GSGSGTDFTLTISSLQPEDFATYYCQQSYSTPPITFGQGTRLEIKGGGGSGGGGSGGGGSEVQ |
| | | LVESGGGLVQPGRSLRLSCAASGFTFDDYAMNWVRQAPGKGLEWVSGISWSSGSMDYADS |
| | | VKGRFTISRDNAKNSLYLQMNSLRTEDTALYYCAKAREVGDYYGMDVWGQGTTVTVSS |
| 12848B | 555 | EIVLTQSPGTLSLSPGERATLSCRASQSVSSSYLAWYQQKPGQAPRLLIYGASSRATGIPDRFS |
| | | GSGSGTDFTLTISRLEPEDFAVYYCQQYGSSPWTFGQGTKVEIKGGGGSGGGGSGGGGSEVQ |
| | | LVESGGGLVQPGRSLTLSCAASGFTFDNFGMHWVRQGPGKGLEWVSGLTWNSGVIGYADS |
| | | VKGRFTISRDNAKNSLYLQMNSLRPEDTALYYCAKDIRNYGPFDYWGQGTLVTVSS |
| 12850B | 556 | EIVLTQSPGTLSLSPGERATLSCRASQSVSSSYLAWYQQKPGQAPRLLIYGASSRATGIPDRFS |
| | | GSGSGTDFTLTISRLEPEDFAVYYCQQYGSSPWTFGQGTKVEIKGGGGSGGGGSGGGGSQV |
| | | QLVQSGAEVKKPGSSVKVSCKASGGTFNTYAITWVRQAPGQGLEWMGGIIPISGIAEYAQKF |
| | | QGRVTITTDDSSTTAYMELNSLRSEDTAVYYCASWNYALYYFYGMDVWGRGTTVTVSS |
| 31863B | 557 | DIQMTQSPSSLSASIGDRVTITCRASQGISNYLAWYQQKPGKVPKLLIYAASTLQSGVPSRFS |
| | | GSGSGTDFTLTISSLQPEDVATYYCQNHNSVPLTFGGGTKVEIKGGGGSGGGGSGGGGSEVQ |
| | | LVESGGGLVQPGGSLRLSCAASGFTFNSYAMTWVRQAPGKGLEWVSFIGGSTGNTYYAGSV |
| | | KGRFTISSDNSKKTLYLQMNSLRAEDTAVYYCAKGGAARRMEYFQHWGQGTLVTVSS |
| 31874B | 558 | DIQMTQSPSSLSASVGDRVTITCRASQGISNYLAWYQQKPGKVPNLLIYAASTLQSGVPSRFS |
| | | GSGSGTDFTLTISSLQPEDVATYYCQKYNSAPLTFGGGTKVEIKGGGGSGGGGSGGGGSEVQ |
| | | LVESGGGLVQPGGSLRLSCAASGFAFSSYAMTWVRQAPGKGLEWVSVISGTGGSTYYADSV |
| | | KGRFTISRDNSKNTLYLQMNSLRAEDTAVYYCAKGGAARRMEYFQYWGQGTLVTVSS |
| 69261 | 559 | DIVMTQSPLSLPVTPGEPASISCRSSQSLLHSNGYNYLDWYLQKPGQSPQFLIYLGSNRASGV |
| | | PDRFSGSGSGTDFTLKINRVEAEDVGVYYCMQALQTPYTFGQGTKLEIKGGGGSGGGGSGG |
| | | GGSQVQLVESGGGLVKPGGSLRLSCAASGFTFSVYYMNWIRQAPGKGLEWVSYISSSGSTIY |
| | | YADSVKGRFTISRDNAKNSLYLQMNSLRAEDTAVYYCGREGYSGTYSYYGMDVWGQGTT |
| | | VTVSS |
| 69263 | 560 | DIQMTQSPSSLSASVGDRVTITCRASQDISHYSAWYQQKPGKLPNLLIYAASTLQSGVPSRFS |
| | | GSGSGTDFSLTTSSLQPEDVATYYCQKYNSVPLTFGGGTKVEIKGGGGSGGGGSGGGGSEV |
| | | QLVESGGGLVQPGRSLRLSCAVSGFTFDDYAMHWVRQAPGKGLEWVSGISWNSGTRGYAD |
| | | SVKGRFTISRDNAKNSLYLQMNSLRGEDTALYYCVKDITISPNYYGMDVWGQGTTVTVSS |
| 69305 | 561 | DIQMTQSPSSLSASVGDRVTITCRASQSIDRYLNWYRQKPGKAPKLLIYTTSSLQSGVPSRFS |
| | | GSGSGTDFTLTLSSLQPEDFATYYCQQSYSPPLTFGGGTKVEIKGGGGSGGGGSGGGGSQVQ |
| | | LVESGGGVVQPGRSLRLSCAASGFTFSSYGMHWVRQAPGKGLEWVAVIWYDGSNKYYAD |
| | | SVKGRFTISRDISKNTLYLQMNSLRAEDTAVYYCAGQLDLFFDYWGQGTLVTVSS |
| 69307 | 562 | DIQMTQSPSSVSASVGDRVTITCRASQGISSWLAWYQQKPGKAPKLLIYAASSLQSGVPSRFS |
| | | GSGSGTDFTLTISSLQPEDFATYYCQKADSLPYAFGQGTKLEIKGGGGSGGGGSGGGGSEVQ |
| | | LVESGGGLVQPGGSLRLSCTASGFTFSNYWMTWVRQAPGKGLEWVANIKEDGSEKEYVDS |
| | | VKGRFTISRDNAKNSLYLQMNSLRGEDTAVYYCARDGEQLVDYYYYYVMDVWGQGTTVT |
| | | VSS |
| 69323 | 563 | DIQMTQSPSSLSASVGDRVTITCRASQSISSYLNWYQQKPGKAPKVLIYAASSLQSGVPSRFS |
| | | GSGSGTDFTLTISSLQPEDFATYYCQQSYSIPLTFGGGTKVEIKGGGGSGGGGSGGGGSEVQL |
| | | VESGGGLVQPGRSLRLSCAASGFTFDDYAMHWVRQAPGKGLEWVSGISWNSGYIGYADSV |
| | | KGRFTISRDNAENSLHLQMNSLRAEDTALYYCARGGSTLVRGVKGGYYGMDVWGQGTTV |
| | | TVSS |
| 69326 | 564 | EIVMTQSPATLSVSPGERATLSCRASQSVSSNFAWYQQKPGQAPRLLIYSASSRATGIPVRFS |
| | | GSGSGTEFTLTISSLQSEDFAVYYCQQYNIWPRTFGQGTKVEIKGGGGSGGGGSGGGGSEVQ |
| | | LVESGGGLVQPGGSLRLSCAVSGFIFSSYEMNWVRQAPGKGLEWVSYISSSGSTIFYADSVK |
| | | GRFTISRDNAKNSLYLQMNSLRAEDTAVYYCVSGVVLFDVWGQGTMVTVSS |
| 69329 | 565 | DIQMTQSPSSVSASVGDRVTITCRASQGISSWLAWYQQKPGKAPKLLIYAASSLQSGVPSRFS |
| | | GSGSGTDFTLTISSLQPEDFATYYCQKANSFPYTFGQGTKLEIKGGGGSGGGGSGGGGSEVQ |
| | | LVESGGGLVQPGGSLRLSCAASGFTFSNYWMTWVRQAPGKGLEWVANIKEDGSEKDYVDS |
| | | VKGRFTISRDNAKNSLYLQMNSLRGEDTAVYYCARDGEQLVDYYYYYVMDVWGQGTTVT |
| | | VSS |
| 69331 | 566 | DIQLTQSPSSLSASVGDRVTITCWASQGISSYLAWYQQKPGKAPKLLIYAASTLQSGVPSRFS |
| | | GSGSGTEFTLTISSLQPEDFATYYCQQLNSYPLTFGGGTKVEIKGGGGSGGGGSGGGGSQVQ |
| | | LVESGGGVVQPGRSLRLSCIASGFTFSVYGIHWVRQAPGKGLEWMAVISHDGNIKHYADSV |
| | | KGRFTISRDNSKNTLYLQINSLRTEDTAVYYCAKDTWNSLDTFDIWGQGTMVTVSS |
| 69332 | 567 | AIQMTQSPSSLSASVGDRVTITCRASQGIRNDLGWYQQKPGKAPKLLIYAASTLQSGVPSRFS |
| | | GSGSGTDFTLTISSLQPEDFATYYCLQDYNYPFTFGPGTKVDIKGGGGSGGGGSGGGGSQVT |
| | | LRESGPALVKPSQTLTLTCTFSGFSLNTYGMFVSWIRQPPGKALEWLAHIHWDDDKYYSTSL |
| | | KTRLTISKDTSKNQVVLTMTNMDPVDTATYYCARGHNNLNYIIHWGQGTLVTVSS |
| 69340 | 568 | EIVLTQSPATLSLSPGERATLSCRASQSVSSYLAWYQQKPGQAPRLLIHDVSNRATGIPARFS |
| | | GSGSGTDFTLTISSLEPEDFVVYYCQQRSDWPITFGQGTRLEIKGGGGSGGGGSGGGGSEVQ |
| | | LVESGGGLVQPGRSLRLSCAASGFTFDDKAMHWVRQVPGKGLEWISGISWNSGTIGYADSV |
| | | KGRFIISRDNAKNSLYLQMNSLRAEDTALYYCAKDGDTSGWYWYGLDVWGQGTTVTVSS |
| 69348 | 569 | DIQMTQSPSSLSASVGDRVTITCRASQSIRNVLGWFQQKPGKAPQRLIYAASSLQSGVPSRFS |
| | | GSGSGTEFTLTISSLQPEDFATYYCLQHNFYPLTFGGGTKVEIKGGGGSGGGGSGGGGSQVQ |
| | | LVESGGGVVQPGRSLRLSCAASGFTFTTYGMHWVRQAPGKGLEWVAVIWYDGSNKYYGD |
| | | SVKGRFTISRDNSKNTLYLQMNSLRVDDTAVYYCTRTHGYTRSSDGFDYWGQGTLVTVSS |

Anti-TfR scFV:GAA Sequences:
(1) 12795B
DIQMTQSPSSLSASVGDRVTITCRASQGIRDHFGWYQQKPGKAPKRLIYAASSLHSGVPSRFSGSGSGTEFTL
TISSLQPEDFATYYCLQYDTYPLTFGGGTKVEIKGGGGSGGGGSGGGGSEVQLVESGGGLVQPGGSLRLSC
ATSGFTFTSYDMKWVRQAPGLGLEWVSAISGSGGNTYYADSVKGRFTISRDNSRNTLYLQMNSLRAEDTA
VYYCTRSHDFGAFDYFDYWGQGTLVTVSSGGGGSGGGGSAHPGRPRAVPTQCDVPPNSRFDCAPDKAITQ
EQCEARGCCYIPAKQGLQGAQMGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVIVRRQLDGRVLLNTTVAPLFFAD
QFLQLSTSLPSQYITGLAEHLSPLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPPYWGLGFHLCRWGYSSTAITR
QVVENMTRAHFPLDVQWNDLDYMDSRRDFTFNKDGFRDFPAMVQELHQGGRRYMMIVDPAISSSGPAGS
YRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 184)
(2) 12798B (REGN16818)
EIVMTQSPATLSVSPGERATLSCRASQTVSSNLAWYQQKPGQAPRLLIYGSSSRATGIPARFSGSGSGTEFTL
TISSLQSEDFAVYYCQQYNNWPPYTFGQGTKLEIKGGGGSGGGGSGGGGSEVQLVESGGDLVQPGRSLRLS
CAASGFTFDDYAMHWVRQAPGKGLEWVSGISWNSATRVYADSVKGRFTISRDNAKNFLYLQMNSLRSED
TALYHCAKDMDISLGYYGLDVWGQGTTVTVSSGGGGSGGGGSAHPGRPRAVPTQCDVPPNSRFDCAPDK
AITQEQCEARGCCYIPAKQGLQGAQMGQPWCFFPPSYPSYKLENLSSSEMGYTATLTRTTPTFFPKDILTLR
LDVMMETENRLHFTIKDPANRRYEVPLETPHVHSRAPSPLYSVEFSEEPFGVIVRRQLDGRVLLNTTVAPLF
FADQFLQLSTSLPSQYITGLAEHLSPLMLSTSWTRITLWNRDLAPTPGANLYGSHPFYLALEDGGSAHGVFL
LNSNAMDVVLQPSPALSWRSTGGILDVYIFLGPEPKSVVQQYLDVVGYPFMPPYWGLGFHLCRWGYSSTA
ITRQVVENMTRAHFPLDVQWNDLDYMDSRRDFTFNKDGFRDFPAMVQELHQGGRRYMMIVDPAISSSGP
AGSYRPYDEGLRRGVFITNETGQPLIGKVWPGSTAFPDFTNPTALAWWEDMVAEFHDQVPFDGMWIDMN
EPSNFIRGSEDGCPNNELENPPYVPGVVGGTLQAATICASSHQFLSTHYNLHNLYGLTEAIASHRALVKARG
TRPFVISRSTFAGHGRYAGHWTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTLFHQAHVAGETVARPLFLEFPK
DSSTWTVDHQLLWGEALLITPVLQAGKAEVTGYFPLGTWYDLQTVPVEALGSLPPPPAAPREPAIHSEGQW
VTLPAPLDTINVHLRAGYIIPLQGPGLTTTESRQQPMALAVALTKGGEARGELFWDDGESLEVLERGAYTQ
VIFLARNNTIVNELVRVTSEGAGLQLQKVTVLGVATAPQQVLSNGVPVSNFTYSPDTKVLDICVSLLMGEQ
FLVSWC
(SEQ ID NO: 185)
(3) 12799B (REGN16819)
DIQMTQSPSSVSASVGDRVTITCRASQGIASWLAWYQQKPGKAPELLIYAASSLQGGVPSRFSGSGSGTDFT
LTISSLQPEDFAIYYCQQANYFPWTFGQGTKVEIKGGGGSGGGGSGGGGSQITLKESGPTLVKPTQTLTLTC
TFSGFSLSTSGVGVVWIRQPPGKALEWLALIYWNDHKRYSPSLGSRLTITKDTSKNQVVLTMTNMDPVDT
ATYYCAHYSGSYSYYYYGLDVWGQGTTVTVSSGGGGSGGGGSAHPGRPRAVPTQCDVPPNSRFDCAPDK
AITQEQCEARGCCYIPAKQGLQGAQMGQPWCFFPPSYPSYKLENLSSSEMGYTATLTRTTPTFFPKDILTLR
LDVMMETENRLHFTIKDPANRRYEVPLETPHVHSRAPSPLYSVEFSEEPFGVIVRRQLDGRVLLNTTVAPLF
FADQFLQLSTSLPSQYITGLAEHLSPLMLSTSWTRITLWNRDLAPTPGANLYGSHPFYLALEDGGSAHGVFL
LNSNAMDVVLQPSPALSWRSTGGILDVYIFLGPEPKSVVQQYLDVVGYPFMPPYWGLGFHLCRWGYSSTA
ITRQVVENMTRAHFPLDVQWNDLDYMDSRRDFTFNKDGFRDFPAMVQELHQGGRRYMMIVDPAISSSGP
AGSYRPYDEGLRRGVFITNETGQPLIGKVWPGSTAFPDFTNPTALAWWEDMVAEFHDQVPFDGMWIDMN
EPSNFIRGSEDGCPNNELENPPYVPGVVGGTLQAATICASSHQFLSTHYNLHNLYGLTEAIASHRALVKARG
TRPFVISRSTFAGHGRYAGHWTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTLFHQAHVAGETVARPLFLEFPK
DSSTWTVDHQLLWGEALLITPVLQAGKAEVTGYFPLGTWYDLQTVPVEALGSLPPPPAAPREPAIHSEGQW
VTLPAPLDTINVHLRAGYIIPLQGPGLTTTESRQQPMALAVALTKGGEARGELFWDDGESLEVLERGAYTQ
VIFLARNNTIVNELVRVTSEGAGLQLQKVTVLGVATAPQQVLSNGVPVSNFTYSPDTKVLDICVSLLMGEQ
FLVSWC
(SEQ ID NO: 570)
(4) 12801B
DIQMTQSPSSLSASVGDRVTITCRASQGIRTDLGWYQQKPGKAPKRLIYAASSLQSGVPSRFSGSGSGTEFTL
TISSLRPEDFATFYCLQYNSYPLTFGGGTKVEIKGGGGSGGGGSGGGGSEVQLLESGGALVQPGGSLRLSCA
ASGFTFTSYAMHWVRQAPGKGLEWVSSIRGSGGGTYSADSVKGRFTISRDNSRDTLYLQMNSVRAEDTAV
YYCARSHDYGAFDFFDYWGQGTLVTVSSGGGGSGGGGSAHPGRPRAVPTQCDVPPNSRFDCAPDKAITQE
QCEARGCCYIPAKQGLQGAQMGQPWCFFPPSYPSYKLENLSSSEMGYTATLTRTTPTFFPKDILTLRLDVM
METENRLHFTIKDPANRRYEVPLETPHVHSRAPSPLYSVEFSEEPFGVIVRRQLDGRVLLNTTVAPLFFADQF
LQLSTSLPSQYITGLAEHLSPLMLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYWGLGFHLCRWGYSSTAITRQV
VENMTRAHFPLDVQWNDLDYMDSRRDFTFNKDGFRDFPAMVQELHQGGRRYMMIVDPAISSSGPAGSYR
PYDEGLRRGVFITNETGQPLIGKVWPGSTAFPDFTNPTALAWWEDMVAEFHDQVPFDGMWIDMNEPSNFI
RGSEDGCPNNELENPPYVPGVVGGTLQAATICASSHQFLSTHYNLHNLYGLTEAIASHRALVKARGTRPFVI
SRSTFAGHGRYAGHWTGDVWSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPA
PLDTINVHLRAGYIIPLQGPGLTTTESRQQPMALAVALTKGGEARGELFWDDGESLEVLERGAYTQVIFLAR
NNTIVNELVRVTSEGAGLQLQKVTVLGVATAPQQVLSNGVPVSNFTYSPDTKVLDICVSLLMGEQFLVSW
C
(SEQ ID NO: 186)
(5) 12802B (REGN16820)
EIVMTQSPATLSVSPGERATLSCRASQSVSINLAWYQQKPGQAPRLLIFVASTRATGIPARFSGSGSGTEFTL
TISSLQSEDFATYYCQQYDIWPYTFGQGTKLEIKGGGGSGGGGSGGGGSQVQLVESGGGLVKPGGSLRLSC
AASGFTFSDYFMSWIRQAPGKGLEWVSYISSTGSTINYADSVKGRFTISRDNVKNSLYLQMTSLRVEDTAV
YYCTRDNWNYEYWGQGTLVTVSSGGGGSGGGGSAHPGRPRAVPTQCDVPPNSRFDCAPDKAITQEQCEA
RGCCYIPAKQGLQGAQMGQPWCFFPPSYPSYKLENLSSSEMGYTATLTRTTPTFFPKDILTLRLDVMMETE
NRLHFTIKDPANRRYEVPLETPHVHSRAPSPLYSVEFSEEPFGVIVRRQLDGRVLLNTTVAPLFFADQFLQLS
TSLPSQYITGLAEHLSPLMLSTSWTRITLWNRDLAPTPGANLYGSHPFYLALEDGGSAHGVFLLNSNAMDV
VLQPSPALSWRSTGGILDVYIFLGPEPKSVVQQYLDVVGYPFMPPYWGLGFHLCRWGYSSTAITRQVVEN
MTRAHFPLDVQWNDLDYMDSRRDFTFNKDGFRDFPAMVQELHQGGRRYMMIVDPAISSSGPAGSYRPYD
EGLRRGVFITNETGQPLIGKVWPGSTAFPDFTNPTALAWWEDMVAEFHDQVPFDGMWIDMNEPSNFIRGS
EDGCPNNELENPPYVPGVVGGTLQAATICASSHQFLSTHYNLHNLYGLTEAIASHRALVKARGTRPFVISRS
TFAGHGRYAGHWTGDVWSSWEQLASSVPEILQFNLLGVPLVGADVCGFLGNTSEELCVRWTQLGAFYPF
MRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDGESLEVLERGAYTQVIFLARNN
TIVNELVRVTSEGAGLQLQKVTVLGVATAPQQVLSNGVPVSNFTYSPDTKVLDICVSLLMGEQFLVSWC
(SEQ ID NO: 187)
(6) 12808B
DIQMTQSPSSLSASVGDRVTINCRASQGIRNDLGWYQQKPGKAPKRLIYAASSLQSGVPLRFSGSGSGTEFT
LTINNLQPEDFATYYCLSHNSYPWTFGQGTKVEIKGGGGSGGGGSGGGGSQLQLQESGPGLVKPSETLSLT
CTVSGESISSNTYYWGWIRQPPGKGLEWIGSIDYSGTTNYNPSLKSRVTISVDTSRNHFSLRLRSVTAADTA
VYYCAREWGNYGYYYGMDVWGQGTTVTVSSGGGGSGGGGSAHPGRPRAVPTQCDVPPNSRFDCAPDKA
ITQEQCEARGCCYIPAKQGLQGAQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFGVIVRRQLDGRVLLNTTVAPLFF
ADQFLQLSTSLPSQYITGLAEHLSPLMLSTSWTRITLWNRDLAPTPGANLYGSHPFYLALEDGGSAHGVFLL
NSNAMDVVLQPSPALSWRSTGGILDVYIFLGPEPKSVVQQYLDVVGYPFMPPYWGLGFHLCRWGYSSTAI
TRQVVENMTRAHFPLDVQWNDLDYMDSRRDFTFNKDGFRDFPAMVQELHQGGRRYMMIVDPAISSSGPA
GSYRPYDEGLRRGVFITNETGQPLIGKVWPGSTAFPDFTNPTALAWWEDMVAEFHDQVPFDGMWIDMNE
PSNFIRGSEDGCPNNELENPPYVPGVVGGTLQAATICASSHQFLSTHYNLHNLYGLTEAIASHRALVKARGT
RPFVISRSTFAGHGRYAGHWTGDVWSSWEQLASSVPEILQFNLLGVPLVGADVCGFLGNTSEELCVRWTQ
LGAFYPFMRNHNSLLSLPQEPYSFSEPAQQAMRKALTLRYALLPHLYTLFHQAHVAGETVARPLFLEFPKD
SSTWTVDHQLLWGEALLITPVLQAGKAEVTGYFPLGTWYDLQTVPVEALGSLPPPPAAPREPAIHSEGQWV
TLPAPLDTINVHLRAGYIIPLQGPGLTTTESRQQPMALAVALTKGGEARGELFWDDGESLEVLERGAYTQVI
FLARNNTIVNELVRVTSEGAGLQLQKVTVLGVATAPQQVLSNGVPVSNFTYSPDTKVLDICVSLLMGEQFL
VSWC
(SEQ ID NO: 188)
(7) 12812B (REGN16821)
DIQMTQSPPSVSASVGDRVTITCRASQGISSWLAWYQQKPGKAPKLLIYAASSLQSGVPSRFSGSGSGTDFT
LTISSLQPEDFATYYCQQANSFPRTFGQGTKVEIKGGGGSGGGGSGGGGSQVQLVQSGAEVKKPGSSVRVS
CKASRGTFSSYAISWVRQAPGQGLEWMGGIIPIFGTANYAQKFLARVTITADESTSTAYMELSSLRSEDTAV
YYCAREKGWNYFDYWGQGTLVTVSSGGGGSGGGGSAHPGRPRAVPTQCDVPPNSRFDCAPDKAITQEQC
EARGCCYIPAKQGLQGAQMGQPWCFFPPSYPSYKLENLSSSEMGYTATLTRTTPTFFPKDILTLRLDVMME
TENRLHFTIKDPANRRYEVPLETPHVHSRAPSPLYSVEFSEEPFGVIVRRQLDGRVLLNTTVAPLFFADQFLQ
LSTSLPSQYITGLAEHLSPLMLSTSWTRITLWNRDLAPTPGANLYGSHPFYLALEDGGSAHGVFLLNSNAM
DVVLQPSPALSWRSTGGILDVYIFLGPEPKSVVQQYLDVVGYPFMPPYWGLGFHLCRWGYSSTAITRQVVE
NMTRAHFPLDVQWNDLDYMDSRRDFTFNKDGFRDFPAMVQELHQGGRRYMMIVDPAISSSGPAGSYRPY
DEGLRRGVFITNETGQPLIGKVWPGSTAFPDFTNPTALAWWEDMVAEFHDQVPFDGMWIDMNEPSNFIRG
SEDGCPNNELENPPYVPGVVGGTLQAATICASSHQFLSTHYNLHNLYGLTEAIASHRALVKARGTRPFVISR
STFAGHGRYAGHWTGDVWSSWEQLASSVPEILQFNLLGVPLVGADVCGFLGNTSEELCVRWTQLGAFYPF
MRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDGESLEVLERGAYTQVIFLARNN
TIVNELVRVTSEGAGLQLQKVTVLGVATAPQQVLSNGVPVSNFTYSPDTKVLDICVSLLMGEQFLVSWC
(SEQ ID NO: 189)
(8) 12816B
DIVMTQSPLSLPVTPGEPASISCRSSQSLLHGNGYNYLTWYLQKPGQSPQLLIYLGSNRASGVPDRFSGSGSG
TDFTLKISRVEAEDVGVYYCMQALQTPYTFGQGTKLEIKGGGGSGGGGSGGGGSQVQLVESGGGLVKPGG
SLRLSCAASGFTFSDYYMNWIRQAPGKGLEWVSYISSSGTTIYYADSVKGRFTISRDNAKKSLYLEMNSLR
AEDTAVYYCAREGYGNDYYYYGIDVWGQGTTVTVSSGGGGSGGGGSAHPGRPRAVPTQCDVPPNSRFDC
APDKAITQEQCEARGCCYIPAKQGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVVGYPFMPPYWGLGFHLCRWGY
SSTAITRQVVENMTRAHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPHLYTLFHQAHVAGETVARPLF
LEFPKDSSTWTVDHQLLWGEALLITPVLQAGKAEVTGYFPLGTWYDLQTVPVEALGSLPPPPAAPREPAIH
SEGQWVTLPAPLDTINVHLRAGYIIPLQGPGLTTTESRQQPMALAVALTKGGEARGELFWDDGESLEVLER
GAYTQVIFLARNNTIVNELVRVTSEGAGLQLQKVTVLGVATAPQQVLSNGVPVSNFTYSPDTKVLDICVSL
LMGEQFLVSWC
(SEQ ID NO: 190)
(9) 12833B
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSQVQLVESGGGVVQPGRSLRLSC
AASGFTFSSFGMHWVRQAPGKGLEWVIFISYDGSDKYYADSVKGRFAISRDSSKNTLYLQMNSLRAEDTA
VYYCAKENGILTDSYGMDVWGQGTTVTVSSGGGGSGGGGSAHPGRPRAVPTQCDVPPNSRFDCAPDKAIT
QEQCEARGCCYIPAKQGLQGAQMGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVIVRRQLDGRVLLNTTVAPLFFAD
QFLQLSTSLPSQYITGLAEHLSPLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPPYWGLGFHLCRWGYSSTAITR
QVVENMTRAHFPLDVQWNDLDYMDSRRDFTFNKDGFRDFPAMVQELHQGGRRYMMIVDPAISSSGPAGS
YRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 191)
(10) 12834B
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSQVQLVQSGAEVKKPGASVKVSC
KASGYTFTSYGISWVRQAPGQGLEWMGWISVYHGNTNYAQKFQGRVTMTTDTSTSTAYMELRSLRSDDT
AVYYCAREGYYDFWSGYYPFDYWGQGTLVTVSSGGGGSGGGGSAHPGRPRAVPTQCDVPPNSRFDCAPD
KAITQEQCEARGCCYIPAKQGLQGAQMGQPWCFFPPSYPSYKLENLSSSEMGYTATLTRTTPTFFPKDILTL
RLDVMMETENRLHFTIKDPANRRYEVPLETPHVHSRAPSPLYSVEFSEEPFGVIVRRQLDGRVLLNTTVAPL
FFADQFLQLSTSLPSQYITGLAEHLSPLMLSTSWTRITLWNRDLAPTPGANLYGSHPFYLALEDGGSAHGVF
LLNSNAMDVVLQPSPALSWRSTGGILDVYIFLGPEPKSVVQQYLDVVGYPFMPPYWGLGFHLCRWGYSST
AITRQVVENMTRAHFPLDVQWNDLDYMDSRRDFTFNKDGFRDFPAMVQELHQGGRRYMMIVDPAISSSG
PAGSYRPYDEGLRRGVFITNETGQPLIGKVWPGSTAFPDFTNPTALAWWEDMVAEFHDQVPFDGMWIDM
NEPSNFIRGSEDGCPNNELENPPYVPGVVGGTLQAATICASSHQFLSTHYNLHNLYGLTEAIASHRALVKAR
GTRPFVISRSTFAGHGRYAGHWTGDVWSSWEQLASSVPEILQFNLLGVPLVGADVCGFLGNTSEELCVRW
TQLGAFYPFMRNHNSLLSLPQEPYSFSEPAQQAMRKALTLRYALLPHLYTLFHQAHVAGETVARPLFLEFP
KDSSTWTVDHQLLWGEALLITPVLQAGKAEVTGYFPLGTWYDLQTVPVEALGSLPPPPAAPREPAIHSEGQ
WVTLPAPLDTINVHLRAGYIIPLQGPGLTTTESRQQPMALAVALTKGGEARGELFWDDGESLEVLERGAYT
QVIFLARNNTIVNELVRVTSEGAGLQLQKVTVLGVATAPQQVLSNGVPVSNFTYSPDTKVLDICVSLLMGE
QFLVSWC
(SEQ ID NO: 192)
(11) 12835B
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSEVQLVESGGGLIQPGGSLRLSCE
ASGFTFRNYEMNWVRQAPGKGLEWVSYISSSGNMKDYAESVKGRFTISRDNVKNSLQLQMNSLRVEDTA
VYYCARDEFPYGMDVWGQGTTVTVSSGGGGSGGGGSAHPGRPRAVPTQCDVPPNSRFDCAPDKAITQEQ
CEARGCCYIPAKQGLQGAQMGQPWCFFPPSYPSYKLENLSSSEMGYTATLTRTTPTFFPKDILTLRLDVMM
ETENRLHFTIKDPANRRYEVPLETPHVHSRAPSPLYSVEFSEEPFGVIVRRQLDGRVLLNTTVAPLFFADQFL
QLSTSLPSQYITGLAEHLSPLMLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYWGLGFHLCRWGYSSTAITRQV
VENMTRAHFPLDVQWNDLDYMDSRRDFTFNKDGFRDFPAMVQELHQGGRRYMMIVDPAISSSGPAGSYR
PYDEGLRRGVFITNETGQPLIGKVWPGSTAFPDFTNPTALAWWEDMVAEFHDQVPFDGMWIDMNEPSNFI
RGSEDGCPNNELENPPYVPGVVGGTLQAATICASSHQFLSTHYNLHNLYGLTEAIASHRALVKARGTRPFVI
SRSTFAGHGRYAGHWTGDVWSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPA
PLDTINVHLRAGYIIPLQGPGLTTTESRQQPMALAVALTKGGEARGELFWDDGESLEVLERGAYTQVIFLAR
NNTIVNELVRVTSEGAGLQLQKVTVLGVATAPQQVLSNGVPVSNFTYSPDTKVLDICVSLLMGEQFLVSW
C
(SEQ ID NO: 193)
(12) 12839B (REGN16822)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSQVQLVESGGGVVQPGRSLRLSC
AASGFPFSNYVMYWVRQAPGKGLEWVALIFFDGKKNYHADSVKGRFTITRDNSKNMLYLQMNSLRPEDA
AVYYCAKIHCPNGVCYKGYYGMDVWGQGTTVTVSSGGGGSGGGGSAHPGRPRAVPTQCDVPPNSRFDC
APDKAITQEQCEARGCCYIPAKQGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVVGYPFMPPYWGLGFHLCRWGY
SSTAITRQVVENMTRAHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPHLYTLFHQAHVAGETVARPLF
LEFPKDSSTWTVDHQLLWGEALLITPVLQAGKAEVTGYFPLGTWYDLQTVPVEALGSLPPPPAAPREPAIH
SEGQWVTLPAPLDTINVHLRAGYIIPLQGPGLTTTESRQQPMALAVALTKGGEARGELFWDDGESLEVLER
GAYTQVIFLARNNTIVNELVRVTSEGAGLQLQKVTVLGVATAPQQVLSNGVPVSNFTYSPDTKVLDICVSL
LMGEQFLVSWC
(SEQ ID NO: 571)
(13) 12841B (REGN16823)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSEVQLVESGGGLVQPGGSLRLSC
AASGFTFSNYWMNWVRQAPGKGLEWVANIKEDGGKKLYVDSVKGRFTISRDNAKNSLFLQMNSLRAEDT
AVYYCAREDTTLVVDYYYYGMDVWGQGTTVTVSSGGGGSGGGGSAHPGRPRAVPTQCDVPPNSRFDCA
PDKAITQEQCEARGCCYIPAKQGLQGAQMGQPWCFFPPSYPSYKLENLSSSEMGYTATLTRTTPTFFPKDIL
TLRLDVMMETENRLHFTIKDPANRRYEVPLETPHVHSRAPSPLYSVEFSEEPFGVIVRRQLDGRVLLNTTVA
PLFFADQFLQLSTSLPSQYITGLAEHLSPLMLSTSWTRITLWNRDLAPTPGANLYGSHPFYLALEDGGSAHG
VFLLNSNAMDVVLQPSPALSWRSTGGILDVYIFLGPEPKSVVQQYLDVVGYPFMPPYWGLGFHLCRWGYS
STAITRQVVENMTRAHFPLDVQWNDLDYMDSRRDFTFNKDGFRDFPAMVQELHQGGRRYMMIVDPAISS
SGPAGSYRPYDEGLRRGVFITNETGQPLIGKVWPGSTAFPDFTNPTALAWWEDMVAEFHDQVPFDGMWID
MNEPSNFIRGSEDGCPNNELENPPYVPGVVGGTLQAATICASSHQFLSTHYNLHNLYGLTEAIASHRALVK
ARGTRPFVISRSTFAGHGRYAGHWTGDVWSSWEQLASSVPEILQFNLLGVPLVGADVCGFLGNTSEELCVR
WTQLGAFYPFMRNHNSLLSLPQEPYSFSEPAQQAMRKALTLRYALLPHLYTLFHQAHVAGETVARPLFLEF
PKDSSTWTVDHQLLWGEALLITPVLQAGKAEVTGYFPLGTWYDLQTVPVEALGSLPPPPAAPREPAIHSEG
QWVTLPAPLDTINVHLRAGYIIPLQGPGLTTTESRQQPMALAVALTKGGEARGELFWDDGESLEVLERGAY
TQVIFLARNNTIVNELVRVTSEGAGLQLQKVTVLGVATAPQQVLSNGVPVSNFTYSPDTKVLDICVSLLMG
EQFLVSWC
(SEQ ID NO: 194)
(14) 12843B (REGN16824)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSEVQLVESGGGLVQPGGSLRLSC
AASGFTFNIFEMNWVRQAPGKGLEWISYISSRGTTTYYADSVRGRFTISRDNAKNSLYLQMNSLRAEDTAV
YYCARDYEATIPFDFWGQGTLVTVSSGGGGSGGGGSAHPGRPRAVPTQCDVPPNSRFDCAPDKAITQEQCE
ARGCCYIPAKQGLQGAQMGQPWCFFPPSYPSYKLENLSSSEMGYTATLTRTTPTFFPKDILTLRLDVMMET
ENRLHFTIKDPANRRYEVPLETPHVHSRAPSPLYSVEFSEEPFGVIVRRQLDGRVLLNTTVAPLFFADQFLQL
STSLPSQYITGLAEHLSPLMLSTSWTRITLWNRDLAPTPGANLYGSHPFYLALEDGGSAHGVFLLNSNAMD
VVLQPSPALSWRSTGGILDVYIFLGPEPKSVVQQYLDVVGYPFMPPYWGLGFHLCRWGYSSTAITRQVVEN
MTRAHFPLDVQWNDLDYMDSRRDFTFNKDGFRDFPAMVQELHQGGRRYMMIVDPAISSSGPAGSYRPYD
EGLRRGVFITNETGQPLIGKVWPGSTAFPDFTNPTALAWWEDMVAEFHDQVPFDGMWIDMNEPSNFIRGS
EDGCPNNELENPPYVPGVVGGTLQAATICASSHQFLSTHYNLHNLYGLTEAIASHRALVKARGTRPFVISRS
TFAGHGRYAGHWTGDVWSSWEQLASSVPEILQFNLLGVPLVGADVCGFLGNTSEELCVRWTQLGAFYPF
MRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDGESLEVLERGAYTQVIFLARNN
TIVNELVRVTSEGAGLQLQKVTVLGVATAPQQVLSNGVPVSNFTYSPDTKVLDICVSLLMGEQFLVSWC
(SEQ ID NO: 572)
(15) 12844B
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSEVQLVESGGSVVRPGGSLRLSCE
ASGFTFDDYGMSWVRQDPGKGLEWVSGINWNGDRTNYADSVKGRFIISRDNAKNSVYLQMNSLRAEDSA
LYHCARDQGLGVAATLDYWGQGTLVTVSSGGGGSGGGGSAHPGRPRAVPTQCDVPPNSRFDCAPDKAIT
QEQCEARGCCYIPAKQGLQGAQMGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVIVRRQLDGRVLLNTTVAPLFFAD
QFLQLSTSLPSQYITGLAEHLSPLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPPYWGLGFHLCRWGYSSTAITR
QVVENMTRAHFPLDVQWNDLDYMDSRRDFTFNKDGFRDFPAMVQELHQGGRRYMMIVDPAISSSGPAGS
YRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 195)
(16) 12845B (REGN16825)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSEVQLVESGGGLVQPGGSLRLSC
AASGFTVSNYEMNWVRQAPGKGLEWVSYISSSTSNIYYADSVKGRFTISRDNAENSLYLQMNSLRVEDTA
VYYCVRDGIVVVPVGRGYYYYGLDVWGQGTTVTVSSGGGGSGGGGSAHPGRPRAVPTQCDVPPNSRFDC
APDKAITQEQCEARGCCYIPAKQGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVVGYPFMPPYWGLGFHLCRWGY
SSTAITRQVVENMTRAHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPHLYTLFHQAHVAGETVARPLF
LEFPKDSSTWTVDHQLLWGEALLITPVLQAGKAEVTGYFPLGTWYDLQTVPVEALGSLPPPPAAPREPAIH
SEGQWVTLPAPLDTINVHLRAGYIIPLQGPGLTTTESRQQPMALAVALTKGGEARGELFWDDGESLEVLER
GAYTQVIFLARNNTIVNELVRVTSEGAGLQLQKVTVLGVATAPQQVLSNGVPVSNFTYSPDTKVLDICVSL
LMGEQFLVSWC
(SEQ ID NO: 196)
(17) 12847B (REGN16826)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIKGGGGSGGGGSGGGGSEVQLVESGGGLVQPGRSLRLSC
AASGFTFDDYAMNWVRQAPGKGLEWVSGISWSSGSMDYADSVKGRFTISRDNAKNSLYLQMNSLRTEDT
ALYYCAKAREVGDYYGMDVWGQGTTVTVSSGGGGSGGGGSAHPGRPRAVPTQCDVPPNSRFDCAPDKAI
TQEQCEARGCCYIPAKQGLQGAQMGQPWCFFPPSYPSYKLENLSSSEMGYTATLTRTTPTFFPKDILTLRLD
VMMETENRLHFTIKDPANRRYEVPLETPHVHSRAPSPLYSVEFSEEPFGVIVRRQLDGRVLLNTTVAPLFFA
DQFLQLSTSLPSQYITGLAEHLSPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMPPYWGLGFHLCRWGYSSTAIT
RQVVENMTRAHFPLDVQWNDLDYMDSRRDFTFNKDGFRDFPAMVQELHQGGRRYMMIVDPAISSSGPAG
SYRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 573)
(18) 12848B (REGN16827)
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSPWTFGQGTKVEIKGGGGSGGGGSGGGGSEVQLVESGGGLVQPGRSLTLS
CAASGFTFDNFGMHWVRQGPGKGLEWVSGLTWNSGVIGYADSVKGRFTISRDNAKNSLYLQMNSLRPED
TALYYCAKDIRNYGPFDYWGQGTLVTVSSGGGGSGGGGSAHPGRPRAVPTQCDVPPNSRFDCAPDKAITQ
EQCEARGCCYIPAKQGLQGAQMGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVIVRRQLDGRVLLNTTVAPLFFAD
QFLQLSTSLPSQYITGLAEHLSPLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPPYWGLGFHLCRWGYSSTAITR
QVVENMTRAHFPLDVQWNDLDYMDSRRDFTFNKDGFRDFPAMVQELHQGGRRYMMIVDPAISSSGPAGS
YRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 197)
(19) 12850B (REGN16828)
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSPWTFGQGTKVEIKGGGGSGGGGSGGGGSQVQLVQSGAEVKKPGSSVKV
SCKASGGTFNTYAITWVRQAPGQGLEWMGGIIPISGIAEYAQKFQGRVTITTDDSSTTAYMELNSLRSEDTA
VYYCASWNYALYYFYGMDVWGRGTTVTVSSGGGGSGGGGSAHPGRPRAVPTQCDVPPNSRFDCAPDKAI
TQEQCEARGCCYIPAKQGLQGAQMGQPWCFFPPSYPSYKLENLSSSEMGYTATLTRTTPTFFPKDILTLRLD
VMMETENRLHFTIKDPANRRYEVPLETPHVHSRAPSPLYSVEFSEEPFGVIVRRQLDGRVLLNTTVAPLFFA
DQFLQLSTSLPSQYITGLAEHLSPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMPPYWGLGFHLCRWGYSSTAIT
RQVVENMTRAHFPLDVQWNDLDYMDSRRDFTFNKDGFRDFPAMVQELHQGGRRYMMIVDPAISSSGPAG
SYRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 198)
(20) 31863B
DIQMTQSPSSLSASIGDRVTITCRASQGISNYLAWYQQKPGKVPKLLIYAASTLQSGVPSRFSGSGSGTDFTL
TISSLQPEDVATYYCQNHNSVPLTFGGGTKVEIKGGGGSGGGGSGGGGSEVQLVESGGGLVQPGGSLRLSC
AASGFTFNSYAMTWVRQAPGKGLEWVSFIGGSTGNTYYAGSVKGRFTISSDNSKKTLYLQMNSLRAEDTA
VYYCAKGGAARRMEYFQHWGQGTLVTVSSGGGGSGGGGSAHPGRPRAVPTQCDVPPNSRFDCAPDKAIT
QEQCEARGCCYIPAKQGLQGAQMGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVIVRRQLDGRVLLNTTVAPLFFAD
QFLQLSTSLPSQYITGLAEHLSPLMLSTSWTRITLWNRDLAPTPGANLYGSHPFYLALEDGGSAHGVFLLNS
NAMDVVLQPSPALSWRSTGGILDVYIFLGPEPKSVVQQYLDVVGYPFMPPYWGLGFHLCRWGYSSTAITR
QVVENMTRAHFPLDVQWNDLDYMDSRRDFTFNKDGFRDFPAMVQELHQGGRRYMMIVDPAISSSGPAGS
YRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 199)
(21) 31874B
DIQMTQSPSSLSASVGDRVTITCRASQGISNYLAWYQQKPGKVPNLLIYAASTLQSGVPSRFSGSGSGTDFT
LTISSLQPEDVATYYCQKYNSAPLTFGGGTKVEIKGGGGSGGGGSGGGGSEVQLVESGGGLVQPGGSLRLS
CAASGFAFSSYAMTWVRQAPGKGLEWVSVISGTGGSTYYADSVKGRFTISRDNSKNTLYLQMNSLRAEDT
AVYYCAKGGAARRMEYFQYWGQGTLVTVSSGGGGSGGGGSAHPGRPRAVPTQCDVPPNSRFDCAPDKAI
TQEQCEARGCCYIPAKQGLQGAQMGQPWCFFPPSYPSYKLENLSSSEMGYTATLTRTTPTFFPKDILTLRLD
VMMETENRLHFTIKDPANRRYEVPLETPHVHSRAPSPLYSVEFSEEPFGVIVRRQLDGRVLLNTTVAPLFFA
DQFLQLSTSLPSQYITGLAEHLSPLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFMPPYWGLGFHLCRWGYSSTAIT
RQVVENMTRAHFPLDVQWNDLDYMDSRRDFTFNKDGFRDFPAMVQELHQGGRRYMMIVDPAISSSGPAG
SYRPYDEGLRRGVFITNETGQPLIGKVWPGSTAFPDFTNPTALAWWEDMVAEFHDQVPFDGMWIDMNEPS
NFIRGSEDGCPNNELENPPYVPGVVGGTLQAATICASSHQFLSTHYNLHNLYGLTEAIASHRALVKARGTRP
FVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGELFWDDGESLEVLERGAYTQVIF
LARNNTIVNELVRVTSEGAGLQLQKVTVLGVATAPQQVLSNGVPVSNFTYSPDTKVLDICVSLLMGEQFLV
SWC
(SEQ ID NO: 200)
(22) 69261
DIVMTQSPLSLPVTPGEPASISCRSSQSLLHSNGYNYLDWYLQKPGQSPQFLIYLGSNRASGVPDRFSGSGSG
TDFTLKINRVEAEDVGVYYCMQALQTPYTFGQGTKLEIKGGGGSGGGGSGGGGSQVQLVESGGGLVKPG
GSLRLSCAASGFTFSVYYMNWIRQAPGKGLEWVSYISSSGSTIYYADSVKGRFTISRDNAKNSLYLQMNSL
RAEDTAVYYCGREGYSGTYSYYGMDVWGQGTTVTVSSGGGGSGGGGSAHPGRPRAVPTQCDVPPNSRFD
CAPDKAITQEQCEARGCCYIPAKQGLQGAQMGQPWCFFPPSYPSYKLENLSSSEMGYTATLTRTTPTFFPK
DILTLRLDVMMETENRLHFTIKDPANRRYEVPLETPHVHSRAPSPLYSVEFSEEPFGVIVRRQLDGRVLLNT
TVAPLFFADQFLQLSTSLPSQYITGLAEHLSPLMLSTSWTRITLWNRDLAPTPGANLYGSHPFYLALEDGGS
AHGVFLLNSNAMDVVLQPSPALSWRSTGGILDVYIFLGPEPKSVVQQYLDVVGYPFMPPYWGLGFHLCRW
GYSSTAITRQVVENMTRAHFPLDVQWNDLDYMDSRRDFTFNKDGFRDFPAMVQELHQGGRRYMMIVDP
AISSSGPAGSYRPYDEGLRRGVFITNETGQPLIGKVWPGSTAFPDFTNPTALAWWEDMVAEFHDQVPFDGM
WIDMNEPSNFIRGSEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGADVCGFLGNTSEEL
CVRWTQLGAFYPFMRNHNSLLSLPQEPYSFSEPAQQAMRKALTLRYALLPHLYTLFHQAHVAGETVARPL
FLEFPKDSSTWTVDHQLLWGEALLITPVLQAGKAEVTGYFPLGTWYDLQTVPVEALGSLPPPPAAPREPAI
HSEGQWVTLPAPLDTINVHLRAGYIIPLQGPGLTTTESRQQPMALAVALTKGGEARGELFWDDGESLEVLE
RGAYTQVIFLARNNTIVNELVRVTSEGAGLQLQKVTVLGVATAPQQVLSNGVPVSNFTYSPDTKVLDICVS
LLMGEQFLVSWC
(SEQ ID NO: 201)
(23) 69263
DIQMTQSPSSLSASVGDRVTITCRASQDISHYSAWYQQKPGKLPNLLIYAASTLQSGVPSRFSGSGSGTDFSL
TTSSLQPEDVATYYCQKYNSVPLTFGGGTKVEIKGGGGSGGGGSGGGGSEVQLVESGGGLVQPGRSLRLS
CAVSGFTFDDYAMHWVRQAPGKGLEWVSGISWNSGTRGYADSVKGRFTISRDNAKNSLYLQMNSLRGED
TALYYCVKDITISPNYYGMDVWGQGTTVTVSSGGGGSGGGGSAHPGRPRAVPTQCDVPPNSRFDCAPDKA
ITQEQCEARGCCYIPAKQGLQGAQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFGVIVRRQLDGRVLLNTTVAPLFF
ADQFLQLSTSLPSQYITGLAEHLSPLMLSTSWTRITLWNRDLAPTPGANLYGSHPFYLALEDGGSAHGVFLL
NSNAMDVVLQPSPALSWRSTGGILDVYIFLGPEPKSVVQQYLDVVGYPFMPPYWGLGFHLCRWGYSSTAI
TRQVVENMTRAHFPLDVQWNDLDYMDSRRDFTFNKDGFRDFPAMVQELHQGGRRYMMIVDPAISSSGPA
GSYRPYDEGLRRGVFITNETGQPLIGKVWPGSTAFPDFTNPTALAWWEDMVAEFHDQVPFDGMWIDMNE
PSNFIRGSEDGCPNNELENPPYVPGVVGGTLQAATICASSHQFLSTHYNLHNLYGLTEAIASHRALVKARGT
RPFVISRSTFAGHGRYAGHWTGDVWSSWEQLASSVPEILQFNLLGVPLVGADVCGFLGNTSEELCVRWTQ
LGAFYPFMRNHNSLLSLPQEPYSFSEPAQQAMRKALTLRYALLPHLYTLFHQAHVAGETVARPLFLEFPKD
SSTWTVDHQLLWGEALLITPVLQAGKAEVTGYFPLGTWYDLQTVPVEALGSLPPPPAAPREPAIHSEGQWV
TLPAPLDTINVHLRAGYIIPLQGPGLTTTESRQQPMALAVALTKGGEARGELFWDDGESLEVLERGAYTQVI
FLARNNTIVNELVRVTSEGAGLQLQKVTVLGVATAPQQVLSNGVPVSNFTYSPDTKVLDICVSLLMGEQFL
VSWC
(SEQ ID NO: 202)
(24) 69305
DIQMTQSPSSLSASVGDRVTITCRASQSIDRYLNWYRQKPGKAPKLLIYTTSSLQSGVPSRFSGSGSGTDFTL
TLSSLQPEDFATYYCQQSYSPPLTFGGGTKVEIKGGGGSGGGGSGGGGSQVQLVESGGGVVQPGRSLRLSC
AASGFTFSSYGMHWVRQAPGKGLEWVAVIWYDGSNKYYADSVKGRFTISRDISKNTLYLQMNSLRAEDT
AVYYCAGQLDLFFDYWGQGTLVTVSSGGGGSGGGGSAHPGRPRAVPTQCDVPPNSRFDCAPDKAITQEQC
EARGCCYIPAKQGLQGAQMGQPWCFFPPSYPSYKLENLSSSEMGYTATLTRTTPTFFPKDILTLRLDVMME
TENRLHFTIKDPANRRYEVPLETPHVHSRAPSPLYSVEFSEEPFGVIVRRQLDGRVLLNTTVAPLFFADQFLQ
LSTSLPSQYITGLAEHLSPLMLSTSWTRITLWNRDLAPTPGANLYGSHPFYLALEDGGSAHGVFLLNSNAM
DVVLQPSPALSWRSTGGILDVYIFLGPEPKSVVQQYLDVVGYPFMPPYWGLGFHLCRWGYSSTAITRQVVE
NMTRAHFPLDVQWNDLDYMDSRRDFTFNKDGFRDFPAMVQELHQGGRRYMMIVDPAISSSGPAGSYRPY
DEGLRRGVFITNETGQPLIGKVWPGSTAFPDFTNPTALAWWEDMVAEFHDQVPFDGMWIDMNEPSNFIRG
SEDGCPNNELENPPYVPGVVGGTLQAATICASSHQFLSTHYNLHNLYGLTEAIASHRALVKARGTRPFVISR
STFAGHGRYAGHWTGDVWSSWEQLASSVPEILQFNLLGVPLVGADVCGFLGNTSEELCVRWTQLGAFYPF
MRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDGESLEVLERGAYTQVIFLARNN
TIVNELVRVTSEGAGLQLQKVTVLGVATAPQQVLSNGVPVSNFTYSPDTKVLDICVSLLMGEQFLVSWC
(SEQ ID NO: 203)
(25) 69307 (REGN16817)
DIQMTQSPSSVSASVGDRVTITCRASQGISSWLAWYQQKPGKAPKLLIYAASSLQSGVPSRFSGSGSGTDFT
LTISSLQPEDFATYYCQKADSLPYAFGQGTKLEIKGGGGSGGGGSGGGGSEVQLVESGGGLVQPGGSLRLS
CTASGFTFSNYWMTWVRQAPGKGLEWVANIKEDGSEKEYVDSVKGRFTISRDNAKNSLYLQMNSLRGED
TAVYYCARDGEQLVDYYYYYVMDVWGQGTTVTVSSGGGGSGGGGSAHPGRPRAVPTQCDVPPNSRFDC
APDKAITQEQCEARGCCYIPAKQGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVVGYPFMPPYWGLGFHLCRWGY
SSTAITRQVVENMTRAHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPHLYTLFHQAHVAGETVARPLF
LEFPKDSSTWTVDHQLLWGEALLITPVLQAGKAEVTGYFPLGTWYDLQTVPVEALGSLPPPPAAPREPAIH
SEGQWVTLPAPLDTINVHLRAGYIIPLQGPGLTTTESRQQPMALAVALTKGGEARGELFWDDGESLEVLER
GAYTQVIFLARNNTIVNELVRVTSEGAGLQLQKVTVLGVATAPQQVLSNGVPVSNFTYSPDTKVLDICVSL
LMGEQFLVSWC
(SEQ ID NO: 204)
(26) 69323 (REGN16816)
DIQMTQSPSSLSASVGDRVTITCRASQSISSYLNWYQQKPGKAPKVLIYAASSLQSGVPSRFSGSGSGTDFTL
TISSLQPEDFATYYCQQSYSIPLTFGGGTKVEIKGGGGSGGGGSGGGGSEVQLVESGGGLVQPGRSLRLSCA
ASGFTFDDYAMHWVRQAPGKGLEWVSGISWNSGYIGYADSVKGRFTISRDNAENSLHLQMNSLRAEDTA
LYYCARGGSTLVRGVKGGYYGMDVWGQGTTVTVSSGGGGSGGGGSAHPGRPRAVPTQCDVPPNSRFDC
APDKAITQEQCEARGCCYIPAKQGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVVGYPFMPPYWGLGFHLCRWGY
SSTAITRQVVENMTRAHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPHLYTLFHQAHVAGETVARPLF
LEFPKDSSTWTVDHQLLWGEALLITPVLQAGKAEVTGYFPLGTWYDLQTVPVEALGSLPPPPAAPREPAIH
SEGQWVTLPAPLDTINVHLRAGYIIPLQGPGLTTTESRQQPMALAVALTKGGEARGELFWDDGESLEVLER
GAYTQVIFLARNNTIVNELVRVTSEGAGLQLQKVTVLGVATAPQQVLSNGVPVSNFTYSPDTKVLDICVSL
LMGEQFLVSWC
(SEQ ID NO: 205)
(27) 69326
EIVMTQSPATLSVSPGERATLSCRASQSVSSNFAWYQQKPGQAPRLLIYSASSRATGIPVRFSGSGSGTEFTL
TISSLQSEDFAVYYCQQYNIWPRTFGQGTKVEIKGGGGSGGGGSGGGGSEVQLVESGGGLVQPGGSLRLSC
AVSGFIFSSYEMNWVRQAPGKGLEWVSYISSSGSTIFYADSVKGRFTISRDNAKNSLYLQMNSLRAEDTAV
YYCVSGVVLFDVWGQGTMVTVSSGGGGSGGGGSAHPGRPRAVPTQCDVPPNSRFDCAPDKAITQEQCEA
RGCCYIPAKQGLQGAQMGQPWCFFPPSYPSYKLENLSSSEMGYTATLTRTTPTFFPKDILTLRLDVMMETE
NRLHFTIKDPANRRYEVPLETPHVHSRAPSPLYSVEFSEEPFGVIVRRQLDGRVLLNTTVAPLFFADQFLQLS
TSLPSQYITGLAEHLSPLMLSTSWTRITLWNRDLAPTPGANLYGSHPFYLALEDGGSAHGVFLLNSNAMDV
VLQPSPALSWRSTGGILDVYIFLGPEPKSVVQQYLDVVGYPFMPPYWGLGFHLCRWGYSSTAITRQVVEN
MTRAHFPLDVQWNDLDYMDSRRDFTFNKDGFRDFPAMVQELHQGGRRYMMIVDPAISSSGPAGSYRPYD
EGLRRGVFITNETGQPLIGKVWPGSTAFPDFTNPTALAWWEDMVAEFHDQVPFDGMWIDMNEPSNFIRGS
EDGCPNNELENPPYVPGVVGGTLQAATICASSHQFLSTHYNLHNLYGLTEAIASHRALVKARGTRPFVISRS
TFAGHGRYAGHWTGDVWSSWEQLASSVPEILQFNLLGVPLVGADVCGFLGNTSEELCVRWTQLGAFYPF
MRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDGESLEVLERGAYTQVIFLARNN
TIVNELVRVTSEGAGLQLQKVTVLGVATAPQQVLSNGVPVSNFTYSPDTKVLDICVSLLMGEQFLVSWC
(SEQ ID NO: 206)
(28) 69329
DIQMTQSPSSVSASVGDRVTITCRASQGISSWLAWYQQKPGKAPKLLIYAASSLQSGVPSRFSGSGSGTDFT
LTISSLQPEDFATYYCQKANSFPYTFGQGTKLEIKGGGGSGGGGSGGGGSEVQLVESGGGLVQPGGSLRLS
CAASGFTFSNYWMTWVRQAPGKGLEWVANIKEDGSEKDYVDSVKGRFTISRDNAKNSLYLQMNSLRGED
TAVYYCARDGEQLVDYYYYYVMDVWGQGTTVTVSSGGGGSGGGGSAHPGRPRAVPTQCDVPPNSRFDC
APDKAITQEQCEARGCCYIPAKQGLQGAQMGQPWCFFPPSYPSYKLENLSSSEMGYTATLTRTTPTFFPKDI
LTLRLDVMMETENRLHFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVVGYPFMPPYWGLGFHLCRWGY
SSTAITRQVVENMTRAHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDMVAEFHDQVPFDGMWI
DMNEPSNFIRGSEDGCPNNELENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPHLYTLFHQAHVAGETVARPLF
LEFPKDSSTWTVDHQLLWGEALLITPVLQAGKAEVTGYFPLGTWYDLQTVPVEALGSLPPPPAAPREPAIH
SEGQWVTLPAPLDTINVHLRAGYIIPLQGPGLTTTESRQQPMALAVALTKGGEARGELFWDDGESLEVLER
GAYTQVIFLARNNTIVNELVRVTSEGAGLQLQKVTVLGVATAPQQVLSNGVPVSNFTYSPDTKVLDICVSL
LMGEQFLVSWC
(SEQ ID NO: 207)
(29) 69331
DIQLTQSPSSLSASVGDRVTITCWASQGISSYLAWYQQKPGKAPKLLIYAASTLQSGVPSRFSGSGSGTEFTL
TISSLQPEDFATYYCQQLNSYPLTFGGGTKVEIKGGGGSGGGGSGGGGSQVQLVESGGGVVQPGRSLRLSC
IASGFTFSVYGIHWVRQAPGKGLEWMAVISHDGNIKHYADSVKGRFTISRDNSKNTLYLQINSLRTEDTAV
YYCAKDTWNSLDTFDIWGQGTMVTVSSGGGGSGGGGSAHPGRPRAVPTQCDVPPNSRFDCAPDKAITQE
QCEARGCCYIPAKQGLQGAQMGQPWCFFPPSYPSYKLENLSSSEMGYTATLTRTTPTFFPKDILTLRLDVM
METENRLHFTIKDPANRRYEVPLETPHVHSRAPSPLYSVEFSEEPFGVIVRRQLDGRVLLNTTVAPLFFADQF
LQLSTSLPSQYITGLAEHLSPLMLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYWGLGFHLCRWGYSSTAITRQV
VENMTRAHFPLDVQWNDLDYMDSRRDFTFNKDGFRDFPAMVQELHQGGRRYMMIVDPAISSSGPAGSYR
PYDEGLRRGVFITNETGQPLIGKVWPGSTAFPDFTNPTALAWWEDMVAEFHDQVPFDGMWIDMNEPSNFI
RGSEDGCPNNELENPPYVPGVVGGTLQAATICASSHQFLSTHYNLHNLYGLTEAIASHRALVKARGTRPFVI
SRSTFAGHGRYAGHWTGDVWSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPA
PLDTINVHLRAGYIIPLQGPGLTTTESRQQPMALAVALTKGGEARGELFWDDGESLEVLERGAYTQVIFLAR
NNTIVNELVRVTSEGAGLQLQKVTVLGVATAPQQVLSNGVPVSNFTYSPDTKVLDICVSLLMGEQFLVSW
C
(SEQ ID NO: 208)
(30) 69332
AIQMTQSPSSLSASVGDRVTITCRASQGIRNDLGWYQQKPGKAPKLLIYAASTLQSGVPSRFSGSGSGTDFT
LTISSLQPEDFATYYCLQDYNYPFTFGPGTKVDIKGGGGSGGGGSGGGGSQVTLRESGPALVKPSQTLTLTC
TFSGFSLNTYGMFVSWIRQPPGKALEWLAHIHWDDDKYYSTSLKTRLTISKDTSKNQVVLTMTNMDPVDT
ATYYCARGHNNLNYIIHWGQGTLVTVSSGGGGSGGGGSAHPGRPRAVPTQCDVPPNSRFDCAPDKAITQE
QCEARGCCYIPAKQGLQGAQMGQPWCFFPPSYPSYKLENLSSSEMGYTATLTRTTPTFFPKDILTLRLDVM
METENRLHFTIKDPANRRYEVPLETPHVHSRAPSPLYSVEFSEEPFGVIVRRQLDGRVLLNTTVAPLFFADQF
LQLSTSLPSQYITGLAEHLSPLMLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYWGLGFHLCRWGYSSTAITRQV
VENMTRAHFPLDVQWNDLDYMDSRRDFTFNKDGFRDFPAMVQELHQGGRRYMMIVDPAISSSGPAGSYR
PYDEGLRRGVFITNETGQPLIGKVWPGSTAFPDFTNPTALAWWEDMVAEFHDQVPFDGMWIDMNEPSNFI
RGSEDGCPNNELENPPYVPGVVGGTLQAATICASSHQFLSTHYNLHNLYGLTEAIASHRALVKARGTRPFVI
SRSTFAGHGRYAGHWTGDVWSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPA
PLDTINVHLRAGYIIPLQGPGLTTTESRQQPMALAVALTKGGEARGELFWDDGESLEVLERGAYTQVIFLAR
NNTIVNELVRVTSEGAGLQLQKVTVLGVATAPQQVLSNGVPVSNFTYSPDTKVLDICVSLLMGEQFLVSW
C
(SEQ ID NO: 209)
(31) 69340
EIVLTQSPATLSLSPGERATLSCRASQSVSSYLAWYQQKPGQAPRLLIHDVSNRATGIPARFSGSGSGTDFTL
TISSLEPEDFVVYYCQQRSDWPITFGQGTRLEIKGGGGSGGGGSGGGGSEVQLVESGGGLVQPGRSLRLSC
AASGFTFDDKAMHWVRQVPGKGLEWISGISWNSGTIGYADSVKGRFIISRDNAKNSLYLQMNSLRAEDTA
LYYCAKDGDTSGWYWYGLDVWGQGTTVTVSSGGGGSGGGGSAHPGRPRAVPTQCDVPPNSRFDCAPDK
AITQEQCEARGCCYIPAKQGLQGAQMGQPWCFFPPSYPSYKLENLSSSEMGYTATLTRTTPTFFPKDILTLR
LDVMMETENRLHFTIKDPANRRYEVPLETPHVHSRAPSPLYSVEFSEEPFGVIVRRQLDGRVLLNTTVAPLF
FADQFLQLSTSLPSQYITGLAEHLSPLMLSTSWTRITLWNRDLAPTPGANLYGSHPFYLALEDGGSAHGVFL
LNSNAMDVVLQPSPALSWRSTGGILDVYIFLGPEPKSVVQQYLDVVGYPFMPPYWGLGFHLCRWGYSSTA
ITRQVVENMTRAHFPLDVQWNDLDYMDSRRDFTFNKDGFRDFPAMVQELHQGGRRYMMIVDPAISSSGP
AGSYRPYDEGLRRGVFITNETGQPLIGKVWPGSTAFPDFTNPTALAWWEDMVAEFHDQVPFDGMWIDMN
EPSNFIRGSEDGCPNNELENPPYVPGVVGGTLQAATICASSHQFLSTHYNLHNLYGLTEAIASHRALVKARG
TRPFVISRSTFAGHGRYAGHWTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTLFHQAHVAGETVARPLFLEFPK
DSSTWTVDHQLLWGEALLITPVLQAGKAEVTGYFPLGTWYDLQTVPVEALGSLPPPPAAPREPAIHSEGQW
VTLPAPLDTINVHLRAGYIIPLQGPGLTTTESRQQPMALAVALTKGGEARGELFWDDGESLEVLERGAYTQ
VIFLARNNTIVNELVRVTSEGAGLQLQKVTVLGVATAPQQVLSNGVPVSNFTYSPDTKVLDICVSLLMGEQ
FLVSWC
(SEQ ID NO: 210)
(32) 69348
DIQMTQSPSSLSASVGDRVTITCRASQSIRNVLGWFQQKPGKAPQRLIYAASSLQSGVPSRFSGSGSGTEFTL
TISSLQPEDFATYYCLQHNFYPLTFGGGTKVEIKGGGGSGGGGSGGGGSQVQLVESGGGVVQPGRSLRLSC
AASGFTFTTYGMHWVRQAPGKGLEWVAVIWYDGSNKYYGDSVKGRFTISRDNSKNTLYLQMNSLRVDD
TAVYYCTRTHGYTRSSDGFDYWGQGTLVTVSSGGGGSGGGGSAHPGRPRAVPTQCDVPPNSRFDCAPDK
AITQEQCEARGCCYIPAKQGLQGAQMGQPWCFFPPSYPSYKLENLSSSEMGYTATLTRTTPTFFPKDILTLR
LDVMMETENRLHFTIKDPANRRYEVPLETPHVHSRAPSPLYSVEFSEEPFGVIVRRQLDGRVLLNTTVAPLF
FADQFLQLSTSLPSQYITGLAEHLSPLMLSTSWTRITLWNRDLAPTPGANLYGSHPFYLALEDGGSAHGVFL
LNSNAMDVVLQPSPALSWRSTGGILDVYIFLGPEPKSVVQQYLDVVGYPFMPPYWGLGFHLCRWGYSSTA
ITRQVVENMTRAHFPLDVQWNDLDYMDSRRDFTFNKDGFRDFPAMVQELHQGGRRYMMIVDPAISSSGP
AGSYRPYDEGLRRGVFITNETGQPLIGKVWPGSTAFPDFTNPTALAWWEDMVAEFHDQVPFDGMWIDMN
EPSNFIRGSEDGCPNNELENPPYVPGVVGGTLQAATICASSHQFLSTHYNLHNLYGLTEAIASHRALVKARG
TRPFVISRSTFAGHGRYAGHWTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTLFHQAHVAGETVARPLFLEFPK
DSSTWTVDHQLLWGEALLITPVLQAGKAEVTGYFPLGTWYDLQTVPVEALGSLPPPPAAPREPAIHSEGQW
VTLPAPLDTINVHLRAGYIIPLQGPGLTTTESRQQPMALAVALTKGGEARGELFWDDGESLEVLERGAYTQ
VIFLARNNTIVNELVRVTSEGAGLQLQKVTVLGVATAPQQVLSNGVPVSNFTYSPDTKVLDICVSLLMGEQ
FLVSWC
(SEQ ID NO: 211);
(1)
MHRPRRRGTRPPPLALLAALLLAARGADADIQMTQSPSSVSASVGDRVTITCRASQGIASWLAWYQQKPG
KAPELLIYAASSLQGGVPSRFSGSGSGTDFTLTISSLQPEDFAIYYCQQANYFPWTFGQGTKVEIKGGGGSG
GGGSGGGGSQITLKESGPTLVKPTQTLTLTCTFSGFSLSTSGVGVVWIRQPPGKALEWLALIYWNDHKRYS
PSLGSRLTITKDTSKNQVVLTMTNMDPVDTATYYCAHYSGSYSYYYYGLDVWGQGTTVTVSSGGGGSGG
GGSAHPGRPRAVPTQCDVPPNSRFDCAPDKAITQEQCEARGCCYIPAKQGLQGAQMGQPWCFFPPSYPSYK
LENLSSSEMGYTATLTRTTPTFFPKDILTLRLDVMMETENRLHFTIKDPANRRYEVPLETPHVHSRAPSPLYS
VEFSEEPFGVIVRRQLDGRVLLNTTVAPLFFADQFLQLSTSLPSQYITGLAEHLSPLMLSTSWTRITLWNRDL
APTPGANLYGSHPFYLALEDGGSAHGVFLLNSNAMDVVLQPSPALSWRSTGGILDVYIFLGPEPKSVVQQY
LDVVGYPFMPPYWGLGFHLCRWGYSSTAITRQVVENMTRAHFPLDVQWNDLDYMDSRRDFTFNKDGFR
DFPAMVQELHQGGRRYMMIVDPAISSSGPAGSYRPYDEGLRRGVFITNETGQPLIGKVWPGSTAFPDFTNP
TALAWWEDMVAEFHDQVPFDGMWIDMNEPSNFIRGSEDGCPNNELENPPYVPGVVGGTLQAATICASSH
QFLSTHYNLHNLYGLTEAIASHRALVKARGTRPFVISRSTFAGHGRY AGHWTGDVWSSWEQLASSVPEILQ
FNLLGVPLVGADVCGFLGNTSEELCVRWTQLGAFYPFMRNHNSLLSLPQEPYSFSEPAQQAMRKALTLRY
ALLPHLYTLFHQAHVAGETVARPLFLEFPKDSSTWTVDHQLLWGEALLITPVLQAGKAEVTGYFPLGTWY
DLQTVPVEALGSLPPPPAAPREPAIHSEGQWVTLPAPLDTINVHLRAGYIIPLQGPGLTTTESRQQPMALAVA
LTKGGEARGELFWDDGESLEVLERGAYTQVIFLARNNTIVNELVRVTSEGAGLQLQKVTVLGVATAPQQV
LSNGVPVSNFTYSPDTKVLDICVSLLMGEQFLVSWC
(SEQ ID NO: 212; optionally lacking the N-terminal MHRPRRRGTRPPPLALLAALLLAARGADA (SEQ ID NO:
596) sequence);
(2)
MHRPRRRGTRPPPLALLAALLLAARGADADIQMTQSPSSLSASVGDRVTITCRASQSISSYLNWYQQKPGK
APKLLIYAASSLQSGVPSRFSGSGSGTDFTLTISSLQPEDFATYYCQQSYSTPPITFGQGTRLEIKGGGGSGGG
GSGGGGSQVQLVESGGGVVQPGRSLRLSCAASGFPFSNYVMYWVRQAPGKGLEWVALIFFDGKKNYHAD
SVKGRFTITRDNSKNMLYLQMNSLRPEDAAVYYCAKIHCPNGVCYKGYYGMDVWGQGTTVTVSSGGGG
SGGGGSAHPGRPRAVPTQCDVPPNSRFDCAPDKAITQEQCEARGCCYIPAKQGLQGAQMGQPWCFFPPSYP
SYKLENLSSSEMGYTATLTRTTPTFFPKDILTLRLDVMMETENRLHFTIKDPANRRYEVPLETPHVHSRAPSP
LYSVEFSEEPFGVIVRRQLDGRVLLNTTVAPLFFADQFLQLSTSLPSQYITGLAEHLSPLMLSTSWTRITLWN
RDLAPTPGANLYGSHPFYLALEDGGSAHGVFLLNSNAMDVVLQPSPALSWRSTGGILDVYIFLGPEPKSVV
QQYLDVVGYPFMPPYWGLGFHLCRWGYSSTAITRQVVENMTRAHFPLDVQWNDLDYMDSRRDFTFNKD
GFRDFPAMVQELHQGGRRYMMIVDPAISSSGPAGSYRPYDEGLRRGVFITNETGQPLIGKVWPGSTAFPDF
TNPTALAWWEDMVAEFHDQVPFDGMWIDMNEPSNFIRGSEDGCPNNELENPPYVPGVVGGTLQAATICAS
SHQFLSTHYNLHNLYGLTEAIASHRALVKARGTRPFVISRSTFAGHGRYAGHWTGDVWSSWEQLASSVPEI
LQFNLLGVPLVGADVCGFLGNTSEELCVRWTQLGAFYPFMRNHNSLLSLPQEPYSFSEPAQQAMRKALTL
RYALLPHLYTLFHQAHVAGETVARPLFLEFPKDSSTWTVDHQLLWGEALLITPVLQAGKAEVTGYFPLGT
WYDLQTVPVEALGSLPPPPAAPREPAIHSEGQWVTLPAPLDTINVHLRAGYIIPLQGPGLTTTESRQQPMAL
AVALTKGGEARGELFWDDGESLEVLERGAYTQVIFLARNNTIVNELVRVTSEGAGLQLQKVTVLGVATAP
QQVLSNGVPVSNFTYSPDTKVLDICVSLLMGEQFLVSWC
(SEQ ID NO: 213; optionally lacking the N-terminal MHRPRRRGTRPPPLALLAALLLAARGADA (SEQ ID NO:
596) sequence);
(3)
MHRPRRRGTRPPPLALLAALLLAARGADADIQMTQSPSSLSASVGDRVTITCRASQSISSYLNWYQQKPGK
APKLLIYAASSLQSGVPSRFSGSGSGTDFTLTISSLQPEDFATYYCQQSYSTPPITFGQGTRLEIKGGGGSGGG
GSGGGGSEVQLVESGGGLVQPGGSLRLSCAASGFTFNIFEMNWVRQAPGKGLEWISYISSRGTTTYYADSV
RGRFTISRDNAKNSLYLQMNSLRAEDTAVYYCARDYEATIPFDFWGQGTLVTVSSGGGGSGGGGSAHPGR
PRAVPTQCDVPPNSREDCAPDKAITQEQCEARGCCYIPAKQGLQGAQMGQPWCFFPPSYPSYKLENLSSSE
MGYTATLTRTTPTFFPKDILTLRLDVMMETENRLHFTIKDPANRRYEVPLETPHVHSRAPSPLYSVEFSEEPF
GVIVRRQLDGRVLLNTTVAPLFFADQFLQLSTSLPSQYITGLAEHLSPLMLSTSWTRITLWNRDLAPTPGAN
LYGSHPFYLALEDGGSAHGVFLLNSNAMDVVLQPSPALSWRSTGGILDVYIFLGPEPKSVVQQYLDVVGYP
FMPPYWGLGFHLCRWGYSSTAITRQVVENMTRAHFPLDVQWNDLDYMDSRRDFTFNKDGFRDFPAMVQ
ELHQGGRRYMMIVDPAISSSGPAGSYRPYDEGLRRGVFITNETGQPLIGKVWPGSTAFPDFTNPTALAWWE
DMVAEFHDQVPFDGMWIDMNEPSNFIRGSEDGCPNNELENPPYVPGVVGGTLQAATICASSHQFLSTHYN
LHNLYGLTEAIASHRALVKARGTRPFVISRSTFAGHGRYAGHWTGDVWSSWEQLASSVPEILQFNLLGVPL
VGADVCGFLGNTSEELCVRWTQLGAFYPFMRNHNSLLSLPQEPYSFSEPAQQAMRKALTLRYALLPHLYT
LFHQAHVAGETVARPLFLEFPKDSSTWTVDHQLLWGEALLITPVLQAGKAEVTGYFPLGTWYDLQTVPVE
ALGSLPPPPAAPREPAIHSEGQWVTLPAPLDTINVHLRAGYIIPLQGPGLTTTESRQQPMALAVALTKGGEA
RGELFWDDGESLEVLERGAYTQVIFLARNNTIVNELVRVTSEGAGLQLQKVTVLGVATAPQQVLSNGVPV
SNFTYSPDTKVLDICVSLLMGEQFLVSWC
(SEQ ID NO: 214; optionally lacking the N-terminal MHRPRRRGTRPPPLALLAALLLAARGADA (SEQ ID NO:
596) sequence);
(4)
MHRPRRRGTRPPPLALLAALLLAARGADADIQMTQSPSSLSASVGDRVTITCRASQSISSYLNWYQQKPGK
APKLLIYAASSLQSGVPSRFSGSGSGTDFTLTISSLQPEDFATYYCQQSYSTPPITFGQGTRLEIKGGGGSGGG
GSGGGGSEVQLVESGGGLVQPGRSLRLSCAASGFTFDDYAMNWVRQAPGKGLEWVSGISWSSGSMDYAD
SVKGRFTISRDNAKNSLYLQMNSLRTEDTALYYCAKAREVGDYYGMDVWGQGTTVTVSSGGGGSGGGG
SAHPGRPRAVPTQCDVPPNSRFDCAPDKAITQEQCEARGCCYIPAKQGLQGAQMGQPWCFFPPSYPSYKLE
NLSSSEMGYTATLTRTTPTFFPKDILTLRLDVMMETENRLHFTIKDPANRRYEVPLETPHVHSRAPSPLYSVE
FSEEPFGVIVRRQLDGRVLLNTTVAPLFFADQFLQLSTSLPSQYITGLAEHLSPLMLSTSWTRITLWNRDLAP
TPGANLYGSHPFYLALEDGGSAHGVFLLNSNAMDVVLQPSPALSWRSTGGILDVYIFLGPEPKSVVQQYLD
VVGYPFMPPYWGLGFHLCRWGYSSTAITRQVVENMTRAHFPLDVQWNDLDYMDSRRDFTFNKDGFRDFP
AMVQELHQGGRRYMMIVDPAISSSGPAGSYRPYDEGLRRGVFITNETGQPLIGKVWPGSTAFPDFTNPTAL
AWWEDMVAEFHDQVPFDGMWIDMNEPSNFIRGSEDGCPNNELENPPYVPGVVGGTLQAATICASSHQFLS
THYNLHNLYGLTEAIASHRALVKARGTRPFVISRSTFAGHGRYAGHWTGDVWSSWEQLASSVPEILQFNLL
GVPLVGADVCGFLGNTSEELCVRWTQLGAFYPFMRNHNSLLSLPQEPYSFSEPAQQAMRKALTLRYALLP
HLYTLFHQAHVAGETVARPLFLEFPKDSSTWTVDHQLLWGEALLITPVLQAGKAEVTGYFPLGTWYDLQT
VPVEALGSLPPPPAAPREPAIHSEGQWVTLPAPLDTINVHLRAGYIIPLQGPGLTTTESRQQPMALAVALTKG
GEARGELFWDDGESLEVLERGAYTQVIFLARNNTIVNELVRVTSEGAGLQLQKVTVLGVATAPQQVLSNG
VPVSNFTYSPDTKVLDICVSLLMGEQFLVSWC
(SEQ ID NO: 215; optionally lacking the N-terminal MHRPRRRGTRPPPLALLAALLLAARGADA (SEQ ID NO:
596) sequence)

In order to validate the anti-human TfR antibodies that were screened for binding in vitro, we performed in vivo mouse studies in Tfrc^{hum/hum} knock-in mice to evaluate blood-brain-barrier (BBB) crossing. Eleven clones that had mature hGAA protein in brain homogenate detected by western blot were selected from this first screen of 31 antibodies.

GAA fusions by hydrodynamic delivery (HDD). Three-month-old human TFRC knock-in mice were injected with DNA plasmids expressing the various anti-hTfR antibodies in the anti-hTfRscfv:2×G4S:hGAA format under the liver-specific mouse TTR promoter. Mice received 50 μg of DNA in 0.9% sterile saline diluted to 10% of the mouse's body weight (0.1 mL/g body weight). 48 hours post-injection, tissues were dissected from mice immediately after sacrifice by CO₂ asphyxiation, snap frozen in liquid nitrogen, and stored at −80° C.

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFLO7810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

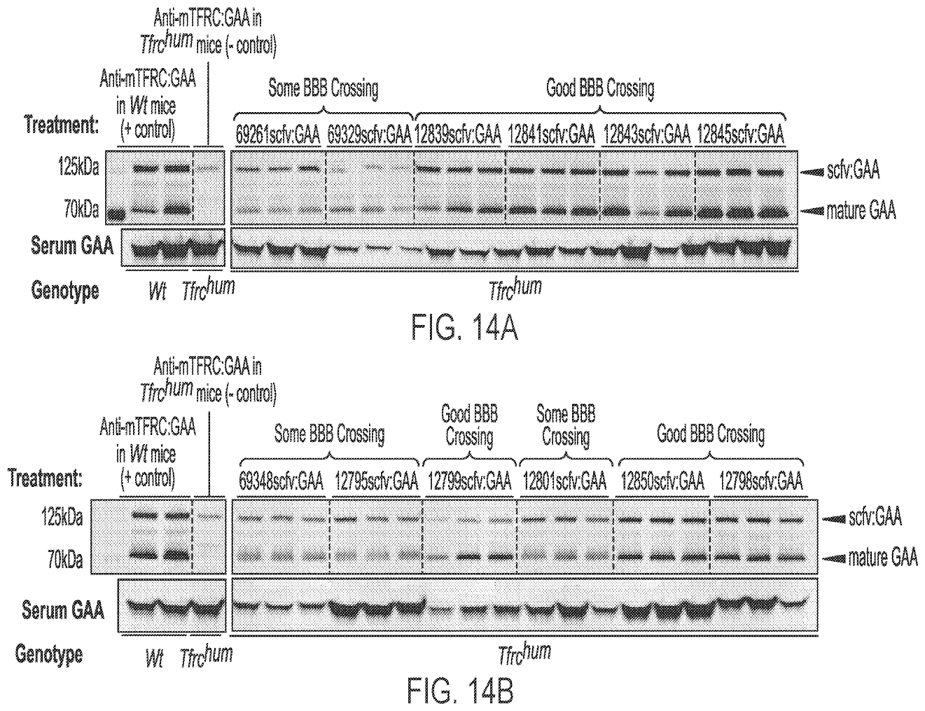

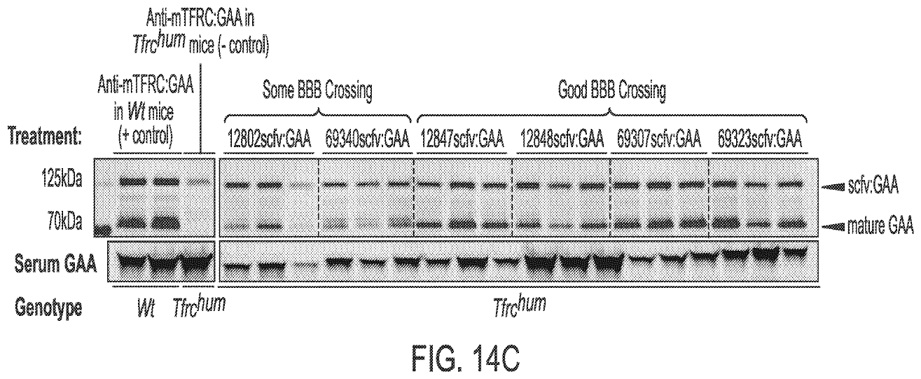

Protein band intensity was quantified in LI-COR Image Studio software. The quantification of the mature 77 kDa GAA band for each sample was determined by first normalizing to the lane's TPS signal, then normalizing to GAA levels in the serum (loading control and liver expression control, respectively). Values were then compared to the positive control group anti-mouse TfRscfv:hGAA in Wt mice, and negative control group anti-mTfRscfv:hGAA in Tfrc^{hum/hum} mice (FIGS. 14A-14C, Table 27). The 8D3 scFv (anti-mouse TfR scFv) has the heavy chain amino acid sequence:

(SEQ ID NO: 536)
EVQLVESGGGLVQPGNSLTLSCVASGFTFSNYGMHWIRQAPKKGLEWIAM
IYYDSSKMNYADTVKGRFTISRDNSKNTLYLEMNSLRSEDTAMYYCAVPT
SHYVVDVWGQGVSVTVSS,

and the light chain amino acid sequence:

(SEQ ID NO: 537)
DIQMTQSPASLSASLEEIVTITCQASQDIGNWLAWYQQKPGKSPQLLIYG
ATSLADGVPSRFSGSRSGTQFSLKISRVQVEDIGIYYCLQAYNTPWTFGG
GTKLELK.

TABLE 27
Quantification of mature hGAA protein in brain homogenate
from mice treated HDD with anti-hTfRscfv:hGAA plasmids.

| | | Mature hGAA |
|---|---|---|
| | | protein in brain |
| | | (normalized |
| | | to positive |
| Treatment group | Genotype | control) |
| anti-mTfRscfv:hGAA (positive control) | Wt | 1.00 ± 0.43* |
| anti-mTfRscfv:hGAA | Tfrc^{hum/hum} | 0.02 ± 0.03 |
| (negative control) | | |
| 69261scfv:hGAA | Tfrc^{hum/hum} | 0.67 ± 0.50 |
| 69307scfv:hGAA | Tfrc^{hum/hum} | 1.08 ± 0.19 |
| 69323scfv:hGAA | Tfrc^{hum/hum} | 0.91 ± 0.46 |
| 69329scfv:hGAA | Tfrc^{hum/hum} | 0.65 ± 0.13 |
| 69340scfv:hGAA | Tfrc^{hum/hum} | 0.55 ± 0.58 |
| 69348scfv:hGAA | Tfrc^{hum/hum} | 0.50 ± 0.05 |
| 12795scfv:hGAA | Tfrc^{hum/hum} | 0.27 ± 0.20 |
| 12798scfv:hGAA | Tfrc^{hum/hum} | 0.72 ± 0.42 |
| 12799scfv:hGAA | Tfrc^{hum/hum} | 1.05 ± 0.51* |
| 12801scfv:hGAA | Tfrc^{hum/hum} | 0.49 ± 0.18 |
| 12802scfv:hGAA | Tfrc^{hum/hum} | 0.29 ± 0.27 |
| 12839scfv:hGAA | Tfrc^{hum/hum} | 1.29 ± 0.27** |
| 12841scfv:hGAA | Tfrc^{hum/hum} | 1.72 ± 0.06*** |
| 12843scfv:hGAA | Tfrc^{hum/hum} | 1.79 ± 0.85*** |
| 12845scfv:hGAA | Tfrc^{hum/hum} | 3.08 ± 0.92*** |
| 12847scfv:hGAA | Tfrc^{hum/hum} | 1.24 ± 0.30 |
| 12848scfv:hGAA | Tfrc^{hum/hum} | 0.59 ± 0.16 |
| 12850scfv:hGAA | Tfrc^{hum/hum} | 0.47 ± 0.05 |

Data were quantified from western blot as arbitrary units (FIGS. 14A-14C). All values are mean ± SD, n = 3-6 per group. One Way ANOVA vs. negative control anti-mTfRscfv:hGAA in Tfrc^{hum/hum} mice;
*p < 0.05;
**p < 0.005;
***p < 0.0001.

Capillary depletion of brain samples following HDD of anti-hTfRscfv:hGAA plasmids. Selected anti-hTfRscfv:hGAA from Table 27 were tested in a secondary screen in Tfrc^{hum} mice to determine whether hGAA was present in the brain parenchyma, and not trapped in the BBB endothelial cells. We selected four scFvs (12799, 12839, 12843, and 12847) from this screen based on mature hGAA in the parenchyma fraction on western blot, as well as high affinity to cynomolgus TfR.

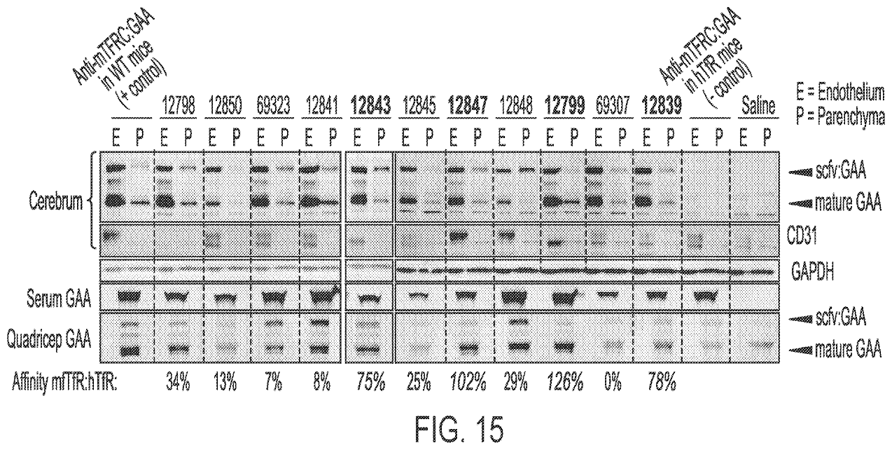

Three-month-old animals were treated HDD as detailed above. 48 hours post-injection, mice were perfused with 30 mL 0.9% saline immediately after sacrifice by CO₂ asphyxiation. A 2 mm coronal slice of cerebrum was taken between bregma and −2 mm bregma and placed in 700 μL physiological buffer (10 mM HEPES, 4 mM KCl, 2.8 mM CaCl₂, 1 mMM MgSO₄, 1 mM NaH₂PO₄, 10 mM D-glucose in 0.9% saline pH 7.4) on ice. Brain slices were gently homogenized on ice with a glass dounce homogenizer. An equivalent volume of 26% dextran (MW 70,000 Da) in physiological buffer was added (final 13% dextran) and homogenized 10 more strokes. Parenchyma (supernatant) and endothelial (pellet) fractions were separated by centrifugation at 5,400 g for 15 min at 4° C. Anti-hGAA western blot was performed on fractions as detailed above (FIG. 15, Table 28). Blots were also probed with anti-CD31 endothelial marker (Abcam ab182982).

TABLE 28
Quantification of mature hGAA protein in brain parenchyma
fractions and BBB endothelial fractions of mice treated HDD
with anti-hTfRscfv:hGAA plasmids.

| | | Mature | Mature | |
|---|---|---|---|---|
| | | hGAA | hGAA | |
| | | protein | protein | Affinity |
| | | in brain | in brain | to |
| | | parenchyma | endothelium | mfTfR |
| | | (normalized | (normalized | (% of |
| Treatment | | to positive | to positive | hTfR |
| group | Genotype | control) | control) | binding) |
| anti- | Wt | 1.00 | 5.82 | ND |
| mTfRscfv:hGAA | | | | |
| (positive control) | | | | |
| anti- | Tfrc^{hum/hum} | 0.00 | 0.01 | ND |
| mTfRscfv:hGAA | | | | |
| (negative control) | | | | |
| 69307scfv:hGAA | Tfrc^{hum/hum} | 1.24 | 10.73 | 0% |
| 69323scfv:hGAA | Tfrc^{hum/hum} | 0.62 | 4.18 | 7% |
| 12798scfv:hGAA | Tfrc^{hum/hum} | 0.91 | 8.37 | 34% |
| 12799scfv:hGAA | Tfrc^{hum/hum} | 0.44 | 3.99 | 126% |
| 12839scfv:hGAA | Tfrc^{hum/hum} | 0.05 | 0.84 | 78% |
| 12841scfv:hGAA | Tfrc^{hum/hum} | 0.78 | 4.23 | 8% |
| 12843scfv:hGAA | Tfrc^{hum/hum} | 1.13 | 12.99 | 75% |
| 12845scfv:hGAA | Tfrc^{hum/hum} | 2.04 | 13.06 | 25% |
| 12847scfv:hGAA | Tfrc^{hum/hum} | 0.60 | 4.96 | 102% |
| 12848scfv:hGAA | Tfrc^{hum/hum} | 0.17 | 1.24 | 29% |
| 12850scfv:hGAA | Tfrc^{hum/hum} | 0.22 | 2.25 | 13% |

hGAA protein was quantified from western blot as arbitrary units (FIG. 15). n = 1 per group. Affinity to cynomolgus macaque TfR Luminex data, calculated as percent of binding to hTfR: (mfTfR binding ÷ hTfR binding) × 100.

TABLE 29
Quantification of hGAA protein in quadricep of mice
treated HDD with anti-hTfRscfv:hGAA plasmids.

| | | hGAA protein |
|---|---|---|
| | | in quadricep |
| | | (normalized to |
| Treatment group | Genotype | positive control) |
| Saline (vehicle) | Tfrc^{hum/hum} | 0.38 ± 0.25 |
| anti-mTfRscfv:hGAA (positive control) | Wt | 1.07 ± 0.27 |
| anti-mTfRscfv:hGAA (negative | Tfrc^{hum/hum} | 0.56 ± 0.17 |
| control) | | |
| 69307scfv:hGAA | Tfrc^{hum/hum} | 0.58 ± 0.18 |
| 69323scfv:hGAA | Tfrc^{hum/hum} | 1.10 ± 0.19 |
| 12798scfv:hGAA | Tfrc^{hum/hum} | 1.33 ± 0.56 |
| 12799scfv:hGAA | Tfrc^{hum/hum} | 0.67 ± 0.18 |
| 12839scfv:hGAA | Tfrc^{hum/hum} | 1.80 ± 0.18 |
| 12841scfv:hGAA | Tfrc^{hum/hum} | 1.15 ± 0.12 |
| 12843scfv:hGAA | Tfrc^{hum/hum} | 1.78 ± 0.43 |
| 12845scfv:hGAA | Tfrc^{hum/hum} | 1.70 ± 1.33 |
| 12847scfv:hGAA | Tfrc^{hum/hum} | 7.74 ± 9.42 |
| 12848scfv:hGAA | Tfrc^{hum/hum} | 0.82 ± 0.18 |
| 12850scfv:hGAA | Tfrc^{hum/hum} | 0.76 ± 0.34 |

Data were quantified from western blot as arbitrary units (FIG. 15). All values are mean ± SD, n = 2-4 per group.

Capillary depletion of mouse brain samples following liver-depot AAV8 anti-hTfRscfv:hGAA treatment. To confirm our HDD screen findings in a more long-term treatment model, we treated Tfrc^{hum} mice with selected anti-hTfRscfv:GAA delivered as episomal liver depot AAV8 anti-hTfRscfv:GAA under the TTR promoter. We found that all 4 anti-hTfRscfv:GAA delivered mature hGAA to the brain parenchyma when delivered as AAV8.

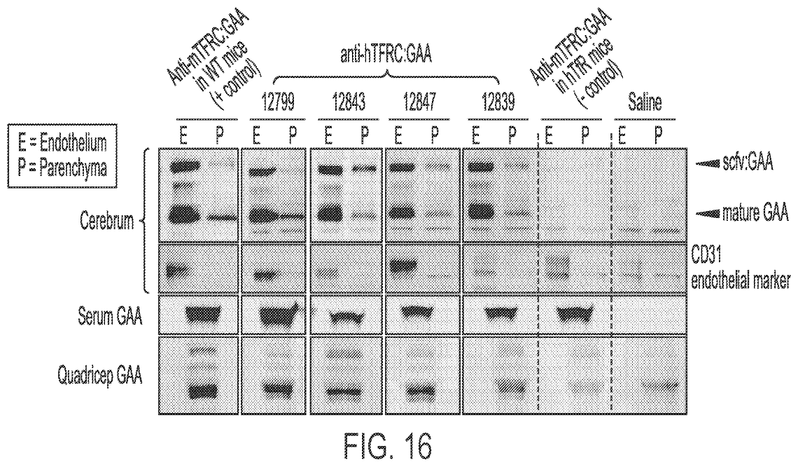

AAV production and in vivo transduction. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR. AAVs were diluted in PBS+0.001% F-68 Pluronic immediately prior to injection. Three-month-old Tfrc^{hum} mice were dosed with 3e12 vg/kg body weight in a volume of ˜100 μL. Mice were sacrificed 4 weeks post injection and capillary depletion and western blotting were performed as described above (FIG. 16, Table 30).

TABLE 30
Quantification of mature hGAA protein in brain parenchyma
fractions and BBB endothelial fractions of mice treated with
liver-depot AAV8 anti-hTfRscfv:hGAA.

| | | Mature hGAA | Mature hGAA |
|---|---|---|---|
| | | protein | protein |
| | | in brain | in brain |
| | | parenchyma | endothelium |
| | | (normalized to | (normalized to |
| Treatment group | Genotype | positive control) | positive control) |
| anti-mTfRscfv:hGAA | Wt | 1.00 | 1.00 |
| (positive control) | | | |
| anti-mTfRscfv:hGAA | Tfrc^{hum/hum} | 0.02 | 0.01 |
| (negative control) | | | |
| 12799scfv:hGAA | Tfrc^{hum/hum} | 0.94 | 0.94 |
| 12839scfv:hGAA | Tfrc^{hum/hum} | 0.49 | 0.62 |
| 12843scfv:hGAA | Tfrc^{hum/hum} | 0.61 | 0.63 |
| 12847scfv:hGAA | Tfrc^{hum/hum} | 1.90 | 1.33 |

Data were quantified from western blot as arbitrary units (FIG. 16). n = 1 per group.

Rescue of glycogen storage phenotype in Gaa^{-/-}/Tfrc^{hum} mice with AAV8 episomal liver depot anti-hTfRscfv:GAA. We tested four of the anti-hTfRscfv:GAA from the above experiment in Pompe disease model mice to determine whether hTfRscfv:GAA rescued the glycogen storage phenotype. We found that all four (12839, 12843, 12847, 12799) normalized glycogen to Wt levels.

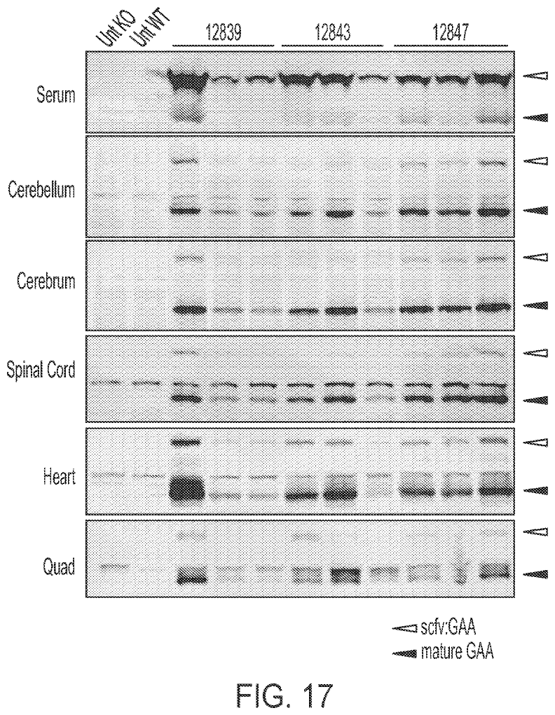

AAV production and in vivo transduction were performed as above. Three-month-old Gaa^{-/-}/Tfrc^{hum} mice were dosed with 2e12 vg/kg AAV8. Tissues were harvested 4 weeks post-injection and flash-frozen as above. hGAA Western blot was performed as above (FIG. 17, Table 31).

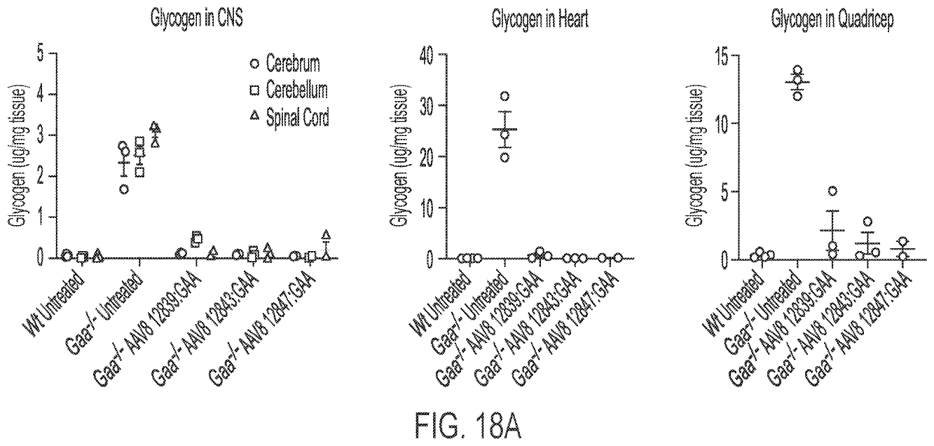

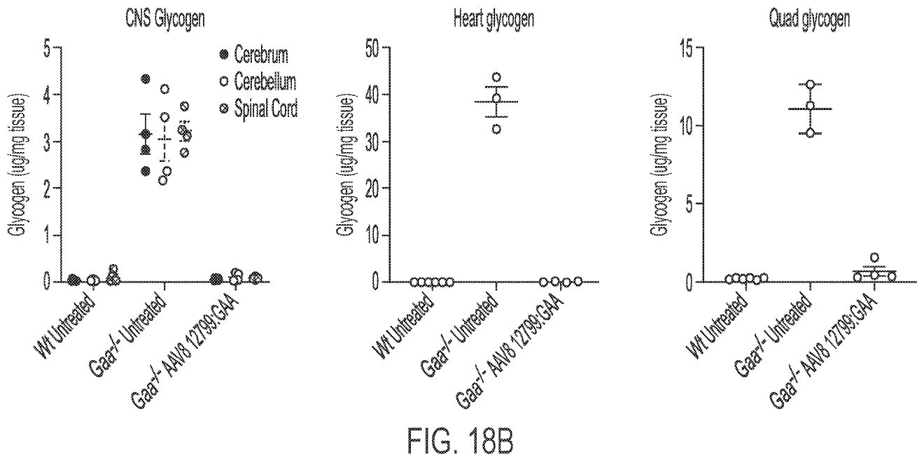

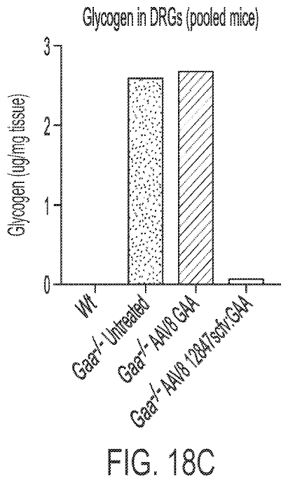

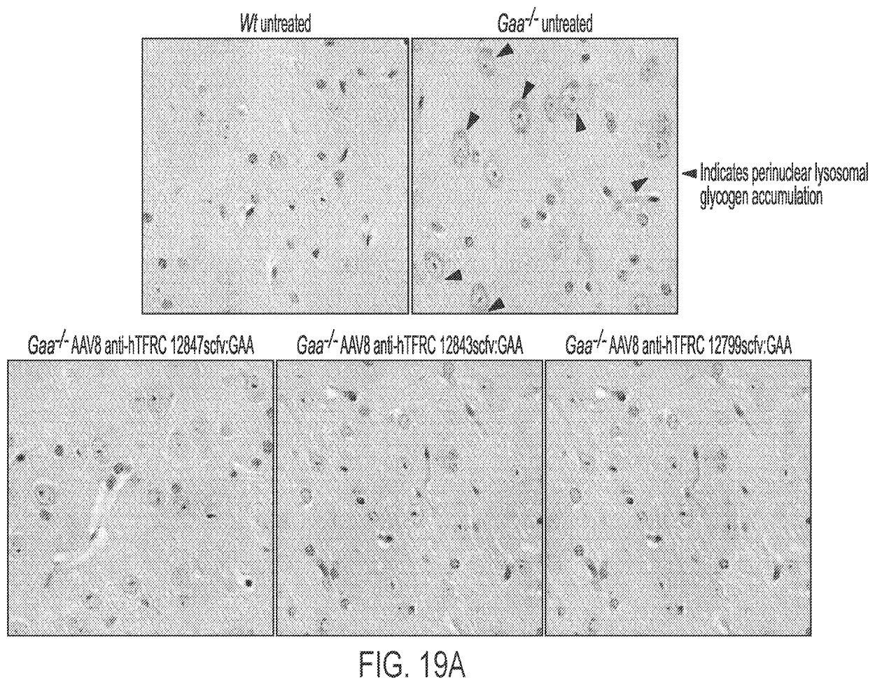

Glycogen quantification (Table 32, FIGS. 18A-18C). Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA). All groups had normal iron homeostasis at 4 weeks post-injection (serum iron, TIBC, hepcidin, tissue iron, tissue transferrin).

TABLE 31
Quantification of hGAA protein in tissues of Gaa^{-/-}/
Tfrc^{hum} mice treated with liver-depot AAV8 anti-hTfRscfv:hGAA.

| | | | | | | Spinal | | |
|---|---|---|---|---|---|---|---|---|
| Treatment group | n | Serum * | Liver * | Cerebrum ** | Cerebellum ** | Cord ** | Heart ** | Quadricep ** |
| Gaa^{-/-} Untreated | 1 | 0.00 | 0.02 | 0.00 | 0.00 | 0.00 | 0.02 | 0.01 |
| Gaa^{-/-} 12839scfv:hGAA | 3 | 2.42 ± | 1.63 ± | 0.14 ± | 0.13 ± | 0.19 ± | 0.53 ± | 0.14 ± |
| | | 2.41 | 0.96 | 0.12 | 0.12 | 0.19 | 0.52 | 0.16 |
| Gaa^{-/-} 12843scfv:hGAA | 3 | 2.07 ± | 2.23 ± | 0.17 ± | 0.11 ± | 0.17 ± | 0.49 ± | 0.18 ± |
| | | 1.35 | 0.08 | 0.07 | 0.05 | 0.09 | 0.31 | 0.06 |
| Gaa^{-/-} 12847scfv:hGAA | 3 | 1.56 ± | 1.40 ± | 0.25 ± | 0.21 ± | 0.42 ± | 0.58 ± | 0.19 ± |
| | | 0.71 | 0.13 | 0.04 | 0.09 | 0.19 | 0.17 | 0.08 |

Data were quantified from western blot as arbitrary units (FIG. 17). All values are mean ± SD, n = 1-3 per group.
* Total hGAA protein;
** Mature hGAA protein.

TABLE 32
Quantification of glycogen in tissues of Gaa^{-/-}/
Tfrc^{hum} mice treated with liver-depot AAV8 anti-hTfRscfv:hGAA.

| Treatment group | Cerebrum | Cerebellum | Spinal Cord | Heart | Quadricep | DRG |
|---|---|---|---|---|---|---|
| Wt Untreated | 0.06 ± 0.04* | 0.01 ± 0.04* | 0.05 ± 0.05* | 0.08 ± 0.02* | 0.34 ± 0.19* | 0.018 |
| Gaa−/− Untreated | 2.34 ± 0.58 | 2.51 ± 0.38 | 3.08 ± 0.23 | 25.30 ± 6.06 | 13.05 ± 0.98 | 2.58 |
| Gaa−/− 12839scfv:hGAA | 0.11 ± 0.03* | 0.46 ± 0.08* | 0.08 ± 0.10* | 0.68 ± 0.68* | 2.15 ± 2.52* | |
| Gaa−/− 12843scfv:hGAA | 0.09 ± 0.02* | 0.09 ± 0.08* | 0.13 ± 0.13* | 0.09 ± 0.01* | 1.22 ± 1.39* | |
| Gaa−/− 12847scfv:hGAA | 0.05 ± 0.01* | 0.02 ± 0.03* | 0.20 ± 0.33* | 0.11 ± 0.11* | 0.80 ± 0.79* | 0.066 |

| Treatment group | Cerebrum | Cerebellum | Spinal Cord | Heart | Quadricep |
|---|---|---|---|---|---|
| Wt Untreated | 0.043 ± 0.016* | 0.023 ± 0.008* | 0.105 ± 0.093* | 0.083 ± 0.022* | 0.247 ± 0.045* |
| Gaa−/− Untreated | 3.16 ± 0.837 | 3.043 ± 0.932 | 3.22 ± 0.414 | 34.48 ± 5.580 | 11.05 ± 2.192 |
| Gaa−/− 12799scfv:hGAA | 0.063 ± 0.023* | 0.107 ± 0.078* | 0.084 ± 0.019* | 0.123 ± 0.113* | 0.725 ± 0.599* |

All values are glycogen μg/mg tissue, mean ± SD, n = 3-4 per group. One Way ANOVA *p < 0.0001 vs. Gaa^{-/-} Untreated group.

Rescue of glycogen storage in brain and muscle in Gaa^{-/-}/Tfrc^{hum} mice with AAV8 episomal liver depot anti-hTfRscfv:GAA. We tested three selected anti-hTfRscfv:GAA (12799, 12843, and 12847) in Pompe disease model mice to determine whether hTfRscfv:GAA rescued the glycogen storage phenotype. In this experiment, we performed histology on brain and muscle sections to visualize glycogen in the tissues. We found that all three selected anti-hTfRscfv:GAA reduced glycogen staining in the brain and muscle. We selected 12847scfv:GAA for further analysis based on these data.

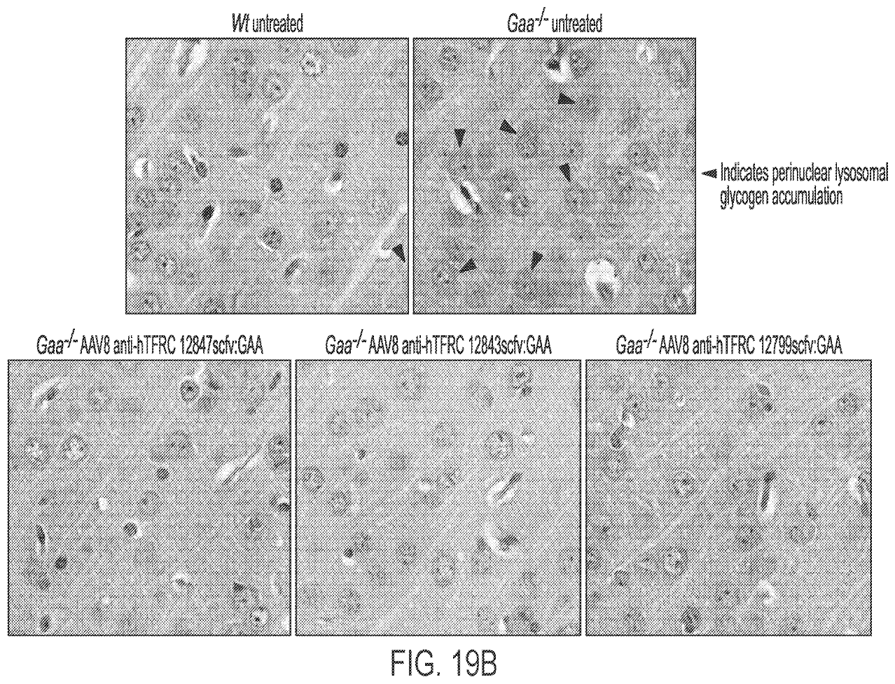

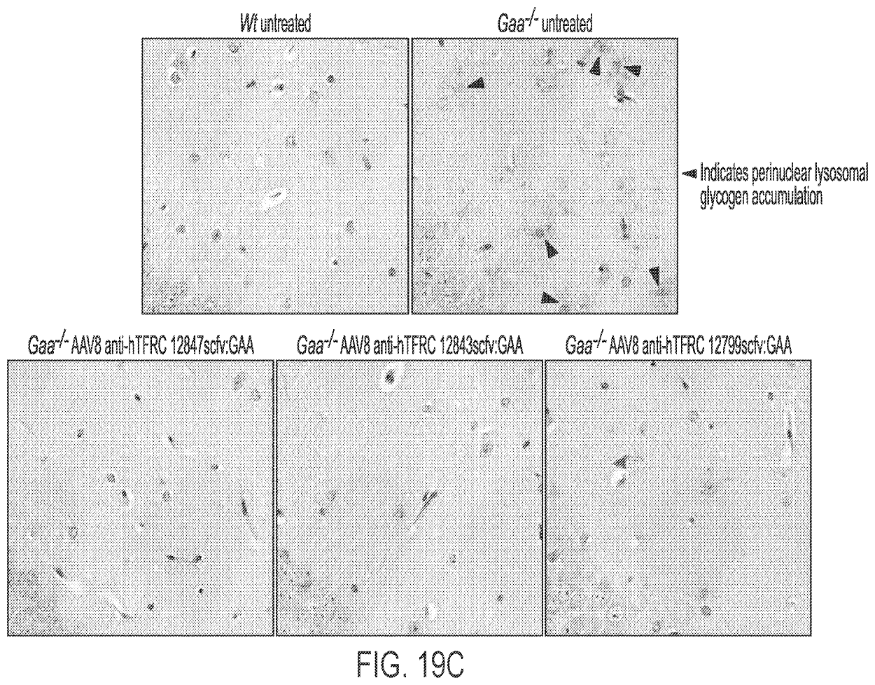

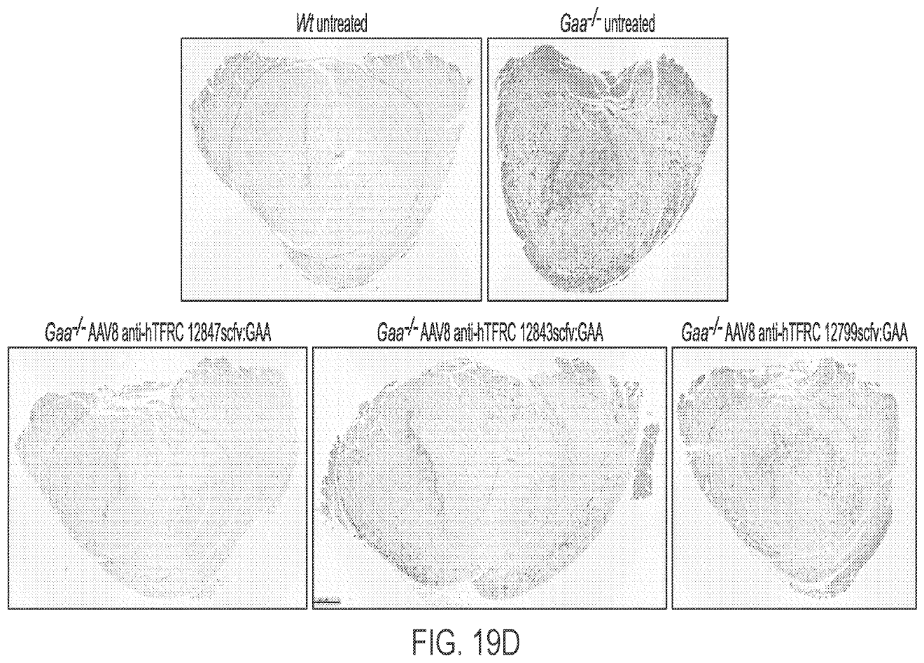

AAV production and in vivo transduction were performed as above. Three-month old Gaa^{-/-}/Tfrc^{hum} mice were dosed with 4e11 vg/kg AAV8. 4 weeks post-injection, tissues were frozen for glycogen analysis as above (Table 33). For histology, animals were perfused with saline (0.9% NaCl), and tissues were drop-fixed overnight in 10% Normal Buffered Formalin. Tissues were washed 3× in PBS and stored in PBS/0.01% sodium azide until embedding. Tissues were embedded in paraffin and 5 um sections were cut from brain (coronal, −2 mm bregma) and quadricep (fiber cross-section). Sections were stained with Periodic Acid-Schiff and Hematoxylin using standard protocols (FIGS. 19A-19D).

TABLE 33
Quantification of glycogen in tissues of Gaa^{-/-}/Tfrc^{hum} mice
treated with liver-depot AAV8 anti-hTfRscfv:hGAA.

| Treatment group | Cerebellum | Quadricep |
|---|---|---|
| Wt Untreated | 0.02 ± 0.03* | 0.55 ± 0.10* |
| Gaa^{-/-} Untreated | 1.91 ± 0.26 | 12.19 ± 3.02 |
| Gaa^{-/-} 12799scfv:hGAA | 0.10 ± 0.06* | 1.34 ± 0.9* |
| Gaa^{-/-} 12843scfv:hGAA | 0.09 ± 0.06* | 1.09 ± 1.27* |
| Gaa^{-/-} 12847scfv:hGAA | 0.07 ± 0.06* | 0.72 ± 0.64* |

All values are glycogen μg/mg tissue, mean ± SD, n = 5-8 per group. One Way ANOVA
*p < 0.0001 vs. Gaa^{-/-} Untreated group.

Insertion of anti-hTfR 12847scfv:GAA in Gaa^{-/-}/Tfrc^{hum} mice. We tested the selected anti-hTfR 12847scfv:GAA in Pompe disease model mice by albumin insertion to determine whether we could replicate the results we saw with episomal AAV8 liver depot expression. Albumin insertion of 12847scfv:GAA delivered mature hGAA protein to the brain and muscle, and rescued the glycogen storage phenotype in Gaa^{-/-}/Tfrc^{hum} mice. These data were produced with the native 12847scfv:GAA sequence that is not optimized.

We compared 12847scfv:GAA to the muscle-targeted anti-hCD63scfv:GAA in Gaa^{-/-}/Cd63^{hum} mice. In this particular experiment, the expression of anti-hCD63scfv:GAA was lower than usual and does not deliver as much GAA protein to the muscle nor normalize glycogen as it usually does. This may make it appear that anti-hCD63scfv:GAA is less effective than 12847scfv:GAA in the muscle but in most experiments we found them to be comparable in the muscle.

AAV production. A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR.

In vivo CRISPR/Cas9 insertion into the albumin locus. 3-month old Gaa^{-/-}/Tfrc^{hum} mice were dosed via tail vein injection with 3e12 vg/kg AAV8 12847scfv:GAA and 3 mg/kg LNP G666/Cas9 mRNA diluted in PBS+0.001% F-68 Pluronic. Mice were sacrificed 3 weeks post injection. Negative control mice received insertion AAV8 without LNP. Positive control mice were dosed with 4e11 vg/kg episomal liver depot AAV8 12847scfv:GAA under the TTR promoter (phenotype rescue data previously shown). Tissues were dissected from mice immediately after sacrifice by CO₂ asphyxiation, snap frozen in liquid nitrogen, and stored at −80° C. Blood was collected from mice by cardiac puncture immediately following CO₂ asphyxiation and serum was separated using serum separator tubes (BD Biosciences, 365967).

TABLE 34
Treatment Groups and Controls.

| Treatment group | Genotype | Function |
|---|---|---|
| Wt Untreated | Tfrc^{hum} | Normal untreated mouse control |
| Gaa^{-/-} untreated | Gaa^{-/-}/Tfrc^{hum} | Untreated Pompe disease mouse |
| Gaa^{-/-} insertion AAV | Gaa^{-/-}/Tfrc^{hum} | Negative control for insertion |
| only | | (no Cas9/gRNA delivered) |
| Gaa^{-/-} episomal AAV8 | Gaa^{-/-}/Tfrc^{hum} | Positive control, previously |
| TTR 12847scfv:hGAA | | shown rescue of |
| | | glycogen storage phenotype |
| Gaa^{-/-} insertion | Gaa^{-/-}/Tfrc^{hum} | Experimental insertion group |
| 12847scfv:hGAA | | |
| Gaa^{-/-} untreated | Gaa^{-/-}/Cd63^{hum} | Untreated Pompe disease |
| | | mouse (CD63 humanized) |
| Gaa^{-/-} insertion anti- | Gaa^{-/-}/Cd63^{hum} | Negative control for BBB- |
| CD63scfv:hGAA | | crossing (muscle targeted) |

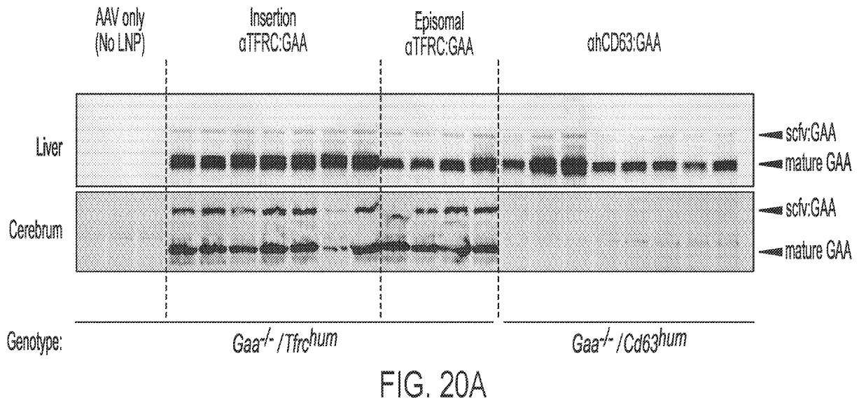

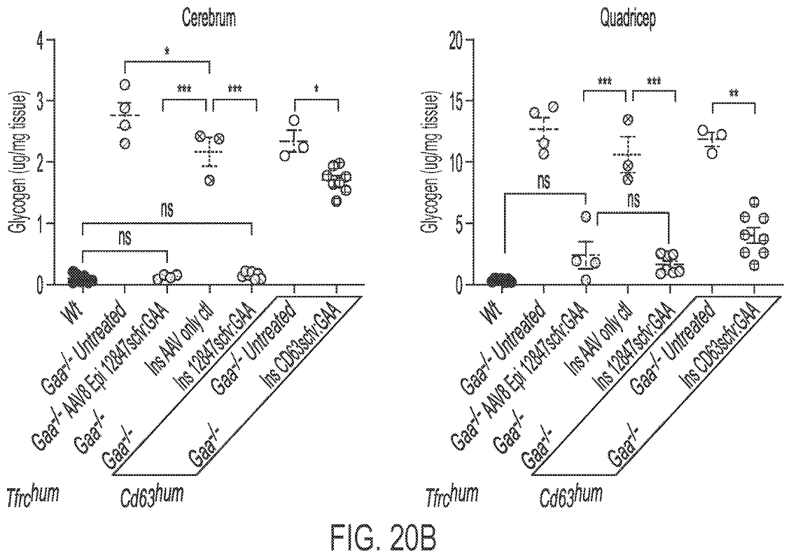

Western blot (Table 35, FIG. 20A). Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFLO7810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane's TPS signal (loading control).

Glycogen quantification (Table 36, FIG. 20B). Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA).

TABLE 35
Quantification of hGAA protein in tissues of Gaa^{-/-}/Tfrc^{hum} mice
treated with insertion anti-hTfR 12847scfv:hGAA.

| | Liver | Serum | Cerebrum | Quadricep |
|---|---|---|---|---|
| | total | total | mature | mature |
| Treatment group | hGAA | hGAA | hGAA | hGAA |
| Gaa^{-/-} insertion AAV only negative control | 0.02 ± 0.003 | 0.03 ± 0.02 | 0.002 ± 0.001 | 0.006 ± 0.002 |
| Gaa^{-/-} episomal AAV8 TTR 12847scfv:hGAA | 2.35 ± 0.72 | 3.65 ± 2.09 | 0.49 ± 0.20^{§§} | 0.148 ± 0.043^{§§} |
| Gaa^{-/-} insertion 12847scfv:hGAA | 4.31 ± 0.87* | 3.47 ± 2.37 | 0.57 ± 0.26^{§§} | 0.141 ± 0.062^{§§} |
| Gaa^{-/-} insertion anti-CD63scfv:hGAA | 2.67 ± 1.04* | 0.93 ± 0.55* | 0.01 ± 0.003 | 0.060 ± 0.037 |

All values are arbitrary units, mean ± SD, n = 3-8 per group. One Way ANOVA
*p < 0.05 vs. Gaa^{-/-} episomal AAV8 TTR 12847scfv:GAA group;
^{§§}p < 0.001 vs. AAV only negative control group.

TABLE 36
Quantification of glycogen in tissues of Gaa^{-/-}/Tfrc^{hum} mice treated with
insertion anti-hTfR 12847scfv:hGAA.

| Treatment group | Cerebrum | Quadricep |
|---|---|---|
| Wt untreated | 0.10 ± 0.07 | 0.37 ± 0.13 |
| Gaa^{-/-}/Tfrc^{hum} untreated (Tfrchum) | 2.76 ± 0.41 | 12.75 ± 1.88 |
| Gaa^{-/-}/Tfrc^{hum} insertion AAV only | 2.17 ± 0.40* | 10.64 ± 2.56 |
| Gaa^{-/-}/Tfrc^{hum} episomal AAV8 TTR 12847scfv:hGAA | 0.13 ± 0.03***^{§} | 2.44 ± 2.21***^{§} |
| Gaa^{-/-}/Tfrc^{hum} insertion 12847scfv:hGAA | 0.16 ± 0.05***^{§} | 1.67 ± 0.76***^{§} |
| Gaa^{-/-}/Cd63^{hum} untreated | 2.34 ± 0.30 | 11.91 ± 1.01 |
| Gaa^{-/-}/Cd63^{hum} insertion anti-CD63scfv:hGAA | 1.71 ± 0.20* | 4.06 ± 0.13** |

All values are glycogen μg/mg tissue, mean ± SD, n = 3-8 per group. One Way ANOVA
*p < 0.01 vs. Gaa^{-/-}/Cd63^{hum} untreated group;
**p <0.001 vs. Gaa^{-/-}/Cd63^{hum} untreated group;
***p < 0.0001 vs. Gaa^{-/-}/Tfrc^{hum} untreated group;
^{§}non-significant vs. Wt untreated group.

Similar experiments are then performed in which neonatal Gaa^{-/-}; Tfrc^{hu/hu} mice are dosed intravenously at P1 with the following: (1) recombinant AAV8 encoding anti-TfR:GAA; or (2) LNP-g666 and recombinant AAV8 anti-TfR:GAA insertion template. Untreated Gaa^{-/-}; Tfrc^{hu/hu} mice and wild type mice are used as controls. Blood is collected and serum prepared at various time points post-administrations, and tissues are collected at various time points post-administration. Serum anti-TfR:GAA levels and glycogen levels in various muscle and CNS tissues are measured over the time course.

To assess whether glycogen reduction translates into improved muscle function, the mice are tested on grip strength apparatuses at a time point post-administration. Limb grip strength is measured with a force meter (Columbus Instruments, Columbus, OH, USA). All tests are performed in triplicate.

In summary, the combination of the highly precise and targeted CRISPR/Cas9 technology delivered by LNP and the anti-TfR:GAA DNA template delivered by the selected rAAV8 vector allows for long-term expression of anti-TfR:GAA protein from hepatocytes and delivery to muscle cells and CNS cells affected in PD, potentially providing a life-long effective treatment to PD patients, including neonatal patients.

TABLE 37
Additional GAA sequences.

| Sequence | |
|---|---|
| name | Amino acids |
| GAA_1 77 | GHILLHDFLLVPRELSGSSPVLEETHPAHQQGASRPGPRDAQAHPGRPRAVPTQCDVPP |
| (SEQ ID NO: | NSRFDCAPDKAITQEQCEARGCCYIPAKQGLQGAQMGQPWCFFPPSYPSYKLENLSSSE |
| 589) | MGYTATLTRTTPTFFPKDILTLRLDVMMETENRLHFTIKDPANRRYEVPLETPHVHSRA |
| | PSPLYSVEFSEEPFGVIVRRQLDGRVLLNTTVAPLFFADQFLQLSTSLPSQYITGLAEHLS |
| | PLMLSTSWTRITLWNRDLAPTPGANLYGSHPFYLALEDGGSAHGVFLLNSNAMDVVL |
| | QPSPALSWRSTGGILDVYIFLGPEPKSVVQQYLDVVGYPFMPPYWGLGFHLCRWGYSS |
| | TAITRQVVENMTRAHFPLDVQWNDLDYMDSRRDFTFNKDGFRDFPAMVQELHQGGR |
| | RYMMIVDPAISSSGPAGSYRPYDEGLRRGVFITNETGQPLIGKVWPGSTAFPDFTNPTA |
| | LAWWEDMVAEFHDQVPFDGMWIDMNEPSNFIRGSEDGCPNNELENPPYVPGVVGGT |
| | LQAATICASSHQFLSTHYNLHNLYGLTEAIASHRALVKARGTRPFVISRSTFAGHGRYA |
| | GHWTGDVWSSWEQLASSVPEILQFNLLGVPLVGADVCGFLGNTSEELCVRWTQLGAF |
| | YPFMRNHNSLLSLPQEPYSFSEPAQQAMRKALTLRYALLPHLYTLFHQAHVAGETVAR |
| | PLFLEFPKDSSTWTVDHQLLWGEALLITPVLQAGKAEVTGYFPLGTWYDLQTVPVEAL |
| | GSLPPPPAAPREPAIHSEGQWVTLPAPLDTINVHLRAGYIIPLQGPGLTTTESRQQPMAL |
| | AVALTKGGEARGELFWDDGESLEVLERGAYTQVIFLARNNTIVNELVRVTSEGAGLQL |
| | QKVTVLGVATAPQQVLSNGVPVSNFTYSPDTKVLDICVSLLMGEQFLVSWC |
| GAA_2 22 | LLVPRELSGSSPVLEETHPAHQQGASRPGPRDAQAHPGRPRAVPTQCDVPPNSRFDCAP |
| (SEQ ID NO: | DKAITQEQCEARGCCYIPAKQGLQGAQMGQPWCFFPPSYPSYKLENLSSSEMGYTATL |
| 590) | TRTTPTFFPKDILTLRLDVMMETENRLHFTIKDPANRRYEVPLETPHVHSRAPSPLYSVE |
| | FSEEPFGVIVRRQLDGRVLLNTTVAPLFFADQFLQLSTSLPSQYITGLAEHLSPLMLSTS |
| | WTRITLWNRDLAPTPGANLYGSHPFYLALEDGGSAHGVFLLNSNAMDVVLQPSPALS |
| | WRSTGGILDVYIFLGPEPKSVVQQYLDVVGYPFMPPYWGLGFHLCRWGYSSTAITRQV |
| | VENMTRAHFPLDVQWNDLDYMDSRRDFTFNKDGFRDFPAMVQELHQGGRRYMMIV |
| | DPAISSSGPAGSYRPYDEGLRRGVFITNETGQPLIGKVWPGSTAFPDFTNPTALAWWED |
| | MVAEFHDQVPFDGMWIDMNEPSNFIRGSEDGCPNNELENPPYVPGVVGGTLQAATICA |
| | SSHQFLSTHYNLHNLYGLTEAIASHRALVKARGTRPFVISRSTFAGHGRYAGHWTGDV |
| | WSSWEQLASSVPEILQFNLLGVPLVGADVCGFLGNTSEELCVRWTQLGAFYPFMRNH |
| | NSLLSLPQEPYSFSEPAQQAMRKALTLRYALLPHLYTLFHQAHVAGETVARPLFLEFPK |
| | DSSTWTVDHQLLWGEALLITPVLQAGKAEVTGYFPLGTWYDLQTVPVEALGSLPPPPA |
| | APREPAIHSEGQWVTLPAPLDTINVHLRAGYIIPLQGPGLTTTESRQQPMALAVALTKG |
| | GEARGELFWDDGESLEVLERGAYTQVIFLARNNTIVNELVRVTSEGAGLQLQKVTVLG |
| | VATAPQQVLSNGVPVSNFTYSPDTKVLDICVSLLMGEQFLVSWC |
| GAA_3 61 | AHPGRPRAVPTQCDVPPNSRFDCAPDKAITQEQCEARGCCYIPAKQGLQGAQMGQPW |
| (SEQ ID NO: | CFFPPSYPSYKLENLSSSEMGYTATLTRTTPTFFPKDILTLRLDVMMETENRLHFTIKDP |
| 591) | ANRRYEVPLETPHVHSRAPSPLYSVEFSEEPFGVIVRRQLDGRVLLNTTVAPLFFADQFL |
| | QLSTSLPSQYITGLAEHLSPLMLSTSWTRITLWNRDLAPTPGANLYGSHPFYLALEDGG |
| | SAHGVFLLNSNAMDVVLQPSPALSWRSTGGILDVYIFLGPEPKSVVQQYLDVVGYPFM |
| | PPYWGLGFHLCRWGYSSTAITRQVVENMTRAHFPLDVQWNDLDYMDSRRDFTFNKD |
| | GFRDFPAMVQELHQGGRRYMMIVDPAISSSGPAGSYRPYDEGLRRGVFITNETGQPLIG |
| | KVWPGSTAFPDFTNPTALAWWEDMVAEFHDQVPFDGMWIDMNEPSNFIRGSEDGCPN |
| | NELENPPYVPGVVGGTLQAATICASSHQFLSTHYNLHNLYGLTEAIASHRALVKARGT |
| | RPFVISRSTFAGHGRYAGHWTGDVWSSWEQLASSVPEILQFNLLGVPLVGADVCGFLG |
| | NTSEELCVRWTQLGAFYPFMRNHNSLLSLPQEPYSFSEPAQQAMRKALTLRYALLPHL |
| | YTLFHQAHVAGETVARPLFLEFPKDSSTWTVDHQLLWGEALLITPVLQAGKAEVTGYF |
| | PLGTWYDLQTVPVEALGSLPPPPAAPREPAIHSEGQWVTLPAPLDTINVHLRAGYIIPLQ |
| | GPGLTTTESRQQPMALAVALTKGGEARGELFWDDGESLEVLERGAYTQVIFLARNNTI |
| | VNELVRVTSEGAGLQLQKVTVLGVATAPQQVLSNGVPVSNFTYSPDTKVLDICVSLLM |
| | GEQFLVSWC |

We claim:

1. A method of inserting a nucleic acid encoding a polypeptide of interest into a target genomic locus in a neonatal cell or a population of neonatal cells, comprising administering to the neonatal cell or the population of neonatal cells:
(a) a nucleic acid construct comprising a coding sequence for the polypeptide of interest; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

2. A method of expressing a polypeptide of interest from a target genomic locus in a neonatal cell or a population of neonatal cells, comprising administering to the neonatal cell or the population of neonatal cells:
(a) a nucleic acid construct comprising a coding sequence for the polypeptide of interest; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the polypeptide of interest is expressed from the modified target genomic locus.

3. The method of claim 1 or 2, wherein the neonatal cell is a liver cell or the population of neonatal cells is a population of liver cells.

4. The method of any preceding claim, wherein the neonatal cell is a hepatocyte or the population of neonatal cells is a population of hepatocytes.

5. The method of any preceding claim, wherein the neonatal cell is a human cell or the population of neonatal cells is a population of human cells.

6. The method of claim 5, wherein the neonatal cell or the population of neonatal cells is from a neonatal subject within 52 weeks after birth.

7. The method of claim 5, wherein the neonatal cell or the population of neonatal cells is from a neonatal subject within 24 weeks after birth.

8. The method of claim 5, wherein the neonatal cell or the population of neonatal cells is from a neonatal subject within 12 weeks after birth.

9. The method of claim 5, wherein the neonatal cell or the population of neonatal cells is from a neonatal subject within 8 weeks after birth.

10. The method of claim 5, wherein the neonatal cell or the population of neonatal cells is from a neonatal subject within 4 weeks after birth.

11. The method of any preceding claim, wherein the neonatal cell is in vitro or ex vivo or the population of neonatal cells is in vitro or ex vivo.

12. The method of any one of claims 1-10, wherein the neonatal cell is in vivo in a neonatal subject or the population of neonatal cells is in vivo in a neonatal subject.

13. A method of inserting a nucleic acid encoding a polypeptide of interest into a target genomic locus in a neonatal cell in a neonatal subject, comprising administering to the neonatal subject:
(a) a nucleic acid construct comprising a coding sequence for the polypeptide of interest; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

14. A method of expressing a polypeptide of interest from a target genomic locus in a neonatal cell in a neonatal subject, comprising administering to the neonatal subject:
(a) a nucleic acid construct comprising a coding sequence for the polypeptide of interest; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the polypeptide of interest is expressed from the modified target genomic locus.

15. A method of expressing a polypeptide of interest from a target genomic locus in a neonatal cell in a neonatal subject, comprising administering to the neonatal subject:
(a) a nucleic acid construct comprising a coding sequence for the polypeptide of interest; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the polypeptide of interest is expressed from the modified target genomic locus,
wherein the subject comprises a mutation in a genome in the subject, wherein the mutation results in reduced activity or expression of an endogenous polypeptide having enzymatic activity.

16. The method of claim 15, wherein the nucleic acid encoding the polypeptide of interest encodes a polypeptide having the enzymatic activity of a wild type polypeptide encoded by the gene in which the subject has a mutation that results in reduced activity or expression of the endogenous polypeptide.

17. The method of any one of claims 13-16, wherein the neonatal cell is a liver cell.

18. The method of any one of claims 13-17, wherein the neonatal cell is a hepatocyte.

19. The method of any one of claims 13-18, wherein the neonatal cell is a human cell.

20. A method of treating an enzyme deficiency in a neonatal subject in need thereof, comprising administering to the neonatal subject:
(a) a nucleic acid construct comprising a coding sequence for a polypeptide of interest, wherein the polypeptide of interest comprises an enzyme to treat the enzyme deficiency; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency.

21. A method of preventing or reducing the onset of a sign or symptom of an enzyme deficiency in a neonatal subject in need thereof, comprising administering to the neonatal subject:
(a) a nucleic acid construct comprising a coding sequence for a polypeptide of interest, wherein the lysosomal storage disease is characterized by a loss-of-function of the polypeptide of interest; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency.

22. The method of claim 20 or 21, wherein the neonatal subject has a disease of a bleeding disorder characterized by the enzyme deficiency.

23. The method of claim 22, wherein the bleeding disorder is selected from hemophilia A, hemophilia B, and von Willebrand disease.

24. The method of claim 20 or 21, wherein the neonatal subject has a disease of an inborn error of metabolism characterized by the enzyme deficiency.

25. The method of claim 20 or 21, wherein the neonatal subject has a disease selected from Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, argininosuccinic aciduria, methylmalonic aciduria, type I citrullinemia/argininosuccinate synthetase deficiency, carbamoyl-phosphate synthase 1 deficiency, propionic acidemia, isovaleric acidemia, glutaric academia I, and progressive familial intrahepatic cholestasis, types 2 and 3, Fabry disease, Gaucher disease type I, Gaucher disease type II, Gaucher disease type III, Niemann-Pick disease type A, Niemann-Pick disease type BGM1-gangliosidosis, Sandhoff disease, Tay-Sachs disease, GM2-activator deficiency, GM3-gangliosidosis, metachromatic leukodystrophy, sphingolipid-activator deficiency, Scheie disease, Hurler-Scheie disease, Hurler disease, Hunter disease, Sanfilippo A, Sanfilippo B, Sanfilippo C, Sanfilippo D, Morquio syndrome A, Morquio syndrome B, Maroteaux-Lamy disease, Sly disease, MPS IX, or Pompe disease.

26. The method of claim 20 or 21, wherein the neonatal subject has a lysosomal storage disease characterized by the enzyme deficiency.

27. A method of treating a lysosomal storage disease in a neonatal subject in need thereof, comprising administering to the neonatal subject:
(a) a nucleic acid construct comprising a coding sequence for a polypeptide of interest, wherein the lysosomal storage disease is characterized by a loss-of-function of the polypeptide of interest; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the polypeptide of interest is expressed from the modified target genomic locus, thereby treating the lysosomal storage disease.

28. A method of preventing or reducing the onset of a sign or symptom of a lysosomal storage disease in a neonatal subject in need thereof, comprising administering to the neonatal subject:
(a) a nucleic acid construct comprising a coding sequence for a polypeptide of interest, wherein the lysosomal storage disease is characterized by a loss-of-function of the polypeptide of interest; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the lysosomal storage disease.

29. The method of any one of claims 12-28, wherein the neonatal subject is a human subject.

30. The method of claim 29, wherein the neonatal subject is within 52 weeks after birth.

31. The method of claim 29, wherein the neonatal subject is within 24 weeks after birth.

32. The method of claim 29, wherein the neonatal subject is within 12 weeks after birth.

33. The method of claim 29, wherein the neonatal subject is within 8 weeks after birth.

34. The method of claim 29, wherein the neonatal subject is within 4 weeks after birth.

35. The method of any one of claims 12-34, wherein the method results in increased expression of the polypeptide of interest in the subject compared to a method comprising administering an episomal expression vector encoding the polypeptide of interest to a control subject.

36. The method of any one of claims 12-35, wherein the method results in increased serum levels of the polypeptide of interest in the subject compared to a method comprising administering an episomal expression vector encoding the polypeptide of interest to a control subject.

37. The method of any one of claims 12-36, wherein the expression or activity of the polypeptide of interest is at least 50% of the expression or activity of the polypeptide at a peak level of expression measured for the human subject at six months after the administering.

38. The method of any one of claims 12-37, wherein the expression or activity of the polypeptide of interest is at least 50% of the expression or activity of the polypeptide at a peak level of expression measured for the human subject at one year after the administering.

39. The method of any one of claims 12-38, wherein the expression or activity of the polypeptide of interest is at least 60% of the expression or activity of the polypeptide at a peak level of expression measured for the human subject at six months after the administering.

40. The method of any one of claims 12-39, wherein expression or activity of the polypeptide of interest is at least 50% of the expression or activity of the polypeptide at a peak level of expression measured for the human subject at two years after the administering.

41. The method of any one of claims 12-40, wherein the expression or activity of the polypeptide of interest is at least 60% of the expression or activity of the polypeptide at a peak level of expression measured for the human subject at two years after the administering.

42. The method of any one of claims 12-41, wherein the expression or activity of the polypeptide of interest is at least 60% of the expression or activity of the polypeptide at a peak level of expression measured for the human subject at six months after the administering.

43. The method of any one of claim 12-42, wherein the method further comprises assessing preexisting AAV immunity in the neonatal subject prior to administering the nucleic acid construct to the subject.

44. The method of claim 43, wherein the preexisting AAV immunity is preexisting AAV8 immunity.

45. The method of claim 43 or 44, wherein assessing preexisting AAV immunity comprises assessing immunogenicity using a total antibody immune assay or a neutralizing antibody assay.

46. The method of any preceding claim, wherein the nucleic acid construct is administered simultaneously with the nuclease agent or the one or more nucleic acids encoding the nuclease agent.

47. The method of any one of claims 1-45, wherein the nucleic acid construct is not administered simultaneously with the nuclease agent or the one or more nucleic acids encoding the nuclease agent.

48. The method of claim 47, wherein the nucleic acid construct is administered prior to the nuclease agent or the one or more nucleic acids encoding the nuclease agent.

49. The method of claim 47, wherein the nucleic acid construct is administered after the nuclease agent or the one or more nucleic acids encoding the nuclease agent.

50. The method of any preceding claim, wherein the polypeptide of interest comprises a therapeutic polypeptide.

51. The method of any preceding claim, wherein the polypeptide of interest is a secreted polypeptide.

52. The method of claim 50 or 51, wherein the polypeptide of interest comprises a hydrolase, α-galactosidase, β-galactosidase, α-glucosidase, β-glucosidase, saposin-C activator, ceramidase, sphingomyelinase, β-hexosaminidase, GM2 activator, GM3 synthase, arylsulfatase, sphingolipid activator, α-iduronidase, iduronidase-2-sulfatase, heparin N-sulfatase, N-acetyl-α-glucosaminidase, α-glucosamide N-acetyltransferase, N-acetylglucosamine-6-sulfatase, N-acetylgalactosamine-6-sulfate sulfatase, N-acetylgalactosamine-4-sulfatase, β-glucuronidase, or a hyaluronidase.

53. The method of claim 52, wherein the polypeptide of interest comprises lysosomal alpha-glucosidase.

54. The method of any preceding claim, wherein the polypeptide of interest comprises a sequence at least 90%, at least 91%, at least 92%, at least 93%, at least 94%, at least 95%, at least 96%, at least 97%, at least 98%, or at least 99% identical to the amino acid sequence of SEQ ID NO: 173, optionally wherein the polypeptide of interest is encoded by a codon-optimized and CpG-depleted nucleotide sequence.

55. The method of any preceding claim, wherein the coding sequence for the polypeptide of interest comprises a nucleotide sequence at least 90%, at least 91%, at least 92%, at least 93%, at least 94%, at least 95%, at least 96%, at least 97%, at least 98%, or at least 99% identical to a nucleotide sequence selected from SEQ ID NOS: 174-182 and 581-588, optionally selected from SEQ ID NOS: 175-179, wherein the nucleotide sequence is codon-optimized and CpG-depleted.

56. The method of any preceding claim, wherein the nucleic acid construct is CpG depleted.

57. The method of any one of claims 52-56, wherein the polypeptide of interest comprises a delivery domain.

58. The method of claim 57, wherein the polypeptide of interest is delivered to and internalized by skeletal muscle and heart tissue in the subject.

59. The method of any preceding claim, wherein the subject has an infantile-onset genetic disorder.

60. The method of any preceding claim, wherein the subject wherein the subject has Pompe disease.

61. The method of any one of claims 1-51, wherein the subject has a bleeding disorder.

62. The method of claim 61, wherein the polypeptide of interest is Factor VIII, Factor IX, or von Willebrand factor.

63. The method of any one of claims 1-50, wherein the polypeptide of interest is an intracellular polypeptide.

64. The method of any preceding claim, wherein the nucleic acid construct comprises a splice acceptor upstream of the coding sequence for the polypeptide of interest.

65. The method of any preceding claim, wherein the nucleic acid construct comprises a polyadenylation signal or sequence downstream of the coding sequence for the polypeptide of interest.

66. The method of any one of claims 1-63, wherein the nucleic acid construct comprises a splice acceptor upstream of the coding sequence for the polypeptide of interest, and the nucleic acid construct comprises a polyadenylation signal or sequence downstream of the coding sequence for the polypeptide of interest.

67. The method of any preceding claim, wherein the nucleic acid construct does not comprise a homology arm.

68. The method of claim 67, wherein the nucleic acid construct is inserted into the target genomic locus via non-homologous end joining.

69. The method of any one of claims 1-66, wherein the nucleic acid construct comprises homology arms.

70. The method of claim 69, wherein the nucleic acid construct is inserted into the target genomic locus via homology-directed repair.

71. The method of any preceding claim, wherein the nucleic acid construct does not comprise a promoter that drives the expression of the polypeptide of interest.

72. The method of any preceding claim, wherein the nucleic acid construct is single-stranded DNA or double-stranded DNA.

73. The method of claim 72, wherein the nucleic acid construct is single-stranded DNA.

74. The method of any preceding claim, wherein the nucleic acid construct is a bidirectional nucleic acid construct.

75. The method of claim 74, wherein the nucleic acid construct comprises:
(I) a first segment comprising the coding sequence for the polypeptide of interest; and
(II) a second segment comprising a reverse complement of a second coding sequence for the polypeptide of interest.

76. The method of claim 75, wherein the nucleic acid construct comprises from 5′ to 3′: a first splice acceptor, the coding sequence for the polypeptide of interest, a first polyadenylation signal or sequence, a reverse complement of a second polyadenylation signal or sequence, the reverse complement of the second coding sequence for the polypeptide of interest, and a reverse complement of a second splice acceptor.

77. The method of claim 75 or 76, wherein the coding sequence for the polypeptide of interest and the second coding sequence for the polypeptide of interest are different.

78. The method of any preceding claim, wherein the nucleic acid construct is in a nucleic acid vector or a lipid nanoparticle.

79. The method of claim 78, wherein the nucleic acid construct is in the nucleic acid vector.

80. The method of claim 79, wherein the nucleic acid vector is a viral vector.

81. The method of claim 79 or 80, wherein the nucleic acid vector is an adeno-associated viral (AAV) vector,
optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160.

82. The method of claim 81, wherein the AAV vector is a single-stranded AAV (ssAAV) vector.

83. The method of claim 81 or 82, wherein the AAV vector is derived from an AAV8 vector, an AAV3B vector, an AAV5 vector, an AAV6 vector, an AAV7 vector, an AAV9 vector, an AAVrh.74 vector, or an AAVhu.37 vector.

84. The method of claim 83, wherein the AAV vector is a recombinant AAV8 (rAAV8) vector.

85. The method of claim 84, wherein the AAV vector is a single-stranded rAAV8 vector.

86. The method of any preceding claim, wherein the nucleic acid construct is CpG-depleted.

87. The method of any preceding claim, wherein the target genomic locus is an albumin gene, optionally wherein the albumin gene is a human albumin gene.

88. The method of claim 87, wherein the nuclease target site is in intron 1 of the albumin gene.

89. The method of any preceding claim, wherein the nuclease agent comprises:
(a) a zinc finger nuclease (ZFN);
(b) a transcription activator-like effector nuclease (TALEN); or
(c) (i) a Cas protein or a nucleic acid encoding the Cas protein; and
(ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

90. The method of any one of claims 1-89, wherein the nuclease agent comprises:
(a) a Cas protein or a nucleic acid encoding the Cas protein; and
(b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

91. The method of claim 90, wherein the guide RNA target sequence is in intron 1 of an albumin gene.

92. The method of claim 91, wherein the albumin gene is a human albumin gene.

93. The method of any one of claims 90-92, wherein:
(I) the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41; and/or
(II) the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41.

94. The method of any one of claims 90-93, wherein the DNA-targeting segment comprises any one of SEQ ID NOS: 30-61, optionally wherein the DNA-targeting segment comprises any one of SEQ ID NOS: 36, 30, 33, and 41.

95. The method of any one of claims 90-94, wherein the DNA-targeting segment consists of any one of SEQ ID NOS: 30-61, optionally wherein the DNA-targeting segment consists of any one of SEQ ID NOS: 36, 30, 33, and 41.

96. The method of any one of claims 90-95, wherein the guide RNA comprises any one of SEQ ID NOS: 62-125, optionally wherein the guide RNA comprises any one of SEQ ID NOS: 68, 100, 62, 94, 65, 97, 73, and 105.

97. The method of any one of claims 90-96, wherein:
(I) the DNA-targeting segment comprises at least 17, at least 18, at least 19, or at least 20 contiguous nucleotides of SEQ ID NO: 36; and/or
(II) the DNA-targeting segment is at least 90% or at least 95% identical to SEQ ID NO: 36.

98. The method of any one of claims 90-97, wherein the DNA-targeting segment comprises SEQ ID NO: 36.

99. The method of any one of claims 90-98, wherein the DNA-targeting segment consists of SEQ ID NO: 36.

100. The method of any one of claims 90-99, wherein the guide RNA comprises SEQ ID NO: 68 or 100.

101. The method of any one of claims 90-100, wherein the method comprises administering the guide RNA in the form of RNA.

102. The method of any one of claims 90-101, wherein the guide RNA comprises at least one modification.

103. The method of claim 102, wherein the at least one modification comprises a 2′-O-methyl-modified nucleotide.

104. The method of claim 102 or 103, wherein the at least one modification comprises a phosphorothioate bond between nucleotides.

105. The method of any one of claims 102-104, wherein the at least one modification comprises a modification at one or more of the first five nucleotides at the 5′ end of the guide RNA.

106. The method of any one of claims 102-105, wherein the at least one modification comprises a modification at one or more of the last five nucleotides at the 3′ end of the guide RNA.

107. The method of any one of claims 102-106, wherein the at least one modification comprises phosphorothioate bonds between the first four nucleotides at the 5′ end of the guide RNA.

108. The method of any one of claims 102-107, wherein the at least one modification comprises phosphorothioate bonds between the last four nucleotides at the 3′ end of the guide RNA.

109. The method of any one of claims 102-108, wherein the at least one modification comprises 2′-O-methyl-modified nucleotides at the first three nucleotides at the 5′ end of the guide RNA.

110. The method of any one of claims 102-109, wherein the at least one modification comprises 2′-O-methyl-modified nucleotides at the last three nucleotides at the 3′ end of the guide RNA.

111. The method of any one of claims 102-110, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

112. The method of any one of claims 90-111, wherein the guide RNA is a single guide RNA (sgRNA).

113. The method of any one of claims 90-112, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

114. The method of any one of claims 90-113, wherein the Cas protein is a Cas9 protein.

115. The method of claim 114, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

116. The method of claim 114, wherein the Cas protein is derived from a Streptococcus pyogenes Cas9 protein.

117. The method of any one of claims 90-116, wherein the Cas protein comprises the sequence set forth in SEQ ID NO: 11.

118. The method of any one of claims 90-117, wherein the nucleic acid encoding the Cas protein is codon-optimized for expression in a mammalian cell or a human cell.

119. The method of any one of claims 90-118, wherein the method comprises administering the nucleic acid encoding the Cas protein, wherein the nucleic acid comprises an mRNA encoding the Cas protein.

120. The method of claim 119, wherein the mRNA encoding the Cas protein comprises at least one modification.

121. The method of claim 119 or 120, wherein the mRNA encoding the Cas protein is modified to comprise a modified uridine at one or more or all uridine positions.

122. The method of claim 121, wherein the modified uridine is pseudouridine or N1-methyl-pseudouridine, optionally N1-methyl-pseudouridine.

123. The method of claim 121 or 122, wherein the mRNA encoding the Cas protein is fully substituted with pseudouridine or N1-methyl-pseudouridine, optionally N1-methyl-pseudouridine.

124. The method of any one of claims 119-123, wherein the mRNA encoding the Cas protein comprises a 5′ cap.

125. The method of any one of claims 119-124, wherein the mRNA encoding the Cas protein comprises a polyadenylation sequence.

126. The method of any one of claims 119-125, wherein the mRNA encoding the Cas protein comprises the sequence set forth in SEQ ID NO: 2, 1, or 12.

127. The method of any one of claims 90-126, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

128. The method of any one of claims 90-127, wherein the method comprises administering the guide RNA in the form of RNA, and the guide RNA comprises SEQ ID NO: 68 or 100, and
wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 2, 1, or 12.

129. The method of any one of claims 90-128, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and
wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence.

130. The method of any one of claims 90-129, wherein the Cas protein or the nucleic acid encoding the Cas protein and the guide RNA or the one or more DNAs encoding the guide RNA are associated with a lipid nanoparticle.

131. The method of claim 130, wherein the lipid nanoparticle comprises a cationic lipid, a neutral lipid, a helper lipid, and a stealth lipid.

132. The method of claim 131, wherein the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate).

133. The method of claim 130 or 131, wherein the neutral lipid is distearoylphosphatidylcholine or 1,2-distearoyl-sn-glycero-3-phosphocholine (DSPC).

134. The method of any one of claims 131-133, wherein the helper lipid is cholesterol.

135. The method of any one of claims 131-134, wherein the stealth lipid is 1,2-dimyristoyl-rac-glycero-3-methoxypolyethylene glycol-2000 (PEG2k-DMG).

136. The method of any one of claims 131-135, wherein the cationic lipid is Lipid A, the neutral lipid is DSPC, the helper lipid is cholesterol, and the stealth lipid is PEG2k-DMG.

137. The method of any one of claims 131-136, wherein the lipid nanoparticle comprises four lipids at the following molar ratios: about 50 mol % Lipid A, about 9 mol % DSPC, about 38 mol % cholesterol, and about 3 mol % PEG2k-DMG.

138. The method of any one of claims 90-137, wherein the albumin gene is a human albumin gene,
wherein the method comprises administering the guide RNA in the form of RNA, and the guide RNA comprises SEQ ID NO: 68 or 100,
wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 2, 1, or 12, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

139. The method of any one of claims 90-137, wherein the albumin gene is a human albumin gene,
wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA,
wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 2, 1, or 12, and the mRNA encoding the Cas protein is fully substituted with pseudouridine or N1-methyl-pseudouridine, optionally N1-methyl-pseudouridine, comprises a 5′ cap, and comprises a polyadenylation sequence, and
wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG.

140. A neonatal cell or a population of neonatal cells made by the method of any preceding claim.

141. A neonatal cell or a population of neonatal cells comprising a nucleic acid construct comprising a coding sequence for a polypeptide of interest inserted into a target genomic locus.

142. The neonatal cell or the population of neonatal cells of claim 140 or 141, wherein the neonatal cell is a liver cell or the population of neonatal cells is a population of liver cells.

143. A cell or a population of cells made by the method of any one of claims 1-139.

144. A cell or a population of cells comprising a nucleic acid construct comprising a coding sequence for a polypeptide of interest inserted into a target genomic locus.

145. The cell or the population of cells of claim 143 or 144, wherein the cell is a liver cell or the population of cells is a population of liver cells.

146. The neonatal cell or the population of neonatal cells of any one of claims 140-142 or the cell or population of cells of any one of claims 143-145, wherein the cell or the neonatal cell is a hepatocyte or the population of cells or the population of neonatal cells is a population of hepatocytes.

147. The neonatal cell or the population of neonatal cells of any one of claims 140-142 and 146 or the cell or population of cells of any one of claims 143-146, wherein the cell or the neonatal cell is a human cell or the population of cells or the population of neonatal cells is a population of human cells.

148. The neonatal cell or the population of neonatal cells of any one of claims 140-142, 146, and 147, wherein the neonatal cell or the population of neonatal cells is from a neonatal subject within 52 weeks after birth.

149. The neonatal cell or the population of neonatal cells of any one of claims 140-142 and 146-148, wherein the neonatal cell or the population of neonatal cells is from a neonatal subject within 24 weeks after birth.

150. The neonatal cell or the population of neonatal cells of any one of claims 140-142 and 146-149, wherein the neonatal cell or the population of neonatal cells is from a neonatal subject within 12 weeks after birth.

151. The neonatal cell or the population of neonatal cells of any one of claims 140-142 and 146-150, wherein the neonatal cell or the population of neonatal cells is from a neonatal subject within 8 weeks after birth.

152. The neonatal cell or the population of neonatal cells of any one of claims 140-142 and 146-151, wherein the neonatal cell or the population of neonatal cells is from a neonatal subject within 4 weeks after birth.

153. The neonatal cell or the population of neonatal cells of any one of claims 140-142 and 146-152 or the cell or population of cells of any one of claims 143-147, wherein the cell or the neonatal cell is in vitro or ex vivo or the population of cells or the population of neonatal cells is in vitro or ex vivo.

154. The neonatal cell or the population of neonatal cells of any one of claims 140-142 and 146-152 or the cell or population of cells of any one of claims 143-147, wherein the cell or the neonatal cell is in vivo in a subject or the population of cells or the population of neonatal cells is in vivo.

155. The neonatal cell or the population of neonatal cells of any one of claims 140-142 and 146-154 or the cell or population of cells of any one of claims 143-147 and 153-154, wherein the polypeptide of interest is expressed.

156. The neonatal cell or the population of neonatal cells of any one of claims 140-142 and 146-155 or the cell or population of cells of any one of claims 143-147 and 153-155, wherein the polypeptide of interest comprises a therapeutic polypeptide, optionally wherein the polypeptide of interest comprises lysosomal alpha-glucosidase.

157. The neonatal cell or population of neonatal cells of claim 140-142 and 146-156 or the cell or population of cells of any one of claims 143-147 and 153-156, wherein the lysosomal alpha-glucosidase comprises the amino acid sequence of SEQ ID NO: 173, optionally wherein the polypeptide of interest is encoded by a nucleic acid is codon-optimized and CpG-depleted nucleotide sequence.

158. The neonatal cell or population of neonatal cells of claim 156 or 157 or the cell or population of cells of claim 156 or 157, wherein the lysosomal alpha-glucosidase is encoded by a nucleotide sequence at least 90%, at least 91%, at least 92%, at least 93%, at least 94%, at least 95%, at least 96%, at least 97%, at least 98%, or at least 99% identical to a nucleotide sequence selected from SEQ ID NOS: 174-182 and 581-588, optionally SEQ ID NOS: 175-179, wherein the nucleotide sequence is codon-optimized and CpG-depleted.

159. The neonatal cell or the population of neonatal cells of any one of claims 140-142 and 146-156 or the cell or population of cells of any one of claims 143-147 and 153-156, wherein the polypeptide of interest is a secreted polypeptide.

160. The neonatal cell or the population of neonatal cells of any one of claims 140-142 and 146-156 or the cell or population of cells of any one of claims 143-147 and 153-156, wherein the polypeptide of interest is an intracellular polypeptide.

161. The neonatal cell or the population of neonatal cells of any one of claims 140-142 and 146-160 or the cell or population of cells of any one of claims 143-147 and 153-160, wherein the nucleic acid construct comprises a splice acceptor upstream of the coding sequence for the polypeptide of interest.

162. The neonatal cell or the population of neonatal cells of any one of claims 140-142 and 146-161 or the cell or population of cells of any one of claims 143-147 and 153-161, wherein the nucleic acid construct comprises a polyadenylation signal or sequence downstream of the coding sequence for the polypeptide of interest.

163. The neonatal cell or the population of neonatal cells of any one of claims 140-142 and 146-160 or the cell or population of cells of any one of claims 143-147 and 153-160, wherein the nucleic acid construct comprises a splice acceptor upstream of the coding sequence for the polypeptide of interest, and the nucleic acid construct comprises a polyadenylation signal or sequence downstream of the coding sequence for the polypeptide of interest.

164. The neonatal cell or the population of neonatal cells of any one of claims 140-142 and 146-163 or the cell or population of cells of any one of claims 143-147 and 153-163, wherein the nucleic acid construct does not comprise a promoter that drives the expression of the polypeptide of interest, and wherein the coding sequence for the polypeptide of interest is operably linked to an endogenous promoter at the target genomic locus.

165. The neonatal cell or the population of neonatal cells of any one of claims 140-142 and 146-164 or the cell or population of cells of any one of claims 143-147 and 153-164, wherein the nucleic acid construct is a bidirectional nucleic acid construct.

166. The neonatal cell or the population of neonatal cells of claim 165 or the cell or the population of cells of claim 165, wherein the nucleic acid construct comprises:
(I) a first segment comprising the coding sequence for the polypeptide of interest; and
(II) a second segment comprising a reverse complement of a second coding sequence for the polypeptide of interest.

167. The neonatal cell or the population of neonatal cells of claim 166 or the cell or the population of cells of claim 166, wherein the nucleic acid construct comprises from 5′ to 3′: a first splice acceptor, the coding sequence for the polypeptide of interest, a first polyadenylation signal or sequence, a reverse complement of a second polyadenylation signal or sequence, the reverse complement of the second coding sequence for the polypeptide of interest, and a reverse complement of a second splice acceptor.

168. The neonatal cell or the population of neonatal cells of claim 166 or 167 or the cell or the population of cells of claim 166 or 167, wherein the coding sequence for the polypeptide of interest and the second coding sequence for the polypeptide of interest are different.

169. The neonatal cell or the population of neonatal cells of any one of claims 140-142 and 146-168 or the cell or population of cells of any one of claims 143-147 and 153-168, wherein the target genomic locus is an albumin gene, optionally wherein the albumin gene is a human albumin gene, optionally wherein the nuclease target site is in intron 1 of the albumin gene.


SEQUENCE LISTING (599 sequences)

SEQ ID NO: 1 - RNA, 4423 nt, synthetic construct
  misc_feature 1..4423: note = Synthetic
  source 1..4423: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 2 - RNA, 4140 nt, synthetic construct
  misc_feature 1..4140: note = Synthetic
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 3 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 4 - DNA, 709 bp, Homo sapiens
  source 1..709: mol_type = other DNA
gtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccag

SEQ ID NO: 5-7 - skipped (3 entries)

SEQ ID NO: 8 - protein, 1391 aa, synthetic construct
  REGION 1..1391: note = Synthetic
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 9 - DNA, 4176 bp, synthetic construct
  misc_feature 1..4176: note = Synthetic
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 10 - DNA, 4218 bp, synthetic construct
  misc_feature 1..4218: note = Synthetic
  source 1..4218: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaatta

SEQ ID NO: 11 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 12 - RNA, 4514 nt, synthetic construct
  misc_feature 1..4514: note = Synthetic
  source 1..4514: mol_type = other RNA
gggtcccgcagtcggcgtccagcggctctgcttgttcgtgtgtgtgtcgttgcaggccttattcggatccatggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggcggtggatctccgaaaaagaagagaaaggtgtaatgagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 13 - protein, 7 aa, synthetic construct
  REGION 1..7: note = Synthetic
PKKKRKV

SEQ ID NO: 14 - protein, 7 aa, synthetic construct
  REGION 1..7: note = Synthetic
PKKKRRV

SEQ ID NO: 15 - protein, 16 aa, synthetic construct
  REGION 1..16: note = Synthetic
KRPAATKKAGQAKKKK

SEQ ID NO: 16 - RNA, 16 nt, synthetic construct
  misc_feature 1..16: note = Synthetic
  source 1..16: mol_type = other RNA
gttttagagctatgct

SEQ ID NO: 17 - RNA, 22 nt, synthetic construct
  misc_feature 1..22: note = Synthetic
  source 1..22: mol_type = other RNA
gttttagagctatgctgttttg

SEQ ID NO: 18 - RNA, 67 nt, synthetic construct
  misc_feature 1..67: note = Synthetic
  source 1..67: mol_type = other RNA
agcatagcaagttaaaataaggctagtccgttatcaacttgaaaaagtggcaccgagtcggtgcttt

SEQ ID NO: 19 - RNA, 72 nt, synthetic construct
  misc_feature 1..72: note = Synthetic
  source 1..72: mol_type = other RNA
aaacagcatagcaagttaaaataaggctagtccgttatcaacttgaaaaagtggcaccgagtcggtgctttt

SEQ ID NO: 20 - RNA, 82 nt, synthetic construct
  misc_feature 1..82: note = Synthetic
  source 1..82: mol_type = other RNA
gttggaaccattcaaaacagcatagcaagttaaaataaggctagtccgttatcaacttgaaaaagtggcaccgagtcggtgc

SEQ ID NO: 21 - RNA, 77 nt, synthetic construct
  misc_feature 1..77: note = Synthetic
  source 1..77: mol_type = other RNA
gttttagagctagaaatagcaagttaaaataaggctagtccgttatcaacttgaaaaagtggcaccgagtcggtgct

SEQ ID NO: 22 - RNA, 82 nt, synthetic construct; identical to SEQ ID NO: 20
  misc_feature 1..82: note = Synthetic
  source 1..82: mol_type = other RNA

SEQ ID NO: 23 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 24 - RNA, 86 nt, synthetic construct
  misc_feature 1..86: note = Synthetic
  source 1..86: mol_type = other RNA
gtttaagagctatgctggaaacagcatagcaagtttaaataaggctagtccgttatcaacttgaaaaagtggcaccgagtcggtgc

SEQ ID NO: 25 - RNA, 83 nt, synthetic construct
  misc_feature 1..83: note = Synthetic
  source 1..83: mol_type = other RNA
gttttagagctagaaatagcaagttaaaataaggctagtccgttatcaacttgaaaaagtggcaccgagtcggtgcttttttt

SEQ ID NO: 26 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 27 - RNA, 92 nt, synthetic construct
  misc_feature 1..92: note = Synthetic
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 28 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 29 - RNA, 100 nt, synthetic construct
  misc_feature 1..100: note = Synthetic
  source 1..100: mol_type = other RNA
  misc_difference 1..20: note = n is a, c, g, or u
nnnnnnnnnnnnnnnnnnnngttttagagctagaaatagcaagttaaaataaggctagtccgttatcaacttgaaaaagtggcaccgagtcggtgctttt

SEQ ID NO: 30 - RNA, 20 nt, synthetic construct
  misc_feature 1..20: note = Synthetic
  source 1..20: mol_type = other RNA
gagcaacctcactcttgtct

SEQ ID NO: 31 - RNA, 20 nt, synthetic construct
  misc_feature 1..20: note = Synthetic
  source 1..20: mol_type = other RNA
atgcatttgtttcaaaatat

SEQ ID NO: 32 - RNA, 20 nt, synthetic construct
  misc_feature 1..20: note = Synthetic
  source 1..20: mol_type = other RNA
tgcatttgtttcaaaatatt

SEQ ID NO: 33 - RNA, 20 nt, synthetic construct
  misc_feature 1..20: note = Synthetic
  source 1..20: mol_type = other RNA
atttatgagatcaacagcac

SEQ ID NO: 34 - RNA, 20 nt, synthetic construct
  misc_feature 1..20: note = Synthetic
  source 1..20: mol_type = other RNA
gatcaacagcacaggttttg

SEQ ID NO: 35 - RNA, 20 nt, synthetic construct
  misc_feature 1..20: note = Synthetic
  source 1..20: mol_type = other RNA
ttaaataaagcatagtgcaa

SEQ ID NO: 36 - RNA, 20 nt, synthetic construct
  misc_feature 1..20: note = Synthetic
  source 1..20: mol_type = other RNA
taaagcatagtgcaatggat

SEQ ID NO: 37 - RNA, 20 nt, synthetic construct
  misc_feature 1..20: note = Synthetic
  source 1..20: mol_type = other RNA
tagtgcaatggataggtctt

SEQ ID NO: 38 - RNA, 20 nt, synthetic construct
  misc_feature 1..20: note = Synthetic
  source 1..20: mol_type = other RNA
tactaaaactttattttact

SEQ ID NO: 39 - RNA, 20 nt, synthetic construct
  misc_feature 1..20: note = Synthetic
  source 1..20: mol_type = other RNA
aaagttgaacaatagaaaaa

SEQ ID NO: 40 - RNA, 20 nt, synthetic construct
  misc_feature 1..20: note = Synthetic
  source 1..20: mol_type = other RNA
aatgcataatctaagtcaaa

SEQ ID NO: 41 - RNA, 20 nt, synthetic construct
  misc_feature 1..20: note = Synthetic
  source 1..20: mol_type = other RNA
taataaaattcaaacatcct

SEQ ID NO: 42 - RNA, 20 nt, synthetic construct
  misc_feature 1..20: note = Synthetic
  source 1..20: mol_type = other RNA
gcatctttaaagaattattt

SEQ ID NO: 43 - RNA, 20 nt, synthetic construct
  misc_feature 1..20: note = Synthetic
  source 1..20: mol_type = other RNA
tttggcatttatttctaaaa

SEQ ID NO: 44 - RNA, 20 nt, synthetic construct
  misc_feature 1..20: note = Synthetic
  source 1..20: mol_type = other RNA
tgtatttgtgaagtcttaca

SEQ ID NO: 45 - RNA, 20 nt, synthetic construct
  misc_feature 1..20: note = Synthetic
  source 1..20: mol_type = other RNA
tcctaggtaaaaaaaaaaaa

SEQ ID NO: 46 - RNA, 20 nt, synthetic construct
  misc_feature 1..20: note = Synthetic
  source 1..20: mol_type = other RNA
taattttcttttgcgcacta

SEQ ID NO: 47 - RNA, 20 nt, synthetic construct
  misc_feature 1..20: note = Synthetic
  source 1..20: mol_type = other RNA
tgactgaaacttcacagaat

SEQ ID NO: 48 - RNA, 20 nt, synthetic construct
  misc_feature 1..20: note = Synthetic
  source 1..20: mol_type = other RNA
gactgaaacttcacagaata

SEQ ID NO: 49 - RNA, 20 nt, synthetic construct
  misc_feature 1..20: note = Synthetic
  source 1..20: mol_type = other RNA
ttcattttagtctgtcttct

SEQ ID NO: 50 - RNA, 20 nt, synthetic construct
  misc_feature 1..20: note = Synthetic
  source 1..20: mol_type = other RNA
attatctaagtttgaatata

SEQ ID NO: 51 - RNA, 20 nt, synthetic construct
  misc_feature 1..20: note = Synthetic
  source 1..20: mol_type = other RNA
aatttttaaaatagtattct

SEQ ID NO: 52 - RNA, 20 nt, synthetic construct
  misc_feature 1..20: note = Synthetic
  source 1..20: mol_type = other RNA
tgaattattcttctgtttaa

SEQ ID NO: 53 - RNA, 20 nt, synthetic construct
  misc_feature 1..20: note = Synthetic
  source 1..20: mol_type = other RNA
atcatcctgagtttttctgt

SEQ ID NO: 54 - RNA, 20 nt, synthetic construct
  misc_feature 1..20: note = Synthetic
  source 1..20: mol_type = other RNA
ttactaaaactttattttac

SEQ ID NO: 55 - RNA, 20 nt, synthetic construct
  misc_feature 1..20: note = Synthetic
  source 1..20: mol_type = other RNA
acctttttttttttttacct

SEQ ID NO: 56 - RNA, 20 nt, synthetic construct
  misc_feature 1..20: note = Synthetic
  source 1..20: mol_type = other RNA
agtgcaatggataggtcttt

SEQ ID NO: 57 - RNA, 20 nt, synthetic construct
  misc_feature 1..20: note = Synthetic
  source 1..20: mol_type = other RNA
tgattcctacagaaaaactc

SEQ ID NO: 58 - RNA, 20 nt, synthetic construct
  misc_feature 1..20: note = Synthetic
  source 1..20: mol_type = other RNA
tgggcaagggaagaaaaaaa

SEQ ID NO: 59 - RNA, 20 nt, synthetic construct
  misc_feature 1..20: note = Synthetic
  source 1..20: mol_type = other RNA
cctcactcttgtctgggcaa

SEQ ID NO: 60 - RNA, 20 nt, synthetic construct
  misc_feature 1..20: note = Synthetic
  source 1..20: mol_type = other RNA
acctcactcttgtctgggca

SEQ ID NO: 61 - RNA, 20 nt, synthetic construct
  misc_feature 1..20: note = Synthetic
  source 1..20: mol_type = other RNA
tgagcaacctcactcttgtc

SEQ ID NO: 62 - RNA, 100 nt, synthetic construct
  misc_feature 1..100: note = Synthetic
  source 1..100: mol_type = other RNA
gagcaacctcactcttgtctgttttagagctagaaatagcaagttaaaataaggctagtccgttatcaacttgaaaaagtggcaccgagtcggtgctttt

SEQ ID NO: 63 - RNA, 100 nt, synthetic construct
  misc_feature 1..100: note = Synthetic
  source 1..100: mol_type = other RNA
atgcatttgtttcaaaatatgttttagagctagaaatagcaagttaaaataaggctagtccgttatcaacttgaaaaagtggcaccgagtcggtgctttt

SEQ ID NO: 64 - RNA, 100 nt, synthetic construct
  misc_feature 1..100: note = Synthetic
  source 1..100: mol_type = other RNA
tgcatttgtttcaaaatattgttttagagctagaaatagcaagttaaaataaggctagtccgttatcaacttgaaaaagtggcaccgagtcggtgctttt

SEQ ID NO: 65 - RNA, 100 nt, synthetic construct
  misc_feature 1..100: note = Synthetic
  source 1..100: mol_type = other RNA
atttatgagatcaacagcacgttttagagctagaaatagcaagttaaaataaggctagtccgttatcaacttgaaaaagtggcaccgagtcggtgctttt

SEQ ID NO: 66 - RNA, 100 nt, synthetic construct
  misc_feature 1..100: note = Synthetic
  source 1..100: mol_type = other RNA
gatcaacagcacaggttttggttttagagctagaaatagcaagttaaaataaggctagtccgttatcaacttgaaaaagtggcaccgagtcggtgctttt

SEQ ID NO: 67 - RNA, 100 nt, synthetic construct
  misc_feature 1..100: note = Synthetic
  source 1..100: mol_type = other RNA
ttaaataaagcatagtgcaagttttagagctagaaatagcaagttaaaataaggctagtccgttatcaacttgaaaaagtggcaccgagtcggtgctttt

SEQ ID NO: 68 - RNA, 100 nt, synthetic construct
  misc_feature 1..100: note = Synthetic
  source 1..100: mol_type = other RNA
taaagcatagtgcaatggatgttttagagctagaaatagcaagttaaaataaggctagtccgttatcaacttgaaaaagtggcaccgagtcggtgctttt

SEQ ID NO: 69 - RNA, 100 nt, synthetic construct
  misc_feature 1..100: note = Synthetic
  source 1..100: mol_type = other RNA
tagtgcaatggataggtcttgttttagagctagaaatagcaagttaaaataaggctagtccgttatcaacttgaaaaagtggcaccgagtcggtgctttt

SEQ ID NO: 70 - RNA, 100 nt, synthetic construct
  misc_feature 1..100: note = Synthetic
  source 1..100: mol_type = other RNA
tactaaaactttattttactgttttagagctagaaatagcaagttaaaataaggctagtccgttatcaacttgaaaaagtggcaccgagtcggtgctttt

SEQ ID NO: 71 - RNA, 100 nt, synthetic construct
  misc_feature 1..100: note = Synthetic
  source 1..100: mol_type = other RNA
aaagttgaacaatagaaaaagttttagagctagaaatagcaagttaaaataaggctagtccgttatcaacttgaaaaagtggcaccgagtcggtgctttt

SEQ ID NO: 72 - RNA, 100 nt, synthetic construct
  misc_feature 1..100: note = Synthetic
  source 1..100: mol_type = other RNA
aatgcataatctaagtcaaagttttagagctagaaatagcaagttaaaataaggctagtccgttatcaacttgaaaaagtggcaccgagtcggtgctttt

SEQ ID NO: 73 - RNA, 100 nt, synthetic construct
  misc_feature 1..100: note = Synthetic
  source 1..100: mol_type = other RNA
taataaaattcaaacatcctgttttagagctagaaatagcaagttaaaataaggctagtccgttatcaacttgaaaaagtggcaccgagtcggtgctttt

SEQ ID NO: 74 - RNA, 100 nt, synthetic construct
  misc_feature 1..100: note = Synthetic
  source 1..100: mol_type = other RNA
gcatctttaaagaattatttgttttagagctagaaatagcaagttaaaataaggctagtccgttatcaacttgaaaaagtggcaccgagtcggtgctttt

SEQ ID NO: 75 - RNA, 100 nt, synthetic construct
  misc_feature 1..100: note = Synthetic
  source 1..100: mol_type = other RNA
tttggcatttatttctaaaagttttagagctagaaatagcaagttaaaataaggctagtccgttatcaacttgaaaaagtggcaccgagtcggtgctttt

SEQ ID NO: 76 - RNA, 100 nt, synthetic construct
  misc_feature 1..100: note = Synthetic
  source 1..100: mol_type = other RNA
tgtatttgtgaagtcttacagttttagagctagaaatagcaagttaaaataaggctagtccgttatcaacttgaaaaagtggcaccgagtcggtgctttt

SEQ ID NO: 77 - RNA, 100 nt, synthetic construct
  misc_feature 1..100: note = Synthetic
  source 1..100: mol_type = other RNA
tcctaggtaaaaaaaaaaaagttttagagctagaaatagcaagttaaaataaggctagtccgttatcaacttgaaaaagtggcaccgagtcggtgctttt

SEQ ID NO: 78 - RNA, 100 nt, synthetic construct
  misc_feature 1..100: note = Synthetic
  source 1..100: mol_type = other RNA
taattttcttttgcgcactagttttagagctagaaatagcaagttaaaataaggctagtccgttatcaacttgaaaaagtggcaccgagtcggtgctttt

SEQ ID NO: 79 - RNA, 100 nt, synthetic construct
  misc_feature 1..100: note = Synthetic
  source 1..100: mol_type = other RNA
tgactgaaacttcacagaatgttttagagctagaaatagcaagttaaaataaggctagtccgttatcaacttgaaaaagtggcaccgagtcggtgctttt

SEQ ID NO: 80 - RNA, 100 nt, synthetic construct
  misc_feature 1..100: note = Synthetic
  source 1..100: mol_type = other RNA
gactgaaacttcacagaatagttttagagctagaaatagcaagttaaaataaggctagtccgttatcaacttgaaaaagtggcaccgagtcggtgctttt

SEQ ID NO: 81 - RNA, 100 nt, synthetic construct
  misc_feature 1..100: note = Synthetic
  source 1..100: mol_type = other RNA
ttcattttagtctgtcttctgttttagagctagaaatagcaagttaaaataaggctagtccgttatcaacttgaaaaagtggcaccgagtcggtgctttt

SEQ ID NO: 82 - RNA, 100 nt, synthetic construct
  misc_feature 1..100: note = Synthetic
  source 1..100: mol_type = other RNA
attatctaagtttgaatatagttttagagctagaaatagcaagttaaaataaggctagtccgttatcaacttgaaaaagtggcaccgagtcggtgctttt

SEQ ID NO: 83 - RNA, 100 nt, synthetic construct
  misc_feature 1..100: note = Synthetic
  source 1..100: mol_type = other RNA
aatttttaaaatagtattctgttttagagctagaaatagcaagttaaaataaggctagtccgttatcaacttgaaaaagtggcaccgagtcggtgctttt

SEQ ID NO: 84 - RNA, 100 nt, synthetic construct
  misc_feature 1..100: note = Synthetic
  source 1..100: mol_type = other RNA
tgaattattcttctgtttaagttttagagctagaaatagcaagttaaaataaggctagtccgttatcaacttgaaaaagtggcaccgagtcggtgctttt

SEQ ID NO: 85 - RNA, 100 nt, synthetic construct
  misc_feature 1..100: note = Synthetic
  source 1..100: mol_type = other RNA
atcatcctgagtttttctgtgttttagagctagaaatagcaagttaaaataaggctagtccgttatcaacttgaaaaagtggcaccgagtcggtgctttt

SEQ ID NO: 86 - RNA, 100 nt, synthetic construct
  misc_feature 1..100: note = Synthetic
  source 1..100: mol_type = other RNA
ttactaaaactttattttacgttttagagctagaaatagcaagttaaaataaggctagtccgttatcaacttgaaaaagtggcaccgagtcggtgctttt

SEQ ID NO: 87 - RNA, 100 nt, synthetic construct
  misc_feature 1..100: note = Synthetic
  source 1..100: mol_type = other RNA
acctttttttttttttacctgttttagagctagaaatagcaagttaaaataaggctagtccgttatcaacttgaaaaagtggcaccgagtcggtgctttt

SEQ ID NO: 88 - RNA, 100 nt, synthetic construct
  misc_feature 1..100: note = Synthetic
  source 1..100: mol_type = other RNA
agtgcaatggataggtctttgttttagagctagaaatagcaagttaaaataaggctagtccgttatcaacttgaaaaagtggcaccgagtcggtgctttt

SEQ ID NO: 89 - RNA, 100 nt, synthetic construct
  misc_feature 1..100: note = Synthetic
  source 1..100: mol_type = other RNA
tgattcctacagaaaaactcgttttagagctagaaatagcaagttaaaataaggctagtccgttatcaacttgaaaaagtggcaccgagtcggtgctttt

SEQ ID NO: 90 - RNA, 100 nt, synthetic construct
  misc_feature 1..100: note = Synthetic
  source 1..100: mol_type = other RNA
tgggcaagggaagaaaaaaagttttagagctagaaatagcaagttaaaataaggctagtccgttatcaacttgaaaaagtggcaccgagtcggtgctttt

SEQ ID NO: 91 - RNA, 100 nt, synthetic construct
  misc_feature 1..100: note = Synthetic
  source 1..100: mol_type = other RNA
cctcactcttgtctgggcaagttttagagctagaaatagcaagttaaaataaggctagtccgttatcaacttgaaaaagtggcaccgagtcggtgctttt

SEQ ID NO: 92 - RNA, 100 nt, synthetic construct
  misc_feature 1..100: note = Synthetic
  source 1..100: mol_type = other RNA
acctcactcttgtctgggcagttttagagctagaaatagcaagttaaaataaggctagtccgttatcaacttgaaaaagtggcaccgagtcggtgctttt

SEQ ID NO: 93 - RNA, 100 nt, synthetic construct
  misc_feature 1..100: note = Synthetic
  source 1..100: mol_type = other RNA
tgagcaacctcactcttgtcgttttagagctagaaatagcaagttaaaataaggctagtccgttatcaacttgaaaaagtggcaccgagtcggtgctttt

SEQ ID NO: 94 - RNA, 100 nt, synthetic construct; identical to SEQ ID NO: 62
  misc_feature 1..100: note = Synthetic
  source 1..100: mol_type = other RNA

SEQ ID NO: 95 - RNA, 100 nt, synthetic construct; identical to SEQ ID NO: 63
  misc_feature 1..100: note = Synthetic
  source 1..100: mol_type = other RNA

SEQ ID NO: 96 - RNA, 100 nt, synthetic construct; identical to SEQ ID NO: 64
  misc_feature 1..100: note = Synthetic
  source 1..100: mol_type = other RNA

SEQ ID NO: 97 - RNA, 100 nt, synthetic construct; identical to SEQ ID NO: 65
  misc_feature 1..100: note = Synthetic
  source 1..100: mol_type = other RNA

SEQ ID NO: 98 - RNA, 100 nt, synthetic construct; identical to SEQ ID NO: 66
  misc_feature 1..100: note = Synthetic
  source 1..100: mol_type = other RNA

SEQ ID NO: 99 - RNA, 100 nt, synthetic construct; identical to SEQ ID NO: 67
  misc_feature 1..100: note = Synthetic
  source 1..100: mol_type = other RNA

SEQ ID NO: 100 - RNA, 100 nt, synthetic construct; identical to SEQ ID NO: 68
  misc_feature 1..100: note = Synthetic
  source 1..100: mol_type = other RNA

SEQ ID NO: 101 - RNA, 100 nt, synthetic construct; identical to SEQ ID NO: 69
  misc_feature 1..100: note = Synthetic
  source 1..100: mol_type = other RNA

SEQ ID NO: 102 - RNA, 100 nt, synthetic construct; identical to SEQ ID NO: 70
  misc_feature 1..100: note = Synthetic
  source 1..100: mol_type = other RNA

SEQ ID NO: 103 - RNA, 100 nt, synthetic construct; identical to SEQ ID NO: 71
  misc_feature 1..100: note = Synthetic
  source 1..100: mol_type = other RNA

SEQ ID NO: 104 - RNA, 100 nt, synthetic construct; identical to SEQ ID NO: 72
  misc_feature 1..100: note = Synthetic
  source 1..100: mol_type = other RNA

SEQ ID NO: 105 - RNA, 100 nt, synthetic construct; identical to SEQ ID NO: 73
  misc_feature 1..100: note = Synthetic
  source 1..100: mol_type = other RNA

SEQ ID NO: 106 - RNA, 100 nt, synthetic construct; identical to SEQ ID NO: 74
  misc_feature 1..100: note = Synthetic
  source 1..100: mol_type = other RNA

SEQ ID NO: 107 - RNA, 100 nt, synthetic construct; identical to SEQ ID NO: 75
  misc_feature 1..100: note = Synthetic
  source 1..100: mol_type = other RNA

SEQ ID NO: 108 - RNA, 100 nt, synthetic construct; identical to SEQ ID NO: 76
  misc_feature 1..100: note = Synthetic
  source 1..100: mol_type = other RNA

SEQ ID NO: 109 - RNA, 100 nt, synthetic construct; identical to SEQ ID NO: 77
  misc_feature 1..100: note = Synthetic
  source 1..100: mol_type = other RNA

SEQ ID NO: 110 - RNA, 100 nt, synthetic construct; identical to SEQ ID NO: 78
  misc_feature 1..100: note = Synthetic
  source 1..100: mol_type = other RNA

SEQ ID NO: 111 - RNA, 100 nt, synthetic construct; identical to SEQ ID NO: 79
  misc_feature 1..100: note = Synthetic
  source 1..100: mol_type = other RNA

SEQ ID NO: 112 - RNA, 100 nt, synthetic construct; identical to SEQ ID NO: 80
  misc_feature 1..100: note = Synthetic
  source 1..100: mol_type = other RNA

SEQ ID NO: 113 - RNA, 100 nt, synthetic construct; identical to SEQ ID NO: 81
  misc_feature 1..100: note = Synthetic
  source 1..100: mol_type = other RNA

SEQ ID NO: 114 - RNA, 100 nt, synthetic construct; identical to SEQ ID NO: 82
  misc_feature 1..100: note = Synthetic
  source 1..100: mol_type = other RNA

SEQ ID NO: 115 - RNA, 100 nt, synthetic construct; identical to SEQ ID NO: 83
  misc_feature 1..100: note = Synthetic
  source 1..100: mol_type = other RNA

SEQ ID NO: 116 - RNA, 100 nt, synthetic construct; identical to SEQ ID NO: 84
  misc_feature 1..100: note = Synthetic
  source 1..100: mol_type = other RNA

SEQ ID NO: 117 - RNA, 100 nt, synthetic construct; identical to SEQ ID NO: 85
  misc_feature 1..100: note = Synthetic
  source 1..100: mol_type = other RNA

SEQ ID NO: 118 - RNA, 100 nt, synthetic construct; identical to SEQ ID NO: 86
  misc_feature 1..100: note = Synthetic
  source 1..100: mol_type = other RNA

SEQ ID NO: 119 - RNA, 100 nt, synthetic construct; identical to SEQ ID NO: 87
  misc_feature 1..100: note = Synthetic
  source 1..100: mol_type = other RNA

SEQ ID NO: 120 - RNA, 100 nt, synthetic construct; identical to SEQ ID NO: 88
  misc_feature 1..100: note = Synthetic
  source 1..100: mol_type = other RNA

SEQ ID NO: 121 - RNA, 100 nt, synthetic construct; identical to SEQ ID NO: 89
  misc_feature 1..100: note = Synthetic
  source 1..100: mol_type = other RNA

SEQ ID NO: 122 - RNA, 100 nt, synthetic construct; identical to SEQ ID NO: 90
  misc_feature 1..100: note = Synthetic
  source 1..100: mol_type = other RNA

SEQ ID NO: 123 - RNA, 100 nt, synthetic construct; identical to SEQ ID NO: 91
  misc_feature 1..100: note = Synthetic
  source 1..100: mol_type = other RNA

SEQ ID NO: 124 - RNA, 100 nt, synthetic construct; identical to SEQ ID NO: 92
  misc_feature 1..100: note = Synthetic
  source 1..100: mol_type = other RNA

SEQ ID NO: 125 - RNA, 100 nt, synthetic construct; identical to SEQ ID NO: 93
  misc_feature 1..100: note = Synthetic
  source 1..100: mol_type = other RNA

SEQ ID NO: 126 - DNA, 20 bp, synthetic construct
  misc_feature 1..20: note = Synthetic
  source 1..20: mol_type = other DNA
gagcaacctcactcttgtct

SEQ ID NO: 127 - DNA, 20 bp, synthetic construct
  misc_feature 1..20: note = Synthetic
  source 1..20: mol_type = other DNA
atgcatttgtttcaaaatat

SEQ ID NO: 128 - DNA, 20 bp, synthetic construct
  misc_feature 1..20: note = Synthetic
  source 1..20: mol_type = other DNA
tgcatttgtttcaaaatatt

SEQ ID NO: 129 - DNA, 20 bp, synthetic construct
  misc_feature 1..20: note = Synthetic
  source 1..20: mol_type = other DNA
atttatgagatcaacagcac

SEQ ID NO: 130 - DNA, 20 bp, synthetic construct
  misc_feature 1..20: note = Synthetic
  source 1..20: mol_type = other DNA
gatcaacagcacaggttttg

SEQ ID NO: 131 - DNA, 20 bp, synthetic construct
  misc_feature 1..20: note = Synthetic
  source 1..20: mol_type = other DNA
ttaaataaagcatagtgcaa

SEQ ID NO: 132 - DNA, 20 bp, synthetic construct
  misc_feature 1..20: note = Synthetic
  source 1..20: mol_type = other DNA
taaagcatagtgcaatggat

SEQ ID NO: 133 - DNA, 20 bp, synthetic construct
  misc_feature 1..20: note = Synthetic
  source 1..20: mol_type = other DNA
tagtgcaatggataggtctt

SEQ ID NO: 134 - DNA, 20 bp, synthetic construct
  misc_feature 1..20: note = Synthetic
  source 1..20: mol_type = other DNA
tactaaaactttattttact

SEQ ID NO: 135 - DNA, 20 bp, synthetic construct
  misc_feature 1..20: note = Synthetic
  source 1..20: mol_type = other DNA
aaagttgaacaatagaaaaa

SEQ ID NO: 136 - DNA, 20 bp, synthetic construct
  misc_feature 1..20: note = Synthetic
  source 1..20: mol_type = other DNA
aatgcataatctaagtcaaa

SEQ ID NO: 137 - DNA, 20 bp, synthetic construct
  misc_feature 1..20: note = Synthetic
  source 1..20: mol_type = other DNA
taataaaattcaaacatcct

SEQ ID NO: 138 - DNA, 20 bp, synthetic construct
  misc_feature 1..20: note = Synthetic
  source 1..20: mol_type = other DNA
gcatctttaaagaattattt

SEQ ID NO: 139 - DNA, 20 bp, synthetic construct
  misc_feature 1..20: note = Synthetic
  source 1..20: mol_type = other DNA
tttggcatttatttctaaaa

SEQ ID NO: 140 - DNA, 20 bp, synthetic construct
  misc_feature 1..20: note = Synthetic
  source 1..20: mol_type = other DNA
tgtatttgtgaagtcttaca

SEQ ID NO: 141 - DNA, 20 bp, synthetic construct
  misc_feature 1..20: note = Synthetic
  source 1..20: mol_type = other DNA
tcctaggtaaaaaaaaaaaa

SEQ ID NO: 142 - DNA, 20 bp, synthetic construct
  misc_feature 1..20: note = Synthetic
  source 1..20: mol_type = other DNA
taattttcttttgcgcacta

SEQ ID NO: 143 - DNA, 20 bp, synthetic construct
  misc_feature 1..20: note = Synthetic
  source 1..20: mol_type = other DNA
tgactgaaacttcacagaat

SEQ ID NO: 144 - DNA, 20 bp, synthetic construct
  misc_feature 1..20: note = Synthetic
  source 1..20: mol_type = other DNA
gactgaaacttcacagaata

SEQ ID NO: 145 - DNA, 20 bp, synthetic construct
  misc_feature 1..20: note = Synthetic
  source 1..20: mol_type = other DNA
ttcattttagtctgtcttct

SEQ ID NO: 146 - DNA, 20 bp, synthetic construct
  misc_feature 1..20: note = Synthetic
  source 1..20: mol_type = other DNA
attatctaagtttgaatata

SEQ ID NO: 147 - DNA, 20 bp, synthetic construct
  misc_feature 1..20: note = Synthetic
  source 1..20: mol_type = other DNA
aatttttaaaatagtattct

SEQ ID NO: 148 - DNA, 20 bp, synthetic construct
  misc_feature 1..20: note = Synthetic
  source 1..20: mol_type = other DNA
tgaattattcttctgtttaa

SEQ ID NO: 149 - DNA, 20 bp, synthetic construct
  misc_feature 1..20: note = Synthetic
  source 1..20: mol_type = other DNA
atcatcctgagtttttctgt

SEQ ID NO: 150 - DNA, 20 bp, synthetic construct
  misc_feature 1..20: note = Synthetic
  source 1..20: mol_type = other DNA
ttactaaaactttattttac

SEQ ID NO: 151 - DNA, 20 bp, synthetic construct
  misc_feature 1..20: note = Synthetic
  source 1..20: mol_type = other DNA
acctttttttttttttacct

SEQ ID NO: 152 - DNA, 20 bp, synthetic construct
  misc_feature 1..20: note = Synthetic
  source 1..20: mol_type = other DNA
agtgcaatggataggtcttt

SEQ ID NO: 153 - DNA, 20 bp, synthetic construct
  misc_feature 1..20: note = Synthetic
  source 1..20: mol_type = other DNA
tgattcctacagaaaaactc

SEQ ID NO: 154 - DNA, 20 bp, synthetic construct
  misc_feature 1..20: note = Synthetic
  source 1..20: mol_type = other DNA
tgggcaagggaagaaaaaaa

SEQ ID NO: 155 - DNA, 20 bp, synthetic construct
  misc_feature 1..20: note = Synthetic
  source 1..20: mol_type = other DNA
cctcactcttgtctgggcaa

SEQ ID NO: 156 - DNA, 20 bp, synthetic construct
  misc_feature 1..20: note = Synthetic
  source 1..20: mol_type = other DNA
acctcactcttgtctgggca

SEQ ID NO: 157 - DNA, 20 bp, synthetic construct
  misc_feature 1..20: note = Synthetic
  source 1..20: mol_type = other DNA
tgagcaacctcactcttgtc

SEQ ID NO: 158 - DNA, 145 bp, synthetic construct
  misc_feature 1..145: note = Synthetic
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 159 - DNA, 141 bp, synthetic construct
  misc_feature 1..141: note = Synthetic
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 160 - DNA, 130 bp, synthetic construct
  misc_feature 1..130: note = Synthetic
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 161 - DNA, 131 bp, synthetic construct
  misc_feature 1..131: note = Synthetic
  source 1..131: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 162 - DNA, 224 bp, synthetic construct
  misc_feature 1..224: note = Synthetic
  source 1..224: mol_type = other DNA
ctgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatg

SEQ ID NO: 163 - DNA, 100 bp, synthetic construct
  misc_feature 1..100: note = Synthetic
  source 1..100: mol_type = other DNA
taggtcagtgaagagaagaacaaaaagcagcatattacagttagttgtcttcatcaatctttaaatatgttgtgtggtttttctctccctgtttccacag

SEQ ID NO: 164 - RNA, 20 nt, synthetic construct
  misc_feature 1..20: note = Synthetic
  source 1..20: mol_type = other RNA
cactcttgtctgtggaaaca

SEQ ID NO: 165 - DNA, 20 bp, synthetic construct
  misc_feature 1..20: note = Synthetic
  source 1..20: mol_type = other DNA
cactcttgtctgtggaaaca

SEQ ID NO: 166 - RNA, 100 nt, synthetic construct
  misc_feature 1..100: note = Synthetic
  source 1..100: mol_type = other RNA
cactcttgtctgtggaaacagttttagagctagaaatagcaagttaaaataaggctagtccgttatcaacttgaaaaagtggcaccgagtcggtgctttt

SEQ ID NO: 167 - RNA, 100 nt, synthetic construct; identical to SEQ ID NO: 166
  misc_feature 1..100: note = Synthetic
  source 1..100: mol_type = other RNA

SEQ ID NO: 168 - DNA, 145 bp, synthetic construct
  misc_feature 1..145: note = Synthetic
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 169 - DNA, 230 bp, synthetic construct
  misc_feature 1..230: note = Synthetic
  source 1..230: mol_type = other DNA
taatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtc

SEQ ID NO: 170 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 171 - DNA, 3751 bp, Homo sapiens
  source 1..3751: mol_type = other DNA
cgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcag

SEQ ID NO: 172 - DNA, 2859 bp, Homo sapiens
  source 1..2859: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 173 - protein, 883 aa, synthetic construct
  REGION 1..883: note = Synthetic
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 174 - DNA, 2649 bp, synthetic construct
  misc_feature 1..2649: note = Synthetic
  source 1..2649: mol_type = other DNA
gcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 175 - DNA, 2649 bp, synthetic construct
  misc_feature 1..2649: note = Synthetic
  source 1..2649: mol_type = other DNA
gcacacccaggcagacctagagcagtgcccacacaatgtgatgtcccccccaactcaagatttgattgtgcccctgacaaggccatcacccaagaacagtgtgaggccagaggctgttgctatatccctgcaaagcaggggttacagggagcccagatggggcagccatggtgcttcttcccacccagctacccttcttacaaactggaaaacctgagctcctctgaaatgggctacacagccacactgaccagaaccacccccaccttctttcccaaagatatcctgaccttaagactggatgtgatgatggaaactgaaaatagattacactttacaattaaagatccagctaacaggagatatgaagtgcccttggagaccccacatgttcattcaagagcacctagcccactctactctgtggagttctcagaagagccatttggggtgattgtgagaagacaattggatggaagagtgctgctgaatacaacagtggctccccttttttttgcagaccaatttcttcagctgtctaccagtctgcccagccagtatatcacaggcctggctgaacatctcagtcccttgatgctcagcaccagctggaccaggattaccttatggaatagagaccttgccccaactccaggtgccaacctctatgggtctcaccctttttacctggcccttgaggatggagggtctgcacatggggtgtttcttctaaacagcaatgcaatggatgtggtcctacagccatctcctgcccttagctggagatccacaggtgggatcctagatgtctatattttcctgggaccagagcctaagtcagtggtgcagcagtacctggatgttgtgggatatccattcatgcctccatactggggcctgggcttccatctgtgcagatggggatattcctccacagctatcacaagacaagtggtggagaatatgaccagggcccacttccccctggatgtccagtggaatgaccttgactacatggactccagaagggatttcacatttaacaaggatggcttcagagacttccctgccatggtgcaggaactgcaccaaggaggcagaagatacatgatgatagtggatcctgccatcagtagctctggccctgctgggagctatagaccctatgatgagggtctgagaaggggggttttcatcacaaatgagactggccaacctctgattgggaaggtatggccagggtccactgctttcccagacttcactaacccaacagccctggcctggtgggaggacatggtagctgaattccatgaccaggtgccttttgatggcatgtggattgacatgaatgaaccttccaacttcatcaggggctctgaggatggctgccccaacaatgagctggaaaacccaccttatgttcctggggtggttggtgggaccctccaggcagccaccatatgtgcctccagccaccagtttctctccacacactataacctccacaacctatatggactgacagaagccattgcctcccatagggcactggtgaaggctagaggtacaagaccatttgtgatatctagaagcacttttgctggccatggcagatatgcaggccactggactggagatgtttggagctcttgggagcagctggcctccagtgtgccagaaattctgcagtttaatctactaggggtgcctttggtaggggctgatgtctgtggctttctgggcaacacctcagaagagctttgtgtgagatggacccaactgggggccttctatcccttcatgagaaaccacaatagcctgctcagtttgccccaggagccatacagcttctcagagccagcccagcaggccatgaggaaggcccttactttaagatatgcactcctcccccacttgtacacactgtttcaccaggcccatgttgcaggagaaactgtggccagacccttgttcctggagttccctaaggactcttcaacctggactgtggaccaccaactcctctggggggaggccctcctcatcacaccagttctccaggcagggaaggctgaagttactggctacttccccttgggcacatggtatgaccttcagacagttccagtagaggcacttggcagcctcccacccccacctgcagctcccagagagccagcaattcactctgaagggcaatgggtgactctgccagccccactggacaccattaatgtccatttaagagctggttatatcatacccctgcagggccctggcctcacaaccacagagtccagacagcagcctatggctctggctgtagcattgactaaaggtggggaggcaagaggggagttattctgggatgatggagagtcactggaagtgttggagagaggggcatacacacaggttatcttcttggcaaggaataacacaatagtgaatgagttagtaagagtaaccagtgagggagctggcctgcagttacagaaggtgactgtccttggagtggccactgccccccagcaagtcctatccaatggtgtccctgtatccaacttcacctacagcccagacacaaaggtcctggatatctgtgtctccctgttgatgggagaacagtttctggtcagctggtgt

SEQ ID NO: 176 - DNA, 2649 bp, synthetic construct
  misc_feature 1..2649: note = Synthetic
  source 1..2649: mol_type = other DNA
gcccatcctggcagacctagagctgtgcccacacagtgtgatgtgcctccaaacagcagatttgactgtgcccctgacaaggccatcacacaagagcagtgtgaagccagaggctgctgttacatccctgccaagcaaggactgcaaggggctcagatgggacagccctggtgcttcttcccaccatcttaccccagctacaagctggaaaacctgagcagctctgagatgggctacactgctacactgaccagaaccacacctacattcttcccaaaggacatcctgacactgaggctggatgtgatgatggaaacagagaacaggctgcacttcaccatcaaggaccctgccaatagaagatatgaggtgcccctggaaacccctcatgtgcactctagagccccatctccactgtactctgtggaattttctgaggaaccctttggggtcatagtcagaaggcagctggatggcagagtgctgctgaacacaactgtggcccctctgttctttgctgatcagttcctgcagctgagcaccagcctgcctagccagtatatcacaggcctggcagagcacctgtctccactgatgctgagcacatcctggaccagaatcaccctgtggaacagagacctggctcccacacctggggccaacctgtatggctctcaccccttctatctggccctggaagatggtggttcagcccatggtgtctttctgctgaactccaatgccatggatgtggtgctgcagccctctcctgctctgtcttggagaagcactggtggcatcctggatgtgtacatctttctgggccctgagcctaagtctgtggtgcagcagtacctggatgttgtgggctaccccttcatgcctccttattggggcctgggcttccacctgtgcagatggggatacagcagcacagccatcaccagacaggttgtggaaaacatgaccagagcacacttcccactggatgtgcagtggaatgacctggactacatggacagcagaagggacttcaccttcaacaaggatggcttcagagacttccctgccatggtgcaagagctgcaccaaggtggtagaagatatatgatgattgtggacccagccatcagctcctctggaccagctggcagctacagaccctatgatgagggactgagaagaggggtgttcatcaccaatgagacaggccagcctctgattggcaaagtgtggccaggctccacagcctttccagatttcacaaaccccacagctctggcttggtgggaagatatggtggcagagttccatgatcaggtgccctttgatggcatgtggattgacatgaatgagcccagcaacttcatcaggggctctgaggatggatgccccaacaatgaactggaaaatcctccttatgtgcctggtgttgttggagggacactgcaggcagccacaatttgtgccagctctcaccagtttctgagcacccactacaacctgcacaatctgtatggcctgactgaggccattgccagccacagggctcttgtgaaggctagaggcaccagaccttttgtgatcagcaggtccacctttgctggccatggcagatatgctggccactggactggggatgtgtggtctagttgggagcagctggctagctctgtgcctgagatcctgcagttcaacctgctgggagtgccccttgttggagcagatgtgtgtggcttcctgggcaacacctcagaggaactgtgtgtcagatggactcagctgggagcattctaccctttcatgagaaaccacaacagcctgctgagcctgcctcaagagccttacagcttttctgagccagcacagcaggccatgagaaaggccctgactctgagatatgccctgctgcctcacctgtacaccctgttccatcaggctcatgtggctggggagactgtggctagacccctgttcctggaattccccaaggacagctccacctggacagtggatcatcagctgctgtggggagaagccctgctgattacacctgtgctgcaggctggcaaggctgaagtgacaggctacttccctcttggcacttggtatgacctgcagacagtgcctgtggaagctctgggatctctgcctccacctcctgctgctcctagagagcctgccattcactctgaaggccagtgggttaccctgcctgctccactggataccatcaatgtgcatctgagagcaggctacatcatccctctgcaaggccctggcctgaccaccacagaatctagacagcagccaatggcactggctgtggccctgaccaaaggtggagaagctagaggggagctgttctgggatgatggggagagcctggaagtgctggaaagaggggcctatacacaagtgatttttctggctaggaacaataccattgtgaatgagcttgtcagagtcacctctgaaggtgctggcctgcagctccagaaagtgacagtgcttggagtggccacagcaccccagcaagtgctgtctaatggggtgccagtgtccaacttcacatacagccctgataccaaggtcctggacatctgtgtgtccctgcttatgggagagcagttcctggtgtcttggtgc

SEQ ID NO: 177 - DNA, 2649 bp, synthetic construct
  misc_feature 1..2649: note = Synthetic
  source 1..2649: mol_type = other DNA
gcccaccctggcagacctagagcagtgcccacccagtgtgatgtgcctcctaacagcaggtttgactgtgcacctgacaaggccatcacccaggagcagtgtgaagccaggggttgctgctatatcccagccaaacagggtctgcagggagcccaaatgggccagccttggtgcttctttccaccctcttacccttcttacaagctggaaaacctatcttcaagtgagatgggctacacagccactctaacaagaaccacacctacctttttccccaaggacatcctcactctgagactggatgtaatgatggagactgaaaacagactccacttcaccattaaagacccagccaacagaagatatgaggtaccattggagaccccccatgtccacagcagagcacccagccccctatactcagtggaattctctgaggaaccttttggtgtcatagtgagaagacagctggatggcagagtgctcctcaacacaactgtggctccactgttttttgctgaccaattcctacagctgagcaccagcctgccctcccaatatatcacagggctggctgaacacctatcccctctgatgctgtccacctcctggaccaggatcactctgtggaacagggacttagcccctacccctggtgccaacctgtatggctctcacccattctacctggccctggaggatggaggttcagcccatggagtctttctcctgaactctaatgccatggatgtggtgctacagccatcccctgcattatcttggaggagcacaggaggaatcctggatgtatacatctttctgggcccagaaccaaagtcagttgtacagcagtaccttgatgtggtaggctaccccttcatgcctccatactggggtctgggcttccacctctgcagatggggctacagcagtacagctatcacaagacaggttgtggagaacatgacaagggctcacttccccctggatgtacagtggaatgacttggactacatggacagtagaagggacttcacattcaacaaggatggcttcagggacttccctgccatggtacaggaactccaccagggagggagaagatacatgatgattgtggacccagctatcagttctagtggtcctgcaggttcctacaggccctatgatgaaggcctcagaagaggagtgttcatcactaatgagacaggacagcctttgataggaaaggtctggcctggcagtacagctttcccagattttaccaatccaactgctttggcttggtgggaggacatggtggcagagttccatgaccaggtgccctttgatggcatgtggattgacatgaatgaaccatctaacttcatcagaggatcagaagatggatgccccaacaatgagctggagaaccctccttatgtgccaggtgtggtgggtggcaccctacaggctgcaaccatctgtgccagctcacaccagttcctgagtacacactacaatctacacaacctgtatggcctgactgaagctattgcctcacatagagcactagtcaaggccagagggaccaggccttttgttattagcagaagtacctttgctgggcatggcagatatgctgggcactggacaggtgatgtatggagcagctgggaacagttggcttcatctgttccagagatcctgcagttcaacctgttgggggtgcctttagtgggggcagatgtctgtggtttcttggggaacacctcagaggaactttgtgtcagatggacccaactgggggccttttacccctttatgagaaaccacaactctctgctcagcttgcctcaggaaccctattccttcagtgagcctgcccaacaggccatgaggaaggccctaaccctcagatatgcactgctgcctcacctgtacaccctttttcaccaggcccatgtggcaggtgagacagtagcaagacctctcttcttggagttccctaaagactcaagcacctggactgtggatcaccaactcctgtggggagaggctctgctgatcacacctgtactacaagctggtaaggctgaggtgacaggttacttccccctgggcacctggtatgatctgcagacagtccctgtagaggcactaggctctctgcctccccctcctgcagcccctagagagcctgctatccacagtgagggccaatgggtcaccctacctgctccactagacacaataaatgtgcacctgagagctggctatatcatccccctccagggaccaggtctgacaaccacagaatctagacaacagcccatggcactagcagttgccctgaccaagggaggtgaagccaggggtgagcttttctgggatgatggggagtccttggaggttctagaaagaggagcctacactcaggtcatcttcctggccagaaataacaccatagtgaatgagttggtgagagtgacctcagagggagctgggctccaactgcaaaaagtgacagtcttgggagtggccacagcaccacagcaggtcctcagcaatggggtgcctgtgagcaacttcacctactctccagacaccaaagtactggacatctgtgtgagcctgctgatgggtgagcaattcctggtgagctggtgc

SEQ ID NO: 178 - DNA, 2649 bp, synthetic construct
  misc_feature 1..2649: note = Synthetic
  source 1..2649: mol_type = other DNA
gcccaccctggcagacctagagcagtgcccacccagtgtgatgtgcctcctaacagcaggtttgactgtgcacctgacaaggccatcacccaggagcagtgtgaagccaggggttgctgctatatcccagccaaacagggtctgcagggagcccaaatgggccagccttggtgcttctttccaccctcttacccttcttacaagctggaaaacctatcttcaagtgagatgggctacacagccactctaacaagaaccacacctacctttttccccaaggacatcctcactctgagactggatgtaatgatggagactgaaaacagactccacttcaccattaaagacccagccaacagaagatatgaggtaccattggagaccccccatgtccacagcagagcacccagccccctatactcagtggaattctctgaggaaccttttggtgtcatagtgagaagacagctggatggcagagtgctcctcaacacaactgtggctccactgttttttgctgaccaattcctacagctgagcaccagcctgccctcccaatatatcacagggctggctgaacacctatcccctctgatgctgtccacctcctggaccaggatcactctgtggaacagggacttagcccctacccctggtgccaacctgtatggctctcacccattctacctggccctggaggatggaggttcagcccatggagtctttctcctgaactctaatgccatggatgtggtgctacagccatcccctgcattatcttggaggagcacaggaggaatcctggatgtatacatctttctgggcccagaaccaaagtcagttgtacagcagtaccttgatgtggtaggctaccccttcatgcctccatactggggtctgggcttccacctctgcagatggggctacagcagtacagctatcacaagacaggttgtggagaacatgacaagggctcacttccccctggatgtacagtggaatgacttggactacatggacagtagaagggacttcacattcaacaaggatggcttcagggacttccctgccatggtacaggaactccaccagggagggagaagatacatgatgattgtggacccagctatcagttctagtggtcctgcaggttcctacaggccctatgatgaaggcctcagaagaggagtgttcatcactaatgagacaggacagcctttgataggaaaggtctggcctggcagtacagctttcccagattttaccaatccaactgctttggcttggtgggaggacatggtggcagagttccatgaccaggtgccctttgatggcatgtggattgacatgaatgaaccatctaacttcatcagaggatcagaagatggatgccccaacaatgagctggagaaccctccttatgtgccaggtgtggtgggtggcaccctacaggctgcaaccatctgtgccagctcacaccagttcctgagtacacactacaatctacacaacctgtatggcctgactgaagctattgcctcacatagagcactagtcaaggccagagggaccaggccttttgttattagcagaagtacctttgctgggcatggcagatatgctgggcactggacaggtgatgtatggagcagctgggaacagttggcttcatctgttccagagatcctgcagttcaacctgttgggggtgcctttagtgggggcagatgtctgtggtttcttggggaacacctcagaggaactttgtgtcagatggacccaactgggggccttttacccctttatgagaaaccacaactctctgctcagcttgcctcaggaaccctattccttcagtgagcctgcccaacaggccatgaggaaggccctaaccctcagatatgcactgctgcctcacctgtacaccctttttcaccaggcccatgtggcaggtgagacagtagcaagacctctcttcttggagttccctaaagactcaagcacctggactgtggatcaccaactcctgtggggagaggctctgctgatcacacctgtactacaagctggtaaggctgaggtgacaggttacttccccctgggcacctggtatgatctgcagacagtccctgtagaggcactaggctctctgcctccccctcctgcagcccctagagagcctgctatccacagtgagggccaatgggtcaccctacctgctccactagacaccatcaatgtgcacctgagagctggctatatcatccccctccaaggaccaggtctgacaaccacagaatctagacaacagcccatggcactagcagttgccctgaccaagggaggtgaagccaggggtgagcttttctgggatgatggggagtccttggaggttctagaaagaggagcctacactcaggtcatcttcctggccagaaataacaccatagtgaatgagttggtgagagtgacctcagagggagctgggctccaactgcaaaaagtgacagtcttgggagtggccacagcaccacagcaggtcctcagcaatggggtgcctgtgagcaacttcacctactctccagacaccaaagtactggacatctgtgtgagcctgctgatgggtgagcaattcctggtgagctggtgc

SEQ ID NO: 179 - DNA, 2649 bp, synthetic construct
  misc_feature 1..2649: note = Synthetic
  source 1..2649: mol_type = other DNA
gcccaccctggcagacccagagcagtccccacccagtgtgatgtacctcccaacagcagatttgactgtgcccctgacaaggccatcacccaggaacagtgtgaggccagggggtgctgctatattcctgccaagcagggcctgcagggtgcccaaatgggccagccttggtgcttcttccctcccagctacccctcttacaagttagaaaacctgtctagctctgaaatgggctacacagccaccctgaccagaacaacccctactttcttcccaaaggacattttaaccctgaggctggatgtgatgatggagactgagaatagactgcacttcaccatcaaggacccagccaacagaaggtatgaagtgcctctggagaccccccatgtgcactctagggcacctagccctctatactcagtggagttctctgaagaaccttttggggtcatagtgaggaggcaactggatggcagagtgcttctgaacaccacagtggcccccctgttctttgctgaccaattcctgcagctgagtaccagcctgccttcacagtacatcacaggcctggctgaacacctgagccctctgatgttgagcaccagctggaccagaatcaccttatggaacagagacttagccccaacccctggagccaacctctatggaagccaccctttctacctggccctggaggatggaggctctgcccatggtgttttcctcctgaactccaatgccatggatgttgtgctgcaaccttcccctgctctgagctggagatcaacagggggtatcttagatgtgtacatcttcctgggccctgaaccaaagtctgtggtacagcagtacctggatgtagtgggctaccccttcatgcccccctactggggcctaggcttccacctgtgcaggtgggggtactctagcactgccatcaccagacaggtggtggagaacatgactagagcccactttccattggatgtgcaatggaatgaccttgactacatggactcaaggagggacttcacattcaacaaggatggcttcagagactttcctgccatggtgcaggagctgcaccaagggggcagaagatacatgatgattgtggacccagccatctcctcttcaggacctgctggttcttatagaccttatgatgagggccttagaagaggtgtgttcattaccaatgaaactggacagcctctgattggcaaggtgtggccaggctccactgccttccctgactttacaaaccctacagccctggcctggtgggaggatatggtagctgagttccatgaccaggtgccttttgatggcatgtggatagacatgaatgaaccaagtaacttcatcagaggtagtgaggatggctgtcccaacaatgagctggagaaccccccatatgttcctggagtggtgggtggtactctccaggctgctaccatttgtgcctcaagtcaccagttcctgagcacccactacaacctgcacaacctgtatggcctgactgaggctattgctagccacagggctctggtgaaggccaggggaacaagaccttttgtgatttctaggtcaacatttgcaggccatggaagatatgctggtcactggacaggtgatgtgtggtcttcctgggaacagctggccagttcagtgccagagatcctgcagttcaacctcctaggagtgccactggtgggggcagatgtttgtggctttctggggaacacctctgaagagctgtgtgtgagatggacccagctgggagccttctacccattcatgaggaaccacaacagtttgctttccctacctcaggagccctacagcttctctgagccagcccaacaggccatgagaaaggccttgacacttagatatgccctactgccccacttgtataccttattccaccaggctcatgtggctggtgagactgtggccaggcccctgttcctggagttccccaaggacagctccacctggacagtagaccatcagctcctgtggggagaggccctgcttatcacccctgtgctccaagctggcaaagcagaggtgacaggttacttccctctgggcacctggtatgatctccagacagtgcctgtggaagcattaggttctttgcctcctccacctgcagcccccagagagcctgccatccactctgaaggccagtgggtcaccctgcctgcccccctggacacaatcaatgtccacctgagagctggttacataatccctctgcaagggcctggcttgaccaccacagaaagcagacagcagcctatggccctggctgtagctctgactaagggaggtgaggcaagaggggagctgttctgggatgatggagagagcctggaagtgctagagagaggagcctacacccaagtcatcttcttggccaggaacaataccatagtgaatgagctggtgagggtgacctctgaaggggctggccttcaactccagaaggtgactgttctgggagtggccacagccccacagcaggtgctgtccaatggtgtgcctgtcagcaacttcacatacagcccagatactaaggtgctggatatctgtgttagccttctcatgggtgaacagttcttagtgtcatggtgc

SEQ ID NO: 180 - DNA, 2649 bp, synthetic construct
  misc_feature 1..2649: note = Synthetic
  source 1..2649: mol_type = other DNA
gcccaccctggcagacctagagctgtgcctacccagtgtgatgtgccacctaactccagatttgactgtgcccctgacaaggccatcacccaagagcagtgcgaggccaggggatgctgctacatccctgccaaacagggcctgcagggagcccagatgggccaaccctggtgctttttccctccctcttacccctcctacaagctggagaacctgagctcctcagagatgggctacacagctaccctgaccagaaccacccccaccttctttcctaaggacatcctgaccctgagactggatgtgatgatggaaacagagaaccggctgcacttcacaatcaaggacccagccaacagaagatatgaagttcccctggagacccctcatgtgcactccagggcccccagccccctatacagcgtggagttctctgaggagccttttggcgtcattgtgagaagacaactggatggcagagtgctgttgaacaccacagtggcccctctcttctttgctgaccaattcctgcagctgagcaccagtctgcccagccagtacatcactggcctggctgagcacctgtctcccttaatgctgtccaccagctggacaaggatcaccctgtggaaccgggacttggcccccacgcctggagccaacctatacggctcccaccccttctacctggccctggaggacggaggctcagcccatggagttttcctgctgaactctaacgccatggatgtggtgctgcaacctagccctgccctgagctggagaagcactggcggaattctcgatgtctacatcttcctgggacctgaaccaaagtcagtggtgcagcagtacctggatgtggtgggctaccccttcatgccaccttactggggcctgggatttcacctgtgcagatggggctactccagcacagccatcaccagacaggtggtggagaacatgacccgggcccacttccccctggatgtccagtggaatgacttagactacatggatagcagaagagacttcaccttcaacaaggatggctttagagacttccctgccatggtgcaggaactgcaccagggtggcagaagatacatgatgattgtggaccctgccatttctagctctggcccagcaggctcctacaggccctatgatgagggactgcggagaggggtcttcatcaccaatgagacaggccagccactgatcggcaaggtgtggcctggatccacagccttcccagacttcaccaaccctacagcccttgcctggtgggaagacatggtggccgaattccacgaccaggtgccctttgatggcatgtggatcgacatgaatgagcctagcaatttcatcagaggaagtgaggatggctgccccaacaatgaattagaaaacccgccctatgtgcctggcgttgtgggaggcaccctccaggctgccaccatctgtgccagcagccaccagttcttatccacacactacaacctgcacaacctgtatggcctgaccgaagccattgccagccacagagcgctggtgaaagccaggggcacccggccttttgtgatcagcagaagcaccttcgctggccatggcaggtatgccggacattggacaggagatgtatggagctcttgggagcagttggccagctctgtgcctgagatcctgcagttcaacctactgggggtccctctggtgggagcagatgtctgcggcttcctaggcaacacctcagaggaactgtgcgtaagatggacccagctgggggccttctaccctttcatgaggaaccacaactctctgctgagcctgcctcaggagccatacagcttctctgagccagcccagcaggccatgagaaaggccctgaccctgagatatgccttactgccccacctgtacacactgttccaccaggcccatgtggctggggagacagtggcacggcccctgttcttggagttccccaaggactccagcacctggacagtggaccaccagctgctctggggagaggccctgctcatcaccccagtgctgcaggctggcaaggctgaagtcacaggctacttccccctgggcacctggtatgacctccaaactgtgccagtggaggccctgggcagcctgcctcctccccctgctgctcctagagagcctgccatccactcggagggccagtgggtgacactgcctgcccccctagacaccatcaatgttcacctgagagcaggctacatcatcccactgcaaggccctggcctgaccaccactgagagcagacagcagcccatggccctggcagttgccctgaccaagggcggcgaagccagaggagaactgttctgggatgatggcgaatctttggaagtgctggagagaggggcctacacccaggtgatcttcctggcccggaacaacactatagtgaatgaactggtgcgggtgacatcagagggtgcaggcctgcagctgcaaaaagtgacagtgctgggtgtggcaacagcccctcagcaggtactatccaatggagtgccagtgagcaacttcacctacagcccagacactaaggtgctagacatctgtgtttctctgctgatgggagagcaattcctggtgagctggtgc

SEQ ID NO: 181 - DNA, 2649 bp, synthetic construct
  misc_feature 1..2649: note = Synthetic
  source 1..2649: mol_type = other DNA
gcccaccctggcagacctagagctgtgcctacccagtgtgatgtgccacctaactccagatttgactgtgcccctgacaaggccatcacccaagagcagtgcgaggccaggggatgctgctacatccctgccaaacagggcctgcagggagcccagatgggccaaccctggtgctttttccctccctcttacccctcctacaagctggagaacctgagctcctcagagatgggctacacagctaccctgaccagaaccacccccaccttctttcctaaggacatcctgaccctgagactggatgtgatgatggaaacagagaaccggctgcacttcacaatcaaggacccagccaacagaagatatgaagttcccctggagacccctcatgtgcactccagggcccccagccccctatacagcgtggagttctctgaggagccttttggcgtcattgtgagaagacaactggatggcagagtgctgttgaacaccacagtggcccctctcttctttgctgaccaattcctgcagctgagcaccagtctgcccagccagtacatcactggcctggctgagcacctgtctcccttaatgctgtccaccagctggacaaggatcaccctgtggaaccgggacttggcccccacgcctggagccaacctatacggctcccaccccttctacctggccctggaggacggaggctcagcccatggagttttcctgctgaactctaacgccatggatgtggtgctgcaacctagccctgccctgagctggagaagcactggcggaattctcgatgtctacatcttcctgggacctgaaccaaagtcagtggtgcagcagtacctggatgtggtgggctaccccttcatgccaccttactggggcctgggatttcacctgtgcagatggggctactccagcacagccatcaccagacaggtggtggagaacatgacccgggcccacttccccctggatgtccagtggaatgacttagactacatggatagcagaagagacttcaccttcaacaaggatggctttagagacttccctgccatggtgcaggaactgcaccagggtggcagaagatacatgatgatcgttgaccctgccatttctagctctggcccagcaggctcctacaggccctatgatgagggactgcggagaggggtcttcatcaccaatgagacaggccagccactgatcggcaaggtgtggcctggatccacagccttcccagacttcaccaaccctacagcccttgcctggtgggaagacatggtggccgaattccacgaccaggtgccctttgatggcatgtggatcgacatgaatgagcctagcaatttcatcagaggaagtgaggatggctgccccaacaatgaattagaaaacccgccctatgtgcctggcgttgtgggaggcaccctccaggctgccaccatctgtgccagcagccaccagttcttatccacacactacaacctgcacaacctgtatggcctgaccgaagccattgccagccacagagcgctggtgaaagccaggggcacccggccttttgtgatcagcagaagcaccttcgctggccatggcaggtatgccggacattggacaggagacgtctggagctcttgggagcagttggccagctctgtgcctgagatcctgcagttcaacctactgggggtccctctggtgggagcagatgtctgcggcttcctaggcaacacctcagaggaactgtgcgtaagatggacccagctgggggccttctaccctttcatgaggaaccacaactctctgctgagcctgcctcaggagccatacagcttctctgagccagcccagcaggccatgagaaaggccctgaccctgagatatgccttactgccccacctgtacacactgttccaccaggcccacgtcgctggggagacagtggcacggcccctgttcttggagttccccaaggactccagcacctggacagtggaccaccagctgctctggggagaggccctgctcatcaccccagtgctgcaggctggcaaggctgaagtcacaggctacttccccctgggcacctggtatgacctccaaactgtgccagtggaggccctgggcagcctgcctcctccccctgctgctcctagagagcctgccatccactcggagggccagtgggtgacactgcctgcccccctagacaccatcaatgttcacctgagagcaggctacatcatcccactgcaaggccctggcctgaccaccactgagagcagacagcagcccatggccctggccgttgccctgaccaagggcggcgaagccagaggagaactgttctgggatgatggcgaatctttggaagtgctggagagaggggcctacacccaggtgatcttcctggcccggaacaacactatcgttaatgaactggtgcgggtgacatcagagggtgcaggcctgcagctgcaaaaagtgacagtgctgggtgtggcaacagcccctcagcaggtactatccaatggagtgccagtgagcaacttcacctacagcccagacactaaggtgctagacatctgtgtttctctgctgatgggagagcaattcctggtgagctggtgc

SEQ ID NO: 182 - DNA, 2649 bp, synthetic construct
  misc_feature 1..2649: note = Synthetic
  source 1..2649: mol_type = other DNA
gctcatcctggaagacctagagctgtgcctacacagtgtgatgtgcctcctaattctagatttgattgtgctcctgataaggctatcacacaggaacagtgtgaagctagaggatgttgttatatccctgctaagcagggactgcagggagctcagatgggacagccttggtgtttttttcctccttcttatccttcttataagctggaaaatctgtcttcttctgaaatgggatatacagctacactgacaagaacaacacctacattttttcctaaggatatcctgacactgagactggatgtgatgatggaaacagaaaatagactgcattttacaatcaaggatcctgctaatagaagatatgaagtgcctctggaaacacctcatgtgcattctagagctccttctcctctgtattctgtggaattttctgaagaaccttttggagtgatcgtgagaagacagctggatggaagagtgctgctgaatacaacagtggctcctctgttttttgctgatcagtttctgcagctgtctacatctctgccttctcagtatatcacaggactggctgaacatctgtctcctctgatgctgtctacatcttggacaagaatcacactgtggaatagagatctggctcctacacctggagctaatctgtatggatctcatcctttttatctggctctggaagatggaggatctgctcatggagtgtttctgctgaattctaatgctatggatgtggtgctgcagccttctcctgctctgtcttggagatctacaggaggaatcctggatgtgtatatctttctgggacctgaacctaagtctgtggtgcagcagtatctggatgtggtgggatatccttttatgcctccttattggggactgggatttcatctgtgtagatggggatattcttctacagctatcacaagacaggtggtggaaaatatgacaagagctcattttcctctggatgtgcagtggaatgatctggattatatggattctagaagagattttacatttaataaggatggatttagagattttcctgctatggtgcaggaactgcatcagggaggaagaagatatatgatgatcgtggatcctgctatctcttcttctggacctgctggatcttatagaccttatgatgaaggactgagaagaggagtgtttatcacaaatgaaacaggacagcctctgatcggaaaggtgtggcctggatctacagcttttcctgattttacaaatcctacagctctggcttggtgggaagatatggtggctgaatttcatgatcaggtgccttttgatggaatgtggatcgatatgaatgaaccttctaattttatcagaggatctgaagatggatgtcctaataatgaactggaaaatcctccttatgtgcctggagtggtgggaggaacactgcaggctgctacaatctgtgcttcttctcatcagtttctgtctacacattataatctgcataatctgtatggactgacagaagctatcgcttctcatagagctctggtgaaggctagaggaacaagaccttttgtgatctctagatctacatttgctggacatggaagatatgctggacattggacaggagatgtgtggtcttcttgggaacagctggcttcttctgtgcctgaaatcctgcagtttaatctgctgggagtgcctctggtgggagctgatgtgtgtggatttctgggaaatacatctgaagaactgtgtgtgagatggacacagctgggagctttttatccttttatgagaaatcataattctctgctgtctctgcctcaggaaccttattctttttctgaacctgctcagcaggctatgagaaaggctctgacactgagatatgctctgctgcctcatctgtatacactgtttcatcaggctcatgtggctggagaaacagtggctagacctctgtttctggaatttcctaaggattcttctacatggacagtggatcatcagctgctgtggggagaagctctgctgatcacacctgtgctgcaggctggaaaggctgaagtgacaggatattttcctctgggaacatggtatgatctgcagacagtgcctgtggaagctctgggatctctgcctcctcctcctgctgctcctagagaacctgctatccattctgaaggacagtgggtgacactgcctgctcctctggatacaatcaatgtgcatctgagagctggatatatcatccctctgcagggacctggactgacaacaacagaatctagacagcagcctatggctctggctgtggctctgacaaagggaggagaagctagaggagaactgttttgggatgatggagaatctctggaagtgctggaaagaggagcttatacacaggtgatctttctggctagaaataatacaatcgtgaatgaactggtgagagtgacatctgaaggagctggactgcagctgcagaaggtgacagtgctgggagtggctacagctcctcagcaggtgctgtctaatggagtgcctgtgtctaattttacatattctcctgatacaaaggtgctggatatctgtgtgtctctgctgatgggagaacagtttctggtgtcttggtgt

SEQ ID NO: 183 - protein, 240 aa, synthetic construct
  REGION 1..240: note = Synthetic
QVQLQESGPKVVKPSETLSLTCTVSGGSISSYYWNWIRQSPGKGLEWIGYTKRGYTDYNPSLRSRVTISEDTSKNQFSLRISSVTAADTAVYYCAQMGWGSHAFDMWGQGTMVAVSSGGSGGGGSGGGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 184 - protein, 1136 aa, synthetic construct
  REGION 1..1136: note = Synthetic
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKGGGGSGGGGSGGGGSEVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 185 - protein, 1138 aa, synthetic construct
  REGION 1..1138: note = Synthetic
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKGGGGSGGGGSGGGGSEVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 186 - protein, 1136 aa, synthetic construct
  REGION 1..1136: note = Synthetic
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKGGGGSGGGGSGGGGSEVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 187 - protein, 1131 aa, synthetic construct
  REGION 1..1131: note = Synthetic
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKGGGGSGGGGSGGGGSQVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 188 - protein, 1138 aa, synthetic construct
  REGION 1..1138: note = Synthetic
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKGGGGSGGGGSGGGGSQLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 189 - protein, 1133 aa, synthetic construct
  REGION 1..1133: note = Synthetic
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKGGGGSGGGGSGGGGSQVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 190 - protein, 1143 aa, synthetic construct
  REGION 1..1143: note = Synthetic
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKGGGGSGGGGSGGGGSQVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 191 - protein, 1138 aa, synthetic construct
  REGION 1..1138: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 192 - protein, 1140 aa, synthetic construct
  REGION 1..1140: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 193 - protein, 1134 aa, synthetic construct
  REGION 1..1134: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 194 - protein, 1141 aa, synthetic construct
  REGION 1..1141: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 195 - protein, 1137 aa, synthetic construct
  REGION 1..1137: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 196 - protein, 1144 aa, synthetic construct
  REGION 1..1144: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 197 - protein, 1135 aa, synthetic construct
  REGION 1..1135: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 198 - protein, 1138 aa, synthetic construct
  REGION 1..1138: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 199 - protein, 1136 aa, synthetic construct
  REGION 1..1136: note = Synthetic
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKGGGGSGGGGSGGGGSEVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 200 - protein, 1136 aa, synthetic construct
  REGION 1..1136: note = Synthetic
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKGGGGSGGGGSGGGGSEVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 201 - protein, 1143 aa, synthetic construct
  REGION 1..1143: note = Synthetic
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKGGGGSGGGGSGGGGSQVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 202 - protein, 1137 aa, synthetic construct
  REGION 1..1137: note = Synthetic
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKGGGGSGGGGSGGGGSEVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 203 - protein, 1132 aa, synthetic construct
  REGION 1..1132: note = Synthetic
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 204 - protein, 1140 aa, synthetic construct
  REGION 1..1140: note = Synthetic
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKGGGGSGGGGSGGGGSEVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 205 - protein, 1142 aa, synthetic construct
  REGION 1..1142: note = Synthetic
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKGGGGSGGGGSGGGGSEVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 206 - protein, 1131 aa, synthetic construct
  REGION 1..1131: note = Synthetic
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKGGGGSGGGGSGGGGSEVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 207 - protein, 1140 aa, synthetic construct
  REGION 1..1140: note = Synthetic
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKGGGGSGGGGSGGGGSEVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 208 - protein, 1135 aa, synthetic construct
  REGION 1..1135: note = Synthetic
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKGGGGSGGGGSGGGGSQVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 209 - protein, 1135 aa, synthetic construct
  REGION 1..1135: note = Synthetic
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKGGGGSGGGGSGGGGSQVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 210 - protein, 1138 aa, synthetic construct
  REGION 1..1138: note = Synthetic
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKGGGGSGGGGSGGGGSEVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 211 - protein, 1137 aa, synthetic construct
  REGION 1..1137: note = Synthetic
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKGGGGSGGGGSGGGGSQVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 212 - protein, 1168 aa, synthetic construct
  REGION 1..1168: note = Synthetic
MHRPRRRGTRPPPLALLAALLLAARGADADIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 213 - protein, 1171 aa, synthetic construct
  REGION 1..1171: note = Synthetic
MHRPRRRGTRPPPLALLAALLLAARGADADIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 214 - protein, 1164 aa, synthetic construct
  REGION 1..1164: note = Synthetic
MHRPRRRGTRPPPLALLAALLLAARGADADIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 215 - protein, 1166 aa, synthetic construct
  REGION 1..1166: note = Synthetic
MHRPRRRGTRPPPLALLAALLLAARGADADIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 216 - DNA, 363 bp, synthetic construct
  misc_feature 1..363: note = Synthetic
  source 1..363: mol_type = other DNA
gaggtgcagctggtggagtctgggggaggcttggtacagcctggggggtccctgagactctcctgtgcagcctctggattcgcctttagcagctatgccatgacctgggtccgacaggctccagggaaggggctggagtgggtctcagttatcagtggtactggtggtagtacatactacgcagactccgtgaagggccggttcaccatctccagagacaattccaagaacacgctgtatctacaaatgaacagcctgagagccgaggacacggccgtatattactgtgcgaaagggggagcagctcgtagaatggaatacttccagtactggggccagggcaccctggtcaccgtctcctca

SEQ ID NO: 217 - protein, 121 aa, synthetic construct
  REGION 1..121: note = Synthetic
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 218 - protein, 8 aa, synthetic construct
  REGION 1..8: note = Synthetic
GFAFSSYA

SEQ ID NO: 219 - protein, 8 aa, synthetic construct
  REGION 1..8: note = Synthetic
ISGTGGST

SEQ ID NO: 220 - protein, 14 aa, synthetic construct
  REGION 1..14: note = Synthetic
AKGGAARRMEYFQY

SEQ ID NO: 221 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcgagtcagggcattagcaattatttagcctggtatcagcagaaaccagggaaagttcctaacctccttatctatgctgcatccactttgcaatcaggggtcccatctcgattcagtggcagtggatctgggacagatttcactctcaccatcagcagcctgcagcctgaagatgttgcaacttattactgtcaaaagtataacagtgcccctctcactttcggcggagggaccaaggtggagatcaaa

SEQ ID NO: 222 - protein, 107 aa, synthetic construct
  REGION 1..107: note = Synthetic
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIK

SEQ ID NO: 223 - protein, 6 aa, synthetic construct
  REGION 1..6: note = Synthetic
QGISNY

SEQ ID NO: 224 - skipped

SEQ ID NO: 225 - protein, 9 aa, synthetic construct
  REGION 1..9: note = Synthetic
QKYNSAPLT

SEQ ID NO: 226 - DNA, 363 bp, synthetic construct
  misc_feature 1..363: note = Synthetic
  source 1..363: mol_type = other DNA
gaggtgcagctggtggagtctgggggaggcttggtacagcctggggggtccctgagactctcctgtgcagcctctggattcacctttaacagctatgccatgacctgggtccgccaggctccagggaaggggctggagtgggtctcatttattggtggtagtactggtaacacatactacgcaggctccgtgaagggccggttcaccatctccagcgacaattccaagaagacgctgtatctgcaaatgaacagcctgagagccgaggacacggccgtatattactgtgcgaaagggggagcagctcgtagaatggaatacttccagcactggggccagggcaccctggtcaccgtctcctca

SEQ ID NO: 227 - protein, 121 aa, synthetic construct
  REGION 1..121: note = Synthetic
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 228 - protein, 8 aa, synthetic construct
  REGION 1..8: note = Synthetic
GFTFNSYA

SEQ ID NO: 229 - protein, 8 aa, synthetic construct
  REGION 1..8: note = Synthetic
IGGSTGNT

SEQ ID NO: 230 - protein, 14 aa, synthetic construct
  REGION 1..14: note = Synthetic
AKGGAARRMEYFQH

SEQ ID NO: 231 - DNA, 321 bp, synthetic construct
  misc_feature 1..321: note = Synthetic
  source 1..321: mol_type = other DNA
gacatccagatgacccagtctccatcctccctgtctgcatctataggagacagagtcaccatcacttgccgggcgagtcagggcattagcaattatttagcctggtatcaacagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaaccataacagtgtccctctcactttcggcggagggaccaaggtggagatcaaa

SEQ ID NO: 232 - protein, 107 aa, synthetic construct
  REGION 1..107: note = Synthetic
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIK

SEQ ID NO: 233 - protein, 6 aa, synthetic construct
  REGION 1..6: note = Synthetic
QGISNY

SEQ ID NO: 234 - skipped

SEQ ID NO: 235 - protein, 9 aa, synthetic construct
  REGION 1..9: note = Synthetic
QNHNSVPLT

SEQ ID NO: 236 - DNA, 366 bp, synthetic construct
  misc_feature 1..366: note = Synthetic
  source 1..366: mol_type = other DNA
caggtgcagctggtggagtctgggggaggcgtggtccagcctgggaggtccctgagactctcctgtgcagcgtctggattcaccttcactacctatggcatgcactgggtccgccaggctccaggcaaggggctggagtgggtggctgttatatggtatgatggaagtaataaatattatggagactccgtgaagggccgattcaccatctccagagacaattccaagaacacactgtatctgcaaatgaacagcctgagagtcgacgacacggctgtttattactgtacgagaacccatggctataccaggtcgtcggacggttttgactactggggccagggaaccctggtcaccgtctcctca

SEQ ID NO: 237 - protein, 122 aa, synthetic construct
  REGION 1..122: note = Synthetic
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 238 - protein, 8 aa, synthetic construct
  REGION 1..8: note = Synthetic
GFTFTTYG

SEQ ID NO: 239 - protein, 8 aa, synthetic construct
  REGION 1..8: note = Synthetic
IWYDGSNK

SEQ ID NO: 240 - protein, 15 aa, synthetic construct
  REGION 1..15: note = Synthetic
TRTHGYTRSSDGFDY

SEQ ID NO: 241 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagagcattagaaatgttttaggctggtttcagcagaaaccagggaaagcccctcagcgcctgatctatgctgcatccagtttgcaaagtggggtcccatcaaggttcagcggcagtggatctgggacagaattcactctcacaatcagcagcctacagcctgaagattttgcaacttattactgtctacagcataatttttacccgctcactttcggcggagggaccaaggtggagatcaaa

SEQ ID NO: 242 - protein, 107 aa, synthetic construct
  REGION 1..107: note = Synthetic
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIK

SEQ ID NO: 243 - protein, 6 aa, synthetic construct
  REGION 1..6: note = Synthetic
QSIRNV

SEQ ID NO: 244 - skipped

SEQ ID NO: 245 - protein, 9 aa, synthetic construct
  REGION 1..9: note = Synthetic
LQHNFYPLT

SEQ ID NO: 246 - DNA, 369 bp, synthetic construct
  misc_feature 1..369: note = Synthetic
  source 1..369: mol_type = other DNA
gaagtgcagctggtggagtctgggggaggcttggtacagcctggcaggtccctgagactctcctgtgcagcctctggattcacctttgatgataaagccatgcactgggtccggcaagttccagggaagggcctggaatggatctcaggtattagttggaatagtggtactataggctatgcggactctgtgaagggccgattcatcatctccagagacaacgccaagaactccctgtatctacaaatgaacagtctgagagctgaggacacggccttgtattactgcgcaaaagatggagataccagtggctggtactggtacggtttggacgtctggggccaagggaccacggtcaccgtctcctca

SEQ ID NO: 247 - protein, 123 aa, synthetic construct
  REGION 1..123: note = Synthetic
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 248 - protein, 8 aa, synthetic construct
  REGION 1..8: note = Synthetic
GFTFDDKA

SEQ ID NO: 249 - protein, 8 aa, synthetic construct
  REGION 1..8: note = Synthetic
ISWNSGTI

SEQ ID NO: 250 - protein, 16 aa, synthetic construct
  REGION 1..16: note = Synthetic
AKDGDTSGWYWYGLDV

SEQ ID NO: 251 - DNA, 321 bp, synthetic construct
  misc_feature 1..321: note = Synthetic
  source 1..321: mol_type = other DNA
gaaattgtgttgacacagtctcctgccaccctgtctttgtctccaggggaaagagccaccctctcctgcagggccagtcagagtgttagcagctacttagcctggtaccaacagaaacctggccaggctcccaggctcctcatccatgatgtatccaacagggccactggcatcccagccaggttcagtggcagtgggtctgggacagacttcactctcaccatcagcagtctagagcctgaagattttgtagtttattactgtcagcagcgtagcgactggcccatcaccttcggccaagggacacgactggagattaaa

SEQ ID NO: 252 - protein, 107 aa, synthetic construct
  REGION 1..107: note = Synthetic
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIK

SEQ ID NO: 253 - protein, 6 aa, synthetic construct
  REGION 1..6: note = Synthetic
QSVSSY

SEQ ID NO: 254 - skipped

SEQ ID NO: 255 - protein, 9 aa, synthetic construct
  REGION 1..9: note = Synthetic
QQRSDWPIT

SEQ ID NO: 256 - DNA, 360 bp, synthetic construct
  misc_feature 1..360: note = Synthetic
  source 1..360: mol_type = other DNA
caggtgcagctggtggagtctgggggaggcgtggtccagcctgggaggtccctgagactctcctgtatagcctctggattcaccttcagtgtctatggcattcactgggtccgccaggctccaggcaaggggctggagtggatggcagtaatatcacatgatggaaatattaaacactatgcagactccgtgaagggccgattcaccatctccagagacaattccaagaacacgctgtatcttcaaattaacagcctgagaactgaggacacggctgtgtattactgtgcgaaagatacctggaactcccttgatacttttgatatctggggccaagggacaatggtcaccgtctcttca

SEQ ID NO: 257 - protein, 120 aa, synthetic construct
  REGION 1..120: note = Synthetic
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 258 - protein, 8 aa, synthetic construct
  REGION 1..8: note = Synthetic
GFTFSVYG

SEQ ID NO: 259 - protein, 8 aa, synthetic construct
  REGION 1..8: note = Synthetic
ISHDGNIK

SEQ ID NO: 260 - protein, 13 aa, synthetic construct
  REGION 1..13: note = Synthetic
AKDTWNSLDTFDI

SEQ ID NO: 261 - DNA, 321 bp, synthetic construct
  misc_feature 1..321: note = Synthetic
  source 1..321: mol_type = other DNA
gacatccagttgacccagtctccatcctccctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggagatcaaa

SEQ ID NO: 262 - protein, 107 aa, synthetic construct
  REGION 1..107: note = Synthetic
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 263 - protein, 6 aa, synthetic construct
  REGION 1..6: note = Synthetic
QGISSY

SEQ ID NO: 264 - skipped

SEQ ID NO: 265 - protein, 9 aa, synthetic construct
  REGION 1..9: note = Synthetic
QQLNSYPLT

SEQ ID NO: 266 - DNA, 360 bp, synthetic construct
  misc_feature 1..360: note = Synthetic
  source 1..360: mol_type = other DNA
caggtcaccttgagggagtctggtcccgcgctggtgaaaccctcacagaccctcacactgacctgcaccttctctggattctcactcaacacttatgggatgtttgtgagctggatccgtcagcctccagggaaggccctagagtggcttgcacacattcattgggatgatgataaatactacagcacatctctgaagaccaggctcaccatctccaaggacacctccaaaaaccaggtggtccttacaatgaccaacatggaccctgtggacacagccacgtattattgtgcacgggggcacaataatttgaactacatcatccactggggccagggaaccctggtcaccgtctcctca

SEQ ID NO: 267 - protein, 120 aa, synthetic construct
  REGION 1..120: note = Synthetic
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 268 - protein, 10 aa, synthetic construct
  REGION 1..10: note = Synthetic
GFSLNTYGMF

SEQ ID NO: 269 - protein, 7 aa, synthetic construct
  REGION 1..7: note = Synthetic
IHWDDDK

SEQ ID NO: 270 - protein, 12 aa, synthetic construct
  REGION 1..12: note = Synthetic
ARGHNNLNYIIH

SEQ ID NO: 271 - DNA, 321 bp, synthetic construct
  misc_feature 1..321: note = Synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccactttacaaagtggggtcccatcaaggttcagcggcagtggatctggcacagatttcactctcaccatcagcagcctgcagcctgaagattttgcaacttattactgtctacaagattacaattacccattcactttcggccctgggaccaaagtggatatcaaa

SEQ ID NO: 272 - protein, 107 aa, synthetic construct
  REGION 1..107: note = Synthetic
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIK

SEQ ID NO: 273 - protein, 6 aa, synthetic construct
  REGION 1..6: note = Synthetic
QGIRND

SEQ ID NO: 274 - skipped

SEQ ID NO: 275 - protein, 9 aa, synthetic construct
  REGION 1..9: note = Synthetic
LQDYNYPFT

SEQ ID NO: 276 - DNA, 348 bp, synthetic construct
  misc_feature 1..348: note = Synthetic
  source 1..348: mol_type = other DNA
gaggtgcagctggtggagtctgggggaggcttggtacagcctggagggtccctgagactctcctgtgcagtctctggattcatcttcagtagttatgaaatgaactgggtccgccaggctccagggaaggggctggagtgggtttcatacattagtagtagtggtagtaccatattctacgcagactctgtgaagggccgattcaccatctccagagacaacgccaagaactcactgtatctgcaaatgaacagcctgagagccgaggacacggctgtttattactgtgtgtctggagtggtcctttttgatgtctggggccaagggacaatggtcaccgtctcttca

SEQ ID NO: 277 - protein, 116 aa, synthetic construct
  REGION 1..116: note = Synthetic
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 278 - protein, 8 aa, synthetic construct
  REGION 1..8: note = Synthetic
GFIFSSYE

SEQ ID NO: 279 - protein, 8 aa, synthetic construct
  REGION 1..8: note = Synthetic
ISSSGSTI

SEQ ID NO: 280 - protein, 9 aa, synthetic construct
  REGION 1..9: note = Synthetic
VSGVVLFDV

SEQ ID NO: 281 - DNA, 321 bp, synthetic construct
  misc_feature 1..321: note = Synthetic
  source 1..321: mol_type = other DNA
gaaatagtgatgacgcagtctccagccaccctgtctgtgtctccgggggaaagagccaccctctcctgcagggccagtcagagtgttagcagcaactttgcctggtaccaacagaaacctggccaggctcccaggctcctcatctatagtgcatcctccagggccactggtatcccagtcaggttcagtggcagtgggtctgggacagagttcactctcaccatcagcagcctgcagtctgaagattttgcagtttattactgtcagcagtataatatctggcctcggacgttcggccaagggaccaaggtggaaatcaaa

SEQ ID NO: 282 - protein, 107 aa, synthetic construct
  REGION 1..107: note = Synthetic
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIK

SEQ ID NO: 283 - protein, 6 aa, synthetic construct
  REGION 1..6: note = Synthetic
QSVSSN

SEQ ID NO: 284 - skipped

SEQ ID NO: 285 - protein, 9 aa, synthetic construct
  REGION 1..9: note = Synthetic
QQYNIWPRT

SEQ ID NO: 286 - DNA, 375 bp, synthetic construct
  misc_feature 1..375: note = Synthetic
  source 1..375: mol_type = other DNA
gaggtgcagctggtggagtctgggggaggcttggtccagcctggggggtccctgagactctcctgtgcagcctctggattcacctttagtaactattggatgacctgggtccgccaggctccagggaaggggctggagtgggtggccaacataaaggaagatggaagtgagaaagactatgtggactctgtgaagggccgattcaccatctccagagacaacgccaagaactcactgtatctgcaaatgaacagcctgagaggcgaggacacggctgtgtattactgtgcgagagatggggagcagctcgtcgattactactactactacgttatggacgtctggggccaagggaccacggtcaccgtctcctca

SEQ ID NO: 287 - protein, 125 aa, synthetic construct
  REGION 1..125: note = Synthetic
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 288 - protein, 8 aa, synthetic construct
  REGION 1..8: note = Synthetic
GFTFSNYW

SEQ ID NO: 289 - protein, 8 aa, synthetic construct
  REGION 1..8: note = Synthetic
IKEDGSEK

SEQ ID NO: 290 - protein, 18 aa, synthetic construct
  REGION 1..18: note = Synthetic
ARDGEQLVDYYYYYVMDV

SEQ ID NO: 291 - DNA, 321 bp, synthetic construct
  misc_feature 1..321: note = Synthetic
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctaacagtttcccgtacacttttggccaggggaccaagctggagatcaaa

SEQ ID NO: 292 - protein, 107 aa, synthetic construct
  REGION 1..107: note = Synthetic
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIK

SEQ ID NO: 293 - protein, 6 aa, synthetic construct
  REGION 1..6: note = Synthetic
QGISSW

SEQ ID NO: 294 - skipped

SEQ ID NO: 295 - protein, 9 aa, synthetic construct
  REGION 1..9: note = Synthetic
QKANSFPYT

SEQ ID NO: 296 - DNA, 381 bp, synthetic construct
  misc_feature 1..381: note = Synthetic
  source 1..381: mol_type = other DNA
gaagtgcagctggtggagtctgggggaggcttggtacagcctggcaggtccctgagactctcctgtgcagcctctggattcacctttgatgactatgccatgcactgggtccggcaagctccagggaagggcctggagtgggtctcaggtattagttggaatagtggttacataggctatgcggactctgtgaagggccgattcaccatctccagagacaacgccgagaactccctacatctgcaaatgaacagtctgagagctgaggacacggccttgtattactgtgcaagagggggatctactctggttcggggagttaagggaggctactacggtatggacgtctggggccaagggaccacggtcaccgtctcctca

SEQ ID NO: 297 - protein, 127 aa, synthetic construct
  REGION 1..127: note = Synthetic
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 298 - protein, 8 aa, synthetic construct
  REGION 1..8: note = Synthetic
GFTFDDYA

SEQ ID NO: 299 - protein, 8 aa, synthetic construct
  REGION 1..8: note = Synthetic
ISWNSGYI

SEQ ID NO: 300 - protein, 20 aa, synthetic construct
  REGION 1..20: note = Synthetic
ARGGSTLVRGVKGGYYGMDV

SEQ ID NO: 301 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagagcataagtagctatttaaattggtatcagcagaaaccaggtaaagcccctaaggtcctgatctatgctgcatccagtttgcaaagtggggtcccatcaaggttcagtggcagtggatctgggacagatttcactctcaccatcagcagtctgcaacctgaagattttgcaacttactactgtcaacagagttacagtattccgctcactttcggcggagggaccaaggtggagatcaaa

SEQ ID NO: 302 - protein, 107 aa, synthetic construct
  REGION 1..107: note = Synthetic
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIK

SEQ ID NO: 303 - protein, 6 aa, synthetic construct
  REGION 1..6: note = Synthetic
QSISSY

SEQ ID NO: 304 - skipped

SEQ ID NO: 305 - protein, 9 aa, synthetic construct
  REGION 1..9: note = Synthetic
QQSYSIPLT

SEQ ID NO: 306 - DNA, 351 bp, synthetic construct
  misc_feature 1..351: note = Synthetic
  source 1..351: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacatttccaagaacacgctgtatctgcaaatgaacagcctgagagccgaggacacggccgtatattactgtgcgggtcaactggatctcttctttgactactggggccagggaaccctggtcaccgtctcctca

SEQ ID NO: 307 - protein, 117 aa, synthetic construct
  REGION 1..117: note = Synthetic
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 308 - protein, 8 aa, synthetic construct
  REGION 1..8: note = Synthetic
GFTFSSYG

SEQ ID NO: 309 - protein, 8 aa, synthetic construct
  REGION 1..8: note = Synthetic
IWYDGSNK

SEQ ID NO: 310 - protein, 10 aa, synthetic construct
  REGION 1..10: note = Synthetic
AGQLDLFFDY

SEQ ID NO: 311 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagagcattgacaggtatttaaattggtatcggcagaaaccagggaaagcccctaagctcctgatctatactacatccagtttgcaaagtggggtcccatcaaggttcagtggcagtggatctgggacagatttcactctcaccctcagcagtctgcagcctgaagattttgcaacttactactgtcagcagagttacagtcccccgctcactttcggcggagggaccaaggtggagatcaaa

SEQ ID NO: 312 - protein, 107 aa, synthetic construct
  REGION 1..107: note = Synthetic
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIK

SEQ ID NO: 313 - protein, 6 aa, synthetic construct
  REGION 1..6: note = Synthetic
QSIDRY

SEQ ID NO: 314 - skipped

SEQ ID NO: 315 - protein, 9 aa, synthetic construct
  REGION 1..9: note = Synthetic
QQSYSPPLT

SEQ ID NO: 316 - DNA, 375 bp, synthetic construct
  misc_feature 1..375: note = Synthetic
  source 1..375: mol_type = other DNA
gaggtgcagctggtggagtctgggggaggcttggtccagcctggggggtccctgagactctcctgtacagcctctggattcacctttagtaactattggatgacctgggtccgccaggctccagggaaggggctggagtgggtggccaacataaaggaagatggaagtgagaaagagtatgtggactctgtgaagggccggttcaccatctccagagacaacgccaagaattcactgtatctgcaaatgaacagcctgagaggcgaggacacggctgtatattactgtgcgagagatggggagcagctcgtcgattactattactactacgttatggacgtctggggccaagggaccacggtcaccgtctcctca

SEQ ID NO: 317 - protein, 125 aa, synthetic construct
  REGION 1..125: note = Synthetic
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 318 - protein, 8 aa, synthetic construct
  REGION 1..8: note = Synthetic
GFTFSNYW

SEQ ID NO: 319 - protein, 8 aa, synthetic construct
  REGION 1..8: note = Synthetic
IKEDGSEK

SEQ ID NO: 320 - protein, 18 aa, synthetic construct
  REGION 1..18: note = Synthetic
ARDGEQLVDYYYYYVMDV

SEQ ID NO: 321 - DNA, 321 bp, synthetic construct
  misc_feature 1..321: note = Synthetic
  source 1..321: mol_type = other DNA
gacatccagatgacccagtctccatcttccgtgtctgcatctgttggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctgacagtctcccgtacgcttttggccaggggaccaagctggagatcaaa

SEQ ID NO: 322 - protein, 107 aa, synthetic construct
  REGION 1..107: note = Synthetic
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIK

SEQ ID NO: 323 - protein, 6 aa, synthetic construct
  REGION 1..6: note = Synthetic
QGISSW

SEQ ID NO: 324 - skipped

SEQ ID NO: 325 - protein, 9 aa, synthetic construct
  REGION 1..9: note = Synthetic
QKADSLPYA

SEQ ID NO: 326 - DNA, 363 bp, synthetic construct
  misc_feature 1..363: note = Synthetic
  source 1..363: mol_type = other DNA
gaggtgcagctggtggagtctgggggaggcttggttcagcctggggggtccctgagactctcctgtgcaacctctggattcacctttaccagctatgacatgaagtgggtccgccaggctccagggctgggcctggagtgggtctcagctattagtggtagtggtggtaacacatactacgcagactccgtgaagggccggttcaccatctccagagacaattccaggaacacgctgtatctgcaaatgaacagcctgagagccgaggacacggccgtatattactgtacgaggtcccatgacttcggtgccttcgactactttgactactggggccagggaaccctggtcaccgtctcctca

SEQ ID NO: 327 - protein, 121 aa, synthetic construct
  REGION 1..121: note = Synthetic
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 328 - protein, 8 aa, synthetic construct
  REGION 1..8: note = Synthetic
GFTFTSYD

SEQ ID NO: 329 - protein, 8 aa, synthetic construct
  REGION 1..8: note = Synthetic
ISGSGGNT

SEQ ID NO: 330 - protein, 14 aa, synthetic construct
  REGION 1..14: note = Synthetic
TRSHDFGAFDYFDY

SEQ ID NO: 331 - DNA, 321 bp, synthetic construct
  misc_feature 1..321: note = Synthetic
  source 1..321: mol_type = other DNA
gacatccagatgacccagtctccatcctccctgtctgcatctgtgggagacagagtcaccatcacttgccgggcaagtcagggcattagagatcattttggctggtatcagcagaaaccagggaaagcccctaagcgcctgatctatgctgcatccagtttgcacagtggggtcccatcaaggttcagcggcagtggatctgggacagaattcactctcacaatcagcagcttgcagcctgaagattttgcaacctattactgtctacagtatgatacttacccgctcactttcggcggagggaccaaggtggagatcaaa

SEQ ID NO: 332 - protein, 107 aa, synthetic construct
  REGION 1..107: note = Synthetic
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIK

SEQ ID NO: 333 - protein, 6 aa, synthetic construct
  REGION 1..6: note = Synthetic
QGIRDH

SEQ ID NO: 334 - skipped

SEQ ID NO: 335 - protein, 9 aa, synthetic construct
  REGION 1..9: note = Synthetic
LQYDTYPLT

SEQ ID NO: 336 - DNA, 366 bp, synthetic construct
  misc_feature 1..366: note = Synthetic
  source 1..366: mol_type = other DNA
gaagtgcagctggtggagtctgggggagacttggtacagcctggcaggtccctgagactctcctgtgcagcctctggattcacctttgatgattatgccatgcactgggtccggcaagctccagggaagggcctggagtgggtctcaggtattagttggaatagtgctaccagagtctatgcggactctgtgaagggccgattcaccatctccagagacaacgccaagaatttcctgtatctgcaaatgaacagtctgagatctgaggacacggccttgtatcactgtgcaaaagatatggatatctcgctagggtactacggtttggacgtctggggccaagggaccacggtcaccgtctcctca

SEQ ID NO: 337 - protein, 122 aa, synthetic construct
  REGION 1..122: note = Synthetic
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 338 - protein, 8 aa, synthetic construct
  REGION 1..8: note = Synthetic
GFTFDDYA

SEQ ID NO: 339 - protein, 8 aa, synthetic construct
  REGION 1..8: note = Synthetic
ISWNSATR

SEQ ID NO: 340 - protein, 15 aa, synthetic construct
  REGION 1..15: note = Synthetic
AKDMDISLGYYGLDV

SEQ ID NO: 341 - DNA, 324 bp, synthetic construct
  misc_feature 1..324: note = Synthetic
  source 1..324: mol_type = other DNA
gaaatagtgatgacgcagtctccagccaccctgtctgtgtctccaggggaaagagccaccctctcctgcagggccagtcagactgttagcagcaacttagcctggtatcagcagaaacctggccaggctcccaggctcctcatctatggttcatcctccagggccactggtatcccagccaggttcagtggcagtgggtctgggacagagttcactctcaccatcagcagcctgcagtctgaagattttgcagtttattactgtcagcagtataataactggcctccctacacttttggccaggggaccaagctggagatcaaa

SEQ ID NO: 342 - protein, 108 aa, synthetic construct
  REGION 1..108: note = Synthetic
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIK

SEQ ID NO: 343 - protein, 6 aa, synthetic construct
  REGION 1..6: note = Synthetic
QTVSSN

SEQ ID NO: 344 - skipped

SEQ ID NO: 345 - protein, 10 aa, synthetic construct
  REGION 1..10: note = Synthetic
QQYNNWPPYT

SEQ ID NO: 346 - DNA, 372 bp, synthetic construct
  misc_feature 1..372: note = Synthetic
  source 1..372: mol_type = other DNA
cagatcaccttgaaggagtctggtcctacgctggtgaaacccacacagaccctcacgctgacctgcaccttctctgggttctcactcagcactagtggagtgggtgtggtctggatccgtcagccccccggaaaggccctggagtggcttgcactcatttattggaatgatcataagcggtacagcccatctctggggagcaggctcaccatcaccaaggacacctccaaaaaccaggtggtccttacaatgaccaacatggaccctgtggacacagccacatattactgtgcacactacagtgggagctattcctactactactatggtttggacgtctggggccaagggaccacggtcaccgtctcctca

SEQ ID NO: 347 - protein, 124 aa, synthetic construct
  REGION 1..124: note = Synthetic
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 348 - protein, 10 aa, synthetic construct
  REGION 1..10: note = Synthetic
GFSLSTSGVG

SEQ ID NO: 349 - protein, 7 aa, synthetic construct
  REGION 1..7: note = Synthetic
IYWNDHK

SEQ ID NO: 350 - protein, 16 aa, synthetic construct
  REGION 1..16: note = Synthetic
AHYSGSYSYYYYGLDV

SEQ ID NO: 351 - DNA, 321 bp, synthetic construct
  misc_feature 1..321: note = Synthetic
  source 1..321: mol_type = other DNA
gacatccagatgacccagtctccatcttccgtgtctgcatctgtaggagacagagtcaccatcacttgtcgggcgagtcagggtattgccagctggttagcctggtatcagcagaaaccagggaaagcccctgagctcctgatctatgctgcatccagtttgcaaggtggggtcccatcaaggttcagcggcagtggatctgggacagatttcactctcaccatcagcagcctgcagcctgaagattttgcaatttactattgtcaacaggctaactatttcccgtggacgttcggccaagggaccaaggtggaaatcaaa

SEQ ID NO: 352 - protein, 107 aa, synthetic construct
  REGION 1..107: note = Synthetic
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIK

SEQ ID NO: 353 - protein, 6 aa, synthetic construct
  REGION 1..6: note = Synthetic
QGIASW

SEQ ID NO: 354 - skipped

SEQ ID NO: 355 - protein, 9 aa, synthetic construct
  REGION 1..9: note = Synthetic
QQANYFPWT

SEQ ID NO: 356 - DNA, 363 bp, synthetic construct
  misc_feature 1..363: note = Synthetic
  source 1..363: mol_type = other DNA
gaggtgcagctgttggagtctgggggagccttggtacagcctggggggtccctgagactctcctgtgcagcctctggattcacctttacctcctatgccatgcactgggtccgccaggctccagggaagggtctggagtgggtctcatctattagaggtagtggtggtggcacatactccgcagactccgtgaagggccggttcaccatctccagagacaattccagggacactctatatctgcaaatgaacagtgtgagagccgaggacacggccgtttattactgtgcgaggtcccatgactacggtgccttcgacttctttgactactggggccagggaaccctggtcaccgtctcctca

SEQ ID NO: 357 - protein, 121 aa, synthetic construct
  REGION 1..121: note = Synthetic
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 358 - protein, 8 aa, synthetic construct
  REGION 1..8: note = Synthetic
GFTFTSYA

SEQ ID NO: 359 - protein, 8 aa, synthetic construct
  REGION 1..8: note = Synthetic
IRGSGGGT

SEQ ID NO: 360 - protein, 14 aa, synthetic construct
  REGION 1..14: note = Synthetic
ARSHDYGAFDFFDY

SEQ ID NO: 361 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagggcattagaactgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcggcctgaagattttgcaactttttactgtctacagtataatagttacccgctcactttcggcggagggaccaaggtggagatcaaa

SEQ ID NO: 362 - protein, 107 aa, synthetic construct
  REGION 1..107: note = Synthetic
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIK

SEQ ID NO: 363 - protein, 6 aa, synthetic construct
  REGION 1..6: note = Synthetic
QGIRTD

SEQ ID NO: 364 - skipped

SEQ ID NO: 365 - protein, 9 aa, synthetic construct
  REGION 1..9: note = Synthetic
LQYNSYPLT

SEQ ID NO: 366 - DNA, 348 bp, synthetic construct
  misc_feature 1..348: note = Synthetic
  source 1..348: mol_type = other DNA
caggtgcagctggtggagtctgggggaggcttggtcaagcctggagggtccctgagactctcctgtgcagcctctggattcaccttcagtgactacttcatgagctggatccgccaggctccagggaaggggctggagtgggtttcatacattagtagtactggtagtaccataaattatgcagactctgtgaagggccgattcaccatctccagggacaatgtcaagaattcactgtatctgcaaatgaccagcctgagagtcgaggacacggccgtgtattactgtacgagagataactggaactatgaatactggggccagggaaccctggtcaccgtctcctca

SEQ ID NO: 367 - protein, 116 aa, synthetic construct
  REGION 1..116: note = Synthetic
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 368 - protein, 8 aa, synthetic construct
  REGION 1..8: note = Synthetic
GFTFSDYF

SEQ ID NO: 369 - protein, 8 aa, synthetic construct
  REGION 1..8: note = Synthetic
ISSTGSTI

SEQ ID NO: 370 - protein, 9 aa, synthetic construct
  REGION 1..9: note = Synthetic
TRDNWNYEY

SEQ ID NO: 371 - DNA, 321 bp, synthetic construct
  misc_feature 1..321: note = Synthetic
  source 1..321: mol_type = other DNA
gaaatagtgatgacgcagtctccagccaccctgtctgtgtctccaggggaaagagccaccctctcctgcagggccagtcagagtgttagcatcaacttagcctggtaccagcagaaacctggccaggctcccaggctcctcatctttgttgcatccaccagggccactggtatcccagccaggttcagtggcagtgggtctgggacagagttcactctcaccatcagcagcctgcagtctgaagattttgcaacttattactgtcagcagtatgatatctggccgtacacttttggccaggggaccaagctggagatcaaa

SEQ ID NO: 372 - protein, 107 aa, synthetic construct
  REGION 1..107: note = Synthetic
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIK

SEQ ID NO: 373 - protein, 6 aa, synthetic construct
  REGION 1..6: note = Synthetic
QSVSIN

SEQ ID NO: 374 - skipped

SEQ ID NO: 375 - protein, 9 aa, synthetic construct
  REGION 1..9: note = Synthetic
QQYDIWPYT

SEQ ID NO: 376 - DNA, 369 bp, synthetic construct
  misc_feature 1..369: note = Synthetic
  source 1..369: mol_type = other DNA
cagctgcagctgcaggagtcgggcccaggactggtgaagccttcggagaccctgtccctcacctgcactgtgtctggtgaatccatcagcagtaatacttactactggggctggatccgccagcccccagggaaggggctggaatggattgggagtatcgattatagtgggaccaccaattataacccgtccctcaagagtcgagtcaccatatccgtagacacgtccaggaatcacttctccctgaggctgaggtctgtgaccgccgcagacacggctgtgtattactgtgcgagagagtggggaaactacggctactattacggtatggacgtttggggccaagggaccacggtcaccgtctcctca

SEQ ID NO: 377 - protein, 123 aa, synthetic construct
  REGION 1..123: note = Synthetic
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 378 - protein, 10 aa, synthetic construct
  REGION 1..10: note = Synthetic
GESISSNTYY

SEQ ID NO: 379 - protein, 7 aa, synthetic construct
  REGION 1..7: note = Synthetic
IDYSGTT

SEQ ID NO: 380 - protein, 15 aa, synthetic construct
  REGION 1..15: note = Synthetic
AREWGNYGYYYGMDV

SEQ ID NO: 381 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcaattgccgggcaagtcagggcattagaaatgatttaggctggtatcagcagaaaccagggaaagcccctaagcgcctgatctatgctgcatccagtttgcaaagtggggtcccattaaggttcagtggcagtggatctgggacagaattcactctcacaatcaacaacctgcagcctgaagattttgcaacttattactgtctatcgcataatagttacccgtggacgttcggccaagggaccaaggtggaaatcaaa

SEQ ID NO: 382 - protein, 107 aa, synthetic construct
  REGION 1..107: note = Synthetic
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIK

SEQ ID NO: 383 - protein, 6 aa, synthetic construct
  REGION 1..6: note = Synthetic
QGIRND

SEQ ID NO: 384 - skipped

SEQ ID NO: 385 - protein, 9 aa, synthetic construct
  REGION 1..9: note = Synthetic
LSHNSYPWT

SEQ ID NO: 386 - DNA, 354 bp, synthetic construct
  misc_feature 1..354: note = Synthetic
  source 1..354: mol_type = other DNA
caggtgcagctggtgcagtctggggctgaggtgaagaagcctgggtcctcggtgagggtctcctgcaaggcttctagaggcaccttcagcagctatgctatcagctgggtgcgacaggcccctggacaaggccttgagtggatgggagggatcatccccatctttggtacagcaaactacgcacagaagttcctggccagagtcacgattaccgcggacgaatccacgagcacagcctacatggagctgagcagcctgagatctgaggacacggccgtgtattactgtgcgagagagaaggggtggaactactttgactactggggccagggaaccctggtcaccgtctcctca

SEQ ID NO: 387 - protein, 118 aa, synthetic construct
  REGION 1..118: note = Synthetic
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 388 - protein, 8 aa, synthetic construct
  REGION 1..8: note = Synthetic
RGTFSSYA

SEQ ID NO: 389 - protein, 8 aa, synthetic construct
  REGION 1..8: note = Synthetic
IIPIFGTA

SEQ ID NO: 390 - protein, 11 aa, synthetic construct
  REGION 1..11: note = Synthetic
AREKGWNYFDY

SEQ ID NO: 391 - DNA, 321 bp, synthetic construct
  misc_feature 1..321: note = Synthetic
  source 1..321: mol_type = other DNA
gacatccagatgacccagtctccaccttccgtgtctgcatctgtaggagacagagtcaccatcacttgtcgggcgagtcagggtattagcagctggttagcctggtatcagcagaaaccagggaaagcccctaaactcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctcggacgttcggccaagggaccaaggtggaaatcaaa

SEQ ID NO: 392 - protein, 107 aa, synthetic construct
  REGION 1..107: note = Synthetic
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIK

SEQ ID NO: 393 - protein, 6 aa, synthetic construct
  REGION 1..6: note = Synthetic
QGISSW

SEQ ID NO: 394 - skipped

SEQ ID NO: 395 - protein, 9 aa, synthetic construct
  REGION 1..9: note = Synthetic
QQANSFPRT

SEQ ID NO: 396 - DNA, 369 bp, synthetic construct
  misc_feature 1..369: note = Synthetic
  source 1..369: mol_type = other DNA
caggtgcagctggtggagtctgggggaggcttggtcaagcctggagggtccctgagactctcctgtgcagcctctggattcaccttcagtgactactacatgaactggatccgccaggctccagggaaggggctggagtgggtttcatacattagtagtagtgggactaccatatactacgcagactctgtgaagggccgattcaccatctccagggacaacgccaagaaatcactgtatctggagatgaacagcctcagagccgaggacacggccgtgtactactgtgcgagagaggggtacggtaatgactactattactacggtatagacgtctggggccaagggaccacggtcaccgtctcctca

SEQ ID NO: 397 - protein, 123 aa, synthetic construct
  REGION 1..123: note = Synthetic
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 398 - protein, 8 aa, synthetic construct
  REGION 1..8: note = Synthetic
GFTFSDYY

SEQ ID NO: 399 - protein, 8 aa, synthetic construct
  REGION 1..8: note = Synthetic
ISSSGTTI

SEQ ID NO: 400 - protein, 16 aa, synthetic construct
  REGION 1..16: note = Synthetic
AREGYGNDYYYYGIDV

SEQ ID NO: 401 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatggtaatggatacaactatttgacttggtacctgcagaagccagggcagtctccacagctcctgatctatttgggttctaatcgggcctccggggtccctgacaggttcagtggcagtggatcaggcacagattttacactgaaaataagcagagtggaggctgaggatgttggggtttattactgcatgcaagctctacaaactccgtacacttttggccaggggaccaagctggagatcaaa

SEQ ID NO: 402 - protein, 112 aa, synthetic construct
  REGION 1..112: note = Synthetic
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIK

SEQ ID NO: 403 - protein, 11 aa, synthetic construct
  REGION 1..11: note = Synthetic
QSLLHGNGYNY

SEQ ID NO: 404 - skipped

SEQ ID NO: 405 - protein, 9 aa, synthetic construct
  REGION 1..9: note = Synthetic
MQALQTPYT

SEQ ID NO: 406 - DNA, 366 bp, synthetic construct
  misc_feature 1..366: note = Synthetic
  source 1..366: mol_type = other DNA
caggtgcagctggtggagtctgggggaggcgtggtccagcctgggaggtccctgagactctcctgtgcagcctctggattcaccttcagtagctttggcatgcactgggtccgccaggctccaggcaaggggctggagtgggtgatatttatatcatatgatggaagtgataaatactatgcagactccgtgaagggccgattcgccatctccagagacagttccaagaacacgctatatctgcaaatgaacagcctgagagctgaggacacggctgtgtattactgtgcgaaagaaaacggtattttgactgattcctacggtatggacgtctggggccaagggaccacggtcaccgtctcctca

SEQ ID NO: 407 - protein, 122 aa, synthetic construct
  REGION 1..122: note = Synthetic
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 408 - protein, 8 aa, synthetic construct
  REGION 1..8: note = Synthetic
GFTFSSFG

SEQ ID NO: 409 - protein, 8 aa, synthetic construct
  REGION 1..8: note = Synthetic
ISYDGSDK

SEQ ID NO: 410 - protein, 15 aa, synthetic construct
  REGION 1..15: note = Synthetic
AKENGILTDSYGMDV

SEQ ID NO: 411 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 412 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 413 - protein, 6 aa, synthetic construct
  REGION 1..6: note = Synthetic
QSISSY

SEQ ID NO: 414 - skipped

SEQ ID NO: 415 - protein, 10 aa, synthetic construct
  REGION 1..10: note = Synthetic
QQSYSTPPIT

SEQ ID NO: 416 - DNA, 372 bp, synthetic construct
  misc_feature 1..372: note = Synthetic
  source 1..372: mol_type = other DNA
caggttcagctggtgcagtctggagctgaggtgaagaagcctggggcctctgtgaaggtctcctgcaaggcttctggttacacctttaccagctatggtatcagctgggtgcgacaggcccctggacaagggcttgagtggatgggatggatcagtgtttaccatggtaacacaaactatgcacagaagttccagggcagagtcaccatgaccacagacacatccacgagcacagcctacatggagctgaggagcctgagatctgacgacacggccgtgtattactgtgcgagagaggggtattacgatttttggagtggttattacccttttgactactggggccagggaaccctggtcaccgtctcctca

SEQ ID NO: 417 - protein, 124 aa, synthetic construct
  REGION 1..124: note = Synthetic
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 418 - protein, 8 aa, synthetic construct
  REGION 1..8: note = Synthetic
GYTFTSYG

SEQ ID NO: 419 - protein, 8 aa, synthetic construct
  REGION 1..8: note = Synthetic
ISVYHGNT

SEQ ID NO: 420 - protein, 17 aa, synthetic construct
  REGION 1..17: note = Synthetic
AREGYYDFWSGYYPFDY

SEQ ID NO: 421 - DNA, 324 bp, synthetic construct; identical to SEQ ID NO: 411
  misc_feature 1..324: note = Synthetic
  source 1..324: mol_type = other DNA

SEQ ID NO: 422 - protein, 108 aa, synthetic construct; identical to SEQ ID NO: 412
  REGION 1..108: note = Synthetic

SEQ ID NO: 423 - protein, 6 aa, synthetic construct
  REGION 1..6: note = Synthetic
QSISSY

SEQ ID NO: 424 - skipped

SEQ ID NO: 425 - protein, 10 aa, synthetic construct
  REGION 1..10: note = Synthetic
QQSYSTPPIT

SEQ ID NO: 426 - DNA, 354 bp, synthetic construct
  misc_feature 1..354: note = Synthetic
  source 1..354: mol_type = other DNA
gaggtgcagctggtggagtctgggggaggcttgatacaacctggagggtccctgagactctcctgtgaagcctctggattcaccttcagaaattatgaaatgaattgggtccgccaggctccagggaaggggctggagtgggtttcatatattagtagtagtggtaatatgaaagactacgcagagtctgtgaagggccgattcaccatctccagagacaatgtcaagaattcactgcagctgcaaatgaacagcctgagagtcgaggacacggctgtttattactgtgcgagagacgagtttccttacggaatggacgtctggggccaagggaccacggtcaccgtctcctca

SEQ ID NO: 427 - protein, 118 aa, synthetic construct
  REGION 1..118: note = Synthetic
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 428 - protein, 8 aa, synthetic construct
  REGION 1..8: note = Synthetic
GFTFRNYE

SEQ ID NO: 429 - protein, 8 aa, synthetic construct
  REGION 1..8: note = Synthetic
ISSSGNMK

SEQ ID NO: 430 - protein, 11 aa, synthetic construct
  REGION 1..11: note = Synthetic
ARDEFPYGMDV

SEQ ID NO: 431 - DNA, 324 bp, synthetic construct; identical to SEQ ID NO: 411
  misc_feature 1..324: note = Synthetic
  source 1..324: mol_type = other DNA

SEQ ID NO: 432 - protein, 108 aa, synthetic construct; identical to SEQ ID NO: 412
  REGION 1..108: note = Synthetic

SEQ ID NO: 433 - protein, 6 aa, synthetic construct
  REGION 1..6: note = Synthetic
QSISSY

SEQ ID NO: 434 - skipped

SEQ ID NO: 435 - protein, 10 aa, synthetic construct
  REGION 1..10: note = Synthetic
QQSYSTPPIT

SEQ ID NO: 436 - DNA, 363 bp, synthetic construct
  misc_feature 1..363: note = Synthetic
  source 1..363: mol_type = other DNA
gaagtgcagctggtggagtctgggggaggcttggttcagcctggcaggtccctgagactctcctgtgcagcctctggattcacctttgatgattatgccatgaactgggtccggcaagctccagggaagggcctggagtgggtctcaggtattagttggagtagtggtagcatggactatgcggactctgtgaagggccgattcaccatctccagagacaacgccaaaaactccctgtatctgcaaatgaacagtctgagaactgaggacacggccttatattactgtgcaaaagctagggaagttggagactactacggtatggacgtctggggccaagggaccacggtcaccgtctcctca

SEQ ID NO: 437 - protein, 121 aa, synthetic construct
  REGION 1..121: note = Synthetic
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 438 - protein, 8 aa, synthetic construct
  REGION 1..8: note = Synthetic
GFTFDDYA

SEQ ID NO: 439 - protein, 8 aa, synthetic construct
  REGION 1..8: note = Synthetic
ISWSSGSM

SEQ ID NO: 440 - protein, 14 aa, synthetic construct
  REGION 1..14: note = Synthetic
AKAREVGDYYGMDV

SEQ ID NO: 441 - DNA, 324 bp, synthetic construct; identical to SEQ ID NO: 411
  misc_feature 1..324: note = Synthetic
  source 1..324: mol_type = other DNA

SEQ ID NO: 442 - protein, 108 aa, synthetic construct; identical to SEQ ID NO: 412
  REGION 1..108: note = Synthetic

SEQ ID NO: 443 - protein, 6 aa, synthetic construct
  REGION 1..6: note = Synthetic
QSISSY

SEQ ID NO: 444 - skipped

SEQ ID NO: 445 - protein, 10 aa, synthetic construct
  REGION 1..10: note = Synthetic
QQSYSTPPIT

SEQ ID NO: 446 - DNA, 357 bp, synthetic construct
  misc_feature 1..357: note = Synthetic
  source 1..357: mol_type = other DNA
gaagtgcagctggtggagtctgggggaggcttggtacagcctggcaggtccctgacactctcctgtgcagcctctggattcacctttgataattttggcatgcactgggtccggcaaggtccagggaagggcctggaatgggtctcaggtcttacttggaatagtggtgtcataggctatgcggactctgtgaagggccgattcaccatctccagagacaacgccaagaactccctgtatctgcaaatgaacagtctgagacctgaggacacggccttatattactgtgcaaaagatatacggaattacggcccctttgactactggggccagggaaccctggtcaccgtctcctca

SEQ ID NO: 447 - protein, 119 aa, synthetic construct
  REGION 1..119: note = Synthetic
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 448 - protein, 8 aa, synthetic construct
  REGION 1..8: note = Synthetic
GFTFDNFG

SEQ ID NO: 449 - protein, 8 aa, synthetic construct
  REGION 1..8: note = Synthetic
LTWNSGVI

SEQ ID NO: 450 - protein, 12 aa, synthetic construct
  REGION 1..12: note = Synthetic
AKDIRNYGPFDY

SEQ ID NO: 451 - DNA, 324 bp, synthetic construct
  misc_feature 1..324: note = Synthetic
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 452 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 453 - protein, 7 aa, synthetic construct
  REGION 1..7: note = Synthetic
QSVSSSY

SEQ ID NO: 454 - skipped

SEQ ID NO: 455 - protein, 9 aa, synthetic construct
  REGION 1..9: note = Synthetic
QQYGSSPWT

SEQ ID NO: 456 - DNA, 357 bp, synthetic construct
  misc_feature 1..357: note = Synthetic
  source 1..357: mol_type = other DNA
gaggtgcagctggtggagtctgggggaggcttagtacagcctggagggtccctaagactctcctgtgcagcctctggattcaccttcaatatttttgaaatgaactgggtccgccaggctccagggaaggggctggagtggatttcctacattagtagtcgtggaactaccacatactacgcagactctgtgaggggccgattcaccatctccagagacaacgccaagaactcactgtatctgcaaatgaacagcctgagagccgaggacacggctgtttattactgtgcgagagattatgaagcaacaatcccttttgacttctggggccagggaaccctggtcaccgtctcctca

SEQ ID NO: 457 - protein, 119 aa, synthetic construct
  REGION 1..119: note = Synthetic
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 458 - protein, 8 aa, synthetic construct
  REGION 1..8: note = Synthetic
GFTFNIFE

SEQ ID NO: 459 - protein, 8 aa, synthetic construct
  REGION 1..8: note = Synthetic
ISSRGTTT

SEQ ID NO: 460 - protein, 12 aa, synthetic construct
  REGION 1..12: note = Synthetic
ARDYEATIPFDF

SEQ ID NO: 461 - DNA, 324 bp, synthetic construct; identical to SEQ ID NO: 411
  misc_feature 1..324: note = Synthetic
  source 1..324: mol_type = other DNA

SEQ ID NO: 462 - protein, 108 aa, synthetic construct; identical to SEQ ID NO: 412
  REGION 1..108: note = Synthetic

SEQ ID NO: 463 - protein, 6 aa, synthetic construct
  REGION 1..6: note = Synthetic
QSISSY

SEQ ID NO: 464 - skipped

SEQ ID NO: 465 - protein, 10 aa, synthetic construct
  REGION 1..10: note = Synthetic
QQSYSTPPIT

SEQ ID NO: 466 - DNA, 363 bp, synthetic construct
  misc_feature 1..363: note = Synthetic
  source 1..363: mol_type = other DNA
gaggtgcagctggtggagtctgggggaagtgtggtacggcctggggggtccctgagactctcctgtgaagcctctggattcacctttgatgattatggcatgagctgggtccgccaagatccagggaaggggctggagtgggtctctggtattaattggaatggtgatagaacaaattatgcagactctgtgaagggccgattcatcatttccagagacaacgccaagaactctgtgtatctacaaatgaacagtctgagagcggaggactcggccttgtatcactgtgcgagagatcagggactcggagtggcagctacccttgactactggggccagggaaccctggtcaccgtctcctca

SEQ ID NO: 467 - protein, 121 aa, synthetic construct
  REGION 1..121: note = Synthetic
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 468 - protein, 8 aa, synthetic construct
  REGION 1..8: note = Synthetic
GFTFDDYG

SEQ ID NO: 469 - protein, 8 aa, synthetic construct
  REGION 1..8: note = Synthetic
INWNGDRT

SEQ ID NO: 470 - protein, 14 aa, synthetic construct
  REGION 1..14: note = Synthetic
ARDQGLGVAATLDY

SEQ ID NO: 471 - DNA, 324 bp, synthetic construct; identical to SEQ ID NO: 411
  misc_feature 1..324: note = Synthetic
  source 1..324: mol_type = other DNA

SEQ ID NO: 472 - protein, 108 aa, synthetic construct; identical to SEQ ID NO: 412
  REGION 1..108: note = Synthetic

SEQ ID NO: 473 - protein, 6 aa, synthetic construct
  REGION 1..6: note = Synthetic
QSISSY

SEQ ID NO: 474 - skipped

SEQ ID NO: 475 - protein, 10 aa, synthetic construct
  REGION 1..10: note = Synthetic
QQSYSTPPIT

SEQ ID NO: 476 - DNA, 384 bp, synthetic construct
  misc_feature 1..384: note = Synthetic
  source 1..384: mol_type = other DNA
gaggtgcagctggtggagtctgggggaggcttggtacagcctggagggtccctgagactctcctgtgcagcctctggattcaccgtcagtaattatgaaatgaactgggtccgccaggctccagggaaggggctggagtgggtttcatacattagtagtagtaccagtaacatatactacgcagactctgtgaagggccgattcaccatctccagagacaacgccgagaactcactgtatctgcagatgaacagcctgagagtcgaggacacggctgtttattactgtgtgagagatgggattgtagtagttccagttggtcgtggatactactattacggtttggacgtctggggccaagggaccacggtcaccgtctcctca

SEQ ID NO: 477 - protein, 128 aa, synthetic construct
  REGION 1..128: note = Synthetic
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSS

SEQ ID NO: 478 - protein, 8 aa, synthetic construct
  REGION 1..8: note = Synthetic
GFTVSNYE

SEQ ID NO: 479 - protein, 8 aa, synthetic construct
  REGION 1..8: note = Synthetic
ISSSTSNI

SEQ ID NO: 480 - protein, 21 aa, synthetic construct
  REGION 1..21: note = Synthetic
VRDGIVVVPVGRGYYYYGLDV

SEQ ID NO: 481 - DNA, 324 bp, synthetic construct; identical to SEQ ID NO: 411
  misc_feature 1..324: note = Synthetic
  source 1..324: mol_type = other DNA

SEQ ID NO: 482 - protein, 108 aa, synthetic construct; identical to SEQ ID NO: 412
  REGION 1..108: note = Synthetic

SEQ ID NO: 483 - protein, 6 aa, synthetic construct
  REGION 1..6: note = Synthetic
QSISSY

SEQ ID NO: 484 - skipped

SEQ ID NO: 485 - protein, 10 aa, synthetic construct
  REGION 1..10: note = Synthetic
QQSYSTPPIT

SEQ ID NO: 486 - DNA, 378 bp, synthetic construct
  misc_feature 1..378: note = Synthetic
  source 1..378: mol_type = other DNA
caggtgcagctggtggagtctgggggaggcgtggtccagcctggaaggtccctgagactctcctgcgcagcctctggattcccctttagtaattatgtcatgtattgggtccgccaggctccaggcaaggggctggagtgggtggctcttattttttttgacggaaagaaaaactatcatgcagactccgtgaagggccgattcaccataaccagagacaattccaaaaatatgttatatctgcaaatgaacagcctgagacctgaggacgcggctgtgtattactgtgcgaaaatccattgtcctaatggtgtatgttacaaggggtattacggaatggacgtctggggccaagggaccacggtcaccgtctcctca

SEQ ID NO: 487 - protein, 126 aa, synthetic construct
  REGION 1..126: note = Synthetic
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 488 - protein, 8 aa, synthetic construct
  REGION 1..8: note = Synthetic
GFPFSNYV

SEQ ID NO: 489 - protein, 8 aa, synthetic construct
  REGION 1..8: note = Synthetic
IFFDGKKN

SEQ ID NO: 490 - protein, 19 aa, synthetic construct
  REGION 1..19: note = Synthetic
AKIHCPNGVCYKGYYGMDV

SEQ ID NO: 491 - DNA, 324 bp, synthetic construct; identical to SEQ ID NO: 411
  misc_feature 1..324: note = Synthetic
  source 1..324: mol_type = other DNA

SEQ ID NO: 492 - protein, 108 aa, synthetic construct; identical to SEQ ID NO: 412
  REGION 1..108: note = Synthetic

SEQ ID NO: 493 - protein, 6 aa, synthetic construct
  REGION 1..6: note = Synthetic
QSISSY

SEQ ID NO: 494 - skipped

SEQ ID NO: 495 - protein, 10 aa, synthetic construct
  REGION 1..10: note = Synthetic
QQSYSTPPIT

SEQ ID NO: 496 - DNA, 375 bp, synthetic construct
  misc_feature 1..375: note = Synthetic
  source 1..375: mol_type = other DNA
gaggtgcagctggtggagtctgggggaggcttggtccagcctggggggtccctaagactctcctgtgcagcctctggattcacctttagtaactattggatgaactgggtccgccaggctccagggaagggactggagtgggtggccaatataaaagaagatggaggtaagaaattgtatgtggactctgtgaagggccgattcaccatctccagagacaacgccaagaactcactgtttctgcaaatgaacagcctgagagccgaggacacggctgtgtattattgtgcgagagaagatacaactttggttgtggactactactactacggtatggacgtctggggccaagggaccacggtcaccgtctcctca

SEQ ID NO: 497 - protein, 125 aa, synthetic construct
  REGION 1..125: note = Synthetic
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 498 - protein, 8 aa, synthetic construct
  REGION 1..8: note = Synthetic
GFTFSNYW

SEQ ID NO: 499 - protein, 8 aa, synthetic construct
  REGION 1..8: note = Synthetic
IKEDGGKK

SEQ ID NO: 500 - protein, 18 aa, synthetic construct
  REGION 1..18: note = Synthetic
AREDTTLVVDYYYYGMDV

SEQ ID NO: 501 - DNA, 324 bp, synthetic construct; identical to SEQ ID NO: 411
  misc_feature 1..324: note = Synthetic
  source 1..324: mol_type = other DNA

SEQ ID NO: 502 - protein, 108 aa, synthetic construct; identical to SEQ ID NO: 412
  REGION 1..108: note = Synthetic

SEQ ID NO: 503 - protein, 6 aa, synthetic construct
  REGION 1..6: note = Synthetic
QSISSY

SEQ ID NO: 504 - skipped

SEQ ID NO: 505 - protein, 10 aa, synthetic construct
  REGION 1..10: note = Synthetic
QQSYSTPPIT

SEQ ID NO: 506 - DNA, 366 bp, synthetic construct
  misc_feature 1..366: note = Synthetic
  source 1..366: mol_type = other DNA
caggtccagctggtgcagtctggggctgaggtgaagaagcctgggtcctcggtgaaggtctcctgcaaggcttctggaggcaccttcaacacctatgctatcacctgggtgcgacaggcccctggacaagggcttgaatggatggggggaatcatccctatctctggcatagcagagtacgcacagaagttccagggcagagtcacgatcaccacggatgactcctcgaccacagcctacatggaactgaacagtctgagatctgaggacacggccgtgtattactgtgcgagctggaactacgcactctactacttctacggtatggacgtctggggccgagggaccacggtcaccgtctcctca

SEQ ID NO: 507 - protein, 122 aa, synthetic construct
  REGION 1..122: note = Synthetic
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 508 - protein, 8 aa, synthetic construct
  REGION 1..8: note = Synthetic
GGTFNTYA

SEQ ID NO: 509 - protein, 8 aa, synthetic construct
  REGION 1..8: note = Synthetic
IIPISGIA

SEQ ID NO: 510 - protein, 15 aa, synthetic construct
  REGION 1..15: note = Synthetic
ASWNYALYYFYGMDV

SEQ ID NO: 511 - DNA, 324 bp, synthetic construct; identical to SEQ ID NO: 451
  misc_feature 1..324: note = Synthetic
  source 1..324: mol_type = other DNA

SEQ ID NO: 512 - protein, 108 aa, synthetic construct; identical to SEQ ID NO: 452
  REGION 1..108: note = Synthetic

SEQ ID NO: 513 - protein, 7 aa, synthetic construct
  REGION 1..7: note = Synthetic
QSVSSSY

SEQ ID NO: 514 - skipped

SEQ ID NO: 515 - protein, 9 aa, synthetic construct
  REGION 1..9: note = Synthetic
QQYGSSPWT

SEQ ID NO: 516 - DNA, 369 bp, synthetic construct
  misc_feature 1..369: note = Synthetic
  source 1..369: mol_type = other DNA
caggtgcagctggtggagtctgggggaggcttggtcaagcctggagggtccctgagactctcctgtgcagcctctggattcaccttcagtgtctattacatgaactggatccgccaggctccagggaagggcctggagtgggtttcatacattagtagtagtggtagtaccatatactacgcagactctgtgaagggccgattcaccatctccagggacaacgccaagaactcactgtatctccaaatgaacagtctgagagccgaggacacggccgtatattactgtgggagagaagggtatagtgggacttattcttattacggtatggacgtctggggccaagggaccacggtcaccgtctcctca

SEQ ID NO: 517 - protein, 123 aa, synthetic construct
  REGION 1..123: note = Synthetic
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 518 - protein, 8 aa, synthetic construct
  REGION 1..8: note = Synthetic
GFTFSVYY

SEQ ID NO: 519 - protein, 8 aa, synthetic construct
  REGION 1..8: note = Synthetic
ISSSGSTI

SEQ ID NO: 520 - protein, 16 aa, synthetic construct
  REGION 1..16: note = Synthetic
GREGYSGTYSYYGMDV

SEQ ID NO: 521 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcaacagagtggaggctgaggatgttggggtttattactgcatgcaagctctacaaactccgtacacttttggccaggggaccaagctggagatcaaa

SEQ ID NO: 522 - protein, 112 aa, synthetic construct
  REGION 1..112: note = Synthetic
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIK

SEQ ID NO: 523 - protein, 11 aa, synthetic construct
  REGION 1..11: note = Synthetic
QSLLHSNGYNY

SEQ ID NO: 524 - skipped

SEQ ID NO: 525 - protein, 9 aa, synthetic construct
  REGION 1..9: note = Synthetic
MQALQTPYT

SEQ ID NO: 526 - DNA, 366 bp, synthetic construct
  misc_feature 1..366: note = Synthetic
  source 1..366: mol_type = other DNA
gaagtgcagctggtggagtctgggggagggttggtacagcctggcaggtccctgagactctcctgtgcagtctctggattcacctttgatgattatgccatgcactgggtccggcaagctccagggaagggcctggagtgggtctcaggtattagttggaatagtggtaccagaggatatgcggactctgtgaagggccgattcaccatctccagagacaacgccaagaactccctgtatctgcaaatgaacagtctgagaggtgaggacacggccttgtattactgtgtaaaagatattacgatatcccccaactactacggtatggacgtctggggccaagggaccacggtcaccgtctcctca

SEQ ID NO: 527 - protein, 122 aa, synthetic construct
  REGION 1..122: note = Synthetic
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 528 - protein, 8 aa, synthetic construct
  REGION 1..8: note = Synthetic
GFTFDDYA

SEQ ID NO: 529 - protein, 8 aa, synthetic construct
  REGION 1..8: note = Synthetic
ISWNSGTR

SEQ ID NO: 530 - protein, 15 aa, synthetic construct
  REGION 1..15: note = Synthetic
VKDITISPNYYGMDV

SEQ ID NO: 531 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcgagtcaggacattagccattattcagcctggtatcagcagaaaccagggaaacttcctaacctcctgatctatgctgcatccactttgcaatcaggggtcccatctcggttcagtggcagtggatctgggacagatttctctctcaccaccagcagcctgcagcctgaagatgttgcaacttattactgtcaaaagtataacagtgtccctctcactttcggcggagggaccaaggtggagatcaaa

SEQ ID NO: 532 - protein, 107 aa, synthetic construct
  REGION 1..107: note = Synthetic
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIK

SEQ ID NO: 533 - protein, 6 aa, synthetic construct
  REGION 1..6: note = Synthetic
QDISHY

SEQ ID NO: 534 - skipped

SEQ ID NO: 535 - protein, 9 aa, synthetic construct
  REGION 1..9: note = Synthetic
QKYNSVPLT

SEQ ID NO: 536 - protein, 118 aa, synthetic construct
  REGION 1..118: note = Synthetic
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 537 - protein, 107 aa, synthetic construct
  REGION 1..107: note = Synthetic
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 538 - protein, 243 aa, synthetic construct
  REGION 1..243: note = Synthetic
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKGGGGSGGGGSGGGGSEVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 539 - protein, 245 aa, synthetic construct
  REGION 1..245: note = Synthetic
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKGGGGSGGGGSGGGGSEVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 540 - protein, 246 aa, synthetic construct
  REGION 1..246: note = Synthetic
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 541 - protein, 243 aa, synthetic construct
  REGION 1..243: note = Synthetic
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKGGGGSGGGGSGGGGSEVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 542 - protein, 238 aa, synthetic construct
  REGION 1..238: note = Synthetic
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKGGGGSGGGGSGGGGSQVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 543 - protein, 245 aa, synthetic construct
  REGION 1..245: note = Synthetic
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKGGGGSGGGGSGGGGSQLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 544 - protein, 240 aa, synthetic construct
  REGION 1..240: note = Synthetic
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKGGGGSGGGGSGGGGSQVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 545 - protein, 250 aa, synthetic construct
  REGION 1..250: note = Synthetic
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKGGGGSGGGGSGGGGSQVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 546 - protein, 245 aa, synthetic construct
  REGION 1..245: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 547 - protein, 247 aa, synthetic construct
  REGION 1..247: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 548 - protein, 241 aa, synthetic construct
  REGION 1..241: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 549 - protein, 249 aa, synthetic construct
  REGION 1..249: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 550 - protein, 248 aa, synthetic construct
  REGION 1..248: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 551 - protein, 242 aa, synthetic construct
  REGION 1..242: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 552 - protein, 244 aa, synthetic construct
  REGION 1..244: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 553 - protein, 251 aa, synthetic construct
  REGION 1..251: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSS

SEQ ID NO: 554 - protein, 244 aa, synthetic construct
  REGION 1..244: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 555 - protein, 242 aa, synthetic construct
  REGION 1..242: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 556 - protein, 245 aa, synthetic construct
  REGION 1..245: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 557 - protein, 243 aa, synthetic construct
  REGION 1..243: note = Synthetic
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKGGGGSGGGGSGGGGSEVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 558 - protein, 243 aa, synthetic construct
  REGION 1..243: note = Synthetic
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKGGGGSGGGGSGGGGSEVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 559 - protein, 250 aa, synthetic construct
  REGION 1..250: note = Synthetic
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKGGGGSGGGGSGGGGSQVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 560 - protein, 244 aa, synthetic construct
  REGION 1..244: note = Synthetic
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKGGGGSGGGGSGGGGSEVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 561 - protein, 239 aa, synthetic construct
  REGION 1..239: note = Synthetic
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 562 - protein, 247 aa, synthetic construct
  REGION 1..247: note = Synthetic
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKGGGGSGGGGSGGGGSEVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 563 - protein, 249 aa, synthetic construct
  REGION 1..249: note = Synthetic
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKGGGGSGGGGSGGGGSEVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 564 - protein, 238 aa, synthetic construct
  REGION 1..238: note = Synthetic
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKGGGGSGGGGSGGGGSEVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 565 - protein, 247 aa, synthetic construct
  REGION 1..247: note = Synthetic
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKGGGGSGGGGSGGGGSEVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 566 - protein, 242 aa, synthetic construct
  REGION 1..242: note = Synthetic
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKGGGGSGGGGSGGGGSQVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 567 - protein, 242 aa, synthetic construct
  REGION 1..242: note = Synthetic
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKGGGGSGGGGSGGGGSQVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 568 - protein, 245 aa, synthetic construct
  REGION 1..245: note = Synthetic
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKGGGGSGGGGSGGGGSEVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 569 - protein, 244 aa, synthetic construct
  REGION 1..244: note = Synthetic
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKGGGGSGGGGSGGGGSQVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 570 - protein, 1139 aa, synthetic construct
  REGION 1..1139: note = Synthetic
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 571 - protein, 1142 aa, synthetic construct
  REGION 1..1142: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 572 - protein, 1135 aa, synthetic construct
  REGION 1..1135: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 573 - protein, 1137 aa, synthetic construct
  REGION 1..1137: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 574 - DNA, 3417 bp, synthetic construct
  misc_feature 1..3417: note = Synthetic
  source 1..3417: mol_type = other DNA
gacatccagatgacccagtctccatcttccgtgtctgcatctgtaggagacagagtcaccatcacttgtcgggcgagtcagggtattgccagctggttagcctggtatcagcagaaaccagggaaagcccctgagctcctgatctatgctgcatccagtttgcaaggtggggtcccatcaaggttcagcggcagtggatctgggacagatttcactctcaccatcagcagcctgcagcctgaagattttgcaatttactattgtcaacaggctaactatttcccgtggacgttcggccaagggaccaaggtggaaatcaaaggcggcggcggaagcggagggggaggatcaggaggaggcggatcccagatcaccttgaaggagtctggtcctacgctggtgaaacccacacagaccctcacgctgacctgcaccttctctgggttctcactcagcactagtggagtgggtgtggtctggatccgtcagccccccggaaaggccctggagtggcttgcactcatttattggaatgatcataagcggtacagcccatctctggggagcaggctcaccatcaccaaggacacctccaaaaaccaggtggtccttacaatgaccaacatggaccctgtggacacagccacatattactgtgcacactacagtgggagctattcctactactactatggtttggacgtctggggccaagggaccacggtcaccgtctcctcaggaggtggtggaagtggaggtggtggaagtgcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 575 - DNA, 3426 bp, synthetic construct
  misc_feature 1..3426: note = Synthetic
  source 1..3426: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaaggcggcggcggaagcggagggggaggatcaggaggaggcggatcccaggtgcagctggtggagtctgggggaggcgtggtccagcctggaaggtccctgagactctcctgcgcagcctctggattcccctttagtaattatgtcatgtattgggtccgccaggctccaggcaaggggctggagtgggtggctcttattttttttgacggaaagaaaaactatcatgcagactccgtgaagggccgattcaccataaccagagacaattccaaaaatatgttatatctgcaaatgaacagcctgagacctgaggacgcggctgtgtattactgtgcgaaaatccattgtcctaatggtgtatgttacaaggggtattacggaatggacgtctggggccaagggaccacggtcaccgtctcctcaggaggtggtggaagtggaggtggtggaagtgcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 576 - DNA, 3405 bp, synthetic construct
  misc_feature 1..3405: note = Synthetic
  source 1..3405: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaaggcggcggcggaagcggagggggaggatcaggaggaggcggatccgaggtgcagctggtggagtctgggggaggcttagtacagcctggagggtccctaagactctcctgtgcagcctctggattcaccttcaatatttttgaaatgaactgggtccgccaggctccagggaaggggctggagtggatttcctacattagtagtcgtggaactaccacatactacgcagactctgtgaggggccgattcaccatctccagagacaacgccaagaactcactgtatctgcaaatgaacagcctgagagccgaggacacggctgtttattactgtgcgagagattatgaagcaacaatcccttttgacttctggggccagggaaccctggtcaccgtctcctcaggaggtggtggaagtggaggtggtggaagtgcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 577 - DNA, 3411 bp, synthetic construct
  misc_feature 1..3411: note = Synthetic
  source 1..3411: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaaggcggcggcggaagcggagggggaggatcaggaggaggcggatccgaagtgcagctggtggagtctgggggaggcttggttcagcctggcaggtccctgagactctcctgtgcagcctctggattcacctttgatgattatgccatgaactgggtccggcaagctccagggaagggcctggagtgggtctcaggtattagttggagtagtggtagcatggactatgcggactctgtgaagggccgattcaccatctccagagacaacgccaaaaactccctgtatctgcaaatgaacagtctgagaactgaggacacggccttatattactgtgcaaaagctagggaagttggagactactacggtatggacgtctggggccaagggaccacggtcaccgtctcctcaggaggtggtggaagtggaggtggtggaagtgcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 578 - DNA, 720 bp, synthetic construct
  misc_feature 1..720: note = Synthetic
  source 1..720: mol_type = other DNA
caggtgcaactacaggagtcgggcccaaaggtggtgaagccttcggagaccctgtccctcacctgcactgtctctggtggctccatcagtagttactactggaattggatccgccagtccccagggaagggactggagtggattggatataccaaaagagggtataccgactacaacccctccctcaggagtcgcgtcactatatcagaagacacgtccaagaaccagttctccctgaggatcagctctgtgaccgccgcagacacggccgtatattactgtgcacaaatggggtggggatcccatgcttttgacatgtggggccaagggacaatggtcgccgtctcttcaggaggaagtggtggaggcgggtcaggaggtggcgggagcggcggtgaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 579 - protein, 1128 aa, synthetic construct
  REGION 1..1128: note = Synthetic
  REGION 1..240: note = MISC_FEATURE - 12450 anti-CD63 scFv
  REGION 241..245: note = MISC_FEATURE - G4S linker
  REGION 246..1128: note = MISC_FEATURE - GAA (70-952)
QVQLQESGPKVVKPSETLSLTCTVSGGSISSYYWNWIRQSPGKGLEWIGYTKRGYTDYNPSLRSRVTISEDTSKNQFSLRISSVTAADTAVYYCAQMGWGSHAFDMWGQGTMVAVSSGGSGGGGSGGGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 580 - DNA, 3384 bp, synthetic construct
  misc_feature 1..3384: note = Synthetic
  misc_feature 1..720: note = 12450 anti-CD63 scFv
  misc_feature 721..735: note = G4S linker
  misc_feature 736..3384: note = GAA (70-952)
  source 1..3384: mol_type = other DNA
caggtgcaactacaggagtcgggcccaaaggtggtgaagccttcggagaccctgtccctcacctgcactgtctctggtggctccatcagtagttactactggaattggatccgccagtccccagggaagggactggagtggattggatataccaaaagagggtataccgactacaacccctccctcaggagtcgcgtcactatatcagaagacacgtccaagaaccagttctccctgaggatcagctctgtgaccgccgcagacacggccgtatattactgtgcacaaatggggtggggatcccatgcttttgacatgtggggccaagggacaatggtcgccgtctcttcaggaggaagtggtggaggcgggtcaggaggtggcgggagcggcggtgaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagggtggtggcggttcagcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 581 - DNA, 2649 bp, synthetic construct
  misc_feature 1..2649: note = Synthetic
  source 1..2649: mol_type = other DNA
gcacatccaggcaggcccagagcagttcccacacaatgtgatgtcccccccaactcaaggtttgattgtgcccctgataaggctatcactcaggaacagtgtgaagctaggggctgttgctacattcctgcaaagcagggactgcagggagcccagatggggcagccttggtgctttttcccaccatcttaccccagctacaaactagagaacctgagctcctctgaaatgggctacactgccactctaaccagaaccacccccactttctttcccaaggacatcctgaccctgagactagatgtgatgatggagactgagaataggctccactttactatcaaagatccagctaacaggaggtatgaggtgcctttggagacaccccatgtccacagcagagcaccctccccactctacagtgttgagtttagtgaggaaccttttggtgtgatagttaggagacagttggatggtagggtgctgttgaacacaactgtggcacccctgttctttgcagaccaattccttcagttgtccacctccctgccttcacagtatatcacaggcctggcagagcacctcagtcccttgatgctctcaacctcttggactagaatcaccctctggaacagggaccttgcacccacacctggtgcaaacctctatgggtctcaccctttttacttggcactggaagatggagggtctgcacatggggtgttcttactaaattcaaatgccatggatgtggtcctgcagccaagccctgcccttagctggaggagcacaggtgggatattggatgtctacattttcctgggcccagagccaaagtcagtggtgcaacagtacctggatgttgttggatatccctttatgcctccatactggggcctgggcttccacctgtgcaggtggggctacagttccacagctataaccagacaggtggtggagaacatgacaagagcccacttcccactggatgtccaatggaatgatctggattatatggattccaggagggacttcacatttaataaggatggcttcagggactttccagccatggtgcaggagctgcatcagggaggcaggagatatatgatgatagtggatcctgccatcagcagcagtggccctgcagggtcatacaggccctatgatgagggtctgaggaggggagtttttataaccaatgagacaggccaacccctgattgggaaagtatggccaggatccactgcttttccagacttcaccaaccccacagccttagcctggtgggaggacatggtggctgagttccatgaccaggttccctttgatggcatgtggattgatatgaatgaacctagtaactttataaggggctctgaagatggctgccccaataatgaactggagaatccaccctatgtgcctggggtggttggggggactctccaggcagccaccatatgtgcatcctcacatcagtttctctccacacactacaaccttcataacctctatggccttacagaagccatagcatcccatagggcactggttaaggctagggggacaaggccatttgtgatctccaggagtacttttgctggccatggcaggtatgctggccattggacaggggatgtgtggtcatcctgggagcagctggcctccagtgttccagaaatactgcagtttaatcttttgggggttcctctggttggggcagatgtatgtggctttctgggcaacacctcagaagaactgtgtgttaggtggacccagctaggtgccttctaccccttcatgaggaatcacaatagcctattaagtctgccacaggagccatattctttcagtgagccagcccaacaggctatgaggaaagccctcaccctgaggtatgcactcctcccccacctttacacattattccaccaggcccatgtggcaggagagacagtggccaggcccctcttccttgaattccccaaagattctagcacctggactgtggaccatcagctcctgtggggggaggcactgctcatcacaccagtgttacaggcagggaaggctgaagtgactggctattttccattgggcacatggtatgacctgcagactgtgccagtagaagcccttggcagcctcccacccccacctgcagctccaagggaaccagccatccacagtgaggggcaatgggtaacactgccagctcccctggacaccatcaatgtccaccttagggctgggtacattattcccttgcagggccctggcttaacaaccacagagtctagacaacagcctatggctctggctgtggccctgaccaagggtggagaggccaggggggagttgttctgggatgatggagaaagcctggaagtgctagagaggggggcctacacacaggtcatttttctggctaggaataatacaattgtgaatgaacttgtaagggtgacaagtgagggagctggcctacagctgcagaaggtgactgtcctgggagtggccacagcacctcagcaagtcctctctaatggtgtccctgtctccaatttcacttacagcccagacaccaaagtcctggacatttgtgtcagcctgttgatgggagagcagtttcttgtctcctggtgt

SEQ ID NO: 582 - DNA, 2649 bp, synthetic construct
  misc_feature 1..2649: note = Synthetic
  source 1..2649: mol_type = other DNA
gcacaccctgggagacccagggcagtacctacccagtgtgatgttcctcccaacagtagatttgactgtgctccagacaaggccatcacacaggaacaatgtgaggccagaggttgctgctacatccctgctaagcaagggctccagggggcccagatggggcagccttggtgcttcttcccacccagctacccctcctacaaactggagaacctgagctcctctgagatgggctacactgccaccctgactagaacaacacctacctttttccctaaggacatcctgactctgaggttagatgtgatgatggaaactgaaaacaggctgcacttcaccatcaaggaccctgccaacaggagatatgaagttcctctggagacacctcatgttcacagcagagccccttcacccctgtattctgtggaattctctgaagaaccatttggagttatagtgagaagacaactggatggcagagtgctcctgaacacaacagtggcccctctgttctttgctgaccagtttctgcagctgagcactagcctgccttcccagtatatcactggcctggctgagcaccttagccctctgatgctcagcacatcttggactagaataacactatggaacagagacctggctccaaccccaggtgctaatctgtatggcagtcatcctttctacctggctctggaggatggagggtctgcccatggtgtgttcctgctgaactcaaatgcaatggatgttgtgttacagcctagccctgcattgagctggagaagtacaggtggaattctggatgtctacatattcttggggcctgagcctaagtctgtagtgcaacagtacctggatgttgttggctacccatttatgcctccctactggggcctaggattccacctgtgcagatggggctactccagtactgccatcaccagacaggtggttgagaacatgaccagggcccactttcctttagatgtgcagtggaatgatctggactacatggatagcagaagggacttcaccttcaacaaggatggttttagagacttccctgccatggtccaggagttgcaccaggggggcagaagatacatgatgatagtagacccagctatcagcagttcaggcccagcagggagctacaggccctatgatgagggcctgagaagaggtgtcttcatcactaatgagacaggccagccactgattggcaaagtgtggcctggctcaactgctttccctgatttcacaaaccctacagccttagcatggtgggaggatatggtggctgaattccatgaccaggtgccatttgatggcatgtggattgacatgaatgagccaagcaacttcatcagaggctctgaggatggctgccccaacaatgaactggaaaaccctccctatgtcccaggagtggtgggaggaaccctgcaggcagctacaatctgtgcctcttcccaccagttcctaagcacccactacaacctccataacctgtatgggttgacagaggccatagcctcccacagagccctggtcaaggctagaggcactaggccatttgtcatctctagatctacctttgctggccatggcaggtatgcaggccactggacaggtgatgtgtggagcagctgggaacaacttgccagcagtgtacctgagatcctccaatttaacctgctgggtgttcccctagtgggagctgatgtatgtgggttcctgggcaatacatcagaagagctctgtgtaaggtggacccagctaggggccttttacccattcatgagaaaccacaactctctcctgagccttccacaagaaccttactctttctctgagcctgctcagcaagccatgaggaaagcactgacattaagatatgcactgctgcctcacctttacaccctgttccaccaggcacatgtggcaggagaaacagtagccagacccctgttcttagagtttcccaaggactcttcaacctggactgtggaccaccagctcctatggggagaggccctgttaatcacccctgtgctgcaggctggtaaggctgaagttacaggctactttcccttaggcacatggtatgatttgcagacagtccctgtggaggccctgggatctcttcctcctcccccagctgctcctagggagcctgcaatccactctgagggacagtgggtaaccctcccagctccactggacaccatcaatgtacaccttagggctggatacattatccccctgcaaggccctggcctgaccacaacagagagcaggcagcagcctatggccctagctgttgcccttaccaagggaggggaagccagaggagagctcttctgggatgatggagagagccttgaagtgcttgaaaggggggcctacacacaagtgatcttcctggccagaaacaacactattgtgaatgagttggtgagagtcacaagtgaaggggctggtcttcaactccagaaagtgactgtgttaggagttgcaacagcaccacagcaggttctctctaatggggtaccagtgtctaacttcacatacagccctgacaccaaggtgctggacatctgtgtgtctttattgatgggtgaacagttcctggtgtcttggtgc

SEQ ID NO: 583 - DNA, 2649 bp, synthetic construct
  misc_feature 1..2649: note = Synthetic
  source 1..2649: mol_type = other DNA
gcacaccctggcaggcccagagcagtgcccacacagtgtgatgtcccaccaaatagcaggtttgattgtgcccctgacaaggctattacccaggaacagtgtgaagccagaggctgttgctacatccctgcaaaacaggggttgcagggagcacagatggggcagccctggtgcttcttcccaccatcttacccaagctataagttagaaaatctgtcatcctctgaaatgggctacactgccaccttgacaaggaccacccccacattcttccccaaagacatcctgacccttaggctggatgtgatgatggagactgagaacaggctccactttactatcaaagatccagctaacaggaggtatgaggtgcccttggagactcctcatgtccattcaagggcaccttccccactctactcagtggagttttcagaggagccctttggggttattgtgaggagacagctggatggcagagtgctgctgaacactactgtggcccctctgttctttgctgaccagttccttcagctgtcaacttctctgccatcacagtatatcacaggcctagctgagcacctcagtcccctgatgctcagcaccagctggacaagaattactctttggaacagagaccttgcacccactccaggtgctaatctctatgggtctcaccctttctacctggccctagaggatgggggctcagcacatggggtgttcttgctaaatagcaatgccatggatgtggtcttacagccttctcctgcccttagctggaggtctacaggtggaatactggatgtctacatctttctgggcccagagcccaagtctgttgtgcagcaatacctggatgttgtaggatatccctttatgccaccatattggggactgggcttccatttatgcaggtggggctactcctccactgctattaccaggcaagtggttgaaaacatgacaagggcccactttcccctggatgtacagtggaatgatcttgattacatggactccaggagggactttacatttaataaagatggcttcagggatttccctgccatggtgcaggagctgcaccagggaggcaggaggtacatgatgatagtggatcctgccatcagcagctcaggccctgctgggtcatataggccttatgatgaaggtcttaggaggggggtttttattaccaatgaaactggccaaccacttattggcaaggtatggcctggttcaactgccttccctgacttcaccaaccccacagccctggcctggtgggaggatatggtggctgaatttcatgaccaggttccttttgatggcatgtggattgatatgaatgagccttccaactttatcaggggctctgaagatggctgccccaataatgagctggagaacccaccctatgttcctggggtggttgggggtaccctccaggctgccactatctgtgcctccagccatcagtttctctccacacactacaacttgcacaacctctatggcctgactgaagccattgcctctcatagggctttggttaaggctagggggacaagaccatttgtgatctccaggtcaacttttgctggccatggcaggtatgctggccattggactggtgatgtgtggagctcctgggagcaactagcttcatcagtgccagaaatcctgcagtttaatctgctaggggttcctctggttggtgctgatgtctgtggctttttgggcaacacctcagaggaactatgtgtgaggtggacccagctgggagccttctaccccttcatgaggaaccataacagcctgctcagtctgccccaggagccttactcattctcagagcctgcccagcaggctatgaggaaggccctcaccctgaggtatgcactcctcccccacctctacacactgttccaccaagcccatgtagctggggaaactgttgccaggcccctctttctggaattccccaaggactctagcacctggactgtggatcatcaactcctttggggggaagcccttctcatcacaccagtgctccaggctgggaaagctgaagtgactggctatttccccttgggcacatggtatgacctgcagactgtgccagtagaggcccttggctctttaccacccccacctgcagctccaagggaaccagcaatccactctgagggtcagtgggtgactttgcctgcccccttagacacaataaatgtccacctcagagctgggtacatcattcccctgcagggtcctggcctcacaaccacagagtccaggcaacagcccatggctctggctgtggccctgacaaagggtggggaggccaggggggagcttttctgggatgatggagagagcctggaagtgctggagaggggggcctacacacaggtcatattcctggctaggaataacactatagtgaatgaattggtaagggtgaccagtgagggagctggcttgcagctgcagaaggtgactgtcctgggggtggccactgcaccccagcaggtcctttccaatggtgtccctgtctccaacttcacttacagccctgacaccaaggtcttggacatttgtgtttcattattgatgggagagcagtttctagtgagctggtgt

SEQ ID NO: 584 - DNA, 2649 bp, synthetic construct
  misc_feature 1..2649: note = Synthetic
  source 1..2649: mol_type = other DNA
gcccaccctggcaggcctagagctgtgcctacacaatgtgatgtgccacctaacagcagatttgactgtgcacctgacaaggccataacccaggaacagtgtgaggctagagggtgctgctacatccctgctaagcagggactgcaaggagctcagatgggtcagccctggtgcttctttcccccatcctaccctagttacaaactagaaaatctgagcagttctgagatgggctacacagccaccctgactagaaccactccaaccttcttcccaaaggacatcttaactctcagactggatgtaatgatggaaactgagaatagattgcacttcactatcaaagacccagccaacagaagatatgaagttcctctagaaacacctcatgtgcactctagagccccttcccctctgtacagtgtagagttctctgaagagccttttggggtgattgttagaaggcagttggatgggagggtgctgttgaacaccacagtggcccccctgttctttgctgaccagttccttcagctgagtacttctctcccaagtcagtatatcacaggtctggctgagcacctgtcccctctcatgctgtctaccagctggacaagaataaccctatggaacagagacctggcaccaacccctggagcaaacttgtatggcagccaccccttctaccttgccctggaggatggtggcagtgcacatggggtgttcctcctcaactccaatgccatggatgtggttctgcaaccctctcctgccctcagctggagaagcacaggaggcattctagatgtctacatcttcctgggccctgaaccaaagtctgtggtccaacaatacctggatgttgtgggatacccctttatgcccccttactggggattaggcttccacctgtgcagatggggttactcaagcacagcaattaccagacaggtggttgagaacatgaccagggctcacttccctttagatgtgcagtggaatgacctggactacatggacagcagaagggactttaccttcaacaaagatggattcagggacttccctgccatggtacaggagctacaccaaggaggcagaagatacatgatgattgttgacccagcaatttcttcctctggcccagcaggcagctacagaccctatgatgagggcctgagaagaggggtgttcatcacaaatgagacaggccaacctctgataggcaaagtgtggcctgggtccactgccttcccagacttcacaaaccccactgcactggcatggtgggaagacatggtggctgagttccatgaccaggtgccttttgatgggatgtggattgatatgaatgagccctctaactttatcagaggaagtgaggatggctgccccaacaatgaacttgagaaccctccttatgttcctggagtggtaggaggcactttgcaggcagccaccatctgtgccagctcacaccagttcctgtccactcactacaacctgcacaacctatatggacttacagaggccatagcttctcacagagccttagtgaaggccagaggcactaggccttttgtcatcagcaggtcaacctttgcaggccatggcagatatgctggccactggactggggatgtttggagttcttgggaacaactggccagctcagtgccagagatcctgcagtttaacctgctgggggtgcctctggtgggagctgatgtatgtggtttcctgggtaacacctctgaggagctctgtgtaagatggacccagttgggagctttctaccctttcatgagaaaccacaactctctcctcagcttgccccaggagccctacagcttttctgaaccagcccagcaggctatgaggaaggcactgacactgagatatgctctactgcctcacctctacaccctattccaccaagcccatgtggcaggtgaaacagtggccaggcccctgttcttagagttccctaaggactcaagtacctggacagttgaccaccagctgttgtggggtgaggccctgctcatcaccccagtactccaggctggaaaggcagaggttactggctacttccctctgggcacctggtatgacctgcagacagtccctgtggaggctctggggtcactgcctccccctccagctgctccaagagaacctgccatccacagtgaaggccaatgggtaaccttgcctgcccctttagacacaatcaatgtgcacttgagagcaggctatatcataccactgcaaggaccaggcctaacaactacagagagcagacagcaacctatggctctggcagttgctttgactaagggtggtgaagcaagaggggaactgttctgggatgatggagaatctctggaagtgttagagagaggagcatacactcaggtgatcttcttagccaggaacaacaccattgtgaatgaactggttagggtgacaagtgaaggtgcaggactgcagctgcagaaggtgactgtcttaggtgtagccacagctccacaacaagtgctgtccaatggtgttcctgtcagcaacttcacctactctcctgacaccaaggttctggacatctgtgtgagcctccttatgggtgagcagtttctagtctcttggtgc

SEQ ID NO: 585 - DNA, 2649 bp, synthetic construct
  misc_feature 1..2649: note = Synthetic
  source 1..2649: mol_type = other DNA
gcacatccaggcaggcccagagcagtgcccacacaatgtgatgtcccccctaattcaaggtttgattgtgcccctgacaaggccatcactcaggaacagtgtgaagctaggggctgttgctatatccctgcaaagcagggtctgcagggagctcagatggggcagccctggtgcttctttccacccagctaccccagctacaaactggagaatctgagctcatctgaaatgggctacactgccaccctgaccaggacaacccccaccttcttccccaaagacatattgactcttagacttgatgtgatgatggagactgaaaacaggttacatttcactattaaagatccagctaacaggaggtatgaggttcccttggagactccacatgttcacagcagggcaccttccccactttactctgtggagttcagtgaagagccctttggtgtgattgtgaggaggcagctggatggcagggtactgttgaacactactgtggctcccttgttctttgctgaccagttccttcagctgtccaccagcctgccctcccagtatatcacaggcttagctgagcacctcagtcccctgatgttgagcacatcatggaccaggattaccctttggaacagggatcttgctcccactcctggtgctaacctctatgggtctcaccctttctatctggctttggaggatggtgggagtgcacatggggtgttcctgctaaatagcaatgccatggatgtggtcctacagcctagccctgccctttcttggaggtctacaggtgggatattagatgtttacatttttctgggaccagaacctaagtcagtagtgcagcagtacctagatgttgtgggatatccttttatgcctccatactggggattaggctttcatctgtgcaggtggggctattcctcaactgctattaccaggcaggtggtggagaacatgaccagagcccattttcccttagatgtccagtggaatgatctggactatatggactccaggagggactttacattcaacaaggatggcttcagggattttcctgccatggtgcaggaactccaccagggtggtaggaggtatatgatgatagttgatcctgctatcagcagcagtggccctgctgggagctacaggccatatgatgagggtctgaggaggggtgttttcataacaaatgagactggccagcctcttattgggaaagtatggcctggttccactgccttccctgatttcaccaatcctacagccctggcctggtgggaggacatggtggctgagttccatgaccaggtgccttttgatggcatgtggattgacatgaatgaaccttccaacttcatcaggggctctgaggatggctgccctaacaatgagctggagaacccaccctatgtgcctggggtggttggggggacactccaggctgctactatatgtgcctccagccaccagtttctctctacacactacaatctgcacaacctctatggcctgactgaagcaattgcctcccacagggctctggtgaaggctagggggacaaggccatttgtgatatccagatccacctttgctggacatggcaggtatgcaggccactggactggggatgtgtggagctcctgggagcagttggcctctagtgtgccagaaattctgcagtttaatctgctgggggttcctctagtaggggctgatgtctgtggctttcttggcaacacctcagaggaactgtgtgtgaggtggactcagctgggggctttctacccctttatgagaaaccacaattcactgctcagtctgccccaggagccatattctttcagtgagcctgcccaacaagccatgaggaaggccctcaccctgaggtatgcacttctcccacacctctatacactgtttcatcaggcccatgttgctggggaaactgttgcaaggcccctcttcctggagttccccaaagactctagcacctggactgtggatcatcaactcctttggggtgaggccttactcatcaccccagtgctccaagctgggaaggcagaagtgactggctacttccccttgggcacatggtatgatttgcagactgtgccagtagaagcccttggcagccttccacccccacctgcagctcctagggagccagccatccatagtgagggtcagtgggtgactctgccagcccccctggacactatcaatgtccacctcagggctgggtacattatccctctgcaaggccctggcctcacaaccacagaatccagacagcaacccatggccctagctgttgccttgaccaagggtggggaagccaggggtgagctcttttgggatgatggagagagcctggaagtgctggagaggggggcctacacacaggtcattttcctagccagaaataacactattgtgaatgaattggtaagggtgaccagtgagggagctggcctgcagctgcagaaggtgactgtccttggtgtggctactgctccacagcaggtcctctccaatggtgtccctgtctcaaattttacctattctccagacaccaaggtcttggacatctgtgtatccctgttgatgggagagcagtttttggttagctggtgt

SEQ ID NO: 586 - DNA, 2649 bp, synthetic construct
  misc_feature 1..2649: note = Synthetic
  source 1..2649: mol_type = other DNA
gcacaccctggcagacctagagctgttcccacacagtgtgatgtccctcctaacagcagatttgactgtgccccagacaaagctatcacccaggagcagtgtgaggctaggggctgctgctacatccctgccaagcagggcctgcaaggagcccaaatgggccagccctggtgtttctttcccccctcttacccatcctacaagctagaaaacctcagctcatctgagatgggatacacagctaccctgaccagaaccacccccaccttcttccctaaggatatactgacattgagactggatgtgatgatggaaactgagaacagactccacttcaccatcaaggatcctgctaacagaaggtatgaggtgcccctggagacacctcatgtccacagtagagcccctagcccactatactctgtggagttctcagaagagccttttggtgtgatagttagaagacaactggatgggagagtgttgctaaatactactgttgctcccttattttttgctgaccagttcttgcagttatccacctctctcccctctcagtacatcacagggctggctgagcacctttcccctctgatgttgtccacttcttggacaagaatcaccttgtggaatagggaccttgccccaactcctggtgccaacctgtatggcagccaccccttctacctggccctggaggatggaggctctgcccatggagtgttcctgctgaactccaatgccatggatgttgtgctgcagccatctcctgccctgtcctggaggagtacaggaggcatcctggatgtgtatatctttctgggacctgagcccaagtctgtggttcagcagtacctggatgtagtgggctaccccttcatgccaccttactggggtctgggcttccacctctgcaggtggggttacagtagcactgctatcactagacaagtggtggagaacatgaccagggcccacttccctctagatgtgcagtggaatgacttagactatatggatagcagaagggacttcacattcaacaaggatggcttcagggacttccctgcaatggtacaggagttacaccaggggggaagaagatacatgatgattgtggaccctgccattagctcttctggccctgcagggagctacaggccttatgatgaagggctgagaaggggtgtgttcattactaatgaaacaggccagcctttaattggcaaggtctggcctggcagtacagccttcccagacttcaccaaccctactgctctagcttggtgggaggacatggtggctgaatttcatgatcaagtgccttttgatggcatgtggattgacatgaatgagcctagcaacttcattagaggcagtgaagatggttgccctaacaatgagctggaaaatccaccctatgttccaggtgtggtaggaggcaccctacaagcagccaccatttgtgcctctagccaccagttcctgagcacccactataacttacataacctgtatggcctcacagaagccattgccagccacagagccttagtgaaagcaaggggcacaagaccctttgtaatctcaaggtccacctttgctggccatggcagatatgcaggccactggacaggtgatgtgtggagctcctgggagcagttagcctcttctgtgccagaaatcctgcagttcaatctcctgggagtgcctctggtgggagctgatgtttgtggctttctgggtaacacaagtgaagagctgtgtgtcaggtggacacagctgggagcattttaccccttcatgaggaaccacaactctttgctcagtctgcctcaagaaccctactccttctctgagcctgcccagcaggctatgagaaaggccctcacccttagatatgctctcctgcctcacctgtacaccctgttccaccaggcccatgttgctggggagactgtggctagaccacttttcttagagttccctaaggacagcagtacctggacagtagaccaccaactgttatggggggaggccctgctcattacccctgttctccaggcaggcaaggcagaagtgacagggtacttccctctgggcacctggtatgacctacagacagttcctgtggaggcccttggcagtctgccaccccctcctgctgcacctagagaacctgcaatccactcagaaggacaatgggttaccctccctgcccctctagacaccatcaatgtgcatttaagagcaggctacatcatacccttgcagggccctggcctgaccaccacagaatctagacagcagcccatggcactggctgttgccctaaccaagggaggagaggccagaggagagctgttctgggatgatggggagagtctggaggtcctggagagaggagcctacacccaggtgatctttcttgctagaaacaatactatagtgaatgagctagtgagagtgactagtgagggtgctggcctccagttgcagaaggtaacagtattaggagtggctacagcccctcagcaggtactgtccaatggtgtgcctgtttccaatttcacttactcccctgacactaaggtactggatatctgtgtgtccctgctgatgggagaacagtttctggtcagctggtgc

SEQ ID NO: 587 - DNA, 2649 bp, synthetic construct
  misc_feature 1..2649: note = Synthetic
  source 1..2649: mol_type = other DNA
gcacatcctggaaggcccagagcagtgcccacacagtgtgatgtcccccccaattctagatttgattgtgcccctgataaggctatcacccaagaacagtgtgaggccagaggctgttgctacatacctgcaaagcaagggcttcagggagctcagatggggcaaccctggtgctttttcccacctagctatcccagctacaagctggaaaacttgagctcctctgaaatgggctacactgccactctgaccaggaccacccccaccttcttccccaaagacatactgactcttagactagatgttatgatggagactgaaaataggctccattttacaatcaaagatccagctaacaggaggtatgaggttcctttggagacccctcatgtccatagcagagcaccatcaccactctactctgtggagttttctgaggaaccctttggggtgattgtgaggaggcagctagatggcagggtgctgttaaatacaactgtggctcccctgttctttgctgaccagtttcttcaactgtccacctccctgccctcccagtatattacaggcctggctgagcacctcagtcccttgatgctcagcaccagctggaccaggataaccttatggaacagggaccttgctccaactcctggtgcaaacctctatgggtctcatcctttctatctagctcttgaggatggggggtctgcacatggggtgttcctgctaaactctaatgcaatggatgttgtcctgcagccaagccctgccctttcttggaggtccacaggtgggatcctggatgtctacatcttcttgggcccagaacccaagtctgtggtgcagcagtaccttgatgttgtgggatacccttttatgcccccatactggggcctgggctttcatctgtgcaggtggggctactcctccactgctatcaccaggcaggtggtggagaacatgaccagggcccacttccctctggatgtccagtggaatgatctggattatatggattccaggagggatttcactttcaacaaagatggctttagggacttccctgccatggtgcaggagctgcaccagggtggcaggaggtatatgatgattgtggatcctgccataagcagctcaggccctgctgggagctacaggccatatgatgaaggtttgaggaggggggttttcataaccaatgagactggccagcctctgattgggaaggtatggcctgggtccactgcattccctgacttcaccaatcccacagctctggcctggtgggaggacatggtagctgagttccatgaccaggtgccctttgatggaatgtggattgacatgaatgaaccttccaacttcatcaggggctctgaggatggctgccccaacaatgagctggaaaacccaccctatgtgcctggggtggttggtgggaccttgcaagctgccaccatctgtgcctcctctcaccagtttctctccacacattacaatctgcacaatctctatggcctgactgaagccattgcttcccacagagctctggtgaaggctaggggcacaaggccatttgttatatcaaggtccacatttgctggccatggcaggtatgctggccactggacaggggatgtatggagctcctgggagcagcttgcatcctctgtgccagaaattctgcagtttaacttattaggggtgcctctggttggtgctgatgtctgtggcttccttggcaacacatcagaggagctttgtgtgaggtggactcagttaggggctttctacccattcatgaggaatcacaactcactcctcagtctaccccaggagccctattcattctctgagccagcccagcaggccatgaggaaggctctcaccctgaggtatgcactcctccctcatttgtacacactgtttcaccaggcccatgttgctggggaaactgttgccaggcccctcttcctggaattccccaaagactctagcacttggactgtggatcatcagctcctgtggggggaagccctgctcattactccagtgctacaggctggtaaggctgaagtgactggctacttccctttgggcacatggtatgacctgcagacagtgccagtagaggcccttggcagcctcccacctccacctgcagctcccagggagccagccatccactctgaaggacagtgggtgactctgcctgcccccttagacactattaatgttcacctcagggctggctacatcattcccctgcaaggccctggcctcacaaccacagaatccaggcagcaacccatggctttggctgtggccctgactaagggtggggaagccaggggggagcttttttgggatgatggagagagcctagaagtgttggagaggggggcctatacacaggtcatctttttggccaggaataatacaattgtgaatgagctggtaagggtgaccagtgaaggagctggactgcagctgcagaaggtgactgtacttggagtggctactgctccacagcaggttctatcaaatggtgtccctgtttccaatttcacctacagcccagacacaaaggtcctggacatttgtgtctccctgttgatgggagagcaatttttggtaagctggtgt

SEQ ID NO: 588 - DNA, 2649 bp, synthetic construct
  misc_feature 1..2649: note = Synthetic
  source 1..2649: mol_type = other DNA
gcccaccctggcagacctagagctgtgccaactcagtgtgatgtccctcctaactctagatttgattgtgctccagacaaggccatcacccaggagcagtgtgaagctaggggctgctgctacatccctgccaagcaaggtctgcagggtgcccagatgggtcagccttggtgcttcttcccaccttcctacccctcatacaagctggaaaatctgagctcttctgagatgggctacacagccaccttgacaaggaccactccaacctttttccctaaagacatcctgacactgagacttgatgtgatgatggagactgagaacagactgcacttcactatcaaagacccagcaaacagaaggtatgaagtgcctctggagaccccccatgtgcacagcagagctccttcacctctgtactcagtggagttcagtgaggagccatttggggtgattgtgaggagacagctggatggcagggtactgctgaacaccacagtggcacccttgttctttgcagaccaattcctccagctcagtacaagcctgccttctcagtacattacaggactggcagagcacttaagcccattgatgttatccaccagctggaccagaattaccttgtggaacagggacctggcccctaccccaggagctaacctctatggcagccacccattttacctagccctggaggatgggggatcagcacatggggttttcctgctaaacagcaatgccatggatgtggtcctgcagccttctcctgccctgagctggaggtccacaggagggatccttgatgtgtacatctttctgggccctgagcctaagagtgtggtgcaacagtacttggatgtggtggggtaccctttcatgcctccctactgggggttgggcttccacttgtgcagatggggatacagctcaacagccatcaccaggcaggtggttgagaacatgactagagcccactttccactagatgtgcagtggaatgacctggactacatggatagtagaagagacttcaccttcaataaggatggcttcagagatttccctgccatggtgcaggaactgcaccagggaggcagaagatacatgatgatagtggaccctgctatcagcagctctggccctgcaggtagctacagaccttatgatgagggcctgaggaggggagtgttcatcaccaatgagacaggccagcccctcattggcaaggtctggccaggcagcacagctttccctgacttcaccaaccccacagccctggcctggtgggaggacatggtggctgagttccatgaccaggtaccctttgatgggatgtggattgacatgaatgaaccctctaacttcatcaggggttctgaagatggctgccctaacaatgagctggagaaccccccttatgtgccaggagttgtgggaggcacccttcaggctgccaccatctgtgccagttcccaccagttcctaagcacccactacaacctgcacaacctgtatggtctcactgaagccatagcctctcacagagctctagtgaaggccagagggaccaggccttttgttatctccaggagtacctttgctggccatggtagatatgcaggccactggacaggagatgtgtggtcctcctgggagcaattagccagcagtgtgccagagatcctgcagttcaacctgctgggggttcctttagttggggcagatgtgtgtggcttcttgggcaacacatctgaagagctgtgtgtgagatggacccagctgggagccttctacccattcatgagaaaccataactcactgctgagcctccctcaggagccctacagcttctcagaacctgcccagcaggctatgaggaaggccctgacactcaggtatgccctgttacctcacctgtacaccctgttccaccaggctcatgtagctggggagactgtggccagaccccttttccttgagtttcctaaggacagctccacctggactgtggaccaccagttgctgtggggagaggccctgctgatcacccctgttctgcaggctggcaaagctgaggtgacaggctacttccccctgggaacctggtatgacctacaaactgtgccagtggaagcactgggctcactacctccacctccagctgcccccagagaacctgctattcactctgaagggcagtgggtgacactgcctgccccattagacaccatcaatgtacacttaagggctggctacatcattccactgcaggggccaggcctgaccaccactgagtccaggcagcagcccatggccctggcagtagccctaaccaagggaggtgaggccagaggagagctgttttgggatgatggtgaaagcctagaggtactagagagaggagcctacacccaggtcatcttcctggccagaaacaacacaatagtgaatgagctggtcagggtcacatctgagggggcaggcctgcagcttcagaaggtcactgtgttgggggtagccacagctccacagcaggtactttccaatggagtgccagtcagcaacttcacctacagtcctgacacaaaggttctggacatctgtgtgagcctgctgatgggagagcagttcttggtcagctggtgc

SEQ ID NO: 589 - protein, 925 aa, synthetic construct
  REGION 1..925: note = Synthetic
GHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 590 - protein, 917 aa, synthetic construct
  REGION 1..917: note = Synthetic
LLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 591 - protein, 883 aa, synthetic construct; identical to SEQ ID NO: 173
  REGION 1..883: note = Synthetic

SEQ ID NO: 592 - DNA, 738 bp, synthetic construct
  misc_feature 1..738: note = Synthetic
  source 1..738: mol_type = other DNA
gacatccagatgacccagtctccatcttccgtgtctgcatctgtaggagacagagtcaccatcacttgtcgggcgagtcagggtattgccagctggttagcctggtatcagcagaaaccagggaaagcccctgagctcctgatctatgctgcatccagtttgcaaggtggggtcccatcaaggttcagcggcagtggatctgggacagatttcactctcaccatcagcagcctgcagcctgaagattttgcaatttactattgtcaacaggctaactatttcccgtggacgttcggccaagggaccaaggtggaaatcaaaggcggcggcggaagcggagggggaggatcaggaggaggcggatcccagatcaccttgaaggagtctggtcctacgctggtgaaacccacacagaccctcacgctgacctgcaccttctctgggttctcactcagcactagtggagtgggtgtggtctggatccgtcagccccccggaaaggccctggagtggcttgcactcatttattggaatgatcataagcggtacagcccatctctggggagcaggctcaccatcaccaaggacacctccaaaaaccaggtggtccttacaatgaccaacatggaccctgtggacacagccacatattactgtgcacactacagtgggagctattcctactactactatggtttggacgtctggggccaagggaccacggtcaccgtctcctca

SEQ ID NO: 593 - DNA, 747 bp, synthetic construct
  misc_feature 1..747: note = Synthetic
  source 1..747: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaaggcggcggcggaagcggagggggaggatcaggaggaggcggatcccaggtgcagctggtggagtctgggggaggcgtggtccagcctggaaggtccctgagactctcctgcgcagcctctggattcccctttagtaattatgtcatgtattgggtccgccaggctccaggcaaggggctggagtgggtggctcttattttttttgacggaaagaaaaactatcatgcagactccgtgaagggccgattcaccataaccagagacaattccaaaaatatgttatatctgcaaatgaacagcctgagacctgaggacgcggctgtgtattactgtgcgaaaatccattgtcctaatggtgtatgttacaaggggtattacggaatggacgtctggggccaagggaccacggtcaccgtctcctca

SEQ ID NO: 594 - DNA, 726 bp, synthetic construct
  misc_feature 1..726: note = Synthetic
  source 1..726: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaaggcggcggcggaagcggagggggaggatcaggaggaggcggatccgaggtgcagctggtggagtctgggggaggcttagtacagcctggagggtccctaagactctcctgtgcagcctctggattcaccttcaatatttttgaaatgaactgggtccgccaggctccagggaaggggctggagtggatttcctacattagtagtcgtggaactaccacatactacgcagactctgtgaggggccgattcaccatctccagagacaacgccaagaactcactgtatctgcaaatgaacagcctgagagccgaggacacggctgtttattactgtgcgagagattatgaagcaacaatcccttttgacttctggggccagggaaccctggtcaccgtctcctca

SEQ ID NO: 595 - DNA, 732 bp, synthetic construct
  misc_feature 1..732: note = Synthetic
  source 1..732: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaaggcggcggcggaagcggagggggaggatcaggaggaggcggatccgaagtgcagctggtggagtctgggggaggcttggttcagcctggcaggtccctgagactctcctgtgcagcctctggattcacctttgatgattatgccatgaactgggtccggcaagctccagggaagggcctggagtgggtctcaggtattagttggagtagtggtagcatggactatgcggactctgtgaagggccgattcaccatctccagagacaacgccaaaaactccctgtatctgcaaatgaacagtctgagaactgaggacacggccttatattactgtgcaaaagctagggaagttggagactactacggtatggacgtctggggccaagggaccacggtcaccgtctcctca

SEQ ID NO: 596 - protein, 29 aa, synthetic construct
  REGION 1..29: note = Synthetic
MHRPRRRGTRPPPLALLAALLLAARGADA

SEQ ID NO: 597 - DNA, 141 bp, synthetic construct
  misc_feature 1..141: note = Synthetic
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 598 - DNA, 130 bp, synthetic construct
  misc_feature 1..130: note = Synthetic
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 599 - DNA, 227 bp, synthetic construct
  misc_feature 1..227: note = Synthetic
  source 1..227: mol_type = other DNA
tcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtc